(19) United States
(12) Patent Application Publication
(10) Pub. No.: US 2002/0094989 A1
(43) Pub. Date: Jul. 18, 2002

(54) Pyrrolidine modulators of CCR5 chemokine receptor activity

(76) Inventors: Jeffrey J. Hale, Westfield, NJ (US); Christopher L. Lynch, Scotch Plains, NJ (US); Charles G. Caldwell, Scotch Plains, NJ (US); Christopher A. Willoughby, Greenbrook, NJ (US); Dooseop Kim, Westfield, NJ (US); Dong-Ming Shen, Edison, NJ (US); Sander G. Mills, Scotch Plains, NJ (US); Kevin T. Chapman, Scotch Plains, NJ (US); Liya Chen, East Brunswick, NJ (US); Amy Gentry, New York, NY (US); Malcolm MacCoss, Freehold, NJ (US); Zenon D. Konteatis, Chatham, NJ (US)

Correspondence Address: MERCK AND CO INC, P O BOX 2000, RAHWAY, NJ, 070650907

(21) Appl. No.: 09/974,000

(22) Filed: Oct. 10, 2001

Related U.S. Application Data
(60) Provisional application No. 60/239,441, filed on Oct. 11, 2000.

(51) Int. Cl. A61K 31/496; A61K 31/4745; A61K 31/4439; C07D 471/02; C 07D 4 3/02; C 07D 4 1/02

(52) U.S. Cl. 514/253.04; 514/254.01; 514/254.06; 514/303; 514/322; 514/326; 544/362; 544/368; 544/372; 546/113; 546/119; 546/208

(57) ABSTRACT

Pyrrolidine compounds of Formula I:

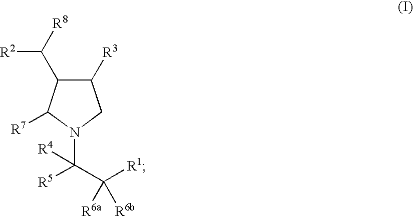

(wherein ¹, R², R³, R⁴, R⁵,R^{6a}, R^{6b}, R⁷ and R⁸ are defined herein) are described. The compounds are modulators of CCR5 chemokine receptor activity. The compounds are useful, for example, in the prevention or treatment of infection by HIV and the treatment of AIDS, as compounds or pharmaceutically acceptable salts, or as ingredients in pharmaceutical compositions, optionally in combination with other antivirals, immunomodulators, antibiotics or vaccines. Methods of treating AIDS and methods of preventing or treating infection by HIV are also described.

This application claims the benefit of U.S. Provisional Application No. 60/239,441, filed Oct. 11, 2000, the disclosure of which is hereby incorporated by reference in its entirety.

### BACKGROUND OF THE INVENTION

Chemokines are chemotactic cytokines that are released by a wide variety of cells to attract macrophages, T cells, eosinophils, basophils and neutrophils to sites of inflammation (reviewed in Schall, Cytokine, 3, 165-183 (1991) and Murphy, Rev. Immun., 12, 593-633 (1994)). There are two classes of chemokines, C-X-C (α) and C-C (β), depending on whether the first two cysteines are separated by a single amino acid (C-X-C) or are adjacent (C-C). The α-chemokines, such as interleukin-8 (IL-8), neutrophil-activating protein-2 (NAP-2) and melanoma growth stimulatory activity protein (MGSA) are chemotactic primarily for neutrophils, whereas D-chemokines, such as RANTES, MIP-1α, MIP-1β, monocyte chemotactic protein-1 (MCP-1), MCP-2, MCP-3 and eotaxin are chemotactic for macrophages, T-cells, eosinophils and basophils (Deng, et al., Nature, 381, 661-666 (1996)).

The chemokines bind specific cell-surface receptors belonging to the family of G-protein-coupled seven-transmembrane-domain proteins (reviewed in Horuk, Trends Pharm. Sci., 15, 159-165 (1994)) which are termed “chemokine receptors.” On binding their cognate ligands, chemokine receptors transduce an intracellular signal though the associated trimeric G protein, resulting in a rapid increase in intracellular calcium concentration. There are at least sixteen human chemokine receptors that bind or respond to β-chemokines with the following characteristic pattern: CCR1 (or “CKR-1” or “CC-CKR-1”) [MIP-1α, MIP-1β, MCP-3, RANTES] (Ben-Barruch, et al., J. Biol. Chem., 270, 22123-22128 (1995); Beote, et al, Cell, 72, 415-425 (1993)); CCR2A and CCR2B (or “CKR-2A”/“CKR-2A” or “CC-CKR-2A”/“CC-CKR-2A”) [MCP-1, MCP-3, MCP-4]; CCR3 (or “CKR-3” or “CC-CKR-3”) [eotaxin, RANTES, MCP-3] (Combadiere, et al., J. Biol. Chem., 270, 16491-16494 (1995); CCR4 (or “CKR-4” or “CC-CKR-4”) [MIP-1α;, RANTES, MCP-1] (Power, et al., J. Biol. Chem., 270, 19495-19500 (1995)); CCR5 (or “CKR-5” or “CC-CKR-5”) [MIP-1α, RANTES, MIP-1β] (Sanson, et al., Biochemistry, 35, 3362-3367 (1996)); and the Duffy blood-group antigen [RANTES, MCP-1] (Chaudhun, et al., J. Biol. Chem., 269, 7835-7838 (1994)). The β-chemokines include eotaxin, MIP (“macrophage inflammatory protein”), MCP (“monocyte chemoattractant protein”) and RANTES (“regulation-upon-activation, normal T expressed and secreted”).

Chemokine receptors, such as CCR1, CCR2, CCR2A, CCR2B, CCR3, CCR4, CCR5, CXCR-3, CXCR-4, have been implicated as being important mediators of inflammatory and immunoregulatory disorders and diseases, including asthma, rhinitis and allergic diseases, as well as autoimmune pathologies such as rheumatoid arthritis and atherosclerosis. A review of the role of chemokines in allergic inflammation is provided by Kita, H., et al., J. Exp. Med. 183, 2421-2426 (1996). Accordingly, agents which modulate chemokine receptors would be useful in such disorders and diseases. Compounds which modulate chemokine receptors would be especially useful in the treatment and prevention of atopic conditions including allergic rhinitis, dermatitis, conjunctivitis, and particularly bronchial asthma.

A retrovirus designated human immunodeficiency virus (HIV-1) is the etiological agent of the complex disease that includes progressive destruction of the immune system (acquired immune deficiency syndrome; AIDS) and degeneration of the central and peripheral nervous system. This virus was previously known as LAV, HTLV-III, or ARV.

Certain compounds have been demonstrated to inhibit the replication of HIV, including soluble CD4 protein and synthetic derivatives (Smith, et al., Science, 238, 1704-1707 (1987)), dextran sulfate, the dyes Direct Yellow 50, Evans Blue, and certain azo dyes (U.S. Pat. No. 5,468,469). Some of these antiviral agents have been shown to act by blocking the binding of gp120, the coat protein of HIV, to its target, the CD4 glycoprotein of the cell.

Entry of HIV-1 into a target cell requires cell-surface CD4 and additional host cell cofactors. Fusin has been identified as a cofactor required for infection with virus adapted for growth in transformed T-cells, however, fusin does not promote entry of macrophagetropic viruses which are believed to be the key pathogenic strains of HIV in vivo. It has recently been recognized that for efficient entry into target cells, human immunodeficiency viruses require a chemokine receptors, most probably CCR5 or CXCR-4, as well as the primary receptor CD4 (Levy, N. Engl. J. Med., 335(20), 1528-1530 (Nov. 14 1996). The principal cofactor for entry mediated by the envelope glycoproteins of primary macrophage-trophic strains of HIV-1 is CCR5, a receptor for the β-chemokines RANTES, MIP-1α and MIP-1β (Deng, et al., Nature, 381, 661-666 (1996)). HIV attaches to the CD4 molecule on cells through a region of its envelope protein, gp120. It is believed that the CD-4 binding site on the gp120 of HIV interacts with the CD4 molecule on the cell surface, and undergoes conformational changes which allow it to bind to another cell-surface receptor, such as CCR5 and/or CXCR-4. This brings the viral envelope closer to the cell surface and allows interaction between gp41 on the viral envelope and a fusion domain on the cell surface, fusion with the cell membrane, and entry of the viral core into the cell. It has been shown that β-chemokine ligands prevent HIV-1 from fusing with the cell (Dragic, et al., Nature, 381, 667-673 (1996)). It has further been demonstrated that a complex of gp120 and soluble CD4 interacts specifically with CCR5 and inhibits the binding of the natural CCR5 ligands MIP-1α and MIP-1β (Wu, et al., Nature, 384, 179-183 (1996); Trkola, et al., Nature, 384, 184-187 (1996)).

Humans who are homozygous for mutant CCR5 receptors which are not expressed on the cell surface appear to be unusually resistant to HIV-1 infection and are not immuno-compromised by the presence of this genetic variant (Nature, 382, 722-725 (1996)). Absence of CCR5 appears to confer substantial protection from HIV-1 infection (Nature, 382, 668-669 (1996)). Other chemokine receptors may be used by some strains of HIV-1 or may be favored by non-sexual routes of transmission. Although most HIV-1 isolates studied to date utilize CCR5 or fusin, some can use both as well as the related CCR2B and CCR3 as co-receptors (Nature Medicine, 2(11), 1240-1243 (1996)). Nevertheless, drugs targeting chemokine receptors may not be unduly compromised by the genetic diversity of HIV-1 (Zhang, et al., Nature, 383, 768 (1996)). Accordingly, an agent which could block chemokine receptors in humans who possess normal chemokine receptors should prevent infection in healthy individuals and slow or halt viral progression in infected patients. By focusing on the host's cellular immune response to HIV infection, better therapies towards all subtypes of HIV may be provided. These results indicate that inhibition of chemokine receptors presents a viable method for the prevention or treatment of infection by HIV and the prevention or treatment of AIDS.

The peptides eotaxin, RANTES, MIP-1α, MIP-1β, MCP-1, and MCP-3 are known to bind to chemokine receptors. As noted above, the inhibitors of HIV-1 replication present in supernatants of CD8+ T cells have been characterized as the β-chemokines RANTES, MIP-1α and MIP-1β.

### SUMMARY OF THE INVENTION

The present invention is directed to compounds which inhibit the entry of human immunodeficiency virus (HIV) into target cells and are of value in the prevention of infection by HIV, the treatment of infection by HIV, the prevention and/or treatment of the resulting acquired immune deficiency syndrome (AIDS), and the delay in the onset of AIDS. The present invention also relates to pharmaceutical compositions containing the compounds and to a method of use of the present compounds and other agents for the prevention and treatment of AIDS and viral infection by HIV.

The present invention is further directed to compounds which are modulators of CCR5 chemokine receptor activity and are useful in the prevention or treatment of certain inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis. The invention is also directed to pharmaceutical compositions comprising these compounds and the use of these compounds and compositions in the prevention or treatment of such diseases in which chemokine receptors are involved.

### DETAILED DESCRIPTION OF THE INVENTION

The present invention includes compounds of Formula I:

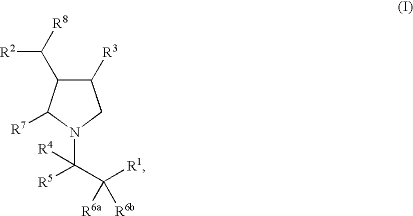

wherein:

R¹ is:

(1) —CO₂H,

(2) —NO₂,

(3) -tetrazolyl,

(4) -hydroxyisoxazole,

(5) —SO₂NHCO—(C₀₋₃ alkyl)—R^{a}, or

(6) —P(O)(OH)(OR^{a});

wherein R^{a} is independently selected from hydrogen, C₁₋₆ alkyl, C⁵⁻⁶ cycloalkyl, benzyl and phenyl, where any one of which except hydrogen is optionally substituted with 1-3 substituents where the substituents are independently selected from halo, C₁₋₃ alkyl, —O-C₁₋₃ alkyl, and —CF₃,

R² is:

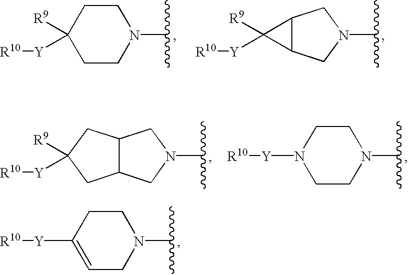

wherein “ [symbol] ” denotes the point of attachment and R⁹ is selected from:

(1) hydrogen,

(2) C₁₋₆ alkyl, which is unsubstituted or substituted with 1-4 substituents where the substituents are independently selected from hydroxy, cyano, and halo,

(3) cyano,

(4) hydroxy, and

(5) halo; and

Y is:

(1) a direct single bond;

(2) —C₁₋₁₀ alkyl- or —(C₀₋₆ alkyl)C₃₋₆cycloalkyl(C₀₋₆ alkyl)—, either of which is optionally substituted with 1-7 substituents independently selected from:

(a) halo,

(b) hydroxy,

(c) —O-C₁₋₃ alkyl,

(d) —CF₃,

(e) —(C₁₋₃ alkyl)hydroxy, and

(f) ethylenedioxy;

(3) —(C₀₋₆ alkyl)—Z¹—(C₀₋₆ alkyl)—, wherein each alkyl is optionally substituted with 1-7 substituents independently selected from:

(a) halo,

(b) hydroxy,

(c) —O-C₁₋₃ alkyl, and

(d) —CF₃;

and where Z¹ is selected from —SO₂—, —N(R^{u})—, —N(R^{u})C(═CHR^{s})N(R^{u})—, —N(R^{u})C(═NR^{s})N(R^{u})—, —S—, —O—, —SO—, SO₂N(R^{u})—, —N(R^{u})SO₂—, and —PO₂—;

R^{u} is hydrogen, C₁₋₆ alkyl, C₂₋₆ alkenyl, benzyl, phenyl, (CO)C₁₋₆ alkyl, —SO₂-C₁₋₆ alkyl, —SO₂-phenyl, —SO₂-heterocycle, or C₁₋₆ alkyl-C₃₋₆ cycloalkyl; wherein any of which except hydrogen is optionally substituted with 1-3 substituents independently selected from halo, C₁₋₃ alkyl, —O-C₁₋₃ alkyl, and —CF₃;

R^{s} is hydrogen, C₁₋₄ alkyl, —NO₂ or 'CN;

(4) —(C₀₋₆ alkyl)—Z²—(C₀₋₆ alkyl)—, wherein each alkyl is optionally substituted with 1-7 substituents independently selected from:

(a) halo,

(b) hydroxy,

(c) —O-C₁₋₃ alkyl, and

(d) —CF₃;

and where:

Z² is selected from —C(═O)—, —C(═O)O—, —OC(═O)—, —C(═O)NR^{v}—, —NR^{v}C(═O)—, —OC(═O)NR^{v}—, —NR^{v}C(═O)O—, and —NR^{w}C(═O)NR^{v}—;

R^{v} is hydrogen, C₁₋₆ alkyl, C₂₋₆ alkenyl, C₂₋₆ alkynyl, benzyl, phenyl, or C₁₋₆ alkyl-C₃₋₆ cycloalkyl; wherein any of which except hydrogen is optionally substituted with 1-3 substituents independently selected from halo, C₁₋₃ alkyl, —O-C₁₋₃ alkyl, and —CF₃; and

R^{w} is hydrogen or C₁₋₆ alkyl;

R¹⁰ is:

phenyl, naphthyl, biphenyl, or heterocycle, any one of which is unsubstituted or substituted with 1-7 of R^{d} where R^{d} is independently selected from:

(a) halo,

(b) cyano,

(c) hydroxy,

(d) C₁₋₆ alkyl, which is unsubstituted or substituted with 1-5 of R^{e} where R^{e} is independently selected from halo, cyano, hydroxy, —O-C₁₋₆ alkyl, —C₃₋₆ cycloalkyl, —CO₂H, —CO₂—(C₁₋₆ alkyl), —CF₃, —SO₂R^{a}, —NR^{a}R^{b} (where R^{a} is independently as defined above and R^{b} is independently selected from the definitions of R^{a}), phenyl, naphthyl, biphenyl, and heterocycle;

wherein phenyl, naphthyl, biphenyl, or heterocycle is unsubstituted or substituted with 1-7 of R^{f} where R^{f} is independently selected from halo, cyano, hydroxy, C₁₋₆ alkyl, C₁₋₆ haloalkyl, —O-C₁₋₆ alkyl, —O-C₁₋₆ haloalkyl, —CO₂H, —CO₂(C₁₋₆ alkyl), —NR^{a}R^{b}, —(C₁₋₆ alkyl)-NR^{a}R^{b}, —SO₂R^{a}, —N(R^{a})SO₂R^{b}, —N(R^{a})COR^{b}, —(C₁₋₆ alkyl)hydroxy, —O-C₃₋₆ cycloalkyl, benzyloxy, phenoxy, and —NO₂,

(e) —O-C₁₋₆ alkyl, which is unsubstituted or substituted with 1-5 of R^{e},

(f) —O-phenyl, which is unsubstituted or substituted with 1-5 of R^{f},

(g) —O-heterocycle, which is unsubstituted or substituted with 1-5 of R^{f},

(h) —NO₂,

(i) phenyl,

(j) —CO₂R^{a},

(k) tetrazolyl,

(l) —NR^{a}R^{b},

(m) —NR^{a}—CO₂R^{b},

(n) —NR^{a}—CO₂R^{b},

(o) —CO—NR^{a}R^{b},

(p) —OCO—NR^{a}R^{b},

(q) —NR^{a}CO—NR^{a}R^{b},

(r) —S(O)ₘ—R^{a}, wherein m is an integer selected from 0, 1 and 2,

(s) —S(O)₂—NR^{a}R^{b},

(t) —NR^{a}S(O)₂R^{b},

(u) —NR^{a}S(O)₂—NR^{a}R^{b},

(v) C₂₋₆ alkenyl,

(w) furanyl, which is unsubstituted or substituted with benzyl which is unsubstituted or substituted with 1-7 of R^{f} wherein R^{f} is independently as defined above,

(x) —C₃₋₆ cycloalkyl, and

(y) —O-C₃₋₆ cycloalkyl;

R³ is phenyl, naphthyl, or heterocycle, any one of which is unsubstituted or substituted with 1-7 substituents where the substituents are independently selected from:

(a) halo,

(b) C₁₋₄ alkyl,

(c) C₁₋₄ haloalkyl,

(d) hydroxy,

(e) —O-C₁₋₄ alkyl,

(f) —O-C₁₋₄ haloalkyl,

(g) —CO₂R^{a},

(h) —NR^{a}R^{b}, and

(i) —CONR^{a}R^{b};

R⁴ is hydrogen, C₁₋₁₀ alkyl, C₃₋₈ cycloalkyl, —(C₁₋₃ alkyl)-C₃₋₈ cycloalkyl, —(C₀₋₂ alkyl)-(C₃₋₈ cycloalkylidenyl)-(C₁₋₂ alkyl), C₂₋₁₀ alkenyl, C₂₋₁₀ alkynyl, cyclohexenyl, phenyl, —(C₁₋₆ alkyl)-phenyl, naphthyl, dihydronaphthyl, tetrahydronaphthyl, octahydronaphthyl, biphenyl, or heterocycle; wherein any one of which except for hydrogen is unsubstituted or substituted with 1-7 of R^{d} where R^{d} is independently as defined above;

R⁵ is hydrogen or C₁₋₆ alkyl, wherein the alkyl is unsubstituted or substituted with 1-7 substituents where the substituents are independently selected from:

(a) halo,

(b) —CF₃,

(c) hydroxy,

(d) C₁₋₃ alkyl,

(e) —O-C₁₋₃ alkyl,

(f) —CO₂R^{a},

(g) —NR^{a}R^{b}, and

(h) —CONR^{a}R^{b};

or alternatively R⁴ and R⁵ together with the carbon atom to which they are attached form a C₃₋₈ cycloalkyl ring which may be unsubstituted or substituted with 1-7 of Rd; R^{6a} and R_{6b} are each independently C₁₋₆ alkyl, C₂₋₆ alkenyl, C₂₋₆ alkynyl, C₃₋₈ cycloalkyl, phenyl, naphthyl, or heterocycle; wherein any one of which is unsubstituted or substituted with 1-7 substituents where the substituents are independently selected from:

(a) halo,

(b) C₁₋₄ haloalkyl,

(c) hydroxy,

(d) C₁₋₄ alkyl,

(e) —O-C₁₋₄ alkyl,

(f) —O-C₁₋₄ haloalkyl,

(g) C₃₋₈ cycloalkyl,

(h) —CO₂R^{a},

(i) —NR^{a}R^{b}, and

(i) —CONR^{a}R^{b};

or alternatively R^{6a} and R^{6b} together with the carbon atom to which they are attached form:

(a) a 3- to 8-membered saturated carbocyclic ring, in which one of the ring carbons is optionally a member of a 3- to 8-membered Spiro ring containing carbon atoms and optionally 1 or 2 heteroatoms independently selected from nitrogen, oxygen and sulfur;

(b) a 4- to 8-membered monocyclic heterocycle containing from 1 to 3 heteroatoms independently selected from nitrogen, oxygen and sulfur, in which one of the ring carbons is optionally a member of a 3- to 8-membered spiro ring containing carbon atoms and optionally 1 or 2 heteroatoms independently selected from nitrogen, oxygen and sulfur;

(c) a 5- to 8-membered saturated carbocyclic ring to which is fused a C3-8 cycloalkyl, or

(d) a 5- to 8-membered heterocyclic ring containing from 1 to 3 heteroatoms independently selected from nitrogen, oxygen and sulfur, to which is fused a C₃₋₈ cycloalkyl,

wherein the ring system of (a), (b), (c) or (d) is optionally substituted with from 1 to 3 substituents independently selected from halo, C₁₋₄ alkyl, C₁₋₄ haloalkyl, —O-C₁₋₄ alkyl, —O-C₁₋₄ haloalkyl, and hydroxy;

R⁷ is hydrogen or C₁₋₆ alkyl; and

R⁸ is hydrogen or C₁₋₆ alkyl;

and with the proviso that

(A) when R¹⁰ is a heterocycle selected from pyrazolyl and imidazolyl, then the heterocycle is unsubstituted or substituted with 1 or 2 of R^{d}; and

(B) when R¹⁰ is a heterocycle selected from:

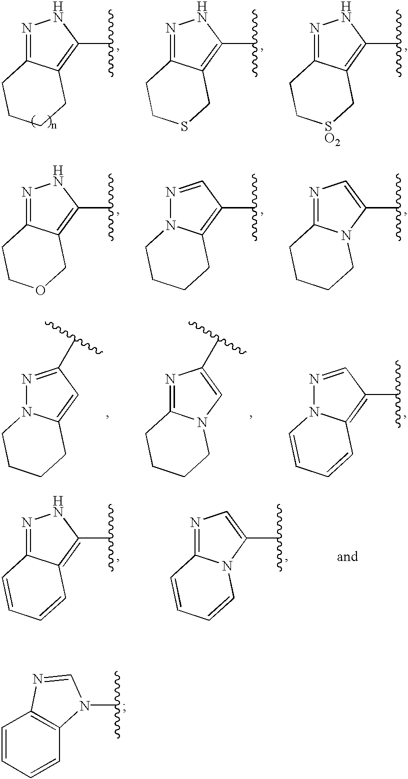

wherein n is an integer equal to zero or 1, then the heterocycle is unsubstituted in the pyrazolyl or imidazolyl ring;

or a pharmaceutically acceptable salt thereof.

A first embodiment of the present invention is a compound of Formula I, wherein R¹ is:

(1) —CO₂H,

(2) —P(O)(OH)₂, or

(3) -tetrazolyl;

and all other variables are as originally defined;

or a pharmaceutically acceptable salt thereof.

In one aspect of the first embodiment, R¹ is (1) —CO₂H or (2) -tetrazolyl. In another aspect of the first embodiment, R¹ is —CO₂H.

A second embodiment of the present invention is a compound of Formula I, wherein R² is:

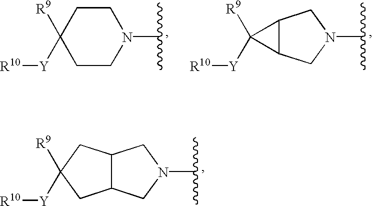

and all other variables are as originally defined;

or a pharmaceutically acceptable salt thereof.

In one aspect of the second embodiment, R² is:

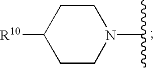

A third embodiment of the present invention is a compound of Formula I, wherein R³ is phenyl, thienyl, pyrazolyl, thiazolyl, thiadiazolyl, furanyl, oxadiazolyl, pyrazinyl, pyrimidinyl, or pyridyl, any one of which is unsubstituted or substituted with 1-5 substituents where the substituents are independently selected from:

(a) halo,

(b) —CF₃,

(c) hydroxy,

(d) C₁₋₃ alkyl, and

(e) —O-C₁₋₃ alkyl;

and all other variables are as originally defined;

or a pharmaceutically acceptable salt thereof.

In one aspect of the third embodiment, R³ is phenyl or thienyl, either of which is unsubstituted or substituted with 1-5 substituents where the substituents are independently selected from:

(a) halo,

(b) —CF₃,

(c) hydroxy, and

(d) C₁₋₃ alkyl.

In another aspect of the third embodiment, R³ is phenyl or thienyl, wherein the phenyl is optionally substituted with 1-5 substituents independently selected from fluoro and chloro.

In still another aspect of the third embodiment, R³ is unsubstituted phenyl, 3-fluorophenyl, or 3-thienyl.

A fourth embodiment of the present invention is a compound of Formula I, wherein R⁴ and R⁵ are both hydrogen; and all other variables are as originally defined; or a pharmaceutically acceptable salt thereof.

A fifth embodiment of the present invention is a compound of Formula I, wherein R^{6a} and R^{6b} are each independently C₁₆ alkyl or C₃₋₆ cycloalkyl, either of which is unsubstituted or substituted with 1-7 substituents independently selected from:

(a) halo,

(b) —CF₃,

(c) hydroxy, and

(d) —O-C₁₋₃ alkyl;

or R^{6a} and R^{6b} together with the carbon atom to which they are attached form:

(a) a 3- to 6-membered saturated carbocyclic ring,

(b) a 4- to 6-membered saturated heterocyclic ring containing one oxygen atom, or

(c) a 5- or 6-membered saturated carbocyclic ring to which is fused a C₃₋₆ cycloalkyl;

wherein the ring system of (a), (b), or (c) is optionally substituted with from 1 to 3 substituents selected from halo, C₁₋₄ alkyl, C₁₋₄ haloalkyl, —O-C₁₋₄ alkyl, —O-C₁₋₄ haloalkyl, or hydroxy;

and all other variables are as originally defined;

or a pharmaceutically acceptable salt thereof.

In one aspect of the fifth embodiment, R^{6a} and R^{6b} are each C1-3 alkyl;

or one of R^{6a} and R^{6b} is C1-3 alkyl, and the other of R^{6a} and R^{6b} is C3-6 cycloalkyl;

or R^{6a} and R^{6b} together with the carbon atom to which they are attached form cyclobutylidenyl, cyclopentylidenyl, cyclohexylidenyl, bicyclo[3.1.0]cyclohexylidenyl, tetrahydropyranylidenyl, or tetrahydrofuranylidenyl.

A sixth embodiment of the present invention is a compound of Formula I, wherein R⁷ is hydrogen;

and all other variables are as originally defined;

or a pharmaceutically acceptable salt thereof.

A seventh embodiment of the present invention is a compound of Formula I, wherein R⁸ is hydrogen;

and all other variables are as originally defined;

or a pharmaceutically acceptable salt thereof.

A eighth embodiment of the present invention is a compound of Formula I, wherein R⁸ is methyl;

and all other variables are as originally defined;

or a pharmaceutically acceptable salt thereof.

A ninth embodiment of the present invention is a compound of Formula I, R⁹ is hydrogen, fluoro, hydroxy or C₁₋₆ alkyl;

and all other variables are as originally defined;

or a pharmaceutically acceptable salt thereof.

In an aspect of the ninth embodiment, R⁹ is hydrogen or fluoro. In another aspect, R⁹ is hydrogen.

A tenth embodiment of the present invention is a compound of Formula I, wherein Y is

(1) a direct single bond;

(2) —C₁₋₆ alkyl-, which is optionally substituted with 1-7 substituents independently selected from:

(a) halo,

(b) hydroxy,

(c) —O-C₁₋₃ alkyl, and

(d) —CF₃;

(3) —(C₀₋₂ alkyl)—Z¹—(C₀₋₂ alkyl)—, wherein the alkyl is unsubstituted;

Z¹ is selected from —SO₂—, —N(R^{u})—, 'SO—, —SO₂N(R^{u})—, —S—, and —O—; and R^{u} is C₁₋₄ alkyl, C₂₋₅ alkenyl, or C₁₋₃ alkyl-C₃₋₆ cycloalkyl; or

(4) —(C₀₋₂ alkyl)—Z²—(CO₂ alkyl)—, wherein the alkyl is optionally substituted with 1-4 substituents independently selected from:

(a) halo,

(b) hydroxy,

(c) —O-C₁₋₃ alkyl, and

(d) —CF₃;

and wherein

Z² is selected from —C(═O)NR^{v}—, —NR^{v}C(═O)—, —OC(═O)NR^{v}—, —NR^{v}C(═O)O—, and —NR^{w}C(═O)NR^{v}—;

R^{v} is hydrogen, C₁₋₆ alkyl, C₂₋₆ alkenyl, C₂₋₆ alkynyl, benzyl, phenyl, or C₁₋₆ alkyl-C₃₋₆ cycloalkyl; wherein any of which except hydrogen is optionally substituted with from 1 to 3 substituents independently selected from halo, C₁₋₃ alkyl, —O-C₁₋₆ alkyl and —CF₃; and

R^{w} is —H or C₁₋₆ alkyl;

and all other variables are as originally defined;

or a pharmaceutically acceptable salt thereof.

In a first aspect of the tenth embodiment, Y is

(1) a direct single bond;

(2) —C₂₋₄ alkyl-, which is optionally substituted with 1-6 substituents independently selected from:

(a) halo,

(b) —O-C₁₋₃ alkyl, and

(c) —CF₃;

(3) selected from

—(C₀₋₂ alkyl)—SO₂—(C₀₋₂ alkyl)—,

—(C₀₋₂ alkyl)—SO₂N(R^{u})—(C₀₋₂ alkyl),

—(C₀₋₂ alkyl)—SO—(C₀₋₂ alkyl)—,

—(C₀₋₂ alkyl)—S—(C₀₋₂ alkyl)—,

—(C₀₋₂ alkyl)—O—(C₀₋₂ alkyl)—, and

—(C₀₋₂ alkyl)—N(R^{u})—(C₀₋₂ alkyl)—; and

where R^{u} is C₂₋₄ alkyl, C₂₋₃ alkenyl or C₁₋₂ alkyl-C₁₋₃ cycloalkyl;

(4) —(C₀-2 alkyl)—Z²—(CO₂ alkyl)—, wherein the alkyl is not substituted; and where

Z² is selected from —C(═O)NR^{v}—, —NR^{v}C(═O)—, —OC(═O)NR^{v}—, —NR^{v}C(═O)O—, and —NR^{w}C(═O)NR^{v}—;

R^{v} is hydrogen, C₁₋₃ alkyl, C₂₋₃ alkenyl, or C₂₋₃ alkynyl; and

R^{w} is —H or C₁₋₄ alkyl.

In a second aspect of the tenth embodiment, Y is

(1) a direct single bond;

(2) C₂₋₄ alkyl, which is optionally substituted with from 1 to 6 fluoros;

(3) selected from:

(a) —SO₂CH₂CH₂—,

(b) —SO₂—N(CH₂CH₃)—,

(c) —CH₂SO₂—N(CH₂CH₃)—,

(d) —SO—CH₂CH₂—,

(e) —SCH₂CH₂—,

(f) —CH₂—O—CH₂—,

(g) —N(CH₂CH₃)—,

(h) —N(CH₂CH₂CH₃)—,

(i) —N(allyl)—, and

(j) —N(CH₂-cyclopropyl)—; or

(4) selected from:

(a) —CH₂OC(═O)—N(C₁₋₄ alkyl)—,

(b) —CH₂—OC(═O)N(allyl)—,

(c) —CH₂NHC(═O)N(C₁₋₄ alkyl)—,

(d) —CH₂NHC(═O)N(allyl), and

(e) —CH₂CH₂NHC(═O)N(CH₂CH₃)—.

In a third aspect of the tenth embodiment, Y is a direct single bond; or a pharmaceutically acceptable salt thereof.

An eleventh embodiment of the present invention is a compound of Formula I, wherein R¹⁰ is phenyl, benzoimidazolyl, imidazolyl, pyridoimidazolyl, isoxazolyl, oxazolyl, pyrazolyl, pyridyl, thiazolyl, imidazothiophenyl, indazolyl, tetrahydropyridoimidazolyl, tetrahydroindazolyl, dihydrothiopyranopyrazolyl, dihydrodioxothiopyranopyrazolyl, dihydropyranopyrazolyl, tetrahydropyridopyrazolyl, benzopyrazolyl, pyridopyrazolyl, or triazolyl (e.g., 1,2,4-triazolyl); any one of which is unsubstituted or substituted with 1-7 substituents where the substituents are independently selected from:

(a) halo,

(b) cyano,

(c) hydroxy,

(d) C₁₋₆ alkyl, which is unsubstituted or substituted with 1-5 of R^{e} where R^{e} is independently selected from halo, cyano, hydroxy, —O-C₁₋₆ alkyl, —C₃₋₅ cycloalkyl, —CO₂H, —CO₂(C₁₋₆ alkyl), —CF₃, —SO₂R^{a}, —NR^{a}R^{b},

where R^{a} and R^{b} are independently selected from hydrogen, C₁₋₆ alkyl, C₅₋₆ cycloalkyl, benzyl or phenyl, which is unsubstituted or substituted with 1-3 substituents where the substituents are independently selected from halo, C₁₋₃ alkyl, —O-C₁₋₃ alkyl, C₁₋₃ fluoroalkyl, and —O-C₁₋₃ fluoroalkyl, phenyl, naphthyl, biphenyl, and heterocycle, wherein the phenyl, naphthyl, biphenyl or heterocycle is unsubstituted or substituted with 1-7 of R^{f} where R^{f} is independently selected from halo, cyano, hydroxy, C₁₋₄ alkyl, —O-C₁₋₄ alkyl, —O-C₃₋₅ cycloalkyl, —CO₂H, —CO₂(C₁₋₆ alkyl), —CF₃, —OCF₃, —SO₂R^{a}, —N(R^{a})SO₂R^{b} and NR^{a}R^{b},

(e) —O-C₁₋₆ alkyl, which is unsubstituted or substituted with 1-5 of R^{e},

(f) —NO₂,

(g) phenyl,

(h) —CO₂R^{a},

(i) tetrazolyl,

(j) —NR^{a}R^{b},

(k) —NR^{a}—COR^{b},

(l) —NR^{a}—CO₂R^{b},

(m) —CO—NR^{a}R^{b},

(n) —OCO—NR^{a}R^{b},

(o) —NR^{a}CO—NR^{a}R^{b},

(p) —S(O)ₘ—R^{a}, wherein m is an integer selected from 0, 1 and 2,

(q) —S(O)₂—NR^{a}R^{b},

(r) —NR^{a}S(O)₂—R^{b},

(s) —NR^{a}S(O)₂—NR^{a}R^{b};

(t) —C₃₋₆ cycloalkyl, and

(u) —O-C₃₋₆ cycloalkyl;

and all other variables are as originally defined;

and with the proviso that

(A) when R¹⁰ is a heterocycle selected from pyrazolyl and imidazolyl, then the heterocycle is unsubstituted or substituted with 1 or 2 substituents independently selected from any of substituents (a) to (u) as defined above; and

(B) when R¹⁰ is a heterocycle selected from:

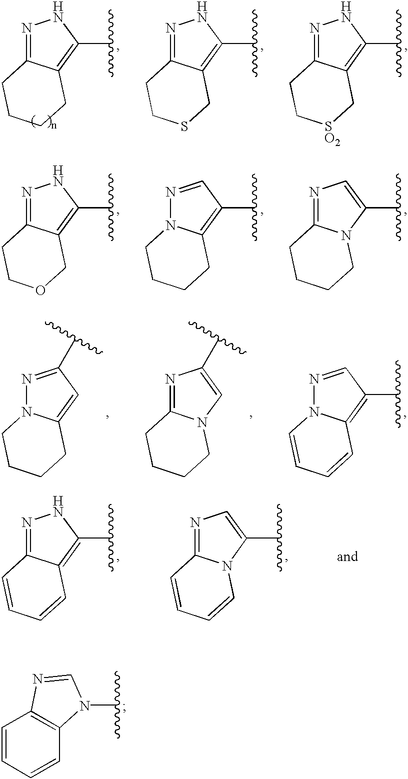

then the heterocycle is unsubstituted in the pyrazolyl or imidazolyl ring, and is either unsubstituted in the other ring or is substituted with 1 or 2 substituents independently selected from any of substituents (a) to (u) as defined above;

or a pharmaceutically acceptable salt thereof.

An aspect of the eleventh embodiment is a compound of Formula I exactly as defined in the eleventh embodiment, except that the definition of R¹⁰ does not include triazolyl.

In another aspect of the eleventh embodiment, R¹⁰ is phenyl, benzimidazolyl, imidazolyl, pyridoimidazolyl, isoxazolyl, oxazolyl, pyrazolyl, pyridyl, thiazolyl, imidazothiophenyl, indazolyl, tetrahydropyridoimidazolyl, tetrahydroindazolyl, dihydrothiopyranopyrazolyl, dihydrodioxothiopyranopyrazolyl, dihydropyranopyrazolyl, tetrahydropyridopyrazolyl, or triazolyl; any one of which is unsubstituted or substituted with 1-5 substituents where the substituents are independently selected from:

(a) halo,

(b) cyano,

(c) —NO₂,

(d) —CF₃,

(e) —CHF₂,

(f) —CH₂F,

(g) —CH₂OH,

(h) —CH₂OCH₃,

(i) —(CH₂)₁₋₂SO₂—(C₁₋₂ alkyl)

(j) phenyl,

(k) C₁₋₆ alkyl, which is unsubstituted or substituted with phenyl, which is unsubstituted or substituted with 1-4 of R^{f} where R^{f} is independently selected from halo, cyano, hydroxy, —O—C₁₋₆ alkyl, —O—C₃₋₅ cycloalkyl, —CO₂H, —CO₂(C₁₋₆ alkyl), —CF₃, —OCF₃, —SO₂—(C₁₋₃ alkyl), and —N(R^{a})SO₂—(C₁₋₃ alkyl),

(l) —O-C₁₋₆ alkyl,

(m) —C₃₋₅ cycloalkyl,

(n) —CH₂—(C₃₋₅ cycloalkyl), and

(o) —O-C₃₋₅ cycloalkyl;

and with the proviso that

(A) when R¹⁰ is a heterocycle selected from pyrazolyl and imidazolyl, then the heterocycle is unsubstituted or substituted with 1 or 2 substituents independently selected from any of substituents (a) to (o) as defined above; and

(B) when R¹⁰ is a heterocycle selected from:

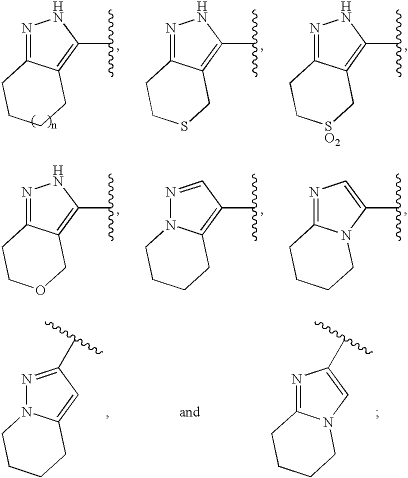

then the heterocycle is unsubstituted in the pyrazolyl or imidazolyl ling, and is either unsubstituted in the other ring or is substituted with 1 or 2 substituents independently selected from any of substituents (a) to (o) as defined above.

In another aspect of the eleventh embodiment, the compound of Formula I is just as defined in the preceding aspect, except that the definition of R¹⁰ does not include triazolyl.

In another aspect of the eleventh embodiment, RIO is:

(i) pyrazolyl or imidazolyl, either of which is unsubstituted or substituted with 1 or 2 substituents independently selected from:

(a) fluoro,

(b) chloro,

(c) C₁₋₆ alkyl,

(d) —CH₂-phenyl, wherein the phenyl is unsubstituted or substituted with 1 or 2 substituents independently selected from chloro, fluoro, —CN, —C₁₋₃ alkyl, —O-C₁₋₃ alkyl, —O-cyclopropyl, —O-cyclobutyl, —CF₃, —OCF₃, —SO₂—(C₁₋₃ alkyl), and —N(H)SO₂—(C₁₋₃ alkyl),

(e) —CH₂CH₂-phenyl, and

(f) phenyl; or

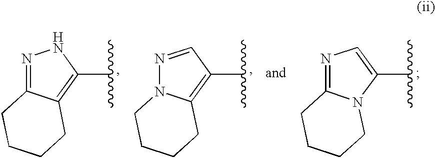

each of which is unsubstituted in the pyrazolyl or imidazolyl ring, and is either unsubstituted in the other ring or is substituted with 1 or 2 substituents independently selected from:

(a) halo,

(b) C₁₋₄ alkyl,

(c) C₁₋₄ haloalkyl,

(d) —OH,

(e) —O-C₁₋₄ alkyl,

(f) —O-C₁₋₄ haloalkyl, and

(g) —CN.

It is to be understood that additional embodiments of the present invention include, but are not limited to, compounds of Formula I wherein each of two or three or more of R¹, R², R³, R⁴, R⁵, R^{6a}, R^{6b}, R⁷, R⁸, R⁹, R¹⁰ and Y is independently defined in accordance with one of the foregoing embodiments or aspects thereof as set forth above. Any and all possible combinations of these variables in Formula I are within the scope of the present invention.

The compounds of the instant invention have at least two asymmetric centers at the ring junction of the substituents bearing R² and R³. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention.

A first class of compounds of the present invention are compounds having the trans orientation, depicted as:

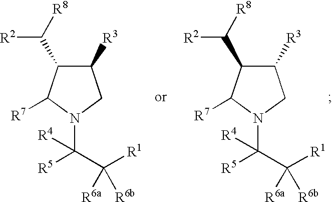

and pharmaceutically acceptable salts thereof.

A second class of the present invention is compounds of Formula (II):

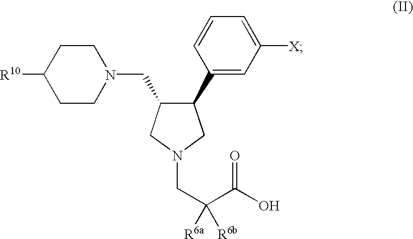

wherein

R^{6a} and R^{6b} are each C₁₋₄ alkyl;

or one of R^{6a} and R^{6b} is C₁₋₄ alkyl, and the other of R^{6a} and R^{6b} is C₃₋₆ cycloalkyl;

or R^{6a} and R^{6b} together with the carbon atom to which they are attached form:

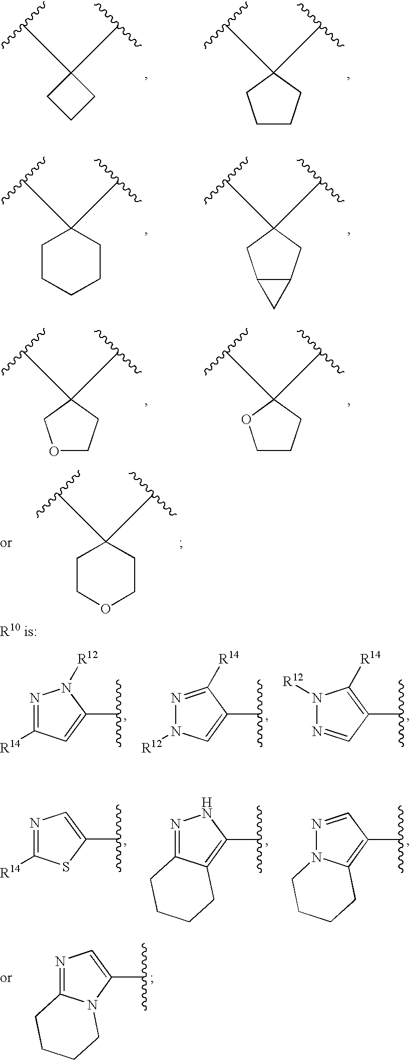

R¹² is hydrogen, C₁₋₄ alkyl, C₁₋₄ fluoroalkyl, —(C₁₋₄ alkyl)—SO₂—(C₁₋₄ alkyl), or —CH₂-phenyl wherein the phenyl is optionally substituted with 1 or 2 substituents independently selected from chloro, fluoro, —CN, —C₁₋₄ alkyl, —O-C₁₋₄ alkyl, —O-cyclopropyl, —O-cyclobutyl, —CF₃, —OCF₃, —SO₂—(C₁₋₄ alkyl), and —NHSO₂—(C₁₋₄ alkyl);

R¹⁴ is hydrogen, —C₁₋₄ alkyl, C₁₋₄ fluoroalkyl, —O-C₁₋₄ alkyl, —O-C₁₋₄ fluoroalkyl, cyclopropyl, cyclobutyl, or —CH₂-phenyl wherein the phenyl is optionally substituted with 1 or 2 substituents independently selected from chloro, fluoro, —CN, —C₁₋₄ alkyl, —O-C₁₋₄ alkyl, —O-cyclopropyl, —O-cyclobutyl, —CF₃, —OCF₃, and —SO₂—(C₁₋₄ alkyl); and

X is hydrogen or fluoro;

or a pharmaceutically acceptable salt thereof.

A first sub-class of the present invention is compounds of Formula (II), wherein

R^{6a} and R^{6b} are each C₁₋₃ alkyl;

or one of R^{6a} and R^{6b} is C₁₋₃ alkyl, and the other of R^{6a} and R^{6b} is C₃₋₆ cycloalkyl;

or R^{6a} and R^{6b} together with the carbon atom to which they are attached form:

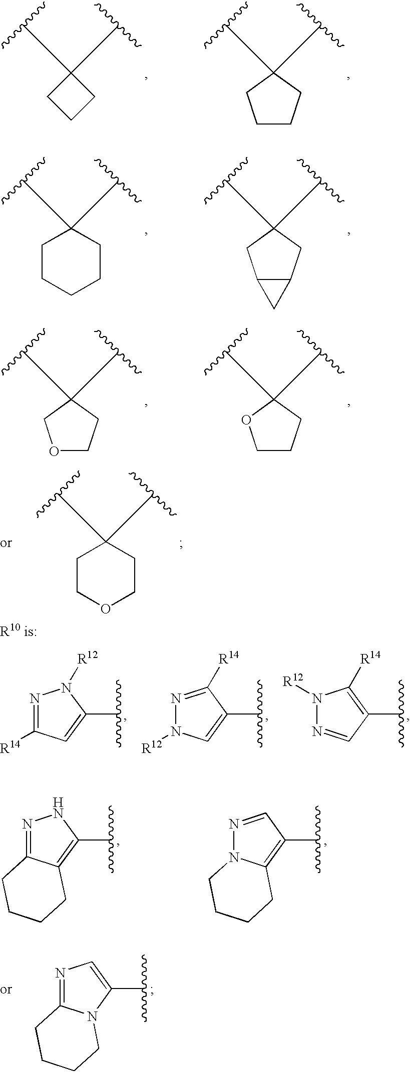

R¹² is hydrogen, C₁₋₃ alkyl, C₁₋₃ fluoroalkyl, or —CH₂-phenyl wherein the phenyl is optionally substituted with 1 or 2 substituents independently selected from chloro, fluoro, —CN, —C₁₋₃ alkyl, —O-C₁₋₃ alkyl, —O-cyclopropyl, —O-cyclobutyl, —CF₃, —OCF₃, —SO₂—(C₁₋₃ alkyl), and —NHSO₂—(C₁₋₃ alkyl);

R¹⁴ is hydrogen, —C₁₋₃ alkyl, C₁₋₃ fluoroalkyl, —O-C₁₋₃ alkyl, —O-C₁₋₃ fluoroalkyl, cyclopropyl, cyclobutyl, or —CH₂-phenyl wherein the phenyl is optionally substituted with 1 or 2 substituents independently selected from chloro, fluoro, —CN, —C₁₋₃ alkyl, —O-C₁₋₃ alkyl, —O-cyclopropyl, —O-cyclobutyl, —CF₃, —OCF₃, and —SO₂—(C₁₋₃ alkyl); and

X is hydrogen or fluoro;

or a pharmaceutically acceptable salt thereof.

A second sub-class of the present invention is compounds of Formula II, wherein R¹⁰ is:

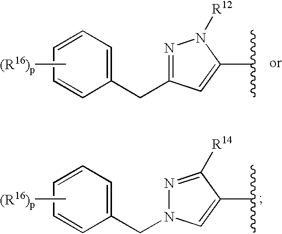

R¹² is C13 alkyl;

R¹⁴ is —C₁₋₃ alkyl;

each R¹⁶ is independently chloro, fluoro, —CN, —C₁₋₃ alkyl, —O-C₁₋₃ alkyl, —O-cyclopropyl, —O-cyclobutyl, —CF₃, —OCF₃, or —SO₂—(C₁₋₃ alkyl); and

p is an integer from zero to 3;

and all other variables are as defined in the first sub-class;

or a pharmaceutically acceptable salt thereof.

In one aspect of the second sub-class, R¹² and R¹⁴ are both ethyl.

The independent syntheses of the diastereomers described above or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

Other embodiments of the present invention include the following:

(a) A pharmaceutical composition comprising a compound of Formula (I) and a pharmaceutically acceptable carrier.

(b) The pharmaceutical composition of (a), further comprising at least one antiviral selected from the group consisting of HIV protease inhibitors, non-nucleoside HIV reverse transcriptase inhibitors, and nucleoside HIV reverse transcriptase inhibitors.

(c) A method for modulating (e.g., inhibiting) CCR5 chemokine receptor activity in a subject which comprises administering to the subject an effective amount of the compound of Formula (I).

(d) A method of preventing or treating infection by HIV in a subject in need thereof which comprises administering to the subject a therapeutically effective amount of a compound of Formula (I).

(e) The method of (d), wherein the compound of Formula (I) is administered in combination with a therapeutically effective amount of at least one antiviral selected from the group consisting of HIV protease inhibitors, non-nucleoside HIV reverse transcriptase inhibitors, and nucleoside HIV reverse transcriptase inhibitors.

(f) A method of delaying the onset or AIDS or treating AIDS in a subject in need thereof which comprises administering to the subject a therapeutically effective amount of a compound of Formula (I).

(g) The method of (f), wherein the compound is administered in combination with a therapeutically effective amount of at least one antiviral selected from the group consisting of HIV protease inhibitors, non-nucleoside HIV reverse transcriptase inhibitors, and nucleoside HIV reverse transcriptase inhibitors

(h) A method of modulating (e.g., inhibiting) CCR5 chemokine receptor acitivity in a subject in need thereof which comprises administering to the subject a therapeutically effective amount of the composition of (a) or (b).

(i) A method of preventing or treating infection by HIV in a subject in need thereof which comprises administering to the subject a therapeutically effective amount of the composition of (a) or (b).

(j) A method of treating AIDS or delaying the onset of AIDS in a subject in need thereof which comprises administering to the subject a therapeutically effective amount of the composition of (a) or (b).

Still other embodiments of the present invention include the following:

(k) A pharmaceutical composition which comprises the product prepared by combining (e.g., mixing) an effective amount of a compound of Formula (I) and a pharmaceutically acceptable carrier.

(l) A combination useful for treating or preventing infection by HIV, or for preventing, treating or delaying the onset of AIDS, which is a therapeutically effective amount of a compound of Formula (I) and a therapeutically effective amount of an HIV infection/AIDS treatment agent selected from the group consisting of HIV/AIDS antiviral agents, immunomodulators, and anti-infective agents.

(m) The combination of (1), wherein the HIV infection/AIDS treatment agent is an antiviral selected from the group consisting of HIV protease inhibitors, non-nucleoside HIV reverse transcriptase inhibitors and nucleoside HIV reverse transcriptase inhibitors.

Additional embodiments of the invention include the pharmaceutical compositions and methods set forth in (a)-(j) above and the compositions and combinations set forth in (k)-(m), wherein the compound employed therein is a compound of one of the embodiments, classes, sub-classes, or aspects of compounds described above. In all of these embodiments, the compound may optionally be used in the form of a pharmaceutically acceptable salt.

As used herein, the term “C₁₋₆ alkyl” (or “C₁-C₆ alkyl”) means linear or branched chain alkyl groups having from 1 to 6 carbon atoms and includes all of the hexyl alkyl and pentyl alkyl isomers as well as n-, iso-, sec- and t-butyl, n- and isopropyl, ethyl and methyl. “C₁₋₄ alkyl” means n-, iso-, sec- and t-butyl, n- and isopropyl, ethyl and methyl. Similar terms such as “C₁₋₁₀ alkyl” have analogous meanings.

The term “C₀” as employed in expressions such as “C₀₋₆ alkyl” means a direct covalent bond.

The term “C₂₋₆ alkenyl” (or “C₂-C₆ alkenyl”) means linear or branched chain alkenyl groups having from 2 to 6 carbon atoms and includes all of the hexenyl and pentenyl isomers as well as 1-butenyl, 2-butenyl, 3-butenyl, isobutenyl, 1-propenyl, 2-propenyl, and ethenyl (or vinyl). Similar terms such as “C₂₋₁₀ alkenyl” have analogous meanings.

The term “C₂₋₆ alkynyl” (or “C₂-C₆ alkynyl”) means linear or branched chain alkynyl groups having from 2 to 6 carbon atoms and includes all of the hexynyl and pentynyl isomers as well as 1-butynyl, 2-butynyl, 3-butynyl, 1-propynyl, 2-propynyl, and ethynyl (or acetylenyl). Similar terms such as “C₂₋₁₀ alkynyl” have analogous meanings.

The term “C₃₋₈ cycloalkyl” (or “C₃-C₈ cycloalkyl”) means a cyclic ring of an alkane having three to eight total carbon atoms (i.e., cyclopropyl, cyclobutyl, cyclopentyl, cyclohexyl, cycloheptyl, or cyclooctyl). The term “C₃₋₆ cycloalkyl” refers to a cyclic ring selected from cyclopropyl, cyclobutyl, cyclopentyl, and cyclohexyl. Similar terms such as “C₅₋₆ cycloalkyl” have analogous meanings.

The term “halogen” (or “halo”) refers to fluorine, chlorine, bromine and iodine (alternatively, fluoro, chloro, bromo, and iodo).

The term “C₁₋₆ haloalkyl” (which may alternatively be referred to as “C₁-C₆ haloalkyl” or “halogenated C₁-C₆ alkyl”) means a C₁ to C₆ linear or branched alkyl group as defined above with one or more halogen substituents. The term “C₁₋₄ haloalkyl” has an analogous meaning. Similarly, “C₁₋₆ fluoroalkyl” means a C₁ to C₆ linear or branched alkyl group as defined above with one or more fluorine substituents. Representative examples of suitable fluoroalkyls include the series (CH₂)₀₋₄CF₃ (i.e., trifluoromethyl, 2,2,2-trifluoroethyl, 3,3,3-trifluoro-n-propyl, etc.), 1-fluoroethyl, 2-fluoroethyl, 2,2-difluoroethyl, 3,3,3-trifluoroisopropyl, 1,1,1,3,3,3-hexafluoroisopropyl, and perfluorohexyl.

The term “-(C₁₋₆ alkyl)hydroxy” refers to a C₁₋₆ alkyl group as defined above which is substituted on one its carbons by a hydroxy group. Exemplary groups include hydroxymethyl, hydroxyethyl, 3-hydroxy-n-propyl, 2-hydroxy-n-propyl, and so forth.

The term “C₃₋₈ cycloalkylidenyl” refers to a C₃₋₈ cycloalkyl group as defined above in which one of the ring carbons is attached to each of two carbon atoms not in the ring such that the three carbon atoms form a carbon chain or part of a carbon chain. Thus, “—(C₀₋₂ alkyl)-(C₃₋₈ cycloalkylidenyl)-(C₁₋₂ alkyl)” refers to and encompasses such groups as:

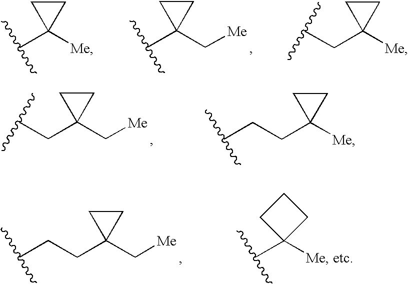

The term “carbocycle” (and variations thereof such as “carbocyclic” or “carbocyclyl”) as used herein broadly refers to a C₃ to C₈ monocyclic, saturated or unsaturated ring or a C₇ to C₁₄ bicyclic ring system in which the rings are independent or fused and in which each ring is saturated or unsaturated.

The term “aryl” refers to aromatic mono- and poly-carbocyclic ring systems, wherein the individual carbocyclic rings in the polyring systems may be fused or attached to each other via a single bond. Suitable aryl groups include, but are not limited to, phenyl, naphthyl, and biphenylenyl.

The term “heterocycle” (and variations thereof such as “heterocyclic” or “heterocyclyl”) broadly refers to a 4- to 8-membered monocyclic ring, 7- to 14-membered bicyclic ring system, or an 11 to 16-membered tricyclic ring system, any ring of which is saturated or unsaturated, and which consists of carbon atoms and one or more heteroatoms (e.g., from 1 to 4 heteroatoms) selected from N, O and S, and wherein the nitrogen and sulfur heteroatoms may optionally be oxidized, and the nitrogen heteroatom may optionally be quaternized. The heterocyclic ring may be attached at any heteroatom or carbon atom, provided that attachment results in the creation of a stable structure.

The term “heterocycle” as used herein is intended to include the following groups: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, methylenedioxybenzyl, tetrahydrofuranyl, and tetrahydrothienyl, and N-oxides thereof.

The term “heterocycle” as used herein is also intended to include, but is not limited to, the following groups: methylenedioxyphenyl, imidazopyridyl, imidazopyrimidinyl, imidazopyridazinyl, imidazopyrazinyl, imidazotriazinyl, imidazothiopheyl, pyrazolopyridyl, pyrazolopyrimidinyl, pyrazolopyridazinyl, pyrazolopyrazinyl, pyrazolotriazinyl, pyrazolothiophenyl, triazolopyridyl, triazolopyrimidinyl, triazolopyridazinyl, triazolopyrazinyl, triazolothiophenyl, tetrahydroimidazopyridinyl, tetrahydropyrazolopyridinyl, tetrahydrotriazopyridinyl, tetrahydrotriazolopyridazinyl, and tetrahydroindazolyl.

The term “heterocycle” as used herein is also intended to include, but is not limited to, the following groups: tetrahydroimidazopyrimidyl, tetrahydroimidazopyrazinyl, tetrahydroimidazopyridazinyl, tetrahydrotriazolopyrimidyl, tetrahydrotriazolopyrazinyl, tetrahydropyrazolopyrimidyl, tetrahydropyrazolopyrazinyl, imidazothiazolyl, and imidazothiadiazolyl.

The term “heterocycle” as used herein is also intended to include, but is not limited to, oxopyridinyl (e.g., 2-oxopyridinyl), oxopiperidinyl, and oxopyrazolyl.

The terms “thiophenyl” and “thienyl” have the same meaning herein and are used interchangeably. Similarly, the following pairs of terms have the same meaning: “indazolyl” and “benzopyrazolyl”; “pyridinyl” and “pyridyl”.

Unless expressly set forth to the contrary, an “unsaturated” ring is a partially or fully unsaturated ring.

The term “substituted” in reference to substitution on alkyl, cycloalkyl, phenyl, heterocycle, or some other chemical group is intended to include mono- and poly-substitution by a named substituent to the extent such single and multiple substitution is chemically allowed in any of the named chemical groups.

It is understood that the definition of a substituent at a particular location in a molecule is independent of its definition at other locations in the molecule. Thus, for example, when Z¹═—N(R^{u})C(═CHR^{s})N(R^{u})—, the value of R^{u} (defined elsewhere) on one of the nitrogens is independent of the value of R^{u} at the other nitrogen; i.e., they can be the same or different.

Exemplifying the invention are the compounds disclosed in the Examples and the use of these compounds as disclosed herein (e.g., for treating HIV infection or AIDS).

One aspect of the present invention is 1-{[(3S,4S)-3-[(4-{3-ethyl-1-[4-(methylsulfonyl)benzyl]-1H-pyrazol-4-yl}piperidin-1-yl)methyl]-4-(3-fluorophenyl)pyrrolidin-1-yl]methyl}cyclohexanecarboxylic acid, which may be represented structurally as

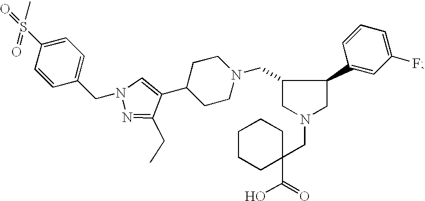

or a pharmaceutically acceptable salt thereof.

The subject compounds are useful in a method of modulating (e.g., inhibiting) CCR5 chemokine receptor activity in a patient in need of such modulation (inhibition) comprising the administration of an effective amount of the compound.

The present invention is directed to the use of the foregoing compounds as modulators (inhibitors) of CCR5 chemokine receptor activity.

The utility of the compounds in accordance with the present invention as modulators of CCR5 chemokine receptor activity may be demonstrated by methodology known in the art, such as the assay for chemokine binding as disclosed by Van Riper, et al., J. Exp. Med., 177, 851-856 (1993) which may be readily adapted for measurement of CCR5 binding. Cell lines for expressing the receptor of interest include those naturally expressing the receptor, such as EOL-3 or THP-1, or a cell engineered to express a recombinant receptor, such as CHO, RBL-2H3, HEK-293. The utility of the compounds in accordance with the present invention as inhibitors of the spread of UIV infection in cells may be demonstrated by methodology known in the art, such as the HIV quantitation assay disclosed by Nunberg, et al., J. Virology, 65 (9), 4887-4892 (1991).

In particular, the compounds of the following examples had activity in binding to the CCR5 receptor in the aforementioned assays, generally with an 1CSO of less than about 5 μM. Such a result is indicative of the intrinsic activity of the compounds in use as modulators of CCR5 chemokine receptor activity.

Mammalian chemokine receptors provide a target for interfering with or promoting eosinophil and/or lymphocyte function in a mammal, such as a human. Compounds which inhibit or promote chemokine receptor function, are particularly useful for modulating eosinophil and/or lymphocyte function for therapeutic purposes. Accordingly, the present invention is directed to compounds which are useful in the prevention and/or treatment of a wide variety of inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis.

For example, an instant compound which inhibits one or more functions of a mammalian chemokine receptor (e.g., a human chemokine receptor) may be administered to inhibit (i.e., reduce or prevent) inflammation. As a result, one or more inflammatory processes, such as leukocyte emigration, chemotaxis, exocytosis (e.g., of enzymes, histamine) or inflammatory mediator release, is inhibited. For example, eosinophilic infiltration to inflammatory sites (e.g., in asthma) can be inhibited according to the present method.

Similarly, an instant compound which promotes one or more functions of a mammalian chemokine receptor (e.g., a human chemokine) is administered to stimulate (induce or enhance) an inflammatory response, such as leukocyte emigration, chemotaxis, exocytosis (e.g., of enzymes, histamine) or inflammatory mediator release, resulting in the beneficial stimulation of inflammatory processes. For example, eosinophils can be recruited to combat parasitic infections.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

Diseases and conditions associated with inflammation and infection can be treated using the method of the present invention. In a preferred embodiment, the disease or condition is one in which the actions of eosinophils and/or lymphocytes are to be inhibited or promoted, in order to modulate the inflammatory response.

Diseases or conditions of humans or other species which can be treated with inhibitors of chemokine receptor function, include, but are not limited to: inflammatory or allergic diseases and conditions, including respiratory allergic diseases such as asthma, particularly bronchial asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersentitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinphilic myositis, eosinophilic fasciitis; cancers with leukocyte infiltration of the skin or organs. Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis.

Diseases or conditions of humans or other species which can be treated with promoters of chemokine receptor function, include, but are not limited to: immunosuppression, such as that in individuals with immunodeficiency syndromes such as AIDS, individuals undergoing radiation therapy, chemotherapy, therapy for autoimmune disease or other drug therapy (e.g., corticosteroid therapy), which causes immunosuppression; immunosuppression due congenital deficiency in receptor function or other causes; and infectious diseases, such as parasitic diseases, including, but not limited to helminth infections, such as nematodes (round worms); (Trichuriasis, Enterobiasis, Ascariasis, Hookworm, Strongyloidiasis, Trichinosis, filariasis); trematodes (flukes) (Schistosomiasis, Clonorchiasis), cestodes (tape worms) (Echinococcosis, Taeniasis saginata, Cysticercosis); visceral worms, visceral larva migrans (e.g., Toxocara), eosinophilic gastroenteritis (e.g., Anisaki spp., Phocanema ssp.), cutaneous larva migrans (Ancylostona braziliense, Ancylostoma caninum).

The compounds of the present invention are accordingly useful in the prevention and treatment of a wide variety of inflammatory and immunoregulatory disorders and diseases, allergic conditions, atopic conditions, as well as autoimmune pathologies.

In another aspect, the instant invention may be used to evaluate putative specific agonists or antagonists of CCR5 chemokine receptors. Accordingly, the present invention is directed to the use of these compounds in the preparation and execution of screening assays for compounds which modulate the activity of CCR5 chemokine receptors. For example, the compounds of this invention are useful for isolating receptor mutants, which are excellent screening tools for more potent compounds. Furthermore, the compounds of this invention are useful in establishing or determining the binding site of other compounds to chemokine receptors, e.g., by competitive inhibition. The compounds of the instant invention are also useful for the evaluation of putative specific modulators of the CCR5 chemokine receptors. As appreciated in the art, thorough evaluation of specific agonists and antagonists of the above chemokine receptors has been hampered by the lack of availability of non-peptidyl (metabolically resistant) compounds with high binding affinity for these receptors. Thus the compounds of this invention are commercial products to be sold for these purposes.

The present invention is further directed to a method for the manufacture of a medicament for modulating CCR5 chemokine receptor activity in humans and animals comprising combining a compound of the present invention with a pharmaceutical carrier or diluent.

The present invention is further directed to the use of these compounds in the prevention or treatment of infection by a retrovirus, in particular, the human immunodeficiency virus (HIV) and the treatment of, and delaying of the onset of consequent pathological conditions such as AIDS. Treating AIDS or preventing or treating infection by HIV is defined as including, but not limited to, treating a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by, e.g., blood transfusion, organ transplant, exchange of body fluids, bites, accidental needle stick, or exposure to patient blood during surgery.

In an aspect of the present invention, a subject compound may be used in a method of inhibiting the binding of a chemokine to a CCR5 chemokine receptor of a target cell, which comprises contacting the target cell with an amount of the compound which is effective at inhibiting the binding of the chemokine to the CCR5 chemokine receptor.

The subject treated in the methods above is a mammal, preferably a human being, male or female, in whom modulation of CCR5 chemokine receptor activity is desired. “Modulation” as used herein is intended to encompass antagonism, agonism, partial antagonism, inverse agonism and/or partial agonism. In an aspect of the present invention, modulation refers to antagonism of CCR5 chemokine receptor activity. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

The term “subject,” (alternatively referred to herein as “patient”) as used herein refers to an animal, preferably a mammal, most preferably a human, who has been the object of treatment, observation or experiment.

Combined therapy to modulate CCR5 chemokine receptor activity and thereby prevent and treat inflammatory and immunoregulatory disorders and diseases, including asthma and allergic diseases, as well as autoimmune pathologies such as rheumatoid arthritis and atherosclerosis, and those pathologies noted above is illustrated by the combination of the compounds of this invention and other compounds which are known for such utilities.

For example, in the treatment or prevention of inflammation, the present compounds may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, asprin, codiene, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine. Likewise, compounds of the present invention may be used in combination with other drugs that are used in the treatment/prevention/suppression or amelioration of the diseases or conditions for which compounds of the pressent invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention. Examples of other active ingredients that may be combined with a compound of the present invention, either administered separately or in the same pharmaceutical compositions, include, but are not limited to: (a) VLA-4 antagonists such as those described in US 5,510,332, WO95/15973, WO96/01644, WO96/06108, WO96/20216, WO96/22966, WO96/31206, WO96/40781, WO97/03094, WO97/02289, WO 98/42656, WO98/53814, WO98/53817, WO98/53818, WO98/54207, and WO98/58902; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (Hi-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as P2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CXCR-4, CCR1, CCR2, CCR3 and CCR5; (j) cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (k) anti-diabetic agents such as insulin, sulfonylureas, biguanides (metfonnin), α-glucosidase inhibitors (acarbose) and glitazones (troglitazone and pioglitazone); (1) preparations of interferon beta (interferon beta-1α, interferon beta-1β); (m) other compounds such as 5-aminosalicylic acid and prodrugs thereof, antimetabolites such as azathioprine and 6-mercaptopurine, and cytotoxic cancer chemotherapeutic agents. The weight ratio of the compound of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

The present invention is further directed to combinations of the present compounds with one or more agents useful in the prevention or treatment of AIDS. For example, the compounds of this invention may be effectively administered, whether at periods of pre-exposure and/or post-exposure, in combination with effective amounts of the antiviral agents, immunomodulators, anti-infectives, or vaccines suitable for treating HIV infection and AIDS, and known to those of ordinary skill in the art, including those listed in the following Table.

| | | |
|---|---|---|
| Drug Name | Manufacturer | Indication |

ANTIVIRALS

| | | |
|---|---|---|
| Amprenavir | Glaxo Wellcome | HIV infection, AIDS, |
| 141 W94 | | ARC |
| GW 141 | | (protease inhibitor) |
| Abacavir | Glaxo Wellcome | HIV infection, AIDS, |
| GW 1592 | | ARC |
| 1592U89 | | (reverse transcriptase |
| | | inhibitor) |
| Acemannan | Carrington Labs | ARC |
| | (Irving, TX) | |
| Acyclovir | Burroughs Wellcome | HIV infection, AIDS, ARC, |
| | | in combination with AZT |
| AD-439 | Tanox Biosystems | HIV infection, AIDS, ARC |
| AD-519 | Tanox Biosystems | HIV infection, AIDS, ARC |
| Adefovir dipivoxil | Gilead Sciences | HIV infection |
| AL-721 | Ethigen | ARC, PGL, HIV positive, |
| | (Los Angeles, CA) | AIDS |
| Alpha Interferon | Glaxo Wellcome | Kaposi's sarcoma, HIV, in |
| | | combination w/Retrovir |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| neutralizes pH | Concepts | |
| labile alpha aberrant | (Rockville, MD) | |
| Interferon | | |
| AR177 | Aronex Pharm | HIV infection, AIDS, ARC |
| beta-fluoro-ddA | Nat'l Cancer Institute | AIDS-associated diseases |
| BMS-232623 | Bristol-Myers Squibb/ | HIV infection, AIDS, |
| (CGP-73547) | Novartis | ARC |
| | | (protease inhibitor) |
| BMS-234475 | Bristol-Myers Squibb/ | HIV infection, AIDS, |
| (CGP-61755) | Novartis | ARC |
| | | (protease inhibitor) |
| CI-1012 | Warner-Lambert | HIV-1 infection |
| Cidofovir | Gilead Science | CMV retinitis, herpes, |
| | | papillomavirus |
| Curdlan sulfate | AJI Pharma U.S.A. | HIV infection |
| Cytomegalovirus immune | MedImmune | CMV retinitis |
| globin | | |
| Cytovene | Syntex | sight threatening CMV |
| Ganciclovir | | peripheral CMV |
| | | retinitis |
| Delaviridine | Pharmacia-Upjohn | HIV infection, AIDS, |
| | | ARC |
| | | (protease inhibitor) |
| Dextran Sulfate | Ueno Fine Chem. | AIDS, ARC, HIV |
| | Ind. Ltd. (Osaka, Japan) | positive asymptomatic |
| ddC | Hoffman-La Roche | HIV infection, AIDS, ARC |
| Dideoxycytidine | | |
| ddI | Bristol-Myers Squibb | HIV infection, AIDS, ARC; |
| Dideoxyinosine | | combination with AZT/d4T |
| mozenavir | AVID | HIV infection, AIDS, |
| (DMP-450) | (Camden, NJ) | ARC |
| | | (protease inhibitor) |
| EL10 | Elan Corp. PLC | HIV infection |
| | (Gainesville, GA) | |
| Efavirenz | DuPont (SUSTIVA ®), | HIV infection, AIDS, |
| (DMP 266) | Merck (STOCRIN ®) | ARC |
| (−) 6-Chloro-4(S)- | | (non-nucleoside RT |
| cyclopropylethynyl- | | inhibitor) |
| 4(S)-trifluoro-methyl- | | |
| 1,4-dihydro-2H-3, 1- | | |
| benzoxazin-2-one, | | |
| Famciclovir | Smith Kline | herpes zoster, herpes |
| | | simplex |
| FTC | Emory University | HIV infection, AIDS, ARC |
| | | (reverse transcriptase |
| | | inhibitor) |
| GS 840 | Gilead | HIV infection, AIDS, ARC |
| | | (reverse transcriptase |
| | | inhibitor) |
| HBY097 | Hoechst Marion Roussel | HIV infection, AIDS, ARC |
| | | (non-nucleoside reverse |
| | | transcriptase inhibitor) |
| Hypericin | VIMRx Pharm. | HIV infection, AIDS, ARC |
| Recombinant Human | Triton Biosciences | AIDS, Kaposi's sarcoma, |
| Interferon Beta | (Almeda, CA) | ARC |
| Interferon alfa-n3 | Interferon Sciences | ARC, AIDS |
| Indinavir | Merck | HIV infection, AIDS, ARC, |
| | | asymptomatic HIV positive, |
| | | also in combination with |
| | | AZT/ddI/ddC |
| Compound A | Merck | HIV infection, AIDS, |
| | | ARC, asymptomatic HIV |
| | | positive |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, AIDS, |
| | | ARC (reverse |
| | | transcriptase inhibitor); |
| | | also with AZT |
| Lobucavir | Bristol-Myers Squibb | CMV infection |
| Nelfinavir | Agouron | HIV infection, AIDS, |
| | Pharmaceuticals | ARC (protease inhibitor) |
| Nevirapine | Boeheringer | HIV infection, AIDS, |
| | Ingleheim | ARC (protease inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV infection, |
| Phosphonoformate | Products, Inc | other CMV |
| | | infections |
| PNU-140690 | Pharmacia Upjohn | HIV infection, AIDS, ARC |
| | | (protease inhibitor) |
| Probucol | Vyrex | HIV infection, AIDS |
| RBC-CD4 | Sheffield Med. Tech | HIV infection, AIDS, |
| | (Houston TX) | ARC |
| Ritonavir | Abbott | HIV infection, AIDS, |
| (ABT-538) | | ARC (protease inhibitor) |
| Saquinavir | Hoffmann-LaRoche | HIV infection, AIDS, |
| | | ARC (protease inhibitor) |
| Stavudine; d4T | Bristol-Myers Squibb | HIV infection, AIDS, ARC |
| Didehydrodeoxy- | | |
| thymidine | | |
| Valaciclovir | Glaxo Wellcome | genital HSV & CMV |
| | | infections |
| Virazole | Viratek/ICN | asymptomatic HIV |
| Ribavirin | (Costa Mesa, CA) | positive, LAS, ARC |
| VX-478 | Vertex | HIV infection, AIDS, ARC |
| Zalcitabine | Hoffmann-La Roche | HIV infection, AIDS, ARC, |
| | | with AZT |
| Zidovudine; AZT | Glaxo Wellcome | HIV infection, AIDS, ARC, |
| | | Kaposi's sarcoma in |
| | | combination with other |
| | | therapies (reverse |
| | | transcriptase inhibitor) |
| ABT-378; Lopinavir | Abbott | HIV infection, AIDS, ARC |
| | | (protease inhibitor) |
| ABT-378/r; contains | Abbott | HIV infection, AIDS, ARC |
| lopinavir and ritonavir; | | (protease inhibitor) |
| Kaletra | | |
| JE2147/AG1776 | Agouron | HIV infection, AIDS, ARC |
| | | (protease inhibitor) |
| T-20 | Trimeris | HIV infection, AIDS, ARC |
| | | (fusion inhibitor) |
| T-1249 | Trimeris | HIV infection, AIDS, ARC |
| | | (fusion inhibitor) |
| atazanavir | Bristol-Myers-Squibb | HIV infection, AIDS, ARC |
| (BMS 232632) | | (protease inhibitor) |
| PRO 542 | Progenics | HIV infection, AIDS, ARC |
| | | (attachment inhibitor) |
| PRO 140 | Progenics | HIV infection, AIDS, ARC |
| | | (CCR5 co-receptor inhibitor) |
| TAK-779 | Takeda | HIV infection, AIDS, ARC |
| | | (injectable CCR5 receptor |
| | | antagonist) |
| DPC 681 & DPC 684 | DuPont | HIV infection, AIDS, ARC |
| | | (protease inhibitors) |
| DPC 961 & DPC 083 | DuPont | HIV infection AIDS, ARC |
| | | (nonnucleoside reverse |
| | | transcriptase inhibitors) |
| Trizivir (contains abacavir, | GlaxoSmithKline | HIV infection, AIDS, ARC |
| lamivudine, and | | (reverse transcriptase |
| zidovudine) | | inhibitors) |
| tipranavir (PNU-140690) | Boehringer Ingelheim | HIV infection, AIDS, ARC |
| | (purchased from | (protease inhibitor) |
| | Pharmacia & Upjohn) | |
| tenofovir disoproxil | Gilead | HIV infection, AIDS, ARC |
| fumarate | | (reverse transcriptase |
| | | inhibitor) |
| TMC-120 & TMC-125 | Tibotec | HIV infections, AIDS, ARC |
| | | (non-nucleoside reverse |
| | | transcriptase inhibitors) |
| TMC-126 | Tibotec | HIV infection, AIDS, ARC |
| | | (protease inhibitor) |

IMMUNO-MODULATORS

| | | |
|---|---|---|
| AS-101 | Wyeth-Ayerst | AIDS |
| Bropirimine | Pharmacia Upjohn | advanced AIDS |
| Acemannan | Carrington Labs, Inc. | AIDS, ARC |
| | (Irving, TX) | |
| CL246,738 | American Cyanamid | AIDS, Kaposi's sarcoma |
| | Lederle Labs | |
| EL10 | Elan Corp, PLC | HIV infection |
| | (Gainesville, GA) | |
| FP-21399 | Fuki ImmunoPharm | blocks HIV fusion with |
| | | CD4+ cells |
| Gamma Interferon | Genentech | ARC, in combination w/TNF |
| | | (tumor necrosis factor) |
| Granulocyte | Genetics Institute | AIDS |
| Macrophage Colony | Sandoz | |
| Stimulating | | |
| Factor | | |
| Granulocyte | Hoeschst-Roussel | AIDS |
| Macrophage Colony | Immunex | |
| Stimulating | | |
| Factor | | |
| Granulocyte | Schering-Plough | AIDS, combination w/AZT |
| Macrophage Colony | | |
| Stimulating Factor | | |
| HIV Core Particle | Rorer | seropositive HIV |
| Immunostimulant | | |
| IL-2 | Cetus | AIDS, in combination |
| Interleukin-2 | | w/AZT |
| IL-2 | Hoffman-La Roche | AIDS, ARC, HIV, in |
| Interleukin-2 | Immunex | combination w/AZT |
| IL-2 | Chiron | AIDS, increase in CD4 cell |
| Interleukin-2 | | counts |
| (aldeslukin) | | |
| Immune Globulin | Cutter Biological | pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's sarcoma, |
| | (New Orleans, LA) | ARC, PGL |
| Imuthiol Diethyl | Merieux Institute | AIDS, ARC |
| Dithio Carbamate | | |
| Alpha-2 | Schering Plough | Kaposi's sarcoma w/AZT, |
| Interferon | | AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte | Amgen | AIDS, in combination |
| Colony Stimulating | | w/AZT |
| Factor | | |
| Remune | Immune Response Corp. | immunotherapeutic |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma, AIDS, |
| Alfa 2a | | ARC, in combination w/AZT |
| SK&F106528 | Smith Kline | HIV infection |
| Soluble T4 | | |
| Thymopentin | Immunobiology | HIV infection |
| | Research Institute | |
| Tumor Necrosis | Genentech | ARC, in combination |
| Factor; TNF | | w/gamma Interferon |
| etanercept | Immunex Corp | rheumatoid arthritis |
| | (Enbrel ®) | |
| infliximab | Centocor (Remicade ®) | rheumatoid arthritis and |
| | | Crohn's disease |

ANTI-INFECTIVES

| | | |
|---|---|---|
| Clindamycin with | Pharmacia Upjohn | PCP |
| Primaquine | | |
| Fluconazole | Pfizer | cryptococcal meningitis, |
| | | candidiasis |
| Pastille | Squibb Corp. | prevention of oral candidiasis |
| Nystatin Pastille | | |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | antibacterial |
| Trimethoprim/sulfa | | antibacterial |
| Piritrexim | Burroughs Wellcome | PCP treatment |
| Pentamidine | Fisons Corporation | PCP prophylaxis |
| isethionate for | | |
| inhalation | | |
| Spiramycin | Rhone-Poulenc | cryptosporidia diarrhea |
| Intraconazole- | Janssen Pharm. | histoplasmosis; cryptococcal |
| R51211 | | meningitis |
| Trimetrexate | Warner-Lambert | PCP |

OTHER

| | | |
|---|---|---|
| Daunorubicin | NeXstar, Sequus | Karposi's sarcoma |
| Recombinant Human | Ortho Pharm. Corp. | severe anemia assoc. with |
| Erythropoietin | | AZT therapy |
| Recombinant Human | Serono | AIDS-related wasting, |
| Growth Hormone | | cachexia |
| Leukotriene B4 Receptor | — | HIV infection |
| Antagonist | | |
| Megestrol Acetate | Bristol-Myers Squibb | treatment of anorexia assoc. |
| | | w/AIDS |
| Soluble CD4 Protein and | — | HIV infection |
| Derivatives | | |
| Testosterone | Alza, Smith Kline | AIDS-related wasting |
| Total Enteral | Norwich Eaton | diarrhea and malabsorption, |
| Nutrition | Pharmaceuticals | related to AIDS |

It will be understood that the scope of combinations of the compounds of this invention with HIV/AIDS antivirals, immunomodulators, anti-infectives or vaccines is not limited to the list in the above Table, but includes in principle any combination with any pharmaceutical composition useful for the treatment of HIV infection or AIDS. When employed in combination with the compounds of the invention, the HIV/AIDS antivirals and other agents are typically employed in their conventional dosage ranges and regimens as reported in the art, including the dosages described in the Physicians'Desk Reference, 54^{th} edition, Medical Economics Company, 2000. The dosage ranges for a compound of the invention in these combinations are the same as those set forth above just before the above Table.

Preferred combinations are simultaneous or alternating treatments with a compound of the present invention and an inhibitor of HIV protease and/or a non-nucleoside inhibitor of HIV reverse transcriptase. An optional fourth component in the combination is a nucleoside inhibitor of HIV reverse transcriptase, such as AZT, 3TC, ddC or ddI. Preferred agents for combination therapy include: Zidovudine, Lamivudine, Stavudine, Efavirenz, Ritonavir, Nelfinavir, Abacavir, Indinavir, 141-W94 (4-amino-N-((2 syn,3S)-2-hydroxy-4-phenyl-3-((S)-tetrahydrofuran-3-yloxycarbonylamino)-butyl)-N-isobutyl-benzenesulfonamide), N-(2(R)-hydroxy-1(S)-indanyl)-2(R)-phenylmethyl-4-(S)-hydroxy-5-(1-(4-(2-benzo[b]furanylmethyl)-2(S)-N′(t-butylcarbox-amido)-piperazinyl))-pentaneamide, and Delavirdine. A preferred inhibitor of HIV protease is indinavir, which is the sulfate salt of N-(2(R)-hydroxy-1(S)-indanyl)-2(R)-phenylmethyl-4-(S)-hydroxy-5-(1-(4-(3-pyridyl-methyl)-2(S)-N′-(t-butylcarbo-xamido)-piperazinyl))-pentane-amide ethanolate, and is synthesized according to U.S. Pat. No. 5,413,999. Indinavir is generally administered at a dosage of 800 mg three times a day. Other preferred inhibitors of HIV protease include nelfinavir and ritonavir. Preferred non-nucleoside inhibitors of HIV reverse transcriptase include (−) 6-chloro-4(S)-cyclopropylethynyl-4(S)-trifluoromethyl-1,4-dihydro-2H-3,1-benzoxazin-2-one, which may be prepared by methods disclosed in EP 0,582,455. The preparation of ddC, ddI and AZT are also described in EPO 0,484,071. These combinations may have unexpected effects on limiting the spread and degree of infection of HIV. Preferred combinations with the compounds of the present invention include the following: (1) Zidovudine and Lamivudine; (2) Stavudine and Lamivudine; (3) Efavirenz; (4) Ritoavir; (5) Nelfinavir; (6) Abacavir; (7) Indinavir; (8) 141-W94; and (9) Delavirdine. Preferred combinations with the compounds of the present invention further include the following (1) indinavir, with efavirenz or (−) 6-chloro-4(S)-cyclopropylethynyl-4(S)-trifluoromethyl-1,4-dihydro-2H-3,1-benzoxazin-2-one, and, optionally, AZT and/or 3TC and/or ddI and/or ddC; (2) indinavir, and any of AZT and/or ddI and/or ddC.

Compound A in the foregoing Table is N-(2(R)-hydroxy-1(S)-indanyl)-2(R)-phenylmethyl-4(S)-hydroxy-5-(1-(4-(2-benzo[b]furanylmethyl)-2(S)-N′-(t-butylcarboxamido)-piperazinyl))pentaneamide, preferably administered as the sulfate salt. Compound A can be prepared as described in U.S. Pat. No. 5,646,148.

In such combinations the compound of the present invention and other active agents may be administered separately or in conjunction. In addition, the administration of one element may be prior to, concurrent to, or subsequent to the administration of other agent(s).

The compounds of the present invention may be administered in the form of pharmaceutically acceptable salts. The term “pharmaceutically acceptable salt” is intended to include all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. Depending on the particular functionality of the compound of the present invention, pharmaceutically acceptable salts of the compounds of this invention include those formed from cations such as sodium, potassium, aluminum, calcium, lithium, magnesium, zinc, and from bases such as ammonia, ethylenediamine, N-methyl-glutamine, lysine, arginine, omithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide. These salts may be prepared by standard procedures, e.g. by reacting a free acid with a suitable organic or inorganic base. Where a basic group is present, such as amino, an acidic salt, i.e. hydrochloride, hydrobromide, acetate, pamoate, and the like, can be used as the dosage form.

Also, in the case of an acid (—COOH) or alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

The compounds of the present invention may be administered by oral, parenteral (e.g., intramuscular, intraperitoneal, intravenous, ICV, intracistemal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.) The pharmaceutical composition and method of the present invention may further comprise other therapeutically active compounds as noted herein which are usually applied in the treatment of the above mentioned pathological conditions.

In the treatment or prevention of conditions which require chemokine receptor modulation an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Abbreviations used in the instant specification, particularly the Schemes and Examples, include the following:

Ac=acetyl

Bn=benzyl

BOC or Boc=t-butyloxycarbonyl

Bu=butyl

t-Bu=tert-butyl

n-BuLi=n-butyl lithium

CBZ=carbobenzoxy (alternatively, benzyloxycarbonyl)

mCPBA=m-chloroperoxybenzoic acid

DAST=(diethylamino)sulfur trifluoride

DIBAL=diisobutylaluminum hydride

DIEA or DIPEA=diisopropylethylamine

DME=1,2-dimethoxyethane

DMF=N,N-dimethylformamide

DMSO=dimethylsulfoxide

Et=ethyl

ether=diethyl ether

h=hour(s)

HMDS=hexamethyldisilazyl

HMPA=hexamethylphosphoramide

HOBT or HOBt=1-hydroxy benzotriazole hydrate

LDA=lithium diisopropylamide

LHNMS or LiHMDS=lithium hexamethyldisilazide

Me=methyl

min=minute(s)

Ph=phenyl

Pr=propyl

i-Pr=isopropyl

PMB=p-methoxybenzyl

rt=room temperature

sat'd=saturated aqueous

TBSO=t-butyldimethylsiloxy

TEA=triethylamine

TFA=trifluoroacetic acid

The compounds of the present invention can be readily prepared according to the following reaction schemes and examples, or modifications thereof. Starting materials can be made from procedures known in the art or as illustrated. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction ED; schemes and examples. Unless otherwise indicated, the variables are as defined above.

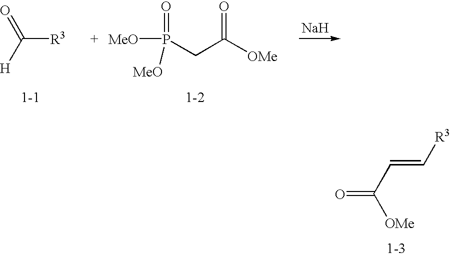

The preparation of cinnamate esters such as 1-3 (wherein R³ =an aromatic group) as intermediates that can be used for the synthesis of compounds within the scope of the instant invention is detailed in Scheme 1. Cinnamate esters of structure 1-3 can be obtained commercially or can be synthesized by reacting a suitable aromatic aldehyde 1-1 with a phosphonoacetate such as 1-2 or a stabilized Wittig reagent in the presence of sodium hydride or other bases such as sodium, lithium or potassium hexamethyldisilazide, potassium t-butoxide, and the like. The aldehyde 1-1 can be obtained commercially or can be prepared in a variety of ways from commercial materials (see March J. “Advanced Organic Chemistry”, 4th ed., John Wiley & Sons, New York, pp. 1270-1271 (1992)).

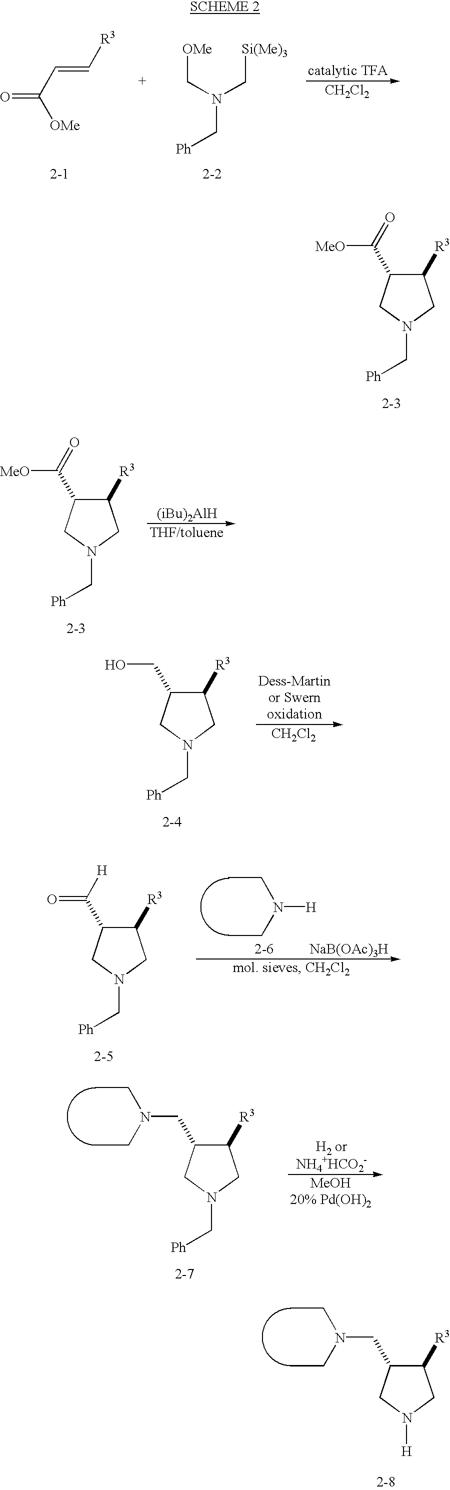

The preparation of compounds within the scope of the instant invention which bear a 1,3,4-trisubstituted pyrrolidine framework is detailed in Scheme 2. Treatment of a trans-cinnamic ester such as 2-1 with commercially available N-benzyl-N-methoxymethyl-N-(trimethylsilyl)-methylamine (2-2) in the presence of a substoichiometric amount of an acid such as TFA, titanium tetrafluoride, lithium fluoride or cesium fluoride, according to the procedure of Padwa et al (J. Org. Chem. 1987, 52, 235) preferentially affords the 3,4-trans pyrrolidine 2-3. Executing this sequence starting from the cis-cinnamic ester results in preferential formation of the 3,4-cis pyrrolidine. Reduction of ester 2-3, for example, with diisobutylaluminum hydride, lithium aluminium hydride, or sodium bis(2-methoxyethoxy)aluminum hydride, provides the primary alcohol 2-4. Oxidation to the aldehyde 2-5 can be carried out under numerous conditions, such as with the Dess-Martin periodinane, with DMSO and oxalyl chloride at low temperature, followed by triethylamine (Swern oxidation), or with various chromium trioxide-based reagents (see March J. “Advanced Organic Chemistry”, 4th ed., John Wiley & Sons, New York, pp. 1167-1171 (1992)). Reductive amination with cyclic amine 2-6 then provides diamine 2-7, which can itself be a chemokine receptor modulator. Alternatively, the N-benzyl group is cleaved in a hydrogen atmosphere or with ammonium formate in the presence of 20% palladium hydroxide to provide the secondary amine 2-8.

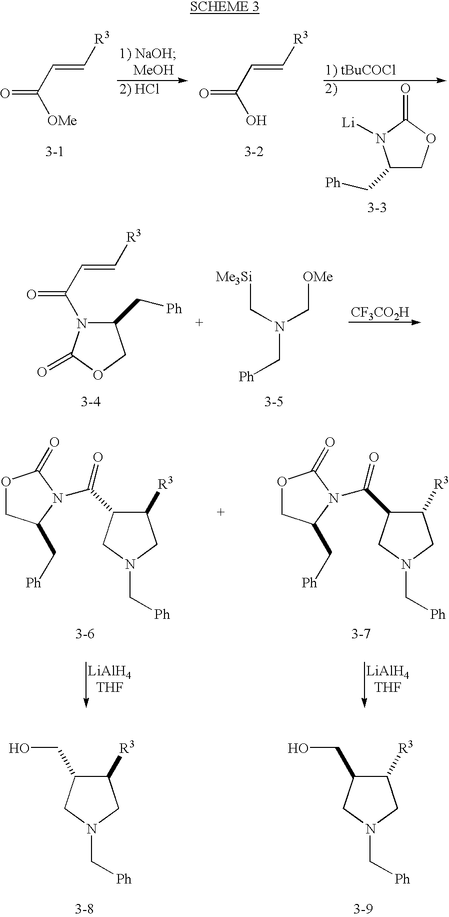

Scheme 3 shows the preparation of optically pure pyrrolidine intermediates. Hydrolysis of unsaturated ester 3-1 provided acid 3-2, which is converted to diacyl derivative 3-4 by activation of the acid group, for example by formation of a mixed anhydride with pivaloyl chloride, followed by reaction with the lithium salt of 4-(S)-benzyloxazolidin-2-one (3-3). Treatment of 3-4 with commercially available N-benzyl-N-methoxymethyl-N-(trimethylsilyl)-methylamine (2-2) in the presence of a substoichiometric amount of an acid such as TFA, titanium tetrafluoride, lithium fluoride or cesium fluoride according to the procedure of Padwa et al (J. Org. Chem. 1987, 52, 235) affords the diastereomeric pyrrolidines 3-6 and 3-7, which can be separated by flash chromatography, preparative thin layer chromatography, medium pressure liquid chromatography, high pressure liquid chromatography, fractional crystallization, or similar methods known in the art. The separated products are then individually reduced, for example with lithium alumium hydride (LAH) or other strong hydride reducing agents, to provide pyrrolidines 3-8 and 3-9 in optically enriched form.

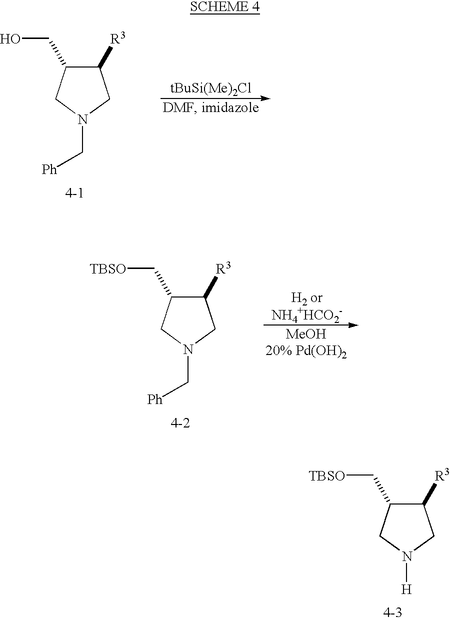

Preparation of a protected pyrrolidine for use as an intermediate in the synthesis of compounds in the instant invention is shown in Scheme 4. The pyrrolidine 4-1 (prepared as shown in Schemes 2 and 3) is protected with a suitable protecting group such as t-butyl-dimethylsilyl to provide silyl ether 4-2. Other silyl groups can also be used in this role, as can other protecting groups for a hydroxy residue (see Greene, T. W.; Wuts, P. G. M. “Protective Groups in Organic Synthesis”, 2nd edition, Wiley-Interscience, New York, pp. 10-143 (1991)), subject to the group being stable to conditions used to remove the benzyl group and being removable under conditions that would not adversely affect the remainder of the molecule. Removal of the benzyl group on nitrogen is then carried out by hydrogenolysis, for example by transfer hydrogenation with ammonium formate in the presence of 20% palladium hydroxide or with catalytic hydrogenation with 10% palladium on carbon under one or more atmospheres of hydrogen. Alternatively, compound 4-1 can be debenzylated first under the conditions noted above and then silylated on the hydroxy group, to provide 4-3.

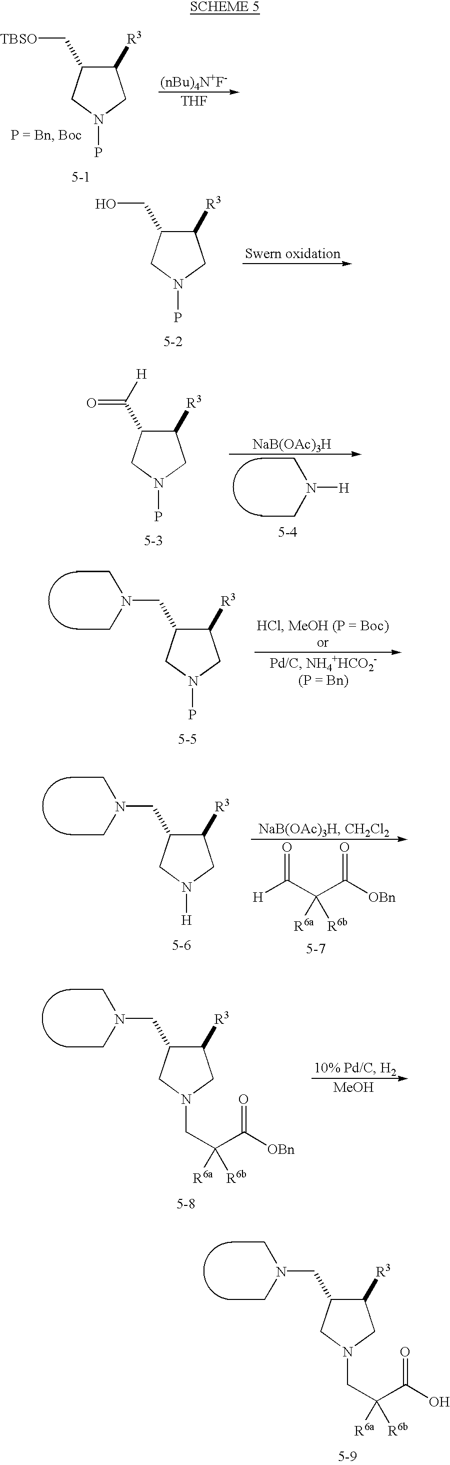

One method of preparing compounds within the scope of the instant invention is given in Scheme 5. Doubly protected pyrrolidine 5-1 (obtained either as shown in Scheme 4 for 4-2 when P=benzyl or by protection of 4-3 with Boc anhydride in THF/water in the presence of triethylamine when P=Boc) is desilylated with tetrabutylammonium fluoride in THF to provide alcohol 5-2. Oxidation of 5-2 to 5-3 is carried out using Swern's oxidation conditions. Other methods for oxidizing a primary hydroxy group to an aldehyde can also be used, for example the Dess-Martin periodinane, or with various chromium trioxide-based reagents (see March J. “Advanced Organic Chemistry”, 4th ed., John Wiley & Sons, New York, pp. 1167-1171 (1992)). Reductive amination with cyclic amine 5-4 then provides diamine 5-5. Deprotection of the pyrrolidine nitrogen, when P=Boc, can be carried out with HCl in methanol or with trifluoroacetic acid and anisole in dichloromethane, to give secondary amine 5-6. When P=benzyl, debenzylation is carried out in the presence of palladium on carbon as a catalyst, using either hydrogen gas or ammonium formate to effect transfer hydrogenation. Reductive amination with formyl ester 5-7 then provides pyrrolidine 5-8. Removal of the benzyl group can be carried out under standard reductive conditions, for example, hydrogen gas in the presence of a supported or unsupported palladium catalyst, to afford acid 5-9. Alternatively, if a 4-methoxybenzyl ester is utilized in place of the benzyl ester of compound 5-7, then the final deprotection can be carried out under acid conditions, for example, formic acid at 55° C. This latter approach is useful if the parent molecule contains functionality sensitive to catalytic hydrogenation.

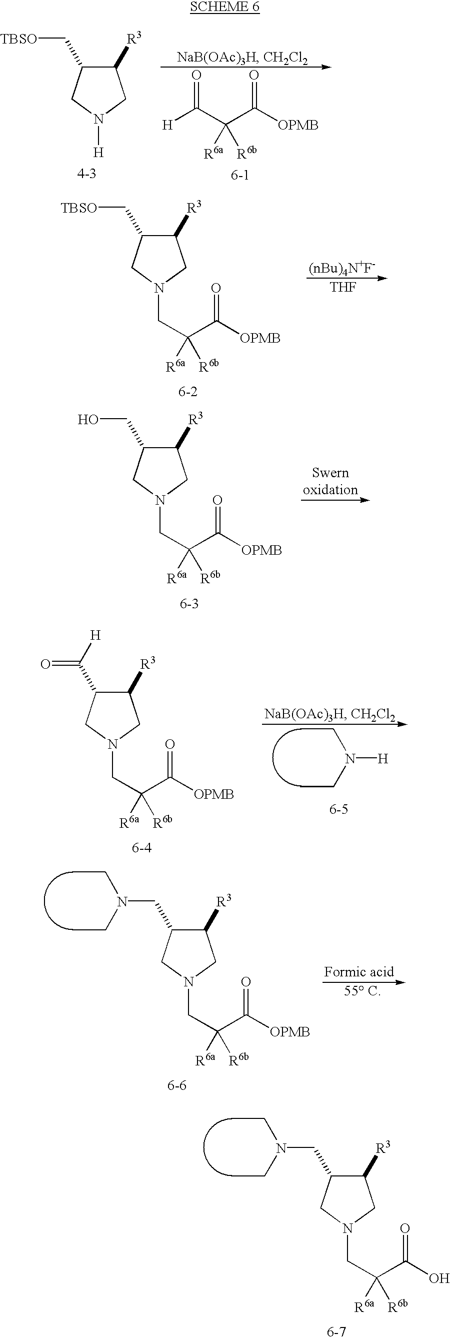

Another method for preparing compounds in the instant invention is shown in Scheme 6. Reductive amination of pyrrolidine 4-3 with aldehyde ester 6-1 affords pyrrolidine 6-2. Removal of the silyl protecting group with tetrabutylammonium fluoride provides alcohol 6-3, which can be oxidized under standard conditions, for example the Swern oxidation, to give aldehyde 6-4. Reductive amination of 6-4 with a suitable secondary amine 6-5 yields ester 6-6 which can be deprotected under acidic conditions, for example, with formic acid, to afford compound 6-7.

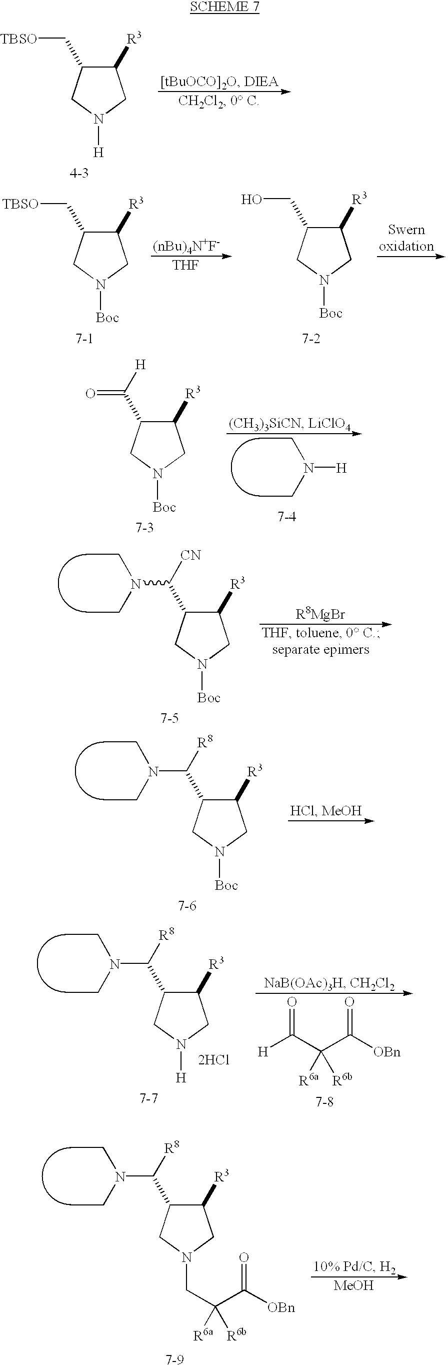

A method for preparing compounds in the instant invention wherein an additional substituent R⁸ is present is given in Scheme 7. Protection of pyrrolidine 4-3 with Boc anhydride under standard conditions provides doubly protected pyrrolidine 7-1, which can be desilylated by exposure to tetrabutylammonium fluoride in TBE, affording 7-2. Oxidation of 7-2 to aldehyde 7-3 is carried out using Swem's oxidation conditions. Other methods for oxidizing a primary hydroxy group to an aldehyde can also be used, for example the Dess-Martin periodinane, or with various chromium trioxide-based reagents (see March J. “Advanced Organic Chemistry”, 4th ed., John Wiley & Sons, New York, pp. 1167-1171 (1992)). Treatment of 7-3 with a secondary amine 7-4, and trimethylsilyl cyanide in the presence of lithium perchlorate affords cyanoamine 7-5. Treatment of 7-5 with a suitable organomagnesium reagent R⁸MgBr yields the branched compound 7-6. The diastereomers formed in this process can be separated at this stage, or at any point later in the synthesis by standard methods, including fractional crystallization, column chromatography, flash chromatography, high pressure liquid chromatograghy (HPLC) or medium pressure liquid chromatography (MPLC), optionally by use of a stationary phase derivatized with chiral, non-racemic groups to enable separation of enantiomers and to enhance separation of isomeric mixtures. The Boc group of 7-6 can be removed under acidic conditions, for example hydrochloric acid in methanol, to afford secondary pyrrolidine 7-7. Reductive amination with aldehyde 7-8 under mild conditions, for example with sodium cyanoborohydride in methylene chloride, provides ester 7-9. Removal of the benzyl protecting group by catalytic reduction then affords compound 7-10.

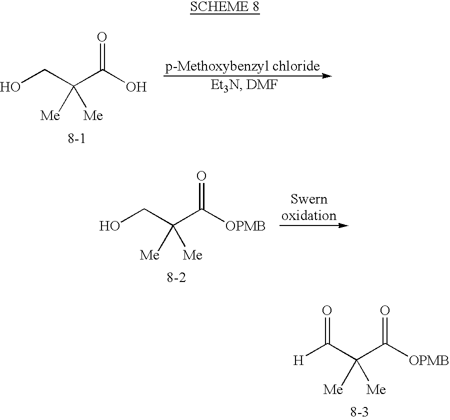

Synthesis of aldehyde esters such as 6-1 and 7-8 can be carried out by a number of routes, one of which is shown in Scheme 8. The available hydroxy acid 8-1 is esterified with a suitable protecting group (such as a para-methoxybenzyl group) in the presence of a suitable base (such as triethylamine or DIEA), to give ester 8-2. Oxidation of 8-2, for example by Swern oxidation, then affords aldehyde 8-3.

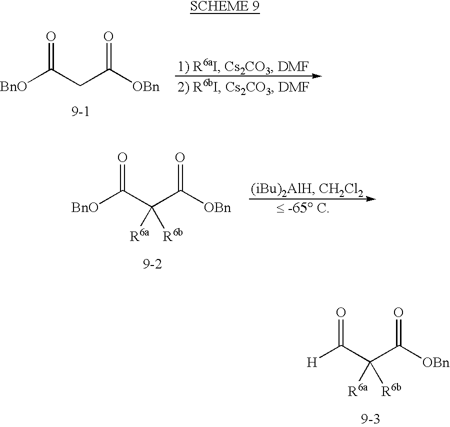

Synthesis of aldehyde ester 6-1 where the R⁶ substituents are distinct or identical can be accomplished as shown in Scheme 9. Alkylation of dibenzyl malonate with a suitable alkylating agent, such as an alkyl iodide, bromide, toluenesufonate and the like, in the presence of a base such as cesium carbonate, potassium carbonate, or other agents of moderate basicity, followed by repetition of the procedure with a second alkyl halide or alkyl toluenesulfonate, provides dialkylated product 9-2. Treatment of diester 9-2 with di-isobutylaluminum hydride at low temperature affords ester aldehyde 9-3.

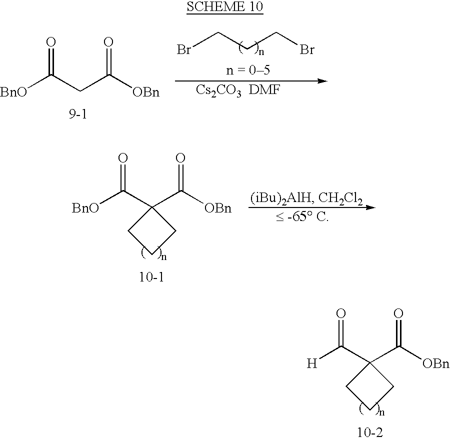

Synthesis of aldehyde ester 6-1 where the R^{6a} and R^{6b} form a ring substituents can be accomplished as shown in Scheme 10. Dialkylation of dibenzyl malonate with a suitable dialkylating agent, such as an alkyl diiodide, dibromide, ditoluenesufonate and the like, in the presence of a base such as cesium carbonate, potassium carbonate, or other agents of moderate basicity, provides cyclic derivative 10-1. Treatment of diester 10-1 with di-isobutylaluminum hydride at low temperature affords ester aldehyde 10-2. An analogous scheme can be used for cyclic derivatives containing a heteroatom in the ring by selection of the appropriate precursor.

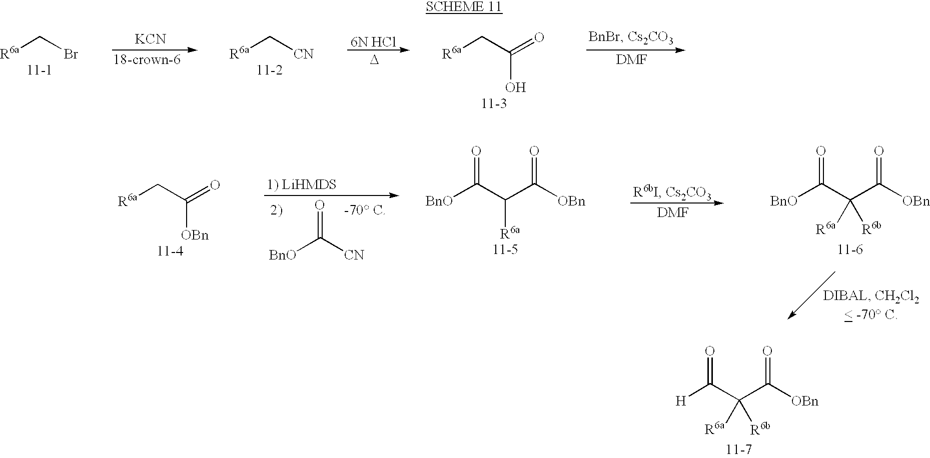

An alternative synthesis of aldehyde ester intermediates is given in Scheme 11. Treatment of a commercially available alkyl methyl bromide with potassium cyanide in the presence of 18-crown-6 provides nitrile 11-2. Hydrolysis under acidic conditions affords acid 11-3. Esterification with benzyl bromide in the presence of cesium carbonate in DMF yields ester 11-4. Deprotonation of ester 11-4 with a strong, non-nucleophilic base, such as lithium hexamethyldisilazide, followed by treatment with benzyl cyanoformate, provides diester 11-5, which can be alkylated with a suitably activated haloalkyl group to provide dialkylated product 11-6. Reduction with DIBAL at low temperature then provides the desired intermediate 11-7.

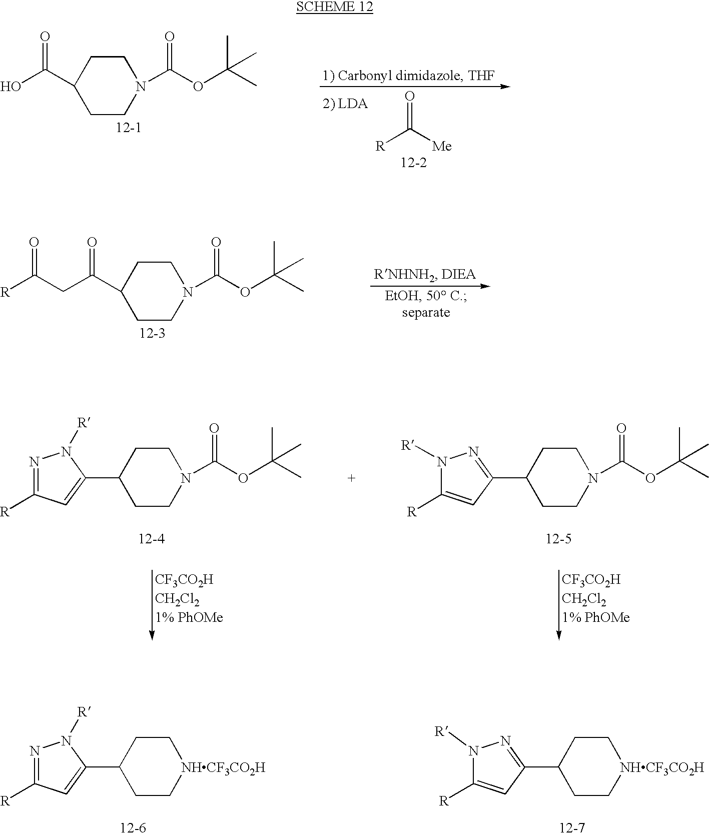

One preparation of piperidine subunits containing functionalized pyrazoles at C4 of the piperidine is given in Scheme 12. Treatment of piperidine 12-1 with carbonyldiimidazole to form the acylimidazole, followed by addition of a dialkyl or alkyl-aryl ketone 12-3. (12-2) in the presence of lithium diisopropylamide (LDA) gives the diketone 12-3. Treatment with a monoalkyhydrazine in an alcohol solvent at temperatures between 0 to 100 degrees C. (preferably about 50 degrees C.) optionally in the presence of a hindered base such as DIEA then provides a mixture of the isomeric pyrazoles 12-4 and 12-5. After separation of these compounds by chromatography or crystallization, the individual products are deblocked under acidic conditions (for example trifluoroacetic acid and anisole with or without methylene chloride as a cosolvent) to provide the piperidine salts 12-6 and 12-7, which are then used as the cyclic secondary amine component as shown above in Schemes 5, 6, and 7.

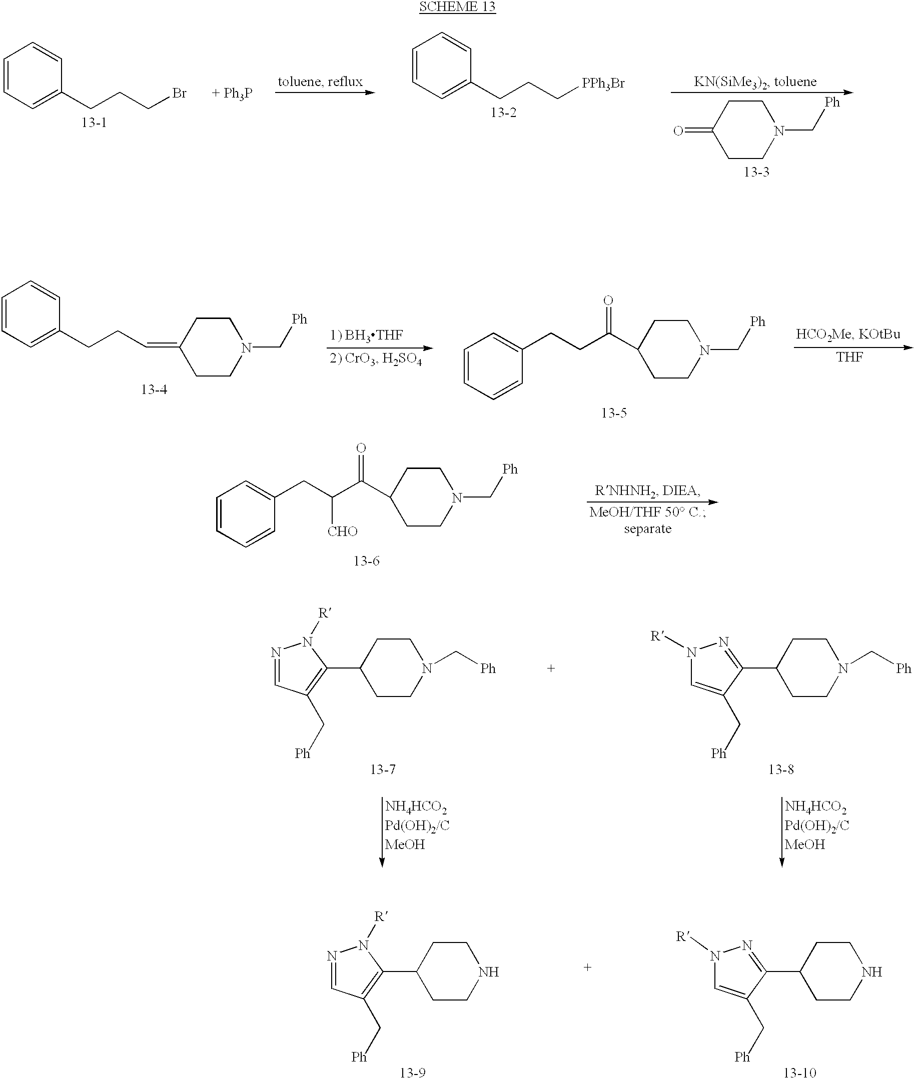

Another preparation of piperidine subunits containing functionalized pyrazoles at C4 of the piperidine is given in Scheme 13. Treatment of commercially available bromide 13-1 with triphenylphosphine in refluxing toluene provides phosphonium salt 13-2, which after treatment with a strong anhydrous base such as potassium hexamethyldisilazide in toluene and the piperidine ketone 13-3 provides the olefin 13-4. Hydroboration followed by an oxidative workup with chromic acid then affords ketone 13-5. Selective formylation of 13-5 with methyl formate in the presence of potassium t-butoxide affords ketoaldehyde 13-6. Heating of 13-6 with a monoalkylhydrazine in methanol optionally in the presence of a hindered (or insoluble) base such as DIEA then provides a mixture of the 1,5-disubstituted pyrazoles 13-7 and 13-8. After separation by chromatography, crystallization or fractional distillation, the purified isomers are deprotected under transfer hydrogenation conditions to provide the piperidines 13-9 and 13-10, which are then used as the cyclic secondary amine component as shown above in Schemes 5, 6, and 7.

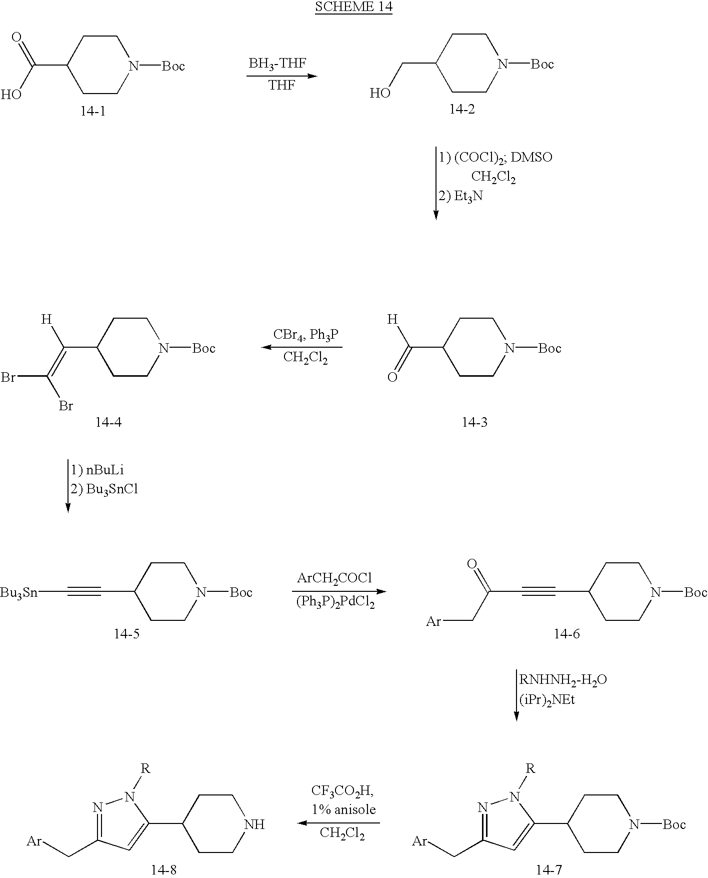

An alternate preparation of piperidine subunits containing functionalized pyrazoles at C4 of the piperidine is given in Scheme 14. Treatment of commercially available isonipecotic acid under reducing conditions with borane-TBf complex provides primary alcohol 14-2. Oxidation under standard conditions, for example using Swern's conditions, yields aldehyde 14-3. Treatment of 14-3 with carbon tetrabromide in the presence of triphenylphosphine affords dibromo-olefin 14-4, which upon treatment with n-butyllithium followed by tributyl tin chloride provides stannyl acetylene 14-5. Coupling of 14-5 with an acid chloride ArCH₂COCl in the presence of a suitable palladium catalyst, such as dichlorobis(triphenylphosphine)palladium, in refluxing dichloromethane provided unsaturated ketone 14-6. Treatment of acetylenic ketone 14-6 with a mono-alkylhydrazine in a suitable solvent, such as ethanol, affords pyrazole 14-7. Deprotection of this compound under acidic conditions, for example with HCl in methanol or with trifluoroacetic acid in dichloromethane in the presence of anisole, provides the desired pyrazole derivative 14-8, which is then used as the cyclic secondary amine component as shown above in Schemes 5, 6, and 7.

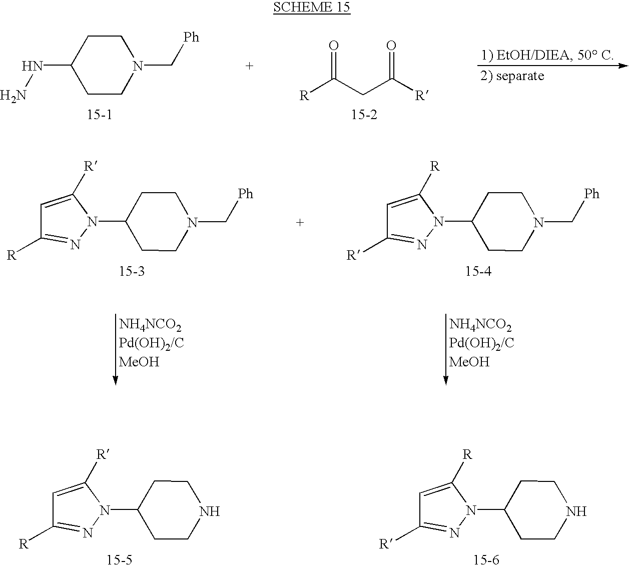

A preparation of piperidine subunits containing 3,5-difunctionalized pyrazoles linked through N1 to C4 of the piperidine is given in Scheme 15. Treatment of commercially available hydrazine 15-1 with diketone 15-2 in ethanol at 0 to 90 degrees C. (prefereably 50 degrees C.) in the presence of DIEA provides a mixture of pyrazoles 15-3 and 15-4, which are separated under standard conditions, for example HPLC. Removal of the benzyl groups by transfer hydrogenation provides the secondary piperidines 15-5 and 15-6, which are then used as the cyclic secondary amine component as shown above in Schemes 5, 6, and 7.

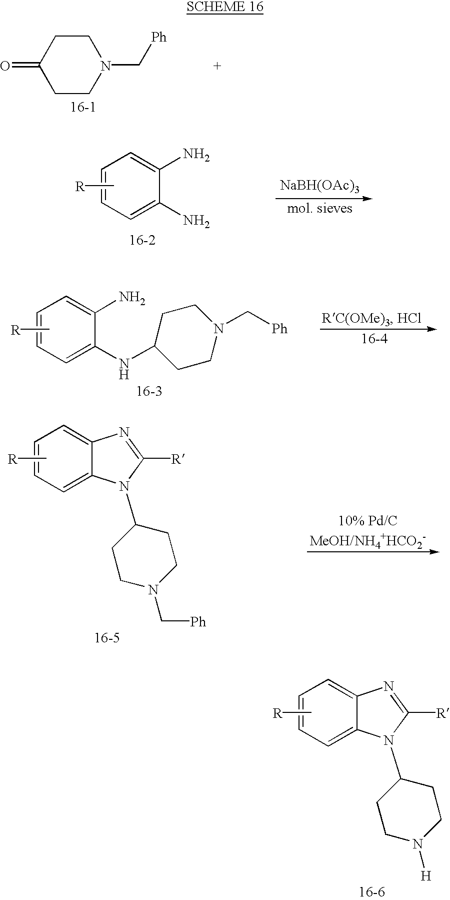

A preparation of 4-(benzimidazol-1-yl)piperidine subunits is given in Scheme 16. Combining piperidone 16-1 and diamine 16-2 in the presence of sodium triacetoxy borohydride under dehydrating conditions provides reductive amination product 16-3. Addition of a suitably substituted ortho ester 16-4 in the presence of a acid catalyst, for example concentrated hydrochloric acid, provides benzimidazole intermediate 16-5. Deprotection under reductive conditions, for example with palladium on carbon under transfer hydrogenation conditions, then provides secondary amine 16-6, which is then used as the cyclic secondary amine component as shown above in Schemes 5, 6, and 7.

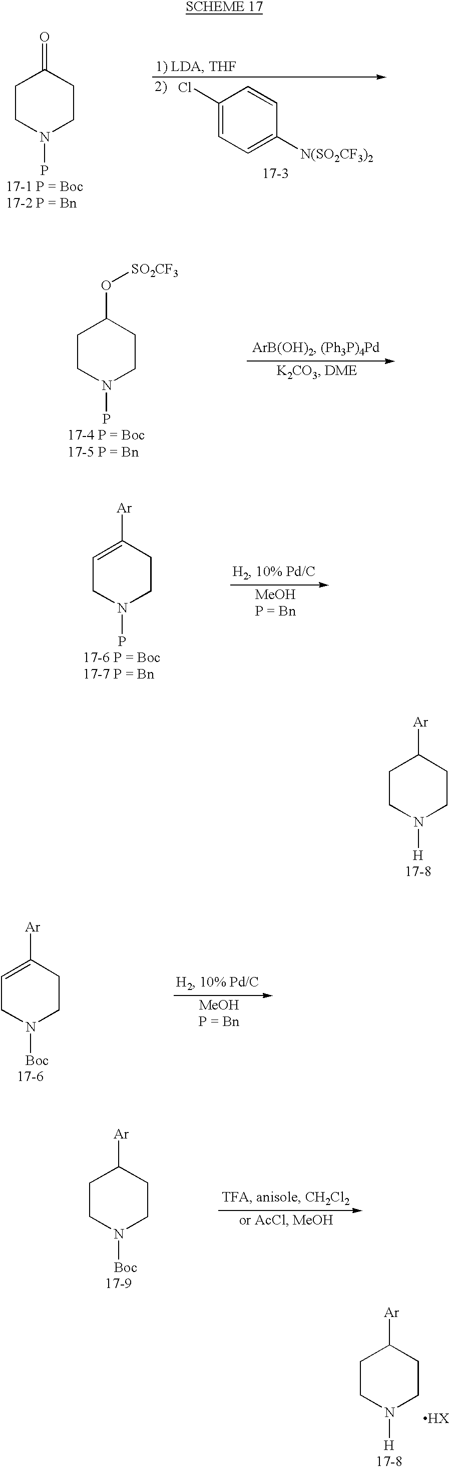

One method of generating 4-aryl piperidines as intermediates is given in Scheme 17. Reaction of commercially available 17-1 or 17-2 with a strong base, such as LDA, LiHDMS, NaHMDS, KHMDS, or NaH followed by treating with a suitable triflating agent, such as 5-chloropyrid-2-yl triflimide (17-3), N-phenyl triflimide or triflic anhydride, provides enol triflates 17-4 or 17-5. Heating with commercially available aryl boronic acids in the presence of a suitable palladium(0) catalyst such as tetrakis triphenylphosphine palladium, a base (such as potasssium carbonate or sodium carbonate), in a solvent such as DME, THF, dioxane or toluene/ethanol, effects coupling to provide the unsaturated products 17-6 or 17-7. In the case of 17-7, treatment with a heterogeneous palladium catalyst in methanol or ethanol in an atmosphere of hydrogen provides the desired intermediate 17-8. Alternatively, the Boc protected derivative 17-6 is hydrogenated under standard conditions to provided the saturated piperidine 17-9, which is then deprotected under acidic conditions (such as trifluoroacetic acid and anisole in methylene chloride), to provide 17-8 as a salt, which is then used as the cyclic secondary amine component as shown above in Schemes 5, 6, and 7.

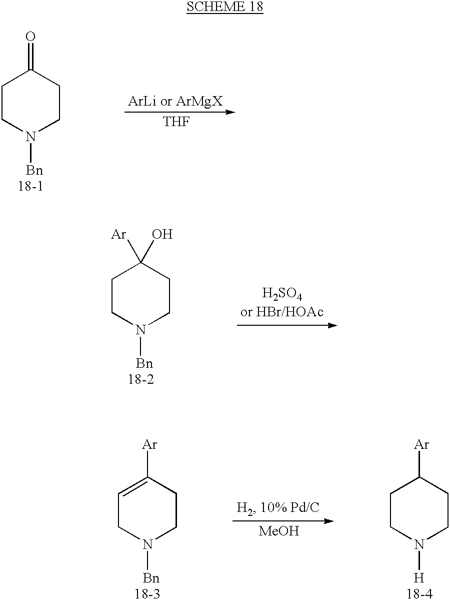

An alternative method of generating 4-aryl piperidines as intermediates is given in Scheme 18. Reaction of commercially available 18-1 with an aryl magnesium halide or with an aryllithium (in the presence or absence of anhydrous cerium trichloride) provides tertiary alcohol 18-2, which upon treatment under acidic conditions (such as sulfuric acid, HBr in acetic acid, HCl in acetic acid) or under dehydrating conditions (such as with thionyl chloride in pyridine or with phosphorus oxychloride) provides olefin 18-3. Hydrogenation under standard conditions using either hydrogen gas or a hydrogen donor (such as ammonium formate or cyclohexene) effects reduction of the double bond and cleavage of the N-benzyl group to provide the desired intermediate 18-4. Under some circumstances it may be preferable to reduce the double bond under non-hydrogenolytic conditions, for example with triethylsilane and trifluoroacetic acid or under dissolving metal conditions (for example, sodium or lithium metal in ammonia or a lower alkyl amine). If the N-benzyl group is not removed under these conditions, it may be cleaved by treatment with either vinyl chloroformate and then hydrogen chloride or by treatment with 2-chloroethyl chloroformate followed by heating in methanol. The product 18-4 is then used as the cyclic secondary amine component as shown above in Schemes 5, 6, and 7.

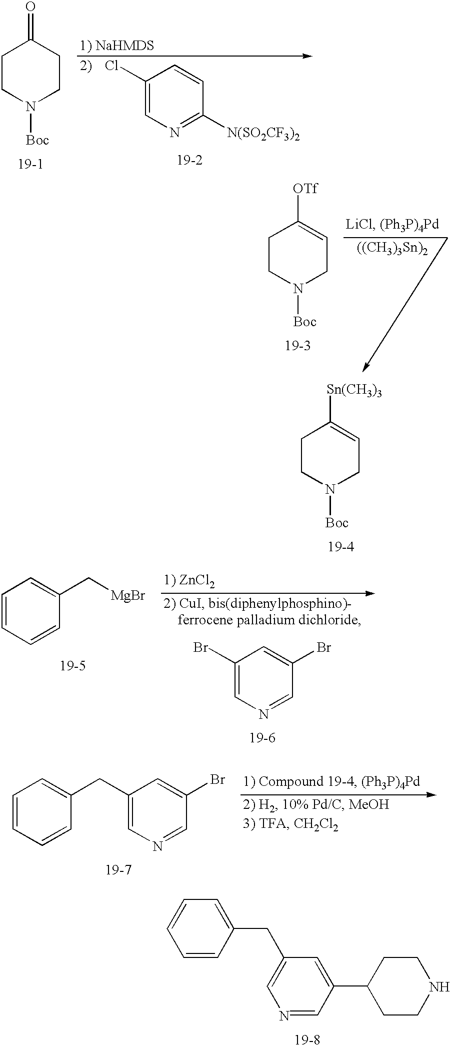

Piperidine intermediates bearing a pyridine substituent can be synthesized as shown in Scheme 19. Enolization of ketone 19-1 with a strong, non-nucleophilic base such as sodium hexamethyldisilazide, followed by treatment with a suitable triflating agent, such as 2-(N,N-bis(trifluoromethanesulfonyl)amino)-5-chloropyridine (19-2), provides vinyl triflate 19-3. Exchange of the triflate for a trimethylstannyl group is carried out under standard conditions to provide 19-4. Separately, treatment of benzyl magnesium chloride with zinc chloride, followed by treatment of the resulting material with 3,5-dibromopyridine, copper iodide and a suitable palladium catalyst, provides coupled product 19-7. Coupling of 19-4 with 19-7 in the presence of a soluble palladium catalyst, followed by hydrogenation of the double bond, and then cleavage of the Boc group under acidic conditions, then gives intermediate 19-8.

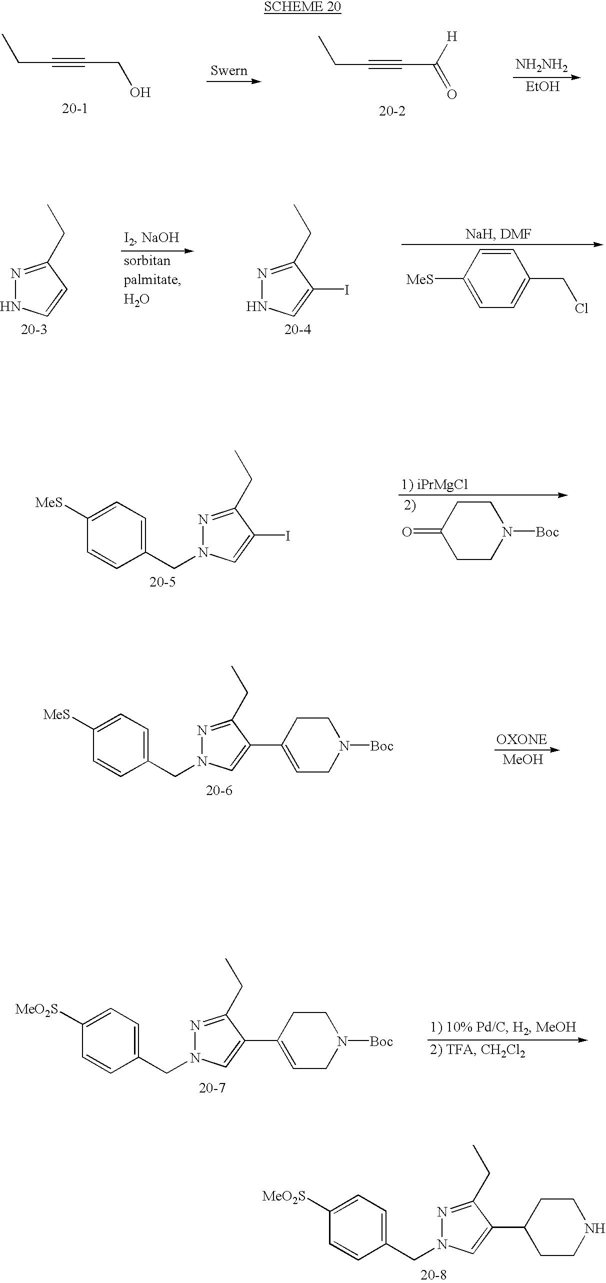

Piperidine intermediates bearing a functionalized pyrazole side chain can be prepared as shown in Scheme 20. Oxidation of 2-pentyl-1-ol under Swern conditions followed by treatment with hydrazine provides pyrazole 20-3. Iodination under phase transfer conditions affords iodopyrazole 20-4. Alkylation with 4-thiomethylbenzyl chloride yields pyrazole 20-5. Halogen-metal exchange with isopropyl magnesium chloride followed by addition of N-Boc-4-pyridone affords pyrazole 20-6, which on oxidation with Oxone® (potassium peroxymonosulfate) provides sulfone 20-7. Hydrogenation and then treatment with trifluoroacetic acid in methylene chloride then affords intermediate piperidine 20-8.

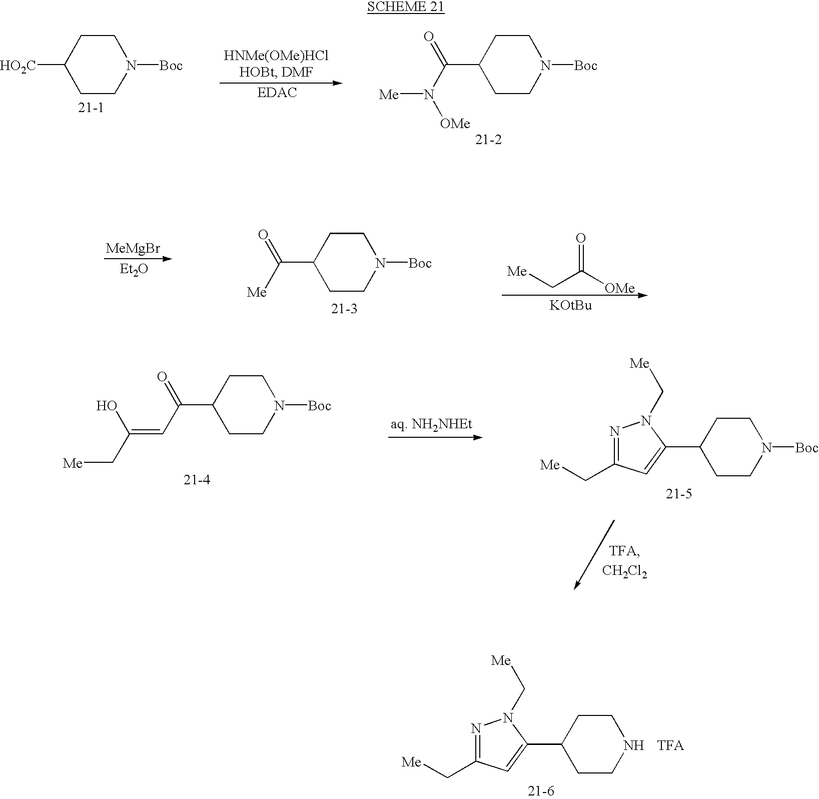

Piperidine intermediates with alkylpyrazole substituents can be prepared as shown in Scheme 21. Treatment of N-Boc-4-carboxypiperidine with EDAC, HOBt and N,O-dimethylhydroxylamine hydrochloride affords amide 21-2, which upon exposure to methyl magnesium bromide provides ketone 21-3. Condensation of 21-3 with methyl propionate in the presence of potassium tert-butoxide provides diketone 21-4, which affords pyrazole 21-5 after treatment with aqueous ethylhydrazine. Deprotection under acidic conditions, for example with trifluoroacetic acid in methylene chloride, then provides intermediate 21-6.

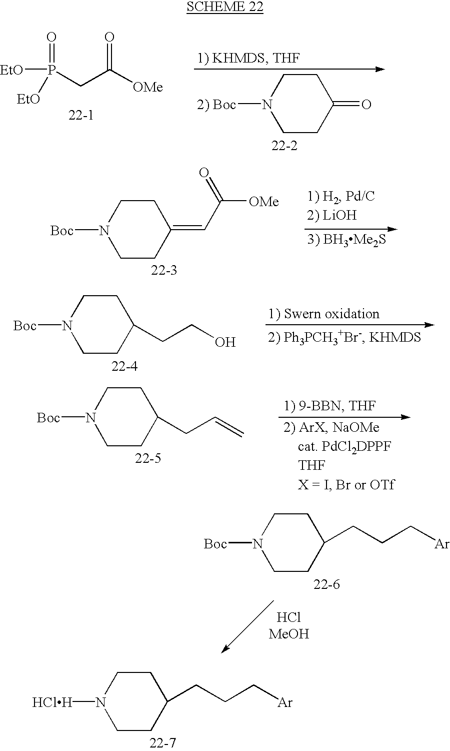

A route for the preparation of 4-(3-arylpropyl)piperidines is given in Scheme 22. Treatment of phosphonoacetate 22-1 with KHMDS followed by addition of commercially available N-Boc -4-piperidone 22-2 provides unsaturated ester 22-3. Hydrogenation of 22-3 followed by hydrolysis to the acid and then reduction with borane-methyl sulfide then affords primary alcohol 22-4. Mild oxidation of 22-4 under Swern conditions provides the corresponding aldehyde, which upon treatment with the Wittig reagent prepared from methyltriphenylphosphonium iodide and r KHMDS yields olefin 22-5. Hydroboration with a dialkylborane, such as 9-borabicyclo[3.3.1]nonane (9-BBN), followed by treatment with an aryl halide (the halides preferably being bromide or iodide) or aryl triflate in the presence of a suitable soluble palladium catalyst, for example Pd(dppf)Cl₂, in warm to refluxing THF, provides the 3-arylpropyl derivative 14-6. Removal of the Boc group under acidic conditions, for example with HCl in methanol or with trifluoroacetic acid in methylene chloride, then affords the 1-unsubstituted piperidine 22-7, which can then be employed as the secondary amine component in the syntheses described above in Schemes 5, 6, and 7.

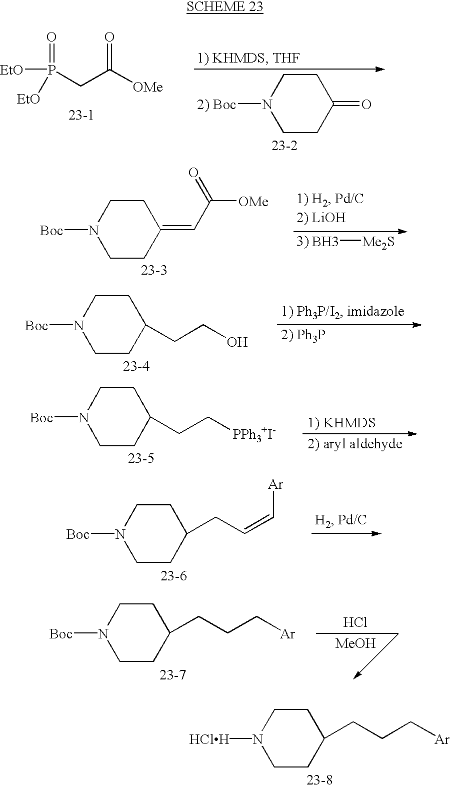

Another route for the preparation of 4-(3-arylpropyl)piperi dines is given in Scheme 23. Treatment of phosphonoacetate 23-1 with KMS followed by addition of commercially available N-Boc -4-piperidone 23-2 provides unsaturated ester 23-3. Hydrogenation of 23-3 followed by hydrolysis to the acid and then reduction with boranemethyl sulfide then affords primary alcohol 23-4. Formation of the alkyl iodide with triphenylphosphine and iodine in the presence of imidazole followed by treatment with triphenyiphosphine provides phosphonium salt 23-5. Deprotonation with a suitable base, for example, KIIS, LiIENMS, NaHMS, Nail, LDA, or KH affords the Wittig agent in situ, which upon treatment with a suitable aromatic aldehyde yields the unsaturated derivative 23-6. Hydrogenation under standard conditions provides 23-7, and removal of the Boc group with HCl in methanol or with other acidic conditions then provides the 1-unsubstituted piperidine 23-8, which can then be employed as the secondary amine component in the syntheses described above in Schemes 5,6 and 7.

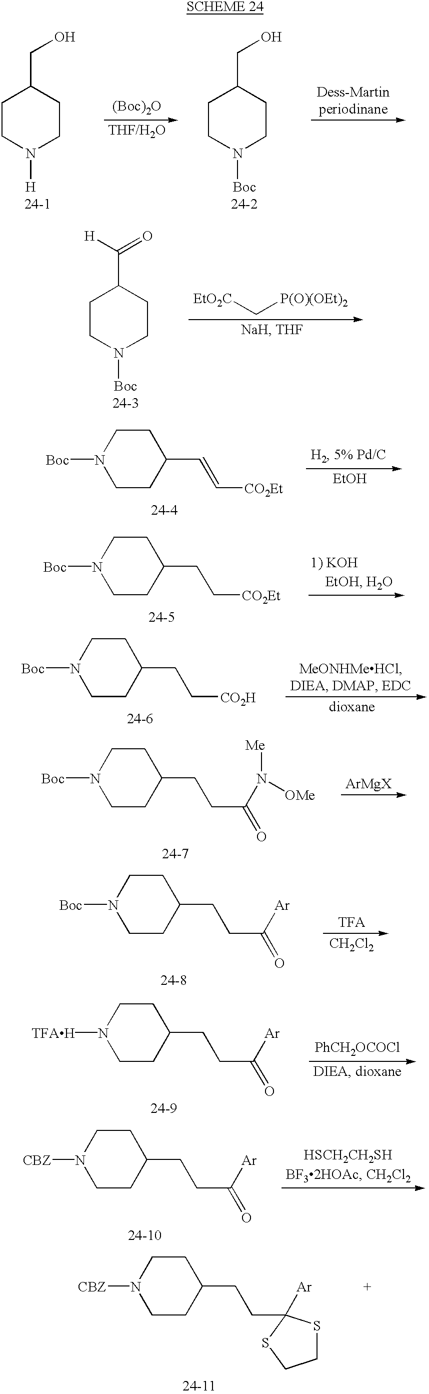

Preparation of piperidines with a 4-(3-aryl-3,3,-difluoropropyl) side chain is given in Scheme 24. Treatment of commercially available 24-1 with Boc anydride provides protected piperidine 24-2. Oxidation, for example with the Dess-Martin reagent, by a Swern oxidation, or other known methods provides aldehyde 24-3. Condensation under Homer-Wadsworth-Emmons conditions affords unsaturated ester 24-4, which is hydrogenated to ester 24-5 and then hydrolyzed to acid 24-6. Formation of the N-methyl-N-methoxy amide 24-7 is carried out employing standard activating agents such as EDC. Weinreb amide 24-7 is then allowed to react with an arylmetal reagent, such as an aryl magnesium halide or an aryllithium, to provide ketone 24-8. Cleavage of the protecting Boc group under acidic conditions yields 24-9, which is reprotected with a carbobenzyloxy group under standard conditions, to afford 24-10. Formation of dithiolane 24-11 with ethanedithiol and boron trifluoride is followed by treatment with 1,3-dibromo-3,3-dimethylhydantoin and pyridine-hydrogen fluoride complex at or around −78 degrees C., to provide gem-difluoro derivative 24-12. Removal of the CBZ group under reductive conditions provides piperidine 24-13, which may be employed directly as the secondary amine in chemistry described above. Alternatively, if additional purification is desired, 24-13 may be protected with a Boc group to afford 24-14. After suitable purification, the Boc group is removed under acidic conditions at or near 0 degrees C. A controlled, basic workup then provides 24-15, suitable for use as described above.

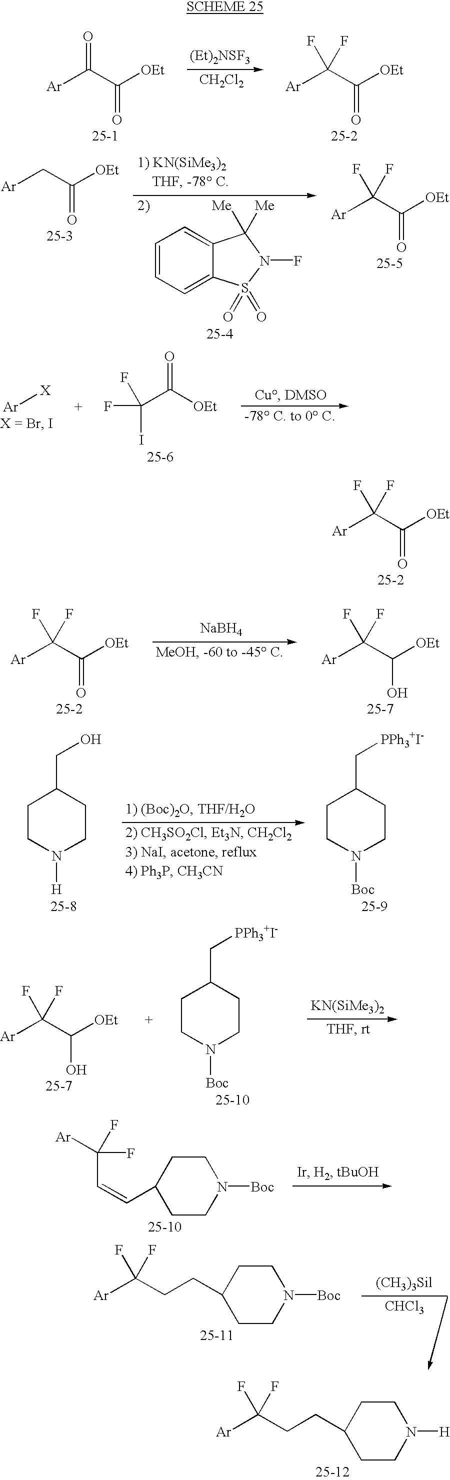

An alternate preparation of piperidines with a 4-(3-aryl-3,3,-difluoropropyl) side chain is given in Scheme 25. Preparation of the intermediate 25-2 can be accomplished in three ways. First, ketoester 25-1 can be fluorinated with diethylaminosulfur trifluoride ODAST) under standard conditions to provide (X(X difluoroester 25-2. Second, arylacetic ester 25-3 can be fluorinated by treatment with a strong base, such as potassium hexamethyldisilazide, followed by addition of a suitable fluorinating agent, such as the N-fluoro reagent 25-4, to give 25-2. Alternatively, an aryl iodide or aryl bromide 25-5 can be treated with ethyl α,α-difluoro-α-iodoacetate (25-6) in the presence of copper metal to provide 25-2. Treatment of ester 25-2 with sodium borohychide at low temperature then provides key intermediate 25-7. Preparation of intermediate 25-9 is carried out by first protecting commercially available 4-(hydroxymethyl)piperidine as the N-Boc derivative, then forming the methanesulfonyl ester under standard conditions, displacing the mesylate group with an iodide, and finally treating the iodide with triphenylphosphine. Coupling of 25-7 with phosphonium salt 25-9 in the presence of a strong base, such as potassium hexamethyldisilazide, sodium hydride, lithium diisopropylamide, or similar reagents, affords olefin 25-10. Reduction of the double bond of 25-10 is effected by treatment with iridium metal in t-butanol or hexane under an atmosphere of hydrogen, to give 25-11. Alternatively, reduction using palladium on carbon, platinum or Raney nickel in the presence of hydrogen can be used, as can diimide, which can be generated from azodicarboxylic acid in situ. The nitrogen protecting group is removed by treatment with trimethylsilyl iodide under anhydrous conditions, to afford piperidine 25-12, which is suitable for use as described above. Alternatively, the Boc group can be removed under acidic, anhydrous conditions, for example with TFA in methylene chloride or with HCl in methanol.

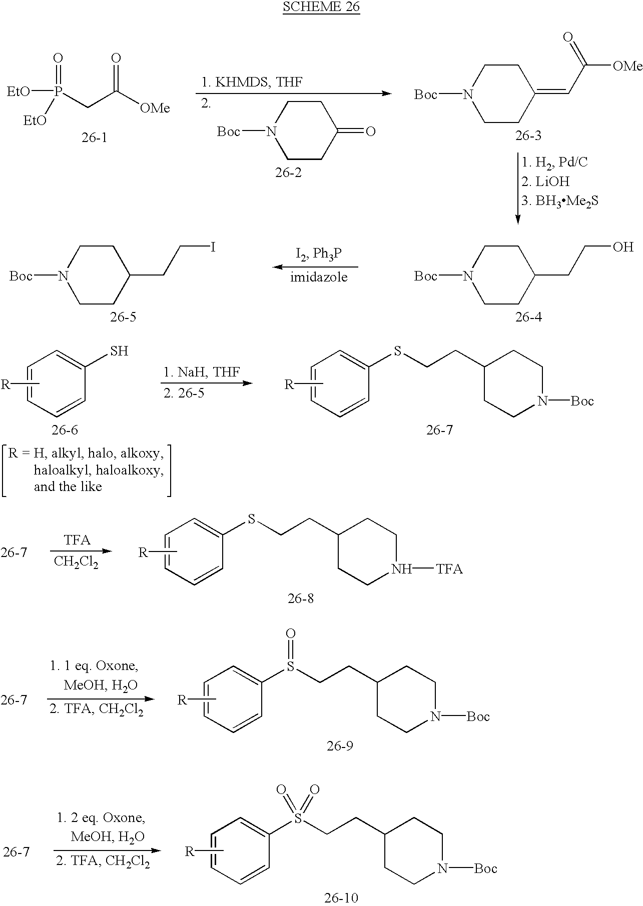

Procedures for synthesizing the present compounds containing 4-(2-(arylthio)ethyl)piperidine functionality are shown in Scheme 26. Treatment of phosphonoacetate 26-1 with KHMDS followed by addition of commercially available N-Boc -4-piperidone 26-2 provides unsaturated ester 26-3. Hydrogenation of 26-3 followed by hydrolysis to the acid and then reduction with boranemethyl sulfide then affords primary alcohol 26-4. Treatment with iodine and triphenylphosphine under standard conditions yields iodide 26-5. Reaction of the anion of a suitable aryl sulfide 26-6 with iodide 26-5 affords 4-(2-(arylthio)ethyl)-piperidine derivative 26-7. Sulfide can be deprotected directly under acidic conditions to give piperidine 26-8. Alternatively, the sulfur may be oxidized with one or two equivalents of a mild oxidizing agent such as Oxone® or mCPBA (m-chloroperoxybenzoic acid) to provide the corresponding sulfoxide or sulfone, respectively. In each case, the Boc group can be removed to provide sulfoxide 26-9 and sulfone 26-10. Each of these N-unsubstituted piperidines are then utilized as the cyclic secondary amine component as shown above in Schemes 5,6 and 7.

The following examples serve only to illustrate the invention and its practice. The examples are not to be construed as limitations on the scope or spirit of the invention.

### GENERAL

Concentration of solutions was carried out on a rotary evaporator under reduced pressure. Conventional flash chromatography was carried out on silica gel (230-400 mesh). Flash chromatography was also carried out using a Biotage Flash Chromatography apparatus (Dyax Corp.) on silica gel (32-63 microns, 60 Å pore size) in pre-packed cartridges of the size noted. NMR spectra were obtained in CDCl₃ solution unless otherwise noted. Coupling constants (J) are in hertz (Hz).

### HPLC CONDITIONS

LC1. Retention time using the following conditions: Column: YMC ODS A, 5μ, 4.6×50 mm; Gradient Eluent: 10:90 to 95:5 v/v acetonitrile/water+0.05% TFA over 4.5 min; Detection: PDA, 200-600 nm; Flow Rate: 2.5 mL/min.

LC2. Retention time using the following conditions: Column: YMC Pro-C18, 5μ, 4.6×50 mm; Gradient Eluent: 10:90 to 95:5 v/v acetonitrile/water+0.05% TFA over 3.0 min; Detection: PDA, 200-600 nm; Flow Rate: 2.5 mL/min.

HPLC A. Retention time using the following conditions: Column: YMC ODS A, 5μ, 4.6×50 mm; Gradient Eluent: 10:90 to 90:10 v/v acetonitrile/water+0.05% TFA over 4.5 min, hold 30 sec; Detection: PDA, 210-400 nm; Flow Rate: 2.5 mL/min.

HPLC B. Retention time using the following conditions: Column: Analytical Sales & Services Advantage HL C18 5μ4.6×100 mm column; Gradient Eluent: 10:90 to 90:10 v/v acetonitrile/water+0.05% TFA over 10 min, hold 2 min; Detection: PDA, 200-400 nm; Flow Rate: 2.25 mL/min.

### Aldehyde 1

2-Formyl-2-methylpropionic Acid, Para-methoxybenzyl Ester

Step A: 2, 2-Dimethyl-3-hydroxypropionic Acid, Para-methoxybenzyl Ester

A solution of 1.03 g (8.7 mmol) of 2, 2-dimethyl, 3-hydroxypropionic acid, 1.8 mL (12.9 mmol), of TEA and 1.3 mL (9.5 mmol) of para-methoxybenzyl chloride in 8 mL of DMF was stirred at rt for 24 h. The mixture was partitioned between 200 mL Et₂O and 100 mL of H₂O. After separating layers, the organic phase was washed with 100 mL of 1 N NaHCO₃, 100 mL of 2 N HCl, 2×100 mL of H₂O, 100 mL of brine, dried over MgSO₄ and concentrated. The residue was purified by flash chromatography using a gradient of 3:1 v/v to 3:2 v/v of hexanes/EtOAc as the eluant to afford the title compound: R_{F}: 0.17 (4:1 v/v hexanes/EtOAc); ¹H-NMR (500 Mhz) δ 1.21 (s, 6H), 3.57 (s, 2H), 3.82 (s, 3H), 5.09 (s, 2H), 6.90 (d, J=8.8, 2H), 7.28 (d, J=8.8, 2H).

Step B: 2-Formyl-2-methylpropionic Acid, Para-methoxybenzyl Ester

A solution of 0.2 mL (2.2 mmol) of oxalyl chloride in 4 mL of CH₂Cl₂ at −78° C was treated with 0.32 mL (4.5 mmol) of DMSO in 0.5 mL of CH₂Cl₂ maintaining the temperature at less than −60° C. The resulting mixture was stirred cold for 5 min. A solution of 211 mg (0.94 mmol) of 2,2-dimethyl-3-hydroxypropionic acid, para-methoxybenzyl ester (from Step A) in 1 mL of CH₂Cl₂ was added maintaining the temperature at less than −60° C. The resulting mixture was stirred cold for 15 min. The mixture was treated with 1.6 mL (9.1 mmol) of DIEA maintaining the temperature at less than −60° C. The reaction was warmed to 0° C., stirred for 30 min and quenched with H₂O. The mixture was partitioned between 50 mL of CH₂Cl₂ and 50 mL of H₂O and the layers were separated. The aqueous layer was extracted with 50 mL of CH₂Cl₂. The combined organic phases were washed with 100 mL of brine, dried over Na₂SO₄ and concentrated to afford the title compound which was used without further purification: R_{F}: 0.43 (4:1 v/v hexanes/EtOAc); ¹H-NMR (500 Mhz) δ 1.35 (s, 6H), 3.81 (s, 3H), 5.12 (s, 2H), 6.89 (d, J=8.7, 2H), 7.27 (d, J=8.7, 2H), 9.66 (s, 1H).

### Aldehyde 2

2-Formyl-2-methylpropionic Acid, Benzyl Ester

The title compound was prepared using procedures analogous to those described to prepare Aldehyde 1, except that benzyl bromide was substituted for para-methoxybenzyl chloride in Step A. ¹H-NMR (500 Mhz) δ 1.38 (s, 6H), 5.20 (s, 2H), 7.27-7.40 (m, 5H), 9.69 (s, 1H).

### Aldehyde 3

2-Ethyl-2-formylbutyric Acid, Benzyl Ester

Step A: Diethylmalonic Acid, Dibenzyl Ester

A solution of 2 mL (7.9 mmol) of dibenzyl malonate, 2 mL (25.0 mmol) of iodoethane and 7.84 g (24.0 mmol) of cesium carbonate in 50 mL of DMF was stirred overnight at rt. The reaction was partitioned between 250 mL of Et₂O and 250 mL of brine. After separating phases, the organic layer was washed with 250 mL of brine, dried over MgSO₄ and concentrated under reduced pressure to afford the title compound, which was used without further purification. R_{F}: 0.47 (9:1 v/v hexanes/EtOAc); ¹H-NMR (500 Mhz) δ 0.80 (t, J=7.5, 6H), 1.99 (q, J=7.5, 4H), 5.13 (s, 4H), 7.27-7.33 (m, 10H).

Step B: 2-Ethyl-2-formylbutyric Acid, Benzyl Ester

A solution of diethylmalonic acid, dibenzyl ester (7.9 mmol, from Step A) in CH₂Cl₂ at −78° C. was treated with 16 mL (16.0 mmol) of 1 M DIBAL in CH₂Cl₂ maintaining the temperature at less than −65° C. (J. Organic Chemistry, 1993, 58, 6843-6850). After stirring for 2.75 h, the reaction was quenched cold with 8 mL of saturated NH₄Cl and 10 mL of 2 N HCl. The reaction was warmed to rt and partitioned between 200 mL of CH₂Cl₂ and 300 mL of saturated Rochelle salts. After separating phases, the aqueous layer was extracted with 200 mL CH₂Cl₂. The combined organics were dried over MgSO₄ and concentrated under reduced pressure. The residue was purified by flash chromatography using 9:1 v/v of hexanes/EtOAc as the eluant to afford the title compound as a colorless oil contaminated with 15% of the starting material: R_{F}: 0.44 (9:1 v/v hexanes/EtOAc); ¹H-NMR (500 Mhz) δ 0.83 (t, J=7.5, 6H), 1.82-1.95 (m, 4H), 5.23 (s, 2H), 7.27-7.39 (m, 5H), 9.85 (s, 1H).

### Aldehyde 4

2-Formyl-3-methylbutyric Acid, Benzyl Ester

Step A: Benzyl Crotonate

A solution of 4.6 mL (43.2 mmol) of crotonyl chloride in 100 mL of CH₂Cl₂ at 0° C. was treated with 6.67 g (48.2 mmol) of K₂CO₃, 4.5 ml (43.5 mmol) of benzyl alcohol and 0.28 g (2.2 mmol) of DMAP. The reaction was warmed to rt and stirred for 3 days. After quenching with 100 mL of H20, phases were separated. The organic layer was washed with 100 mL of 2 N HCl, 100 mL of 1 N NaHCO₃ and 100 mL of brine, dried over Na₂SO₄ and concentrated under reduced pressure. The residue was purified on a 40M Biotage column using 19:1 v/v of hexanes/Et₂O as the eluant to afford the title compound as a colorless oil: R_{F}: 0.25 (19:1 v/v hexanes/Et₂O); ¹H-NMR (500 Mhz) δ 1.90 (dd, J 7.0, 1.6, 3H), 5.19 (s, 2H), 5.92 (dq, J=15.6, 1.6, 1H), 7.04 (dq, J=15.6, 7.0, 1H), 7.27-7.40 (m, 5H).

Step B: (R/S)-2-(Prop-2-yl)-3-butenoic Acid, Benzyl Ester

A solution of 0.78 g (4.4 mmol) of benzyl crotonate (from Step A) in 2 mL of THF was added to a solution of 5 mL of 1 M LDA/HMPA (prepared according to Herrmann et. al. Tetrahedron Letters, 1973, 2433-2436) at −78° C. maintaining the internal temperature less than −68° C. After stirring for 20 min, 0.5 mL (5.0 mmol) of isopropyl iodide was added. The reaction was allowed to stir for 5 hours at −78° C. After quenching the cold reaction with saturated NH₄Cl, volatiles were removed under reduced pressure. The residue was partitioned between 100 mL of Et₂O and 100 mL of saturated NH₄Cl. After separating phases, the organic layer was washed with 100 mL of saturated NH₄Cl and 100 mL of brine, dried over MgSO₄ and concentrated under reduced pressure. The residue was purified on a 40S Biotage column using 24:1 v/v of hexanes/EtOAc as the eluant to afford the title compound: R_{F}: 0.53 (19:1 v/v hexanes/ EtOAc); ¹H-NMR (500 Mhz) δ 0.91 (d, J=6.9, 3H), 0.93 (d, J=6.9, 3H), 2.05 (m, 1H), 2.78 (m, 1H), 5.12-5.21 (m, 4H), 5.85 (m, 1H), 7.27-7.40 (m, 5H).

Step C: 2-Formyl-3-methylbutyric Acid, Benzyl Ester

Ozone was bubbled through a solution of 0.14 g (0.64 mmol) of (R/S)-2-isopropyl-3-butenoic acid, benzyl ester (from Step B) in 6 mL of CH₂Cl₂ at −78° C. until a blue color persisted. After dissipation of the excess ozone with nitrogen, 3 mL of dimethyl sulfide was added. The reaction was warmed to rt and stirred overnight. Volatiles were removed under reduced pressure. . The residue was purified by flash chromatography using 9:1 v/v of hexanes/EtOAc as the eluant to afford the title compound: R_{F}: 0.38 (9:1 v/v hexanes/ EtOAc); ¹H-NMR (500 Mhz) (3:2 mixture of aldehyde:enol tautomers) δ 1.01, 1.03, 1.10 (3d, 6H), 2.46, 2.67 (2m, 1H), 3.05 (m, 0.4H), 5.19-5.26 (m, 2H), 7.08 (d, J=12.6, 0.6H), 7.27-7.40 (m, 5H), 9.74 (d, J=3.9, 0.4H), 11.56 (d, J=12.6, O—H).

### Aldehyde 5

(R/S)-2-Formyl-2-methylcyclobutyl Acetic Acid, Benzyl Ester

Step A: Cyclobutylacetonitrile

A solution of 9.05 g (138 mmol) of KCN and 1.05 g (3.9 mmol) of 18-crown-6 in 40 mL of DMSO was treated with 10 mL (88.9 mmol) of (bromomethyl)cyclobutane. The reaction was stirred at 60° C. overnight. The reaction was partitioned between 250 mL of 1 N NaOH and 250 mL of Et₂O. After separating phases, the organic layer was washed with 250 mL of 1 N NaOH and 250 mL of brine, dried over MgSO₄ and concentrated under reduced pressure to obtain the title compound as a yellow oil, which was used without further purification. ¹H-NMR (500 Mhz) δ 1.81-1.96 (m, 4H), 2.14-2.20 (m, 2H), 2.41 (d, J=6.6, 2H), 2.64 (m, 1H).

Step B: Cyclobutylacetic Acid

A mixture of 2.01 g (21.1 mmol) of cyclobutylacetonitrile (from Step A) and 40 mL of 6 N HCl was refluxed for 18 hours. The reaction was cooled to rt and extracted with 2×50 mL of Et₂O. The combined organic layers were washed with 2×50 mL of H₂O, dried over MgSO₄ and concentrated under reduced pressure to obtain the title compound as a yellow oil, which was used without further purification. ¹H-NMR (500 Mhz) δ 1.71-1.77 (m, 2H), 1.85-1.94 (m, 2H), 2.13-2.19 (m, 2H), 2.46 (d, J=7.6, 2H), 2.70 (m, 1H).

Step C: Cyclobutylacetic Acid, Benzyl Ester

A solution of 2.27 g (19.8 mmol) of cyclobutylacetic acid (from Step B) in 50 mL of DMF was treated with 7.77 g (23.8 mmol) of Cs₂CO₃ and 2.8 mL (23.5 mmol) of benzyl bromide. After stirring overnight at rt, the reaction mixture was poured into 100 mL of H₂O and extracted with 100 mL of Et₂O. After separating phases, the organic layer was washed with 100 mL of brine, dried over MgSO₄ and concentrated under reduced pressure. The residue was purified on a 40M Biotage column using 19:1 v/v of hexanes/Et₂O as the eluant to afford the title compound as a colorless oil: R_{F}: 0.54 (9:1 v/v hexanes/Et₂O); ¹H-NMR (500 Mhz) δ 1.71-1.76 (m, 2H), 1.82-1.93 (m, 2H), 2.11-2.17 (m, 2H), 2.48 (d, J=7.6, 2H), 2.72 (m, 1H), 5.11 (s, 2H), 7.32-7.40 (m, 5H).

Step D: Cyclobutylmalonic Acid, Dibenzyl Ester

A solution of 1.95 g (9.5 mmol) of cyclobutylacetic acid, benzyl ester (from Step C) in 10 mL of TUF at −78° C. was treated with 20 mL (20.0 mmol) of 1 M LiHMDS in THF maintaining the internal temperture less than −70° C. After stirring for 45 min, a solution of 1.6 mL (10.9 mmol) of benzyl cyanoformate in 10 mL of THF was added maintaining the internal temperture less than −68° C. After stirring for 1 h, the reaction was quenched cold with saturated NH₄Cl. Volatiles were removed under reduced pressure. The reaction mixture was suspended in 200 mL of Et₂O, washed with 2×200 mL of saturated NH14C and 200 mL of brine, dried over MgSO₄ and concentrated under reduced pressure. The residue was purified on a 40M Biotage column using 24:1 v/v of hexanes/Et₂O as the eluant to afford the title compound: R_{F}: 0.30 (9:1 v/v hexanes/Et₂O); ¹H-NMR (500 Mhz) δ 1.81-1.95 (m, 4H), 2.09-2.15 (m, 2H), 2.98 (m, 1H), 3.51 (d, J=10.6, 1H), 5.15 (s, 4H), 7.29-7.37 (m, 10H).

Step E: Cyclobutyl-methylmalonic Acid, Dibenzyl Ester

A solution of 1.78 g (5.2 mmol) of cyclobutylmalonic acid, dibenzyl ester (from Step D) in 10 mL of DMF was treated with 0.5 mL (8.0 mmol) of iodomethane and 2.59 g (7.9 mmol) of cesium carbonate. After stirring overnight at rt, the reaction was poured into 200 mL of H₂O and extracted with 200 mL of Et₂O. After separating phases, the organic layer was washed with 200 mL of 10% Na₂S₂O₃ and 200 mL of brine, dried over MgSO₄ and concentrated under reduced pressure. The residue was purified on a 40M Biotage column using 19:1 v/v of hexanes/Et₂O as the eluant to afford the title compound as a colorless oil: R_{F}: 0.42 (9: 1 v/v hexanes/Et₂O); ¹H-NMR (500 Mhz) δ 1.44 (s, 3H), 1.66 (m, 1H), 1.82 (m, 1H), 1.90-1.95 (m, 4H), 3.00 (m, 1H), 5.12 (ABq, J=12.3, 4H), 7.27-7.35 (m, 10H).

Step F: (R/S)-2-Formyl-2-methylcyclobutyl Acetic Acid, Benzyl Ester

The title compound was prepared from cyclobutyl-methylmalonic acid, dibenzyl ester (from Step E) using a procedure analogous to that described for Aldehyde 3, Step B. R_{F}: 0.30 (9:1 v/v hexanes/Et₂O); ¹H-NMR (500 Mhz) δ 1.28 (s, 3H), 1.72 (m, 1H), 1.83-1.98 (m, 5H), 2.90 (m, 1H), 5.20 (ABq, J=12.4, 21), 7.27-7.40 (m, 5H), 9.78 (s, 1H).

### Aldehyde 6

1-Formylcyclohexane Carboxylic Acid, Benzyl Ester

The title compound was prepared using procedures analogous to those described to prepare Aldehyde 3, except that 1,5-dibromopentane was substituted for iodoethane in Step A. 1H-NMR (500 Mhz) δ 1.43-1.55 (m, 6H), 1.88-1.93 (m, 2H), 2.02-2.07 (m, 2H), 5.19 (s, 4H), 7.27-7.39 (m, 5H), 9.55 (s, 1H).

### Aldehyde 7

1-Formylcyclobutane Carboxylic Acid, Benzyl Ester

Step A: 1,1-Cyclobutanedicarboxylic Acid, Dibenzyl Ester

A solution of 2.0 g (13.8 mmol) of cyclobutanedicarboxylic acid in 20 mL of DMF at 0° C. was treated with 7.7 mL (55.5 mmol) of triethylamine and 5.0 mL (41.6 mmol) of benzyl bromide. The reaction was warmed to rt and stirred overnight. The reaction was partitioned between H₂O and CH₂Cl₂. The residue was purified on a 40M Biotage column using 1: 1v/v of hexanes/EtOAc as the eluant to afford the title compound: ¹H-NMR (500 Mhz) δ 1.95-2.03 (m, 2H), 2.55-2.58 (m, 4H), 5.14 (s, 4H), 7.24-7.37 (m, 10H).

Step B: 1-Formylcyclobutane Carboxylic Acid, Benzyl Ester

The title compound was prepared from 1,1-cyclobutanedicarboxylic acid, dibenzyl ester (from Step A) using a procedure analogous to that described for Aldehyde 3, Step B. 1H-NMR (500 Mhz) δ 1.90-2.05 (m, 2H), 2.47-2.50 (m, 4H), 5.21 (s, 2H), 7.25-7.39 (m, 5H), 9.79 (s, 1H).

### Aldehyde 8

1-Formylcyclopentane Carboxylic Acid, Benzyl Ester

The title compound was prepared using procedures analogous to those described to prepare Aldehyde 3, except that 1,4-dibromobutane and potassium carbonate were substituted for iodoethane and cesium carbonate in Step A. ¹H-NMR (500 Mhz) δ 1.55-1.76 (m, 4H), 1.98-2.23 (m, 4H), 5.19 (s, 2H), 7.23-7.38 (m, 5H), 9.67 (s, 1H).

### Aldehyde 9

4-(4-Formyl-tetrahydropyranyl)-Carboxylic Acid, Benzyl Ester

The title compound was prepared using procedures analogous to those described to prepare Aldehyde 8, except that 2-chloroethyl ether was substituted for 1,4-dibromobutane. ¹H-NMR (500 Mhz) δ 1.99-2.04 (m, 2H), 2.12-2.17 (m, 2H), 3.61-3.68 (m, 4H), 5.21 (s, 2H), 7.25-7.39 (m, 5H), 9.56 (s, 1H).

### Aldehyde 10

1-Formylcyclopentane Carboxylic Acid, Para-methoxybenzyl Ester

Step A: Malonic Acid, Bis-para-methoxybenzyl Ester

A solution of 1.90 g (18.2 mmol) of malonic acid, 4.5 mL (36.0 mmol) para-methoxybenzyl alcohol and 0.28 g (2.2 mmol) 4-(dimethylamino)pyridine in 30 mL of CH₂Cl₂ and 2 mL of DMF at 0° C. was treated with 7.46 g (36.1 mmol) of dicyclohexylcarbodiimide. After warming to rt and stirring for 45 minutes, volatiles were removed under reduced pressure. The residue was suspended in 50 mL of 1:1 v/v hexane/Et₂O and placed in the freezer. After filtering the solids, the filtrate was concentrated under reduced pressure. The residue was partitioned between 200 mL of Et₂O and 200 mL of 1 N HCl. After separating phases, the organic layer was washed with 200 mL of brine, dried over MgSO₄ and concentrated under reduced pressure. The residue was adsorbed onto silica gel, filtered and washed with 4:1 v/v hexanes/EtOAc. Volatiles were removed under reduced pressure. The residue was purified on a 40M Biotage column using 17:3 v/v of hexanes/EtOAc and 4:1 v/v hexnaes/EtOAc as the eluant to afford the title compound: ¹H-NMR (500 Mhz) 6 3.43 (s, 2H), 3.82 (s, 6H), 5.11 (s, 4H), 6.88 (d, J=8.6, 4H), 7.27 (d, J=8.6, 4H).

Step B: 1-Formylcyclopentane Carboxylic Acid, Para-methoxybenzyl Ester

The title compound was prepared using procedures analogous to those described to prepare Aldehyde 8. ¹H-NMR (500 Mhz) δ 1.59-1.75 (m, 4H), 2.05-2.18 (m, 4H), 3.82 (s, 3H), 5.13 (s, 2H), 6.89 (d, J=8.4, 4H), 7.28 (d, J=8.4, 4H), 9.67 (s, 1H).

The following are representative procedures for the preparation of the piperidines used in the following Examples or which can be substituted for the piperidines used in the following Examples and which are not commercially available.

### Piperidine 1

4-(3-Benzyl-1-ethyl-(1H)-pyrazol-5-yl)piperidine di-trifluoroacetic Acid Salt

Step A: 1-(1-(t-Butoxycarbonyl)piperidin-4-yl)-4-phenylbutane-1,3-dione

Method A:

n-Butyl lithium (100 mL, 0.16 mole) was added to a stirred solution of diisopropylamine (16.16 g, 22.4 mL, 0.16 mole, distilled) in THF (450 mL) at 0° C. over 45 min under nitrogen. Stirring was continued for 10 min at 0° C. after the addition was complete. After cooling to −78° C., phenylacetone (21.45 g, 21.13 mL, 0.16 mole) in THF (100 mL) was added dropwise over 15 min with stirring. This solution was stirred at −78° C. for 1 h. Meanwhile, a solution of N-Boc isonipecotic acid (18.32 g, 0.080 mole) and carbonyl diimidazole (12.98 g, 0.080 mole) in TUF (150 mL) was prepared. After stirring for 15 min, this solution was canulated into the enolate solution dropwise over 15 min. The reaction was stirred at <−70° C. for 1 h and then allowed to warm to rt over 3 h. The reaction was quenched with IM citric acid (250 mL ) and stirred for 16 h. The organic layer was separated and washed with 250 mL each of saturated sodium bicarbonate solution, water and brine. After drying over sodium sulfate, the organic layer was concentrated to give an oil. The residue was purified by FC on silica gel (10% ethyl acetate in 60-80° C. petroleum ether) to give separation of the two isomers. The first higher R_{f} fractions afforded pure title compound as the minor product as an oil.

¹H NMR (500 MHz, CDCl₃): δ 7.34-7.37 (m, 2H), 7.25-7.31 (m, 3H), 5.46 (s, 1H), 4.11-4.17 (m, 2H), 3.63 (s, 2H), 2.70-2.76 (m, 2H), 2.29 (tt, J=11.7 and 3.7 Hz, 1H), 1.75-1.80 (m, 2H), 1.47-1.61 (m, 2H), 1.47 (s, 9H).

MS (ESI): m/z 346 (M+1).

The lower R_{f} fractions contained phenylacetone and major product 1-(1-(t-butoxycarbonyl)piperidin-4-yl)-2-phenylbutane-1,3-dione from which the latter crystallized on standing to give a white solid (m.p. 105-106° C.).

¹H NMR (360 MHz, CDCl₃): δ 15.23 (s, 1H), 7.3-7.45 (m, 3H), 7.15-7.2 (m, 2H), 4-4.1 (m, 2H), 2.35-2.50 (m, 2H), 2.2-2.3 (m, 1H), 1.87 (s, 3H), 1.5-1.75 (m, 4H), 1.43 (s, 9H).

MS (ESI): m/z 346 (M+1).

Method B:

Step B1: 1-(t-Butoxycarbonyl)piperidine-4-N-methyl-N-methoxycarboxamide

N-Boc isonipecotic acid (13.56 g, 59.2 mmol), N,O-dimethyl hydroxylamine hydrochloride (8.65 g, 88.7 mmol), and 1-hydroxybenzotriazole hydrate (15.9 g, 118 mmol) were dissolved in DMF (225 mL) in a 500 mL round-bottom flask and diisopropylethylamine (15.3 g, 20.6 mL, 118.3 mmol) was then added with stirring at rt. 1-Ethyl-3-(3-dimethylaminopropyl)carbodiimide (17.01 g, 88.74 mmol) was added in several portions over 10 min with stirring. After 22 h, the reaction mixture was poured into a water and ice mixture (600 mL ) and was extracted with ethyl acetate (5×125 mL). The combined organic layers were washed with 1N HCl (2×200 mL), 5% sodium bicarbonate (2×200 mL), water and brine, dried over sodium sulfate and concentrated to give the title compound as a yellowish oil.

¹H NMR (500 MHz, CDCl₃): δ 4.11-4.20 (m, 2H), 3.72 (br s, 3H), 3.20 (br s, 3H), 2.75-2.86 (m, 3H), 1.63-1.76 (m, 4H), 1.47 (s, 9H).

Step B2: 4-Acetyl-1-(t-butoxycarbonyl)piperidine

After dissolving the Weinreb amide from Step B 1 in anhydrous ether (400 mL) under nitrogen and cooling the solution in an ice bath, 1.4M methyl magnesium bromide (55 mL) in 3:1 toluene and THF was added with stirring and cooling over 30 min. After stirring at 0° C. for 1 h, the reaction was poured into a mixture of ice water (400 mL ) and acetic acid (8 mL , 150 mmol). The layers were separated and the aqueous layer was extracted twice with ether. The combined organic layers were washed with 0.1N HCl (200 mL), 3% sodium bicarbonate (200 mL), water (200 mL) and brine (200 mL), dried over sodium sulfate, and concentrated to give the crude product. FC (20-80% ethyl acetate in hexanes) gave the title compound as a yellowish oil. R_{f}: 0.27 (25% ethyl acetate in hexanes). Some starting Weinreb amide was also recovered. R_{f}: 0.10 (25% ethyl acetate in hexanes).

¹H NMR (500 MHz, CDCl₃): δ 4.07-4.14 (m, 2H), 2.75-2.83 (m, 2H), 2.46 (tt, J=11.3 and 3.8 Hz, 1H), 2.17 (s, 3H), 1.82-1.87 (m, 2H), 1.48-1.57 (m, 2H), 1.46 (s, 9H).

Step B3: 1-(1-(t-Butoxycarbonyl)piperidin-4-yl)-4-phenylbutane-1,3-dione

To a suspension of 60% sodium hydride (1.07 g) in THF (15 mL) at 0° C. was added a solution of 4-acetyl-1-(t-butoxycarbonyl)piperidine from Step B2 (3.03 g, 13.3 mmol) and methyl phenylacetate (6.01 g, 39.9 mmol) in THF (6 mL) over 20 min. The reaction was stirred for another 4 h as it was allowed to warm to rt. The mixture was diluted with ether (30 mL) and poured into 1N HCl. The layers were separated and the aqueous layer was extracted three times with ether. The combined organic layers were washed with brine (150 mL ), dried over sodium sulfate and concentrated. The crude product was purified by FC (20% ethyl acetate in hexanes) to give the title compound. R_{f}: 0.30 (20% ethyl acetate in hexane). The ¹H NMR data was the same as that obtained from the product of Method A.

Step B: 4-(5-Benzyl-1-ethyl-(1H)-pyrazol-3-yl)-1-(t-butoxycarbonyl)piperidine (Higher R_{f} isomer) and 4-(3-benzyl-1-ethyl-(1H)-pyrazol-5-yl)-1-(t-butoxycarbonyl)piperidine (Lower R_{f} isomer)

Method A:

1-(1-(t-Butoxycarbonyl)piperidin-4-yl)-4-phenylbutane-1,3-dione from Step A, from Method A or Method B, Step B3, (0.851 g, 2.46 mmol) in methanol (25 mL) was added over 10 min to a suspension of ethylhydrazine oxalate (0.444 g, 2.96 mmol) in methanol (5 mL ) in a 60° C. oil bath. After 15 h, the reaction was concentrated in vacuo and the residue was purified by repeated FC using a gradient of 50-100% ethyl acetate in hexanes to give first 4-(5-benzyl-1-ethyl-(1H)-pyrazol-3-yl)-1-(t-butoxycarbonyl)piperidine as the higher R_{f} product isomer and then the title compound as the lower R_{f}.

Higher R_{f} Isomer:

¹H NMR (500 MHz, CDCl₃): δ 7.2-7.3 (m, 2H), 7.3-7.4 (m, 1H), 7.17 (d, J=7.5 Hz, 2H), 5.77 (s, 1H), 4.0-4.25 (m, 2H), 3.97 (q, J=7.3 Hz, 2H), 3.95 (s, 2H), 2.7-2.9 (m, 2H), 2.76 (tt, J=11.3 and 3.8 Hz, 1H), 1.92 (br d, J=13 Hz, 1H), 1.5-1.65 (m, 2 ), 1.47 (s, 9H), 1.29 (t, J=7.3 Hz, 3H).

Lower R_{f} Isomer:

¹H NMR (500 MHz, CDCl₃): δ 7.25-7.4 (m, 3H), 7.2 (m, 2H), 5.72 (s, 1H), 4.1-4.3 (m, 2H), 4.08 (q, J=7.1 Hz, 2H), 3.95 (s, 2H), 2.7-2.9 (m, 2H), 2.66 (tt, J=11.3 and 3.8 Hz, 1H), 1.82 (br d, J=12.8 Hz, 1H), 1.4-1.6 (m, 2H), 1.48 (s, 9H), 1.47 (t, J=7.1 Hz, 3H).

Method B:

Step B 1: 1-(t-Butoxycarbonyl)-4-hydroxymethylpiperidine

A solution of 25.03 g (109.2 mole) N-Boc isonipecotic acid was dissolved in 200 mL THF and treated with 200 mL 1 M borane-tetrahydrofuran complex in THF, and the mixture was stirred overnight. The mixture was concentrated under vacuum, diluted with 750 mL ethyl acetate, and washed with 150 mL 1 N HCl (6×) and then saturated brine. The organic layer was dried over sodium sulfate and concentrated to give crude product as a white solid. This was used as is in the next step.

¹H NMR (500 MHz) 64.15 (br d, J=13.7 Hz, 2H), 3.52 (d, J=6.2 Hz, 2H), 2.69˜2.75 (m, 2H), 1.71˜1.75 (m, 2H), 1.62˜1.70 (m, 1H), 1.47 (s, 9H), 1.12˜1.21 (m, 2H).

Step B2: 1-(t-Butoxycarbonyl)-4-formylpiperidine

A mixture of 17.62 g (135.6 mmole) oxalyl chloride and 250 mL methylene chloride in a dry ice acetone bath. was treated with a solution of 21.19 g (271.2 mmole) DMSO in 150 mL methylene chloride over 20 minutes. After stirring for 20 minutes, a solution of 24.327 g 1-(t-butoxycarbonyl)-4-hydroxymethylpiperidine (from Step B1 above) in 150 mL methylene chloride was added over 1 h. After an additional 15 minutes, 57.17 (565 mmole) triethylamine in 150 mL methylene chloride was added over half an hour. The reaction mixture was allowed to warm up over night in the cooling bath. The reaction mixture was concentrated under vacuum to remove about 400 mL methylene chloride, and the residue was partitioned between 1 L ether and 300 mL water. To this was added 200 mL 1 N NaOH, the layers were separated, and the organic layer was washed with 150 mL 1 N NaOH (2×), water (3×), and saturated brine, dried over sodium sulfate, and concentrated to give 22.562 g crude product. FC (10˜60% ethyl acetate in hexanes) gave the title compound as slightly yellowish oil.

R_{F}: 0.29 (3:1 v/v hexanes/ethyl acetate).

¹H NMR (500 MHz) δ 9.68 (d, J=0.7 Hz, 1H), 3.96˜4.02 (m, 2H), 2.92˜2.97 (m, 2H), 2.40˜2.45 (m, 1H), 1.88˜1.94 (m, 2H), 1.53-1.64 (m, 2H), 1.47 (s, 9H).

Step B3: 1-(t-Butoxycarbonyl)-4-(2,2-dibromoethen-1-yl)piperidine

A solution of 48.615 g (146.6 mmole) carbon tetrabromide in 150 mL methylene chloride was added dropwise with stirring to a solution of 76.895 g (293.2 mmole) triphenylphosphine in 150 mL methylene chloride in a 1-L rb flask with ice bath cooling over 1.75 h. After 40 minutes, a solution of 15.631 g (73.29 mmole) 1-(t-butoxycarbonyl)-4-formylpiperidine (from Step B2 above) in 100 mL methylene chloride was added to the resulting brown suspension with stirring and cooling over 40 minutes. After one hour, 200 mL ether and 400 mL hexanes was added. The top suspension was filtered through Celite, and the residue was resuspended in 150 mL methylene chloride and treated with 300 mL ether. The mixture was filtered, and the solid was washed with hexanes until the total filtrate was 2 L. The filtrate was filtered again through Celite and washed with hexanes. The filtrate was washed with 100 mL 5% sodium bicarbonate, 300 mL water (2×), and 150 mL brine. The organic layer was dried over sodium sulfate and concentrated under vacuum to give 53.5 g crude product as a yellowish solid. Flash chromatography (FC) on 250 g silica gel (0˜15% ethyl acetate in hexanes) gave the title compound as a white solid.

R_{f}: 0.57 (15% ethyl acetate in hexanes).

¹H NMR (500 MHz) δ 6.25 (d, J=8.9 Hz, 1H), 4.04˜4.12 (m, 2H), 2.75˜2.83 (m, 2H), 2.42˜2.50 (m, 1H), 1.69˜1.75 (m, 2H), 1.47 (s, 9H), 1.29˜1.37 (m, 2H).

Step B4: 1-(t-Butoxycarbonyl)-4-(2-tributylstannylethyn-1-yl)piperidine

A mixture of 23.199 g (62.85 mmole) 1-(t-butoxycarbonyl)-4-(2,2-dibromoethen-1-yl)piperidine (prepared as in Step B3 above) and 600 mL anhydrous THF was cooled with dry ice acetone bath under nitrogen. To this mixture was added 88 mL of a 1.6 M butyl lithium solution in hexanes dropwise with stirring and cooling over 50 minutes. After one hour, the flask was transferred into an ice bath. After another hour, a solution of 28.64 g (87.99 mmole) tributyltin chloride in 100 mL THF was added with stirring and cooling over 35 minutes. After three h, the mixture was concentrated under vacuum to remove some THF, and the residue was partitioned between 600 mL ice water and 800 mL ether. The organic layer was washed with 200 mL of water (1×), 2% sodium bicarbonate (1×), water (2×), and saturated brine (1×), dried over sodium sulfate and concentrated under vacuum to give 30.104 g crude product as a green-yellowish liquid. FC on 275 g silica gel using cold 2.5˜15% ethyl acetate in hexanes as quickly as possible to give the title compound as a colorless liquid.

R_{f}: 0.45 (10% ethyl acetate in hexanes).

¹H NMR (500 MHz) δ 3.63˜3.67 (m, 2H), 3.25˜3.30 (m, 2H), 2.64˜2.69 (m, 1H), 1.74˜1.79 (m, 2H), 1.54˜1.64 (m, 8H), 1.47 (s, 9H), 1.32˜1.39 (m, 6H), 0.96˜0.99 (m, 6H), 0.92 (t, J=7.3 Hz, 9H).

Step B 5: 4-(1-(t-Butoxycarbonyl)piperidin-4-yl)-1-phenylbutan-2-on-3-yne

To a mixture of 1.727 g (3.466 mmole) 1-(t-butoxycarbonyl)-4-(2-tributyl-stannylethyn-1-yl)piperidine (prepared in Step B4 above) in 18 mL 1,2-dichloroethane was added 0.536 g (3.466 mmole) phenylacetyl chloride and 50 mg dichlorobis-(triphenylphosphine)palladium (II). The mixture was refluxed under nitrogen for 2 h, then concentrated under vacuum. Purification of the residue on silica gel (5˜35% ethyl acetate in hexanes) gave the title compound as a yellow oil.

R_{f}: 0.27 (20% ethyl acetate in hexanes).

¹H NMR (500 MHz) δ 7.34˜7.38 (m, 2H), 7.28˜7.32 (m, 1H), 7.24˜7.27 (m, 2H), 3.82 (s, 2H), 3.49˜3.54 (m, 2H), 3.17˜3.23 (m, 2H), 2.68˜2.73 (m, 1H), 1.72˜1.77 (m, 2H), 1.51˜1.57 (m, 2H), 1.47 (s, 9H).

Tetrakis(triphenylphosphine)palladium gave a similar result.

Step B6: 4-(3-Benzyl-1-ethyl-(1H)-pyrazol-5-yl)-1-(tert-butoxycarbonyl) Piperidine

Heating 1.204 g (3.677 mmole) 4-(1-(t-butoxycarbonyl)piperidin-4-yl)-1-phenylbutan-2-on-3-yne (prepared in Step B5 above) with 0.662 g (4.413 mmole) ethylhydrazine oxalate and 1.252 g (9.687 mmole) DIEA in 20 mL ethanol over night gave an 8:1 ratio of the title compound and its isomer 4-(5-benzyl-1-ethyl-(1H)-pyrazol-3-yl)-1-(tert-butoxycarbonyl)piperidine. Use of ethylhydrazine free base gave even more favorable ratios of the desired title compound. The desired isomer can be isolated by recrystallization using hexanes or by silica gel chromatography using 5˜10% acetonitrile in methylene chloride in addition to the procedure described in Method A above.

Step C: 4-(3-Benzyl-1-ethyl-(1H)-pyrazol-5-yl)piperidine di-TFA salt

To a solution of 4-(3-benzyl-1-ethyl-(1H)-pyrazol-5-yl)-1-(t-butoxycarbonyl)piperidine from Step B (lower R_{f} isomer) (0.373 g, 1.01 mmol) and anisole (0.219 mL, 2.02 mmol) in methylene chloride (15 mL) was added trifluoroacetic acid (1.555 mL, 20.2 mmol). The reaction was stirred at rt for 2.5 h and then concentrated. The residue was purified on preparative reverse-phase HPLC using 9.4×250 mm Semi-preparative Zorbax SB-C18 column with 17.5-35% acetonitrile gradient in water having 0.5% (v/v) TFA over 15 min at 6.05 mL per minute to give the title di-TFA salt compound as an oil. When a mixture of isomers from Step B is used, separation is also possible at this step with the above Prep HPLC conditions in which the title isomer elutes prior to 4-(5-benzyl-1-ethyl-(1H)-pyrazol-3-yl)piperidine.

### Piperidine 2

4-(3,3-Difluoro-3-(4-fluorophenyl)prop-1-yl)piperidine

Step A: 1-(t-Butoxycarbonyl)-4-(hydroxymethyl)piperidine

Di-t-butyl dicarbonate (4.69 g, 21.5 mmol) was transferred in methylene chloride (9 mL) over 10 min. to a solution of 4-(hydroxymethyl)piperidine (2.47 g, 21.4 mmol) in methylene chloride (16 mL). After stirring at rt for 1 h, the solution was diluted with ether (50 mL) and washed with 2 N aq. HCl, saturated aq. sodium bicarbonate, and saturated aq. brine (25 mL of each). The organic layer was dried (sodium sulfate), decanted, and evaporated to give the title compound as a crystalline solid.

¹H NMR (500 MHz, CD3OD): δ 4.08 (d, J=14 Hz, 2H), 3.40 (d, J=6 Hz, 2H), 2.81-2.67 (m, 2H), 1.71 (d, J=13 Hz, 2H), 1.67-1.58 (m, 1H), 1.44 (s, 9H), 1.09 (qd, J=12 and 4 Hz, 2H).

Step B: 1-(t-Butoxycarbonyl)-4-(iodomethyl)piperidine

Methanesulfonyl chloride (4.10 mL, 6.07 g, 52.9 mmol) was added dropwise to a solution of 1-(t-butoxycarbonyl)-4-(hydroxymethyl)piperidine from Step A (10.0 g, 46.4 mmol) and triethylamine (9.80 mL, 7.11 g, 70.3 mmol) in methylene chloride (140 mL) at 5-8° C. After 1 h, the mixture was diluted with ethyl acetate (400 mL) and washed with water (200 mL). The aqueous layer was extracted with ethyl acetate (2×150 mL) and the combined organic layers were washed with 1 N aq. HCl (200 muL), saturated aq. sodium bicarbonate (200 mL), and saturated aq. brine (200 mL). The organic layer was dried (sodium sulfate), decanted, and evaporated to give 1-(t-butoxycarbonyl)piperidin-4-yl methanesulfonate as a pale yellow solid.

A mixture of 1-(t-butoxycarbonyl)piperidin-4-yl methanesulfonate (13.58 g, 46.4 mmol) and sodium iodide (34.68 g, 232 mmol) in acetone (80 mL) was heated to reflux for 3 h. The mixture was partitioned between ether (350 mL) and water (350 mL). The organic layer was washed with saturated aq. brine (250 mL ), and the aqueous layers were extracted in succession with ether (250 mL). The combined organic layers were dried (sodium sulfate), decanted, and evaporated to give the title compound as a pale yellow oil.

¹H NMR (500 MHz, CDCl₃): δ 4.25-4.00 (m, 2H), 3.12 (d, J=4 Hz, 2H), 2.78-2.52 (m, 2H), 1.85 (d, J=13 Hz, 2H), 1.68-1.56 (m, 1H), 1.48 (s, 9H), 1.15 (qd, J=12 and 4 Hz, 2H).

Step C: ((1-(t-Butoxycarbonyl)piperidin-4-yl)methyl)triphenylphosphonium Iodide

A solution of triphenylphosphine (6.63 g, 25.3 mmol) and 1-(t-butoxycarbonyl)-4-(iodomethyl)piperidine from Step B (7.96 g, 24.5 mmol) in acetonitrile (40 mL) was heated to reflux for 72 h. The solution was evaporated to give 13.35 g of white solid. A portion (12.34 g) of this material was dissolved in acetonitrile (25 mL ) at 65° C. Ethyl acetate (35 mL) was added and the mixture was allowed to cool slowly to rt and then to −20° C. The supernatant was decanted, and the colorless crystals were washed with ethyl acetate (5×5 mL) and dried under vacuum to give the title compound.

¹HNMR(500 MHz, CD₃OD): δ 7.89(t, J=8 Hz, 3H),7.86(dd, J=12and 8 Hz, 6 H), 7.76 (td, J=8 and 4 Hz, 6H), 3.91 (bd, J=13 Hz, 2H), 3.44 (dd, J=14 and 6 Hz, 2H), 2.72-2.58 (m, 2H), 2.08-1.96 (m, 1H), 1.49 (bd, J=12 Hz, 2H), 1.41 (s, 9 H), 1.43 (qd, J=13 and 4 Hz, 2H).

Step D: Methyl (4-fluorobenzoyl)formate

Dimethyl oxalate (5.90 g, 50 mmol) was dissolved in THF (50 mL) and ether (50 mL) in a 3-neck round bottom flask fitted with a mechanical stirrer. The solution was stirred vigorously at −65° C. as a 1.0 M THF solution of 4-fluorophenylmagnesium bromide (60 mL, 60 mmol) was added dropwise over 40 min. The mixture was stirred 30 min at −65° C. and allowed to warm to −20° C. over 30 min before being poured into 2N aq. HCl (50 mL) with stirring. The layers were separated and the aq. layer was extracted with ether (3×50 mL). The combined organic layers were washed with saturated aq. brine (2×50 mL), dried (sodium sulfate), decanted, and evaporated. The residue was dissolved in ethyl acetate, dried (sodium sulfate), filtered, and evaporated to give a yellow solid. The crude product was dissolved in warm hexane (25 mL), filtered, and cooled to −20° C. Filtration followed by washing with cold hexane (15 mL) gave the title compound as light tan crystals.

¹H NMR (500 MHz, CDCl₃): δ 8.11 (dd, J=9 and 5, Hz, 2H), 7.21 (t, J=9 Hz, 2 H), 4.00 (s, 3H).

Step E: Methyl difluoro(4-fluorophenyl)acetate

Methyl (4-fluorobenzoyl)formate from Step D(4.75 g, 26.1 mmol) was added to (diethylamino)sulfur trifluoride (7.0 mL, 8.5 g, 53 mmol). The mixture was stirred rapidly and an ice bath was used briefly to reduce the temperature to 15° C. After the ice bath was removed, the reaction temperature rose to 48° C. over 10 min and then slowly returned to rt. After a total of 2.75 h, the solution was carefully poured onto crushed ice (30 g) and the mixture was extracted with methylene chloride (2×25 mL). The organic layers were washed in succession with saturated aq. sodium bicarbonate (2×25 mL) and saturated aq. brine (10 mL), combined, dried (sodium sulfate) decanted, and evaporated. The residue was distilled to give the title compound as a light yellow liquid, B.P. 46-48° C. (0.5 mm Hg). ¹HNMR (500 MHz, CDCl₃): 87.63 (dd, J=9,5 Hz, 2H), 7.16 (d, J=9 Hz, 2H), 3.88 (s, 3H).

Step F: 1-(t-Butoxycarbonyl)-4-(3,3-difluoro-3-(4-fluorophenyl)prop-1-en-1-yl)piperidine

A solution of methyl difluoro(4-fluorophenyl)acetate (2.04 g, 10.0 mmol) from Step E in methanol (10.0 mL) was cooled to −60° C. Sodium borohydride (380 mg, 10.0 mmol) was added in 5 portions at 10 to 15 min. intervals. The mixture was cooled to −60 to −55° C. prior to each addition and allowed to warm to −45° C. following each addition. After the last addition, the mixture was stirred 1.25 h at −50 to −45 OC. The mixture was cooled to −60° C. and quenched with 1 N aq. HCl (30 mL), with the temperature rising to −20° C. near the end of the addition. After warming to 0° C., the mixture was extracted with ether (3×20 mL). The combined ether layers were washed with water (2×20 mL), dried (sodium sulfate), decanted, and evaporated to give crude 2,2-difluoro-2-(4-fluorophenyl)-1-methoxyethanol as a pale yellow oil.

A suspension of ((1-(t-butoxycarbonyl)piperidin-4-yl)methyl)triphenylphosphonium iodide (500 mg, 0,92 mmol) from Step C in THF (7.2 mL) was stirred at rt for 30 min. A 0.5 M toluene solution of potassium bis(trimethylsilyl)amide (1.8 mL, 0.90 mmol) was added over 3 min., giving an orange suspension. After 30 min., crude 2,2-difluoro-2-(4-fluorophenyl)-1-methoxyethanol (95 mg, 0.46 mmol) was added in TBF (1.0 mL). After an additional 30 min, the mixture was quenched by the addition of saturated aq. NH₄Cl (2 mL). The mixture was partitioned between ethyl acetate (50 mL) and water (75 mL), and the aqueous layer was extracted with ethyl acetate (50 mL). The organic layers were washed in succession with saturated aq. brine (25 mL), dried (sodium sulfate), decanted, and evaporated. The crude product was purified by FC, eluting with 10% ether in hexane to give the title compound as a 95:5 mixture of cis and trans isomers, respectively.

¹H NMR (500 MHz, CDCl₃): δ 7.55 (dd, J=9 and 5 Hz, 2H), 7.13 (t, J=9 Hz, 2 H), 5.76 (q, J=12 Hz, 1H), 5.64 (dd, J=12 and 10 Hz, 1H), 4.20-3.95 (m, 2H), 2.80-2.54 (m, 3H), 1.54 (bd, J=12 Hz, 2H), 1.47 (s, 9H), 1.26 (qd, J=12 and 4 Hz, 2H).

Step G: 1-(t-Butoxycarbonyl)-4-(3,3-difluoro-3-(4-fluorophenyl) propyl)piperidine

Potassium azodicarboxylate (695 mg, 3.58 mmol) was added to a solution of 1-(t-butoxycarbonyl)-4-(3,3-difluoro-3-(4-fluorophenyl)prop-1-en-1-yl)piperidine from Step F (424 mg, 1.19 mmol) in methanol (3.3 mL). The mixture was stirred at rt as a 9.0 M solution of acetic acid in methanol (0.80 mL, 7.2 mmol) was added over 3 h using a syringe pump. After 30 min., a second portion of potassium azodicarboxylate (695 mg, 3.58 mmol) was added followed by the addition of 9.0 M acetic acid in methanol (0.80 mL, 7.2 mmol) over 3 h. After 20 min, a third portion of potassium azodicarboxylate (695 mg, 3.58 mmol) was added followed by the addition of 9.0 M acetic acid in methanol (0.80 mL, 7.2 mmol) over 3 h. After stirring for 20 h at rt, the mixture was diluted with ethyl acetate (80 mL), and washed with 2 N aq. HCl (40 mL), saturated aq. sodium bicarbonate (40 mL), and saturated aq. brine (40 mL ). The organic layer was dried (sodium sulfate), decanted, and evaporated to give a mixture containing the title compound and 20-25% of unreduced 1-(t-butoxycarbonyl)-4-(3,3-difluoro-3-(4-fluorophenyl)prop-1-en-1-yl)piperidine.

A portion (365 mg) of the crude mixture containing residual olefin was hydrogenated at atmospheric pressure for 16 h using iridium black (30 mg) in a mixture of t-butanol (24 mL) and ethyl acetate (2.4 mL). The mixture was filtered, the catalyst was washed with methanol, and the filtrate was evaporated to give the title compound as a pale yellow syrup. R_{f}: 0.2 (5% ethyl acetate in hexane).

¹HNMR (500 MHz, CDCl₃): 67.46 (dd, J=9 and 5 Hz, 2H), 7.12 (t, J=9 Hz, 2 H), 4.18-4.00 (m, 2H), 2.73-2.61 (m, 2H), 2.14 (tm, J=16 Hz, 2H), 1.64 (bd, J=12 Hz, 2H), 1.46 (s, 9H), 1.46-1.33 (m, 3H), 1.08 (qd, J=12 and 4 Hz, 2H).

Step H: 4-(3,3-Difluoro-3-(4-fluorophenyl)prop-1-yl)piperidine

1-(t-Butoxycarbonyl)-4-(3,3-difluoro-3-(4-fluorophenyl)prop-1-yl)piperidine from Step G (122 mg, 0.34 mmol) was dried by evaporation of a toluene solution at reduced pressure. The residue was dissolved in chloroform (7.6 mL) and iodotrimethylsilane (0.100 mL, 141 mg, 0.70 mmol) was added. After stirring 30 min at rt, the solution was poured into a mixture of saturated aqueous sodium bicarbonate (15 mL) and 2.5 N aq. NaOH (5 mL), and extracted with ether (50 mL). The organic layer was washed with saturated aq. brine (15 mL), dried (sodium sulfate), decanted, and evaporated to give the title compound as a colorless oil.

¹H NMR (500 MHz, CD₃OD): δ 7.51 (dd, J=9 and 5 Hz, 2H), 7.17 (t, J=9 Hz, 2 H), 2.98 (dm, J=12 Hz, 2H), 2.52 (td, 3=1,2,3 Hz, 2H), 2.17 (tm, J=16 Hz, 2H), 1.65 (bd, J=13 Hz, 2H), 1.42-1.26 (m, 3H), 1.07 (qd, J=12 and 4 Hz, 2H). HPLC/MS (ESI): m/z 258 (M+1H); HPLC: 2.64 min.

### Piperidine 3

4-(2-((4-Fluorophenyl)sulfonyl)eth-1-yl)piperidine Trifluoroacetic Acid Salt

Step A: 4-(2-Hvdroxyeth-1-yl)piperidine Acetic Acid Salt

Combined 4-(2-hydroxyeth-1-yl)pyridine (25 g, 0.2 mol) and platinum oxide (1 g, 4.4 mmol) in 400 mL acetic acid. Placed under 45 psi hydrogen at 60° C. for 24 h. Decanted, then filtered through Celite and removed the solvent to afford the crude product, which was used without further purification.

Step B: 4-(2-Hydroxyeth-1-yl)-1-tert-butoxycarbonylpiperidine

Dissolved sodium bicarbonate (134 g, 1.6 mol) and 4-(2-hydroxyeth-1-yl)piperidine acetic acid salt (38 g, 0.2 mol, from Step A) in 500 mL of 50% tetrahydrofuran in water. Added di-tert-butyl dicarbonate (35 g, 0.2 mol) and stirred at rt overnight. Diluted with ethyl acetate and extracted the aq. layer with 2×300 mL of ethyl acetate. Washed the combined organic layers with 2×300 mL of 1 N HCl and brine. Dried over magnesium sulfate and concentrated to afford the title compound. ESI-MS: 230 (M+H); HPLC A: 2.76 min.

Step C: 4-(2-Iodoeth-1-yl)-1-tert-butoxycarbonylpiperidine

Combined 4-(2-hydroxyeth-1-yl)-1-tert-butoxylcarbonylpiperidine (37.4 g, 0.16 mol, from Step B), triphenylphosphine (55 g, 0.21 mol) and imidazole (14 g, 0.21 mol) in 800 mL of 33% acetonitrile in ether. Cooled to 0° C. and added iodine (56 g, 0.22 mol) portionwise. The iodine is de-colored until the endpoint of the reaction. Diluted with 1 L of ether. Washed organic layer with 2×500 mL each of sat'd. aq. Na₂S₂O₃, sat. aq. CuSO₄ and brine. Dried over magnesium sulfate, filtered and concentrated. Triphenylphosphine oxide precipitates. Added ether and filtered the slurry through a plug of silica gel. Purified a portion of the crude material by flash chromatography (5% ethyl acetate in hexane eluent) to afford the title compound.

¹H-NMR (400 MHz, CDCl₃): δ 4.10 (br s, 2H), 3.23 (t, 2 H, J=7.2 Hz), 2.72 (br t, 2 H, 12.3 Hz), 1.79 (q, 2 H, J=7 Hz), 1.67 (br d, 2 H, 14 Hz), 1.61 (m, 1H), 1.47 (s, 9 H), 1.14 (qd, 2 H, J=4.3, 12 Hz); ESI-MS: 340 (M+H); HPLC A: 3.74 min.

Step D: 4-(2-(4-Fluorophenylthio)eth-1-yl)-1-tert-butoxycarbonylpiperidine

To a slurry of sodium hydride (47 mg, 60% in mineral oil, 1.2 mmol) in tetrahydrofuran at 0° C. was added 4-fluorothiophenol (0.1 mL, 0.94 mmol). The reaction mixture was warmed to rt. for 20 min, followed by addition of 4-(2-iodoeth-1-yl)-1-tert-butoxycarbonylpiperidine (265 mg, 0.78 mmol, from Step C). The reaction was then heated to reflux for 10 min, cooled and diluted with ether. The organic layer was washed with 1 N NaOH, dried over magnesium sulfate and concentrated to provide the title compound. ESI-MS: 340.0 (M+H); HPLC A: 4.07 min.

Step E: 4-(2-(4-Fluorophenylsulfonyl)eth-1-yl)piperidine trifluoroacetic acid salt

Added a solution of Oxone® (1.14 g, 1.86 mmol) in water to a solution of 4-(2-(4-fluorophenylthio)eth-1-yl)-1-tert-butoxycarbonylpiperidine (252 mg, 0.74 mmol, from Step D) in methanol at 0° C. Warmed to rt. After 90 min., added an additional 0.5 g of Oxone®. After 3 h, the reaction mixture was diluted with methylene chloride and washed with 1 N NaOH containing sodium bisulfite. The aq. layer was extracted twice with methylene chloride, and the combined organic layers were dried over magnesium sulfate. The solution was concentrated and dissolved in 5% trifluoroacetic acid in methylene chloride for 1 h. The solvent was evaporated to afford the title compound.

ESI-MS: 239.8 (M+H); HPLC A: 2.54 min.

### Piperidine 4

4-((5-Benzyl)pyrid-3-yl)piperidine di-TFA Salt

Step A: N-tert-Butoxycarbonyl-1,2,5,6-tetrahydropyridine-4-trifluoromethane Sulfonate

A dry flask under nitrogen was charged with a solution of sodium hexamethyldisilazide (11 mL , 1.0 M in THF) and was cooled to −78° C. A solution of N-tert-butoxycarbonyl-4-piperidone (2.0 g, 10 mmol) in 10 mL THF was added dropwise via cannula. After 30 min. a solution of 2-(N,N-bis(trifluoromethanesulfonyl)amino-5-chloropyridine (4.7 g, 12 mmol) in 15 mL THF was added. The mixture was warmed to rt, quenched with sat'd ammonium chloride and extracted with ethyl acetate. The ethyl acetate layer was separated and washed with sat'd brine then dried over sodium sulfate and concentrated. Flash chromatography (100 g silica, 10/1 Hexane/ethyl acetate) afforded the title compound. ¹H NMR (400 MHz, CDCl₃). δ 1.5 (s, 9H), 2.4-2.48 (m, 2H), 3.62-3.68 (t, 2H), 4.05-4.07 (m, 2H), 5.77-5.8 (bs, 1H).

Step B: N-tert-Butoxycarbonyl-4-trimethylstannyl-1,2,5,6-tetrahydropyridine

A dry flask under nitrogen was charged with 20 mL THF, lithium chloride (1.6 g, 37.3 mmol), tetrakistriphenylphosphine palladium(0), (331 mg, 0.28 mmol) and hexamethyldistannane (1.2 mL, 5.7 mmol). N-tert-butoxycarbonyl-1,2,5,6-tetrahydropyridine-4-trifluoromethane sulfonate (1.9 g, 5.7 mmol) was added and the mixture was stirred overnight at 60° C. The mixture was diluted with water and extracted with ethyl acetate (3×150 mL). The combined organic layers were dried over sodium sulfate and concentrated. Flash chromatography (100 g silica, 20/1 Hexane/ethyl acetate) afforded the title compound.

¹H NMR (300 MHz, CDCl₃). δ 0.5 (s, 9H), 1.5 (s, 9H), 2.25-2.35 (m, 2H), 3.62-3.68 (t, 2H), 3.95-3.97 (m, 2H), 5.77-5.8 (bs, 1H).

Step C: 3-Bromo-5-benzylpyridine

A dry flask under nitrogen was charged with zinc chloride (16 ML, 0.5 M in THF, 8 mmol), and a solution of phenylmagnesium chloride (4 mL, 2.0 M in THF, 8 mmol). The mixture was heated to 50° C. for 3h then cooled to rt and transferred via cannula to a solution of 3,5-dibromopyridine (1.26 g, 5.3 mmol), copper iodide (61 mg, 0.32 mmol), and bis(diphenylphosphino)ferrocene palladium dichloride (218 mg, 0.27 mmol) in 15 mL THF. The resulting mixture was heated to 50° C. overnight. Sat'd ammonium chloride was added and the mixture was extracted with ethyl acetate. The organic portion was dried over sodium sulfate and concentrated. Flash chromatography (8/1 hexane/ethyl acetate) afforded the title compound. ¹H NMR (400 MHz, CDCl₃). δ 4.02 (s, 2H), 7.18-7.4 (m, 8H), 7.65 (s, 1H).

Step D: 4-((5-Benzyl)pyrid-3-yl)piperidine di-TFA

A flask was purged with nitrogen and charged with DMF, 3-bromo-5-benzylpyridine (618 mg, 2.5 mmol, from Step C), tetrakis triphenylphosphine palladium (58 mg, 0.05 mmol), and N-tert-butoxycarbonyl-4-trimethylstannyl-1,2,5,6-tetrahydropyridine (1.04 g, 3 mmol). The mixture was heated to 100° C. and stirred for 10 h. An additional portion of tetrakis triphenylphosphine palladium (40 mg, 0.03 mmol) was added and stirring was continued for 14 h. The solution was cooled and diluted with ethyl acetate then washed with water, dried over sodium sulfate and concentrated. Flash chromatography (2.5/1 hexane/ethyl acetate) afforded 590 mg (67%) of the coupling product. The product was dissolved in 4 mL methanol and 50 mg 10% Pd/C was added. The mixture was stirred under 1 atm of hydrogen for 3h. The catalyst was filtered off and the residue was dissolved in 1/1 TFA/methylene chloride. Removal of the solvent and drying under vacuum afforded the title compound as its TFA salt.

¹H NMR (500 MHz, CDCl₃). δ 1.55-1.64 (m, 2H), 1.75-1.8 (d, 2H), 2.57-2.62 (m, 1H), 2.68-2.73 (t, 2H), 3.15-3.2 (d, 2H), 7.14-7.15 (d, 2H), 7.19-7.21 (m, 1H), 7.26-7.32 (m, 3H), 8.30-8.31 (d, 2H).

### Piperidine 5

4-(1-(4-Methylsulfonylbenzyl)-3-ethyl-(1H)-pyrazol-4-yl)piperidine di-TFA salt

Step A: 3-Ethyl Pyrazole

A solution of oxalyl chloride (24 mL, 280 mmol) in 500 mL dry dichloromethane was cooled to −78° C. and DMSO (34 mL, 480 mmol) was added. After stirring for 10 min 2-pentyne-1-ol (18.5 mL, 200 mmol) was added dropwise. The resulting mixture was stirred at −78° C. for 20 min then N,N-diisopropylethyl amine (104 mL, 600 mmol) was added and the mixture was brought to rt. After 30 min tlc analysis (3/1 hexane/EtOAc) indicated no remaining alcohol. A solution of hydrazine (63 mL , 2 mol) in 100 mL of ethanol was added and the dichloromethane was then distilled off (500 mL collected). An additional 400 mL of ethanol was added and the mixture was refluxed overnight, keeping the bath temperature at 120° C. The mixture was concentrated and diluted with EtOAc and water. The layers were separated and the organic layer was washed with sat'd NaCl then dried over sodium sulfate and concentrated. Flash chromatography (500 g silica, 1→3% methanol/ CH₂Cl₂) gave the title compound.: ¹H NMR (500 MHz, CDCl₃) δ 1.3 (t, 3H), 2.75 (q, 2H), 6.13 (s, 1H), 7.54 (s, 1H).

Step B: 3-Ethyl-4-iodopyrazole

3-Ethyl pyrazole (17 g, 177 mmol from Step A), sodium hydroxide (7 g, 177 mmol) and sorbitan palmitate (1.1 g, 2.65 mmol) were suspended in 180 mL water. Iodine (45 g, 177 mmol) was added to the stirred suspension in portions over 20 min. After stirring for an additional 45 min the mixture was diluted with EtOAc and washed with water and sat'd NaCl. The organic portion was dried over sodium sulfate and concentrated. Flash chromatography (500 g silica, 1 e 25% ether/ CH₂Cl₂) afforded the desired product: ¹H NMR (500 MHz, CDCl₃) δ 1.3 (t, 3H), 2.73 (q, 2H), 7.55 (s, 1H).

Step C: 1-(4-Thiomethylbenzyl)-3-ethyl-4-iodopyrazole

Sodium hydride (2.3 g, 58.6 mmol, 60% dispersion in mineral oil) was suspended in 100 mL DMF and a solution of 3-Ethyl-4-iodopyrazole (10 g, 45 mmol, from Step B) in 50 mL DMF was added dropwise. After stirring the resulting mixture for 30 min the mixture was cooled to 0° C. and a solution of 4-thiomethylbenzyl chloride (8.6 g, 49.5 mmol) in 50 mL DMF) was added. The resulting mixture was warmed to rt and stirred for 2 h. Sat'd ammonium chloride was added and the mixture was poured into 200 mL EtOAc. The layers were separated and the organic layer was washed with water (2×) and sat'd NaCl. The organic fraction was dried over sodium sulfate and concentrated. Flash chromatography (500 g silica, 20/1→5/1 hexane/EtOAc) gave product as a 2.5/1 mixture of isomers. ¹H NMR (500 MHz, CDCl₃) major isomer δ 1.27 (t, 3H), 2.51 (s, 3H), 2.64 (q, 2H), 5.21 (s, 2H), 7.18 (d, 2H), 7.27 (d, 2H), 7.31 (s, 1H), minor isomer δ 1.05 (t, 3H), 2.47 (s, 3H), 2.64 (q, 2H), 5.34 (s, 2H), 7.05 (d, 2H), 7.22 (d, 2H), 7.52 (s, 1H).

Step D: 1-(4-Thiomethylbenzyl)-3-ethyl-4-(N-tert-butoxycarbonyl-1,2,3,6-tetrahydropyrid-4-yl)pyrazole

A dry flask was charged with a solution of isopropyl magnesium chloride (28 mL, 2.0 M in THF, 56 mmol) and a solution of 1-(4-Thiomethylbenzyl)-3-ethyl-4-iodopyrazole (15.5 g, 43.3 mmol, from Step C) in 25 mL THF was added. After stirring for 1 h a solution of N-tertbutoxylcarbonylpiperid-4-one (9.5 g, 47.6 mmol) in 20 mL THF was added. The mixture was stirred at 45° C. for 2h. The mixture was quenched with sat'd ammonium chloride and 1 M HCl then extracted with EtOAc. The organic portion was stirred over magnesium sulfate for 48 h. After solvent removal the product was isolated by flash chromatography (500 g silica, 3/1 hexane/EtOAc) to afford the material. ¹H NMR (500 MHz, CDCl₃). δ 1.25 (t, 3H), 1.48 (s, 9H), 2.3-2.35 (bs, 2H), 2.44 (s, 3H), 2.72-2.77 (q, 2H), 3.56 (t, 2H), 4.03 (bs, 2H), 5.15 (s, 2H), 5.7 (bs, 1H), 7.12-7.13 (d, 2H), 7.16 (s, 1H), 7.19-7.21 (d, 2H).

Step E: 1-(4-Methanesulfonylbenzyl)-3-ethyl-4-(N-tert-butoxycarbonyl-1,2,3,6-tetrahydropyrid-4-yl)pyrazole

A solution of 1-(4-Thiomethylbenzyl)-3-ethyl-4-(N-tert-butoxycarbonyl-1,2,3,6-tetrahydropyrid-4-yl)pyrazole (4 grams, 9.7 mmol, from Step D) in 50 mL methanol was cooled to 0° C. A solution of Oxone® (8.3 g, 13.6 mmol) in 25 mL water was added slowly. The solution was warmed to 10° C. and stirred for 40 min then quenched with sat'd sodium thiosulfite. The mixture was extracted with dichloromethane and the organic portion was dried over magnesium sulfate and concentrated. Flash chromatography (500 g silica, 2.5/1 CH₂Cl₂/ether) gave of the desired product. ¹H NMR (500 MHz, CDCl₃). δ 1.29 (t, 3H), 1.51 (s, 9H), 2.38 (bs, 2H), 2.74-2.79 (q, 2H), 3.06 (s, 3H), 3.61-3.63 (t, 2H), 4.06 (bs, 2H), 5.34 (s, 2H), 5.7 (bs, 1H), 7.29 (s, 1H), 7.35-7.37 (d, 2H), 7.92-7.94 (d, 2H).

Step F: 4-(1-(4-Methylsulfonylbenzyl)-3-ethyl-(1H)-pyrazol-4-yl)piperidine di-TFA Salt

A solution of 1.1 g (0.5 mmol) of 1-(4-methanesulfonylbenzyl)-3-ethyl-4-(N-tert-butoxycarbonyl-1,2,3,6-tetrahydropyrid-4-yl)pyrazole (from Step E) was stirred with 10% Pd/C (500 mg, 0.5 mmol) in 15 mL methanol under 1 atmosphere of hydrogen. After 1 h the mixture was filtered and concentrated. The product was dissolved in 20 mL 1/1 TFA/ CH₂Cl₂, stirred for 1 hour and evaporated to afford the title compound as the TFA salt.

### Piperidine 6 (Method A)

4-(2-Benzylthiazol-5-yl)piperidine di-HCl Salt

Step A: 1-t-Butyloxycarbonyl-4-(nitromethylcarbonyl)piperidine

To a solution of 1-t-butyloxycarbonylpiperidine-4-carboxylic acid (22.9 g, 100 mmol) in 200 mL of anhydrous THF was added carbonyl diimidazole (20.0 g, 125 mmol) under nitrogen. Effervescence was observed and the reaction mixture was stirred 1 h at ambient temperature. Freshly distilled nitromethane (7.4 mL, 135 mmol) followed by DBU (21.0 mL, 140 mmol) were added. The resulting reaction mixture was stirred for 1 day at rt. After dilution with ethyl acetate, the mixture was washed with 2N HCl and brine. The organic phase was dried over anhydrous magnesium sulfate. Evaporation of the solvent followed by the purification of the residue on silica gel using 1:1 mixture of ethyl acetate -hexane with 1% acetic acid as an eluent gave the nitroketone as a semi solid. After removal of last traces of acetic acid by azeotroping with toluene.

¹H NMR (CDCl₃): δ 1.48(9H, s); 1.65,1.90,2.65,2.80,4.15 (all multiplets); 5.36(2H, s).

Step B: 1-t-Butyloxycarbonyl-4-(1-hydroxy-2-nitro)ethyl)piperidine

Sodium borohydride (1.52 g, 40 mmol) was added portionwise to a suspension of 1-t-butyloxycarbonyl-4-(nitromethylcarbonyl) piperidine (10.5 g, 40 mmol) from Step A in methanol (80 mL) at 0° C. After 6.5 h, the solvent was removed in vacuo. The residue was diluted with ethyl acetate and stirred with 2N HCl and the layers were separated. The organic phase was washed with brine and dried over magnesium sulfate. Solvent removal gave the desired product as amorphous solid.

¹H NMR (CDCl₃): δ 1.45(9H, s); 4.45(2H, m); 1.3,1.65,1.85,2.7,4.2 (multiplets)

Step C: 1-t-Butyloxycarbonyl-4-(1-hydroxy-2-amino)ethylpiperidine

To a stirred suspension of 1-t-butyloxycarbonyl-4-(1-hydroxy-2-nitro)ethyl)piperidine (9.0 g, 33 mmol) from Step B in anhydrous methanol (100 mL),10% Pd-C (2.0 g) followed by ammonium formate (12.6 g, 200 mmol) were cautiously added. The reaction mixture was stirred 1.5 days at ambient temperature. The catalyst was filtered through a pad of celite and washed with methanol. The filtrate was concentrated after adding 42 mL of triethylamine to free the product from any formic acid salts. The residue was purified on silica gel using 10:10:1 mixture of ethyl acetate, hexane and NH₄OH as solvent to yield the desired amino alcohol as a white solid after azeotroping with toluene. ¹H NMR (CDCl₃): δ 1.5(9H, s); 3.6(2H, s)1.2, 1.75, 2.6, 3.24, 3.4, 4.15 (all multiplets).

Step D: 1-t-Butyloxycarbonyl-4-(1-hydroxy-2-phenylacetylamino)ethylpiperidine

Phenylacetyl chloride (0.44 mL, 3.3 mmol) was added dropwise to a mixture of 1-t-butyloxycarbonyl-4-(1-hydroxy-2-amino)ethylpiperidine (0.732 g, 3 mmol) from Step C and triethylamine (0,465 mL, 3.3 mmol) in methylene chloride (15 mL) at ice bath temperature and the bath was removed. After stirring for 3 h at rt, the reaction mixture was diluted with ethyl acetate and washed with saturated sodium bicarbonate and brine. The organic phase was dried over anhydrous magnesium sulfate. Solvent removal gave a crude product which was used in the next step without further purification.

¹H NMR (CDCl₃): δ 1.45(9H, s); 3.42(2H, s); 1.2, 1.75, 2.6, 3.2, 3.42, 4.12 (all multiplets).

Step E: 1-t-Butyloxycarbonyl-4-(2-phenylacetamido)acetylpiperidine

To a stirred solution of 1-t-butyloxycarbonyl-4-(1-hydroxy-2-phenylacetylamino)ethylpiperidine from Step D in acetone at ice bath temperature 8 N Jones reagent was added until the orange color of the reagent persisted. After stirring for 0.5 h, 0.2 mL of isopropanol was added and the stirring was continued for 0.5 h. Solvent was removed in vacuo and the residue was partitioned between water and ethyl acetate. The organic phase was washed with brine and dried over anhydrous magnesium sulfate. Solvent removal gave an oil which was purified on silica gel using 1: ethyl acetate -hexane as solvent to yield the desired ketone as an oil.

¹H NMR (CDCl₃): δ 1.46(9H, s); 3.62(2H, s); 4.18(2H, d, J=2); 1.45, 1.8, 2.5, 2.78, 4.1, 7.35, 7.4 (all multiplets)

Step F: 1-t-Butyloxycarbonyl-4-(2-benzylthiazol-5-yl)piperidine

A mixture of 1-t-butyloxycarbonyl-4-(2-phenylacetamido)acetylpiperidine (595 mg, 1.653 mmol) from Step E and Lawesson's reagent (607 mg, 1.66 mmol) in 5 mL of toluene was heated to 120° C. for 3.5 h. After cooling, 3:1 mixture of ethyl acetate and methylene chloride and saturated sodium bicarbonate solution were added and the mixture was stirred for 0.5 h. The organic phase was separated and washed with brine. Solvent removal gave a crude product which was purified on silica gel using 2:3 mixture of ethyl acetate-hexane as solvent to give the desired product.

¹H NMR (CDCl₃): δ 1.45(9H, s); 4.4(2H, s); 7.46(1H, s); 1.58, 1.95, 2.85, 2.95, 4.2 (all multiplets).

Step G: 4-(2-Benzylthiazol-5-yl)piperidine di-hydrochloride

Acetyl chloride (0.3 mL) was added dropwise to a solution 1-t-butyloxycarbonyl-4-(2-benzylthiazol-5-yl)piperidine from Step F in methanol (2 mL) at ice bath temperature. The reaction mixture was stirred 3.5 h as it warmed to rt. Solvent removal in vacuo gave the desire amine as glassy solid. ¹H NMR (CDCl₃): δ 4.58(2H, s); 8.02(1H, s); 1.94, 2.24. 3.15, 3.35, 3.45 (all multiplets)

### Piperidine 6 (Method B)

4-(2-B enzylthiazol-5-yl)piperidine di-HCl salt

Step A: 1-t-Butyloxycarbonyl-4-(2-hydroxyethyl)piperidine

A mixture of 4-(2-hydroxyethyl) piperidine (5.0 g, 40 mmol), diA-t butyl dicarbonate (10.9 g, 50 mmol), and triethylamine (7 mL, 50 mmol) in 100 mL of anhydrous methylene chloride was stirred overnight at rt. Volatiles were removed in vacuo and the resulting oil was purified on a silica gel column using 20% ethyl acetate in hexane as eluent to give the desired product as a colorless oil.

Step B: 1-t-Butyloxycarbonyl-4-formylylmethylpiperidine

Oxalyl chloride (2.2 mL, 25 mmol) was added to 75 mL of anhydrous methylene chloride at −78° C. DMSO (3.5 mL, 50 mmol) was then added dropwise over 5 min, and the resulting mixture was stirred for 15 min. 1-t-Butyloxycarbonyl-4-(2-hydroxyethyl)piperidine (2.29 g, 10 mmol, Step A) was dissolved in 5 mL of anhydrous methylene chloride and added over 10 min to the above mixture. After stirring 30 min, DIEA (17.4 mL, 100 mmol) was added over 10 min. The mixture was then warmed to 0° C. and maintained at that temperature for 1 h. After quenching with water, the reaction mixture was diluted with 75 mL of methylene chloride and the layers were separated. The organic phase was washed with 3×50 mL of water and dried over anhydrous magnesium sulfate. Solvent removal gave an oil, which was purified on silica gel using 20% ethyl acetate in hexane to give the desired aldehyde which hardened overnight into an oily solid.

NMR: (CDCl₃): δ 2.15 (2H, d, J=3); 9.8 (1H, s); 1.2, 1.5, 1.7, 2.75, 4.1 (all multiplets)

Step C: 1-t-Butyloxycarbonyl-4-(α-bromo-formylmethyl)piperidine

A mixture of 1-t-butyloxycarbonyl-4-formylylmethylpiperidine (0.57 g, 2.25 mmol, step B), 3,3-dibromo-Meldrum's acid (0.75 g, 2.5 mmol) in 10 mL of anhydrous ether was stirred for 2 days at rt under nitrogen. The reaction mixture was diluted with ethyl acetate and washed with sat'd. sodium bicarbonate solution. The organic phase was dried over anhydrous magnesium sulfate. Solvent removal and purification on silica gel using 20% ethyl acetate in hexane as solvent gave the pure bromo aldehyde as a colorless oil.

¹H NMR: (CDCl₃): 8: 4.04 (1H, dd; J=1.5;2); 9.46 (1H, d; J=1.5) 1.35, 1.7, 1.95, 2.1, 2.75, 4.2 (all multiplets)

Step D: 1-t-Butyloxycarbonyl-4-(2-benzylthiazol-5-yl)piperidine

A mixture of 1-t-butyloxycarbonyl-4-(α-bromo-formylmethyl)piperidine (612 mg, 2 mmol), benzyl thioamide (500 mg, 2.55 mmol) in 10 mL of anhydrous toluene was heated to reflux for 6 h. Solvent was then removed and the residue was purified on silica gel using 25% ethyl acetate in hexane as solvent to give the desired thiazole as an oil.

¹H NMR (CDCl₃): δ 1.45(9H, s); 4.4(2H, s); 7.46(1H, s); 1.58,1.95,2.85,2.95,4.2 (all multiplets).

Step E: 4-(2-Benzylthiazol-5-yl)piperidine di-hydrochloride

The title compound was prepared by removal of the protecting group of 1-t-butyloxycarbonyl-4-(2-benzylthiazol-5-yl)piperidine as described above in Method A, Step G.

### Piperidine 7

4-(3,3-Difluoro-3-(3-pyridyl)propyl)piperidine

Step A: Dimethyl (2-oxo-2-(3-pyridyl)ethyl)phosphonate

A solution of n-butyl lithium in hexanes (9.0 mL, 1.6 M, 14 mmol) was added over 10 min. to a solution of dimethyl methylphosphonate (1.50 mL , 1.72 g, 13.8 mmol) in THF (60 mL) cooled in a dry ice/isopropanol bath. After 30 min., a solution of methyl nicotinate (757 mg, 5.52 mmol) in TEF (6 ML) was added over 2 min. The solution was stirred in the cooling bath for 45 min. before being allowed to warm to 0° C. over 1 h. The reaction was quenched with saturated aq. NH₄Cl (50 mL) and then partitioned between saturated aq. brine (50 mL) and methylene chloride (200 mL). The aq. layer was extracted with methylene chloride (2×100 mL). The combined organic layers were dried (sodium sulfate) decanted, and evaporated. Purification by flash column chromatography on silica gel, eluting with ethyl acetate followed by 97:3 v/v methylene chloride/CH₃0H, gave material containing some residual impurity. Further purification by flash column chromatography on silica gel, eluting with 50:50:5 v/v/v to 50:50:10 v/v/v toluene/ethyl acetate /CH₃OH gave the title compound. For the title compound:

¹H NMR (500 MHz, CDCl₃) δ 9.26-9.20 (bs, 1H), 8.83 (d, J=4, 1H), 8.34 (dt, J=8, 2, 1H), 7.70 (dd, J=8, 4, 1H), 3.82 (d, J=11, 6H), 3.67 (d, J=24, 2H).

Step B: 1-(t-Butoxycarbonyl)-4-(3-oxo-3-(3-pyridyl)prop-1-enyl)piperidine

1,1,1-Triacetoxy-1,1-dihydro-1,2-benzoiodoxol-3(1H)-one (750 mg, 1.77 mmol) was added to a solution of 1-(t-butoxycarbonyl)-4-(hydroxymethyl)piperidine (339 mg, 1.57 mmol, from Procedure 17, Step A) in methylene chloride (10 mL) and the mixture was stirred at rt. After 45 min., and additional portion of 1,1,1-triacetoxy-1,1-dihydro-1,2-benzoiodoxol-3(1H)-one (150 mg, 0.35 mmol) was added. After an additional 30 min., ether (30 mL) and 1.3 N NaOH (10 mL) were added and stirring was continued for 20 min. The mixture was transferred to a separatory funnel with additional ether (30 mL) and 1.3 N NaOH (15 mL). The organic layer was separated, washed with water (20 mL), dried (sodium sulfate), decanted, and evaporated to give 1-(t-butoxycarbonyl)-4-piperidinecarboxaldehyde as a colorless oil.

A solution of dimethyl (2-oxo-2-(3-pyridyl)ethyl)phosphonate (150 mg, 0.65 mmol, from Procedure 38, Step A) in TIfF (1.8 mL) was added to a stirred suspension of sodium hydride (60% oil dispersion, 15 mg of sodium hydride, 0.63 mmol) in THF (3.0 mL). The resulting suspension was warmed in a 45° C. oil bath for 30 min. After the mixture had cooled to rt, 1-(t-butoxycarbonyl)-4-piperidinecarboxaldehyde (112 mg, 0.53 mmol) was added in THF (1.5 mL). After stirring overnight at rt, the mixture was diluted with ether (20 mL) and washed with 2.5 N NaOH (20 mL) followed by saturated aq. brine (20 mL). The aq. layers were extracted in succession with ether (20 mL), and the combined organic layers were dried (sodium sulfate), decanted, and evaporated. Purification by flash column chromatography on silica gel, eluting with 80:20 v/v to 60:40 v/v hexanes/ethyl acetate, gave the title compound (trans isomer) as a yellow syrup. For the title compound:

¹H NMR (500 MHz, CDCl₃) δ 9.17-9.13 (bs, 1H), 8.81 (bd, J=4, 1H), 8.27 (d, J=8, 1H), 7.49 (dd, J=8, 4, 1H), 7.07 (dd, J=15, 7, 1H), 6.85 (dd, J=15, 1, 1H), 4.25-4.13 (bs, 2H), 2.87-2.78 (m, 2H), 2.51-2.41 (m, 1H), 1.83 (d, J=12, 2H), 1.49 (s, 9H), 1.45 (qd, J=12, 4, 2H).

ESI-MS 261 (M+H-56), 217 (M+H−100); HPLC A: 1.73 min.

Step C: 1-(t-Butoxycarbonyl)-4-(3-oxo-3-(3-pyridyl)propyl)piperidine

1-(t-Butoxycarbonyl)-4-(3-oxo-3-(3-pyridyl)prop-1-enyl)piperidine (940 mg, 2.97 mmol, from Procedure 38, Step B) was hydrogenated using 5% Pd/C in 95% ethanol at atmospheric pressure. Purification by flash column chromatography on silica gel, eluting with 90:10 v/v to 50:50 v/v hexanes/ethyl acetate gave the title compound as a colorless syrup. For the title compound:

¹H NMR (500 MHz, CDCl₃) δ 9.23-9.15 (bs, 1H), 8.81 (bd, J=4, 1H), 8.28 (dt, J=8, 1, 1H), 7.48 (dd, J=8, 4, 1H), 4.19-4.04 (bs, 2H), 3.04 (t, J=8, 2H), 2.70 (bt, J=11, 2H), 1.78-1.70 (m, 4H), 1.56-1.45 (m, 1H), 1.47 (s, 9H), 1.17 (qd, J=12, 4, 2H). ESI-MS 263 (M+H-56), 219 (M+H-100); HPLC A: 1.78 min.

Step D: 1-(t-Butoxycarbonyl)-4-(3,3-difluoro-3-(3-pyridyl)propyl)piperidin

A solution of 1-(t-butoxycarbonyl)-4-(3-oxo-3-(3-pyridyl)propyl)piperidine (810 mg, 2.54 mmol, from Procedure 38, Step C) in (diethylamino)sulfur trifluoride (3.30 mL, 3.66 g, 23 mmol) was stirred in a teflon tube at 40° C. for 2 days. The reaction was diluted with methylene chloride (20 mL) and the resulting solution was added in portions to a stirred mixture of water (150 mL), ice (150 g) and sodium bicarbonate (29.3 g). After the resulting reaction had subsided, the mixture was extracted with ethyl acetate (2×200 mL). The organic layers were washed in succession with saturated aq. brine (100 mL ), dried (sodium sulfate), decanted, and evaporated. Flash column chromatography on silica gel, eluting with 80:20 v/v to 50:50 v/v toluene/ether, gave material containing some residual impurity. Further purification by preparative HPLC on a 20×250 mm Chiracel OD column, eluting with 80:20 v/v hexanes/isopropanol, gave the title compound:

¹H NMR (500 MHz, CD₃OD) δ 8.69 (s, 1H), 8.64 (d, J=5, 1H), 7.97 (d, I =8, 1H), 7.54 (dd, J=8, 5, 1H), 4.04 (d, J=13, 2H), 2.78-2.62 (bs, 2H), 2.31-2.20 (m, 2H), 1.68 (d, J=12, 2H), 1.50-1.40 (m, 1H), 1.43 (s, 9H), 1.40-1.34 (m, 2H), 1.02 (qd, J=12,4,2H).

ESI-MS 285 (M+H-56), 241 (M+H-100); BPLC A: 2.10 min.

Step E: 4-(3,3-Difluoro-3-(3-pyridyl)propyl)piperidine

The title compound was prepared using procedures analogous to those described for Piperidine 2, Step H, substituting 1-(t-butoxycarbonyl)-4-(3,3-difluoro-3-(3-pyridyl)propyl)piperidine (from Piperidine 7, Step D) for 1-(t-butoxycarbonyl)-4-(3,3-difluoro-3-(4-fluorophenyl)propyl)piperidine. For the title compound:

¹H NMR (500 MHz, CD₃OD) δ 8.68 (s, 1H), 8.64 (d, J=4, 1H), 7.97 (d, J=8, 1H), 7.54 (dd, J=8, 4, 1H), 2.98 (bd, J=12, 2H), 2.52 (td, J=12, 3, 2H), 2.30-2.18 (m, 2H), 1.66 (bd, J=13, 2H), 1.44-1.30 (m, 3H), 1.08 (qd, J=12, 3, 2H); ESI-MS 241 (M+H).

### Piperidine 8

4-(3,3-Difluoro-3-(6-methylpyridazin-3-yl)propyl)piperidine

Step A: 3-Bromo-6-methylpyridazine

A solution (3.0 mL) containing 30% BBr in acetic acid was added to 3-(trifluoromethanesulfonyloxy)-6-methylpyridazine (prepared as described by M. Rohr, et al., Heterocycles, 1996, 43, 1459-64) and the mixture was heated in a 100° C. oil bath for 2.5 h. The mixture was cooled in an ice bath, adjusted to pH ≧9 (as determined using pH paper) by the careful addition of 20% aqueous NaOH, and extracted with ether (3×20 mL). The organic layers were dried (sodium sulfate), decanted, and evaporated to give the title compound as pale tan crystals. For the title compound:

¹H NMR (500 MHz, CDCl₃) δ 7.56 (d, J=9, 1H), 7.22 (d, J=9, 1H), 2.70 (s, 3H).

Step B: Ethyl difluoro(6-methylpyridazin-3-yl)acetate

This procedure is derived from the general method of T. Taguchi, et al. (Tetrahedron Lett., 1986, 27, 6103-6106). Ethyl difluoroiodoacetate (0.355 mL, 651 mg, 2.60 mmol) was added to a rapidly stirred suspension of copper powder (333 mg, 5.24 mmol) in DMSO (6.5 mL) at rt. After 50 min., 3-bromo-6-methylpyridazine (300 mg, 1.73 mmol) was added in DMSO (1.0 mL). After 20 h, the mixture was transferred to a separatory funnel containing water (25 mL) and saturated aq. NH₄Cl (25 mL), and extracted with ethyl acetate (2×50 mL). The organic extracts were washed with saturated aq. brine, dried (sodium sulfate), decanted, and evaporated. Purification by flash column chromatography on silica gel, eluting with 70:30 v/v hexanes/ethyl acetate, gave the title compound as an amber liquid. For the title compound:

¹H NMR (500 MHz, CDCl₃) δ 7.79 (d, J=9, 1H), 7.53 (d, J=9, 1H), 4.43 (q, J=7, 2H), 2.82 (s, 3H), 1.38 (t, J=7, 3H).

Steps C-E: 4-(3,3-Difluoro-3-(6-methylpyridazin-3-yl)propyl)piperidine

The title compound was prepared using procedures analogous to those described for Piperidine 2, Steps F-H, substituting ethyl difluoro(6-methylpyridazin-3-yl)acetate (from Procedure 36 Step B) for ethyl difluoro(2-pyridyl)acetate in Step C. For the title compound:

¹H NMR (500 MHz, CD₃OD) δ 7.86 (d, J=9, 1H), 7.74 (d, J=9, 1H), 2.99 (dm, J=12, 2H), 2.74 (s, 3H), 2.54 (td, J=12, 3, 2H), 2.51-2.40 (m, 2H), 1.69 (bd, J=12, 2H), 1.47-1.34 (m, 3H), 1.10 (qd, J=12, 4, 2H).

### Piperidine 9

4-(3,3-Difluoro-3-(5-(trifluoromethyl)pyrid-2-yl)propyl)piperidine

The title compound was prepared using procedures analogous to those described in Piperidine 8, substituting 2-bromo-5-(trifluoromethyl)pyridine for 3-bromo-6-methylpyridazine in Step B. For the title compound:

¹H NMR (500 MHz, CD₃OD) δ 8.96 (s, 1H), 8.28 (dd, J=8, 2, 1H), 7.88 (d, J=8, 1H), 2.99 (bd, J=12, 2H), 2.53 (td, J=12, 2, 2H), 2.43-2.31 (m, 2H), 1.68 (bd, J=13, 2H), 1.44-1.28 (m, 3H), 1.09 (qd, J=12, 3, 2H);

ESI-MS 309 (M+H); HPLC A: 2.32 min.

Using essentially the same methods as described for Piperidine 1 and substituting the appropriate starting material and/or hydrazine reagent, the following representative 4-(3-(substituted)-1-(H or alkyl)-(1H)-pyrazol-5-yl)piperidines can be prepared, usually as the di-hydrochloride salts, and utilized in the following Examples as required.

### Piperidine 10

4-(3-(3-Methoxybenzyl)-1-(ethyl)-(1H)-pyrazol-5-yl)piperidine

### Piperidine 11

4-(3-(4-Methoxybenzyl)-1-(ethyl)-(1H)-pyrazol-5-yl)piperidine

### Piperidine 12

4-(3-(3-Ethoxybenzyl)-1-(ethyl)-(1H)-pyrazol-5-yl)piperidine

### Piperidine 13

4-(3-(4-Ethoxybenzyl)-1-(ethyl)-(1H)-pyrazol-5-yl)piperidine

### Piperidine 14

4-(3-(4-Isopropoxybenzyl)-1-(ethyl)-(1H)-pyrazol-5-yl)piperi dine

### Piperidine 15

4-(3-(4-Cyclopropoxybenzyl)-1-(ethyl)-(1H)-pyrazol-5-yl)piperidine

### Piperidine 16

4-(3-(4-Cyclobutoxybenzyl)-1-(ethyl)-(1H)-pyrazol-5-yl)piperidine

### Piperidine 17

4-(3-(4-Trifluoromethoxybenzyl)-1-(ethyl)-(1H)-pyrazol-5-yl)piperidine

### Piperidine 18

4-(3-(3,4-Methylenedioxybenzyl)-1-(ethyl)-(1H)-pyrazol-5-yl)piperidine

### Piperidine 19

4-(3-(3,4-Dimethoxybenzyl)-1-(ethyl)-(1H)-pyrazol-5-yl)piperidine

### Piperidine 20

4-(3-(3-Fluoro-4-methoxybenzyl)-1-(ethyl)-(1H)-pyrazol-5-yl)piperi dine

### Piperidine 21

4-(3-(3-Fluoro-4-ethoxybenzyl)-1-(ethyl)-(1H)-pyrazol-5-yl)piperidine

### Piperidine 22

4-(3-(Benzofuran-6-yl)-1-(ethyl)-(1H)-pyrazol-5-yl)piperidine

### Piperidine 23

4-(3-(4-Methylbenzyl)-1-(ethyl)-(1H)-pyrazol-5-yl)piperi dine

### Piperidine 24

4-(3-(4-Isopropylbenzyl)-1-(ethyl)-(1H)-pyrazol-5-yl)piperidine

### Piperidine 25

4-(3-(4-t-Butylbenzyl)-1-(ethyl)-(1H)-pyrazol-5-yl)piperidine

### Piperidine 26

4-(3-(1-Naphthyl)-1-(ethyl)-(1H)-pyrazol-5-yl)piperidine

### Piperidine 26a

4-(3-(3-Pyrid-methyl)-1-(ethyl)-(1H)-pyrazol-5-yl)piperidine

Using essentially the same methods as described for Piperidine 6 and substituting the appropriate starting material and/or reagent, the following representative 4-(2-(substituted)-4-(H or alkyl)thiazol-5-yl)piperidines can be prepared, usually as the di-hydrochloride salts, and utilized in the following Examples as required.

### Piperidine 27

4-(2-(4-Chlorobenzyl)thiazol-5-yl)piperidine

### Piperidine 28

4-(2-(4-Ethoxybenzyl)-4-(ethyl)thiazol-5-yl)piperidine

### Piperidine 29

4-(2-(4-Ethoxybenzyl)thiazol-5-yl)piperidine

### Piperidine 30

4-(2-(4-Trifluoromethoxybenzyl)-4-(ethyl)thiazol-5-yl)piperidine

### Piperidine 31

4-(2-(4-Trifluoromethoxybenzyl)thiazol-5-yl)piperidine

### Piperidine 32

N′-Benzyl-N″-cyano-N-ethyl-N-(piperidin-4-yl)guanidine

Step A: N-Benzyl-N′-cyano-thiourea

Freshly prepared sodium ethoxide solution (0.192 g, 8.35 mmol of sodium in 10 mL of EtOH) was reacted with 349 mg (8.3 mmol) of cyanamide. Benzyl isothiocyanate (1.1 mL, 8.2 mmol) was added and the reaction was refluxed for 30 min. After cooling to rt, volatiles were removed under reduced pressure. The crude product was partitioned between 100 mL of EtOAc and 100 mL of 1 N HCl. After separating phases, the organic layer was washed with brine, dried over Na₂SO₄ and concentrated under reduced pressure to give the title compound as white solid, which was used without further purification.

Step B: N′-Benzyl-N″-cyano-N-ethyl-N-(1-tert-butoxycarbonylpiperidin-4-yl)guanidine

A solution of 100 mg (0.52 mmol) of N-benzyl-N′-cyano-thiourea (from Step A) and 147 mg (0.64 mmol) of 1-(tert-butoxycarbonyl)-4-(ethylamino)-piperidine (from Piperidine 38, Step A) at rt was treated with 120 mg (0.62 mmol) of 1-(3-dimethylaminopropyl)-3-ethylcarbodiimide (Tetrahedron Lett. 1989, 30, 7313-7316). After 30 min the reaction was diluted with 50 mL of EtOAc and washed with 50 mL of 1 N HCl and 50 mL of H₂O. The organic phase was dried over MgSO₄ and concentrated under reduced pressure. The residue was purified by flash chromatography using 99:1 and 97:3 v/v of CH₂Cl₂/MeOH as the eluant to afford an impure product, which was rechromatographed using 7:3 v/v hexane/acetone to afford the title compound: R_{F}: 0.20 (7:3 v/v hexanes/acetone); ¹H-NMR (500 Mhz) δ 1.10 (t, J=7.1, 3H), 1.44 (s, 9H), 1.45-1.56 (m, 2H), 1.65-1.67 (m, 2H), 2.61-2.71 (m, 2H), 3.20 (q, J=7.1, 2H), 4.14-4.32 (m, 3H), 4.68 (d, J=7.1, 2H), 5.90 (m, 1H), 7.24-7.33 (m, 5H).

Step C: N′-Benzyl-N″-cyano-N-ethyl-N-(piperidin-4-yl)guanidine

A solution of 54 mg (0.14 mmol) of N′-benzyl-N″-cyano-N-ethyl-N-(1-tert-butoxycarbonylpiperidin-4-yl)guanidine (from Step B) in 4 N HCl in dioxane was stirred at rt for 20 min. Volatiles were removed under reduced pressure. The crude product was partitioned between 25 mL of 1 N NaOH and 25 mL of CH₂Cl₂. After separating phases, the aqueous layer was extracted with 2×25 mL of CH₂Cl₂. The combined organic layers were dried over Na₂SO₄ and concentrated under reduced pressure to afford the title compound as a colorless film: ¹H-NMR (500 Mhz) δ 1.10 (t, J=7.1, 3H), 1.55-1.70 (m, 4H), 2.38 (m, 1H), 2.62-2.66 (m, 2H), 3.07-3.10 (m, 2H), 3.25 (q, J=7.1, 2H), 4.21 (m, 1H), 4.68 (d, J=5.7, 2H), 5.72 (m, 1H), 7.24-7.33 (m, 5H).

### Piperidine 33

(E/Z)-N^{1′}-Benzyl-2-nitro-N^{1″}-(piperidin-4-yl)ethene-1,1-diamine

Step A: 1-Methylthio-2-nitro-1-(piperidin-4-yl)amino-ethene

A solution of 201 mg (1.0 mmol) of 4-amino-1-tert-butoxycarbonylpiperidine and 170 mg (1.0 mmol) of 1,1-bis(methylthio)-2-nitroethylene in 5 mL of CH₃CN was refluxed for 6 hours. Volatiles were removed under reduced pressure. After preabsorbing the crude product onto silica gel, the residue was purified by flash chromatography using 1:1 v/v hexane/EtOAc to afford the title compound as a white solid: R_{F}: 0.22 (3:2 v/v hexanes/EtOAc); ¹H-NMR (500 Mhz) δ 1.47 (s, 9H), 1.48-1.61 (m, 2H), 1.99-2.04 (m, 2H), 2.46 (s, 3H), 2.95-3.00 (m, 2H), 3.81 (m, 111), 4.01-4.03 (m, 2H), 6.55 (s, 1H).

Step B: (E/Z-N^{1′}-Benzyl-2-nitro-N^{1″}-(1-tert-butoxycarbonylpiperidin-4-yl)ethene-1,1-diamine

A mixture of 50 mg (0.15 mmol) of 1-methylthio-2-nitro-1-(piperidin-4-yl)amino-ethene (from Step A) in 0.1 mL of benzyl amine was warmed to 90° C. in a sealed vial for 15 min. After cooling to rt, the reaction was dissolved in 50 mL of CH₂Cl₂ and washed with 50 mL of 1 N HCl and 50 mL of 1 N NaHCO₃. The organic layer was dried over Na₂SO₄ and concentrated under reduced pressure. The residue was purified by flash chromatography using 97:3 v/v CH₂Cl₂/MeOH to afford the title compound as a colorless film: R_{F}: 0.40 (19:1 v/v CH₂Cl₂/MeOH); ¹H-NMR (500 Mhz) δ 1.26-1.50 (m, 111H), 1.79-1.96 (m, 2H), 2.80-2.86 (m, 2H), 3.82-3.92 (m, 3H), 4.32-4.48 (m, 2H), 6.42-6.58 (m, 1H), 7.20-7.35 (m, 5H), 7.79 (m, 1H).

Step C: (E/)-N^{1′}-Benzyl-2-nitro-N^{1″}-(piperidin-4-yl)ethene-1,1-diamine

The title compound was prepared from (E/Z)-N^{1′}-benzyl-2-nitro-N^{1″}-(1-tert-butoxycarbonylpiperidin-4-yl)ethene-1,1-diamine (from Step B) using a procedure analogous to Piperidine 32, Step C. ¹H-NMR (500 Mhz) δ 1.25-2.01 (m, 4H), 2.57-2.65 (m, 2H), 2.96-3.23 (m, 2H), 4.32-4.48 (m, 2H), 5.10 (m, 1H), 6.53 (m, 1H), 7.23-7.30 (m, 5H).

### Piperidine 34

4-(1-Ethyl-(1H)-pyrazol-5-yl)piperidine

Step A: 1-(1-(tert-Butoxycarbonyl)piperidin-4-yl)-3-hydroxy-propenone

A suspension of 0.51 g (2.2 mmol) of 4-acetyl-1-(tert-butoxycarbonyl)piperidine (from Piperidine 1, Step A, Method B, Step B2), 0.36 mL (4.4 mmol) of ethylformate and 0.11 g (4.3 mmol) of 95% sodium hydride in 6 mL of THF was refluxed for 30 min. After cooling to rt, the reaction was partitioned between 100 mL of Et₂O and 100 mL of 1 N HCl. The phases were separated. The organic layer was dried over MgSO₄ and concentrated under reduced pressure to yield the title compound as a yellow oil, which was used without further purification. 1H-NMR (500 Mhz) δ 1.46 (s, 9H), 1.47-1.60 (m, 2H), 1.80-1.85 (m, 2H), 2.38 (m, 1H), 2.73-2.79 (m, 2H), 4.10-4.15 (m, 2H), 5.55 (d, J=4.3, 1H), 7.98 (d, J=4.3, 1H).

Step B: 1-(tert-Butoxycarbonyl)-4-(1-ethyl-(1H)-pyrazol-5-yl)piperidine

To a solution of 175 mg (0.68 mmol) of 1-(1-(tert-butoxycarbonyl)piperidin-4-yl)-3-hydroxy-propenone (from Step A) in 3 mL of CH₃CN and 1.5 mL of H₂O was added 0.125 mL (0.70 mmol) of 34% aqueous ethylhydrazine. After stirring at rt for 45 min, the reaction was partitioned between 75 mL of Et₂O and 25 mL of brine. After separating layers, the organic phase was dried over MgSO₄ and concentrated under reduced pressure. The residue was purified on a 40S Biotage column using 9:1 v/v of hexanes/acetone as the eluant to afford the title compound as a white solid and the undesired regioisomer as a colorless oil. Title Compound: R_{F}: 0.52 (3:2 v/v of hexanes/acetone); 1H-NMR (500 Mhz) δ 1.41-1.64 (m, 14H), 1.85-1.87 (m, 2H), 2.70-2.84 (m, 3H), 4.14 (q, J=7.4, 2H), 4.18-4.24 (m, 2H), 6.02 (d, J=2.0, 1H), 7.44 (d, J=2.0, 1H).

Step C: 4-(1-Ethyl-(1H)-pyrazol-5-yl)piperidine

The title compound was prepared from 1-(t-butoxycarbonyl)-4-(1-ethyl-(1H)-pyrazol-5-yl)piperidine (from Step B) using a procedure analogous to Piperidine 32, Step C, except 3/2 v/v CH₂Cl₂/trifluoroacetic acid was substituted for 4 N HCl in dioxane. ¹H-NMR (500 Mhz) δ 1.44 (br t, 3H), 1.59-1.66 (m, 2H), 1.85-1.88 (m, 2H), 2.38 (br m, 1H), 2.67-2.77 (m, 3H), 3.18-3.21 (m, 2H), 4.10 (br q, 2H), 6.02 (br s, 1H), 7.41 (br s, 1H).

### Piperidine 35

4-(1,3-Diethyl-(1H)-pyrazol-5-yl)piperidine

Step A: 1-(1-(tert-Butoxycarbonyl)piperidin-4-yl)-3-hydroxy-pent-2-en-1-one

A solution of 2.07 g (9.1 mmol) of 4-acetyl-1-(t-butoxycarbonyl)piperidine (from Piperidine 1, Step A, Method B, Step B2) and 1.7 mL (17.6 mmol) of methyl propionate in 20 mL of methyl, tert-butylether at 0° C. was treated with 1.96 g (16.5 mmol) of potassium tert-butoxide. After 10 minutes at 0° C., the reaction was stirred at rt for 22 hours. The reaction was quenched with 1 N HCl and partitioned between 100 mL of Et₂O and 100 mL of 1 N HCl. After separating phases, the organic layer was washed with 100 mL of brine, dried over MgSO₄ and concentrated under reduced pressure. The residue was purified on a 40M Biotage column using 19:1 v/v of hexanes/acetone as the eluant to afford the title compound as a yellow oil: R_{F}: 0.53 (4:1 v/v of hexanes/acetone); ¹H-NMR (500 Mhz) δ 1.14 (t, J=7.5, 3H), 1.46 (s, 9H), 1.47-1.61 (m, 2H), 1.80-1.83 (m, 2H), 2.29-2.37 (m, 3H), 2.72-2.78 (m, 2H), 4.12-4.17 (m, 2H), 5.50 (s, 1H).

Step B: 1-(tert-Butoxycarbonyl)-4-(1,3-diethyl-(1H)-pyrazol-5-yl)piperidine

The title compound was prepared from 1-(1-(tert-butoxycarbonyl)piperidin-4-yl)-3-hydroxy-pent-2-en-1-one (from Step A) using a procedure analogous to Piperidine 34, Step B. R_{F}: 0.34 (4:1 v/v of hexanes/acetone); ¹H-NMR (500 Mhz) δ 1.23 (t, J=7.5, 3H), 1.40-1.62 (m, 14H), 1.84-1.86 (m, 2H), 2.58-2.81 (m, 511), 4.05-4.23 (m, 4H), 5.82 (s, 1H).

Step C: 4-(1,3-Diethyl-(1H)-pyrazol-5-yl)piperidine

The title compound was prepared from 1-(tert-butoxycarbonyl)-4-(1,3-diethyl-(1H)-pyrazol-5-yl)piperidine (from Step B) using a procedure analogous to Piperidine 34, step C. ¹H-NMR (500 Mhz) δ 1.22 (t, J=7.5, 3H), 1.41 (t, J=7.2, 3H), 1.57-1.66 (m, 2H), 1.85-1.87 (m, 2H), 2.41 (br m, 1H), 2.58-2.77 (m, 5H), 3.18-3.21 (m, 2H), 4.03 (q, J=7.2, 2H), 5.83 (s, 1H).

### Piperidine 36

4-(1-Ethyl-4-methyl-(1H)-pyrazol-5-yl)piperidine

The title compound was prepared using procedures analogous to Piperidine 35, except ethyl formate and 4-propionyl-1-(tert-butoxycarbonyl)piperidine were used in Step A. ¹H-NMR (500 Mhz) δ 1.38 (t, J=7.0, 3H), 1.71 (m, 2H), 1.87-2.09 (m, 3H), 2.10 (s, 3H), 2.68-2.80 (m, 3H), 3.19-3.21 (m, 2H), 4.11 (q, J=7.0, 2H), 7.20 (s, 1H).

### Piperidine 37

4-(1-Ethyl-3-methyl-(1H)-pyrazol-5-yl)piperidine

The title compound was prepared using procedures analogous to Piperidine 35, except ethyl acetate was substituted for methyl propionate in Step A. ¹H-NMR (500 Mhz) δ 1.41 (t, J=7.1, 3H), 1.55-1.63 (m, 2H), 1.83-1.86 (m, 2H), 1.97 (br m, 1H), 2.23 (s, 3H), 2.61-2.76 (m, 3H), 3.17-3.19 (m, 2H), 4.03 (q, J=7.1, 2H), 5.80 (s, 1H).

### Piperidine 38

4-(N-Ethyl-N-phenylsulfonylamino)piperidine, hydrochloride salt

Step A: 1-(tert-Butoxycarbonyl)-4-(ethylamino)-piperidine

A solution of 5.0 g (25.0 mmol) of 1-(tert-butoxycarbonyl)-4-piperidone and 2.48 g (30.4 mmol) of ethylamine hydrochloride salt in 100 mL of CH₂Cl₂ at rt was treated with 7.9 g (37.2 mmol) of sodium triacetoxyborohydride. After stirring for 1.5 h at the reaction was purified using an analogous procedure in Example 1, Step A. ¹H-NMR (500 Mhz) δ 1.10 (t, J=7.1, 3H), 1.19-1.27 (m, 2H), 1.44 (s, 9H), 1.82-1.84 (m, 2H), 2.57-2.78 (m, 5H), 4.03 (m, 2H).

Step B: 1-(tert-Butoxycarbonyl)-4-(N-ethyl-N-phenylsulfonylamino)piperidine

A solution of 100 mg (0.44 mmol) of 1-(tert-butoxycarbonyl)-4-(ethylamino)-piperidine (from Step A), 11 mg (0.088 mmol) of 4-(dimethylamino)pyridine and 0.06 mL (0.44 mmol) of triethylamine in 1 mL of CH₂Cl₂ at 0° C. was treated with 0.056 mL (0.44 mmol) of benzenesulfonyl chloride. After warming to rt, the reaction was stirred for 1 h. The reaction was quenched with H₂O and partitioned between 25 mL of CH₂Cl₂ and 25 mL of 1 N HCl. After separating phases, the aqueous layer was extracted with 25 mL of CH₂Cl₂. The combined organic layers were washed with 25 mL 1 N NaHCO₃ and 25 mL of brine, dried over Na₂SO₄ and concentrated under reduced pressure. The residue was purified by flash chromatography using 4:1 v/v of hexanes/EtOAc as the eluant to afford the title compound as an off-white solid: R_{F}: 0.56 (3:2 v/v hexanes/EtOAc); ¹H-NMR (500 Mhz) δ 1.24 (t, J=7.1, 3H), 1.45 (s, 9H), 1.50-1.59 (m, 4H), 2.67 (m, 2H), 3.24 (q, J=7.1, 2H), 3.82 (m, 1H), 4.07-4.15 (m, 2H), 7.49-7.58 (m, 3H), 7.84-7.86 (m, 2H).

Step C: 4-(N-Ethyl-N-phenylsulfonylamino)piperidine, Hydrochloride Salt

The title compound was prepared using an analogous procedure for Piperidine 6, Method A, Step G.

### Piperidine 39

4-(N-Ethyl-N-benzylsulfonylamino)piperidine, Hydrochloride Salt

The title compound was prepared using analogous procedures for Piperidine 38, except α-toluenesulfonyl chloride was substituted for benzenesulfonyl chloride in Step B.

### Piperidine 40

4-(3-(3,4-Dimethoxyphenyl)propyl)piperidine, Para-toluenesulfonic Acid Salt

Step A: 2-(3,4-Dimethoxyphenyl)-1-iodoethane

A solution of 5.47 g (30.0 =mol) of 2-(3,4-dimethoxyphenyl)ethanol and 7.80 mL (45.0 mmol) of DIEA in 100 mL of CH₂Cl₂ at 0° C. was treated with 3.10 mL (40.0 mmol) of methanesulfonyl chloride and the resulting mixture was stirred cold for 30 min. The mixture was partitioned between 400 mL of ether and 200 mL of 1 N HCl and the layers were separated. The organic layer was washed with 200 mL of sat'd NaHCO₃, 200 mL of brine, dried over MgSO₄ and concentrated.

A mixture of the crude mesylate and 22.5 g (150 mmol) of sodium iodide in 100 mL of acetone was heated at reflux for 1 h. The mixture was cooled and partitioned between 500 mL of ether and 200 mL of water. The organic layer was separated and washed with 250 mL of 5% NaS₂O₃, 250 mL of brine, dried over MgSO₄ and concentrated. Flash chromatography on 200 g of silica gel using 4:1 v/v hexanes/ether as the eluant afforded the title compound: ¹H-NMR (400 MHz, CDCl₃): δ 3.12 (t, J=8.0, 2H), 3.33 (t, J=8.0, 2H), 3.86 (s, 3H), 3.88 (s, 3H), 6.07-6.82 (3H).

Step B: 4-(3-(3,4-Dimethoxyphenyl)propyl)pyridine

A solution 1.95 mL (20.0 mmol) of 4-picoline in 30 mL of THF at −78° C. was treated with 12.5 mL of 1.6 M n-butyllithium solution in hexanes. The resulting mixture was warmed to rt, stirred for 1 h, then recooled to −78° C. A solution of 8.50 g (19.0 mmol) of 2-(3,4-dimethoxyphenyl)-1-iodoethane (from Step A) in 25 mL of TUF was added via cannula. The resulting mixture was warmed to 0° C. and stirred 1 h. The reaction was quenched with 200 mL of water, then extracted with 300 mL of ether. The organic layer was separated and extracted 2×150 mL of 1.0 N HCl. The combined acid extracts were made basic (pH =9) with aqueous NHand extracted with 300 mL of ether. The ether extract was dried over MgSO₄ and concentrated. Flash chromatography on 175 g of silica gel using 1:1 hexaneslethyl acetate+1% TEA as the eluant afforded the title compound: ¹H-NMR (500 MHz, CDCl₃): δ 1.92-1.98 (m, 2H), 2.59-2.65 (4H), 3.86 (s, 3H), 3.87 (s, 31), 6.68-6.72 (2H), 6.80 (d, J=8.5, 1H), 7.11 (d, J=5.5), 8.49 (d, J=5.5); ESI-MS 258 (M+H); LC-1: 1.79min.

Step C: 4-(3-(3,4-Dimethoxyphenyl)propyl)piperidine, Para-toluenesulfonic Acid Salt

A mixture of 2.04 g (7.9 mmol) of 4-(3-(3,4-dimethoxyphenyl) propyl)pyridine (from Step C) and 200 mg of 5% platinum on carbon in 25 mL of HOAc was hydrogenated at 40 psi on a Paar shaker for 20 h. The catalyst was filtered. Toluene (200 mL) was added to the filtrate and the resulting mixture was concentrated. The residue was partitioned between 200 mL of ether and 100 mL of 20% aqueous NH₃ solution and the layers were separated. The organic layer was dried over MgSO₄ and concentrated to afford the free base of the title compound. The free based (1.16 g) and 835 mg of p-toluene sulfonic acid monohydrate was combined in 25 mL of MeOH and concentrated. The resulting solid was recrystallized from EtOAc to afford the title compound:: ¹H-NMR (500 MHz, CD₃OD): δ 1.26-1.35 (m, 2H), 1.55-1.65 (m, 2H), 1.89 (app d, J=13.5, 1H), 2.35 (s, 3H), 2.54 (t, J=7.5, 2H), 2.92 (dt, J=2.5, 13.0, 2H), 3.32 (app d, J=13.0), 3.77 (s, 3H), 3.79 (s, 3H), 6.70 (dd, J=2.0, 8.0, 1H), 6.77 (d, J=2.0, 111), 6.83 (d, J=8.0, 1H), 7.21 (d, J=7.5, 2H), 7.69 (d, J=7.5, 2H); ESI-MS 264 (M+H); LC-1: 2.00 min.

### Piperidine 41

4-(2-(4-Ethoxyphenylsulfonyl)ethyl)piperidine, Hydrochloride Salt

Step A: 4-(2-(4-Ethoxyphenylsulfonyl)ethyl)-1-tert-butoxycarbonylpiperidine

Sodium metal (230 mg, 10.0 mmol) was dissolved in 10 mL of EtOH. 4-(2-(4-fluorophenylsulfonyl)ethyl)-1-tert-butoxycarbonylpiperidine (371 mg, 1.0 mmol, Piperidine 3, Step E) was added and the resulting mixture was heated at reflux or 30 min. The mixture was cooled and concentrated. The residue was partitioned between 75 mL of ether and 25 mL of water and the layers were separated. The organic layer was dried over MgSO₄ and concentrated. Flash chromatography on 15 g of silica gel using 3:1 v/v hexanes/EtOAc afforded the title compound: ¹H-NMR (400 MHz, CDCl₃): δ 1.06 (dq, J=4.4, 12.4, 2H), 1.44 (s, 9H), 1.46 (t, J=7.2, 3H), 1.48-1.60 (4H), 2.63 (br t, J=11.6, 2H), 3.05-3.09 (m, 2H), 4.05-4.15 (4H), 7.00 (d, J=9.2, 2H), 7.80 (d, J=9.2, 2H).

Step B: 4-(2-(4-Ethoxyphenylsulfonyl)ethyl)piperidine, Hydrochloride Salt

A solution of 372 mg (0.94 mmol) of 4-(2-(4-ethoxyphenylsulfonyl) ethyl)-1-tert-butoxycarbonylpiperidine (from Step A) in EtOH saturated with HCl gas was stirred at rt for 2 h. The reaction mixture was concentrated. The residue was triturated with EtOAc and the solid was filtered and dried to afford the title compound: ESI-MS 298 (M+H); LC-1: 1.60 min.

### Piperdine 42

4-(Imidazo[1,2-a]pyridin-3-yl)piperidine di-TFA Salt

Step A: 1-t-Butyloxycarbonyl-4-(2-hydroxyethyl)piperidine

A mixture of 4-(2-hydroxyethyl) piperidine (5.0 g, 40 mmol), di-t-butyl dicarbonate (10.9 g, 50 mmol), and triethylamine (7 mL, 50 mmol) in 100 mL of anhydrous methylene chloride was stirred overnight at room temperature. Volatiles were removed in vacuo and the resulting oil was purified on a silica gel column using 20% ethyl acetate in hexane as eluent to give the desired product as a colorless oil.

Step B: 1-t-Butyloxycarbonyl-4-formylylmethylpiperidine

Oxalyl chloride (2.2 mL, 25 mmol) was added to 75 mL of anhydrous methylene chloride at −78° C. DMSO (3.5 mL, 50 mmol) was then added dropwise over 5 min, and the resulting mixture was stirred for 15 min. 1-t-Butyloxycarbonyl-4-(2-hydroxyethyl)piperidine (2.29 g, 10 mmol, Step A) was dissolved in 5 mL of anhydrous methylene chloride and added over 10 min to the above mixture. After stirring 30 min, DIEA (17.4 mL, 100 mmol) was added over 10 min. The mixture was then warmed to 0° C. and maintained at that temperature for 1 h. After quenching with water, the reaction mixture was diluted with 75 mL of methylene chloride and the layers were separated. The organic phase was washed with 3×50 mL of water and dried over anhydrous magnesium sulfate. Solvent removal gave an oil, which was purified on silica gel using 20% ethyl acetate in hexane to give the desired aldehyde which hardened overnight into an oily solid.

NMR: (CDCl₃): δ 2.15 (2H, d, J=3); 9.8 (1H, s); 1.2, 1.5, 1.7, 2.75, 4.1(all multiplets)

Step C: 1-t-Butyloxycarbonyl-4-(1-bromo-formylmethyl)piperidine

A mixture of 1-t-butyloxycarbonyl-4-formylylmethylpiperidine (0.57 g, 2.25 mmol, step B), 3,3-dibromo-Meldrum's acid (0.75 g, 2.5 mmol) in 10 mL of anhydrous ether was stirred for 2 days at room temperature under nitrogen. The reaction mixture was diluted with ethyl acetate and washed with sat'd. sodium bicarbonate solution. The organic phase was dried over anhydrous magnesium sulfate. Solvent removal and purification on silica gel using 20% ethyl acetate in hexane as solvent gave the pure bromo aldehyde as a colorless oil.

¹H NMR: (CDCl₃): δ: 4.04 (1H, dd; J=1.5; 2); 9.46 (1H, d; J=1.5) 1.35, 1.7, 1.95, 2.1, 2.75, 4.2 (all multiplets)

Step D: 1-(tert-Butoxycarbonyl)-4-(imidazo[1,2a]-alpyridin-3-yl)piperidine

To a solution of 1.15 g of 1-t-butyloxycarbonyl-4-(1-bromoformylmethyl)piperidine (from Step C) in 15 mL ethanol was added 388 mg of 2-aminopyridine. After refluxing for 18 h, the solvent was evaporated. The mixture was partitioned between ethyl acetate and saturated sodium bicarbonate solution. Aqueous layer was extracted with ethyl acetate (3×). The combined organic phase was washed with brine, dried over magnesium sulfate and concentrated. The residue was purified by flash chromatography with 50% ethyl acetate in hexanes, followed by 100% ethyl acetate to give the title compound as a solid. ¹H NMR (500 MHz, CDCl₃) δ 1.48 (s, 9H), 1.70 (m, 2H), 2.06 (d, J=13 Hz, 2H), 2.93-3.02 (m, 3H), 4.26 (br, 2H), 6.87 (t, J=6.8 Hz, 1H). 7.21(m, 1H), 7.44(s, 1H), 7. 69(d, J=9.2 Hz, 1H), 7.99 (d, J=6.9 Hz, 1H).

Step E: 4-nirdazo[1,2-a]pyridin-3-yl)piperidine di-TFA Salt

To 100 mg of 1-(tert-butoxycarbonyl)-4-(imidazo[1,2-a]pyridin-3-yl)piperidine from Step D was added 2 mL TFA. The reaction was stirred at rt for 1 h. The mixture was concentrated to afford 180 mg of a viscous oil.

### Piperidine 43

4-(7-tert-butylimidazo[1,2-a]pyridin-3-yl)piperidine, TFA Salt

Step A: 2-Amino-4-tert-butylpyridine

To 790 mg of sodium amide were added 20 mL of N,N-dimethylaniline and 2.74 g of 4-tert-butyl pyridine at rt. The mixture was stirred at 150° C. for 6 h. During this period, 3 more portions of sodium amide (790 mg each) were added. The reaction was cooled down to rt. The mixture was partitioned between ethyl acetate and water. Aqueous layer was extracted with ethyl acetate (3×). The combined organic phase was washed with brine, dried over magnesium sulfate and concentrated. The residue was purified by flash chromatography with 50% ethyl acetate in hexanes followed by 100% ethyl acetate to give the title compound as a solid:

¹H NMR (500 MHz, CDCl₃) δ 1.21 (s, 9H), 6.44 (t, 1H), 6.6.62 (dd, 3=5.5 Hz and, 1H), 7.94 (d, J=5.5 Hz, 1H).

Step B: 1-(tert-Butoxycarbonyl)-4-(7-tert-butylimidazo[1,2-a]pyridin-3-yl)piperidine

The title compound was prepared from 470 mg of 1-t-butyloxycarbonyl-4-(1-bromo-formylmethyl)piperidine (from Piperidine 42, Step C) and 277 mg of 2-amino-4-tert-butyl pyridine (from Step A) in 12 mL ethanol using a procedure analogous to that described in Piperidine 42, Step D to provide the title compound as a solid.

Step C: 4-(7-tert-butylimidazo[1,2-a]pyridin-3-yl)piperidine, TFA Salt

The title compound was prepared from 35 mg of 1-(tert-butoxycarbonyl)-4-((7-tert-butyl)imidazo[1,2-a]pyridin-3-yl)piperidine (from Step B) in 2 mL of TFA, using a procedure analogous to that described in Piperidine 42, Step E to provide the title compound as a viscous oil.

### Piperidine 44

4-(7-Chloroimidazo[1,2-a]pyridin-3-yl)piperidine, TFA Salt

The title compound was prepared from 350 mg of 1-t-butyloxycarbonyl-4-(1-bromo-formylmethyl)piperidine (from Piperidine 42, Step C) and 162 mg of 2-amino-4-chloropyridine (prepared using procedures analogous to those described by R. J. Sundberg et al, Org. Preparations & Procedures Int. 1997, 29, (1), 117-122) in 10 mL ethanol using a procedure analogous to that described in Piperidine 42, Step D to provide the BOC intermediate as a solid prior to the cleavage of the Boc-group to give the title TFA salt.

### Piperidine 45

4-(7-n-Propylimidazo[1,2-a]pyridin-3-yl)piperidine, TFA Salt

The title compound was prepared according to the general procedures of Piperidines 42 and 43, employing 2-amino-4-n-propylpyridine (prepared using a procedure analogous to that described in Piperidine 43, Step A) in place of 2-aminopyridine in Piperidine 42, Step D.

### Pyrrolidine 1

3-(R)-(tert-Butyldimethylsilyloxymethyl)-4-(S)-phenyl Pyrrolidine

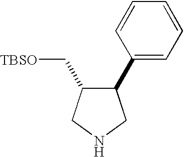

Step A: 3-((E)-Cinnamoyl)-4-(S)-benzyl oxazolidin-2-one

A solution of 222 g (1.5 mol) of trans-cinnamic acid and 250 mL (1.77 mol) of TEA in 3 L of TM at −78° C. was treated with 200 mL of trimethylacetyl chloride maintaining the internal temperature at less than −65° C. The resulting mixture was warmed to 0° C., then cooled to −78° C.

In a separate flask, a solution of 4-(S)-benzyl-oxazolidin-2-one in 2.05 L of TBF at −20° C. was treated with 660 mL of 2.5 M n-butyllithium in hexanes over 45 min. The resulting turbid mixture was cooled to −78° C. and then transferred via cannula to the flask containing the mixed anhydride. The resulting mixture was allowed to warm to rt and was stirred for 20 h. The reaction was quenched with 300 mL of sat'd NH₄Cl; the resulting mixture was partitioned between EtOAc and H20 and the layers were separated. The organic layer was dried over MgSO₄. The aqueous layer was extracted with 2× EtOAc; the extracts were dried and all of the organic extracts were combined. Partial concentration in vacuo caused precipitation of a solid; the mixture was diluted with hexanes and allowed to stand at rt for 1.5 h. The precipitate was filtered and dried to afford the title compound: ¹H NMR (500 MHz) δ 2.86 (dd, J=13.5, 9.5, 1H), (3.38, J=13.5, 3.5, 1H), 4.20-4.27 (m, 2H), 4.78-4.83 (m, 1H), 7.24-7.42 (5H), 7.63-7.65 (m, 1H), 7.92 (app d, J=2.5, 1H).

Step B: 3-(1-Benzyl-4-(S)-phenylpyrrolidine-3-(R)-carbonyl)-4-(S)-benzyl oxazolidin-2-one and 3-(1-benzyl-4-(R)-phenyl-pyrrolidine-3-(S)-carbonyl)-4-(S)-benzyl oxazolidin-2-one

A solution of 402 g (1.3 mol) of 3-((E)-cinnamoyl)-4-(S)-benzyl oxazolidin-2-one (from Step A) and 474 g (2.0 mol) of N-methoxymethyl-N-trimethylsilylmethyl benzyl amine in 4 L of CH₂Cl₂ at −10° C. was treated with 6 mL of trifluoroacetic acid. The resulting mixture was stirred cold for 4 h and then was treated with an additional 4 mL of trifluoroacetic acid. The reaction mixture was warmed to rt and stirred for 20 h. The reaction was quenched with 2 L of sat'd NaHCO₃ and the layers were separated. The organic layer was washed with 1 L of sat'd NaCl and concentrated. Chromatography on 10 kg of silica gel using 4:1 v/v hexanes/EtOAc (24 L), then 7:3 v/v hexanes/EtOAc (36 L), then 3:2 v/v hexanes/EtOAc (32 L) afforded 3-(1-benzyl-4-(S)-phenylpyrrolidine-3-(R)-carbonyl)-4-(S)-benzyl oxazolidin-2-one and 3-(1-benzyl-4-(R)-phenylpyrrolidine-3-(S)-carbonyl)-4-(S)-benzyl oxazolidin-2-one. For 3-(1-benzyl-4-(S)-phenylpyrrolidine-3-(R)-carbonyl)-4-(S)-benzyl oxazolidin-2-one: ¹H NMR (500 MHz) δ 2.66 (t, J=8.0, 1H), 2.78 (dd, J=13.0, 9.0, 1H), 2.87 (dd, J=9.0, 4.5, 1H), 3.21-3.27 (m, 2H), 3.64 (d, J=11.5, 1H), 3.77 (d, J=11.5, 1H), 4.10-4.15 (m, 2H), 4.61-4.65 (m, 1H), 7.16-7.38 (15H). For 3-(1-benzyl-4-(R)-phenylpyrrolidine-3-(S)-carbonyl)-4-(S)-benzyl oxazolidin-2-one: ¹H NMR (500 MHz) δ 2.69-2.76 (m, 2H), 2.82 (dd, J=9.5, 5.5, 1H), 3.14-3.22 (3H), 3.64 (d, J=13.0, 1H), 3.74 (d, J=13.0, 1H), 4.07-4.12 (m, 211), 4.16 (t, J=9.0, 1H), 4.26-4.30 (m, 1H), 4.65-4.69 (m, 1H), 7.03-7.40 (15H).

Step C: 1-Benzyl-3-(R)-hydroxymethyl-4-(S)-phenylpyrrolidine

A solution of 3-(1-benzyl-4-(S)-phenylpyrrolidine-3-(R)-carbonyl)-4-(S)-benzyl oxazolidin-2-one (from Step B) in 2.5 L of THF at 10° C. was treated with 1.18 L of 1.0 M lithium aluminum hydride solution in THF over a period of 2 h. The resulting mixture was warmed to rt and stirred for 20 h. The reaction was quenched by adding 40 mL of H20, then 40 mL of 2.0 N NaOH, then 115 mL of H₂O and then was stirred at rt for 1.5 h. The mixture was filtered and the filtrate was concentrated. Chromatography on 4 kg of silica using 4:1 hexanes/acetone (14 L), then 7:3 hexanes/acetone as the eluant to afford the title compound: ¹H NMR (400 MHz) 62.38-2.46 (m, 2H), 2.78-2.88 (3H), 3.20-3.26 (2H), 3.65 (dd, J=12.0, 4.0, 1H), 3.66 (app s, 211), 3.74 (dd, J=12.0, 4.0, 1H), 7.18-7.34 (10H); ESI-MS 268 (M+H); HPLC A: 2.35 min.

Step D: 1-Benzyl-3-(R)-(tert-butyldimethylsilyloxymethyl)-4-(S)-phenyl pyrrolidine

A solution of 82.0 g (0.31 mol) of 1-benzyl-3-(R)-hydroxymethyl-4-(S)-phenyl pyrrolidine (from Step C) and 46.5 g (0.36 mol) of N,N-diisopropylethylamine in 1 L of CH₂Cl₂ was treated with 54.2 g (0.36 mol) of tert-butyldimethylsilyl chloride and the resulting mixture was stirred at rt for 20 h. The reaction was quenched with 750 mL of sat'd NaHCO₃ and the layers were separated. The organic layer was combined with 150 g of silica gel and aged for 45 min. The mixture was filtered and the filtrate was concentrated to afford the title compound.

Step E: 3-(R)-(tert-Butyldimethylsilyloxymethyl)-4-(S)-phenylpyrrolidine

A mixture of 117 g (0.31 mol) of 1-benzyl-3-(R)-(tert-butyldimethylsilyloxymethyl)-4-(S)-phenyl pyrrolidine (from Step D), 31.5 g (0.50 mol) ammonium formate, 20.0 g of 20% palladium hydroxide on carbon in 1.5 L of MeOH was heated at 55° C. for 2.5 h. The mixture was cooled and filtered through a pad of Celite. The filtrate was concentrated. The residue was dissolved in 1 L of CH₂Cl₂, washed with 300 mL of 10% NH₄OH solution, 200 mL of sat'd NaCl, dried over MgSO₄ and concentrated to afford the title compound: ¹H NMR (400 MHz) δ −0.09 (s, 3H), -0.08 (s, 3H), 0.77 (s, 9H), 2.25-2.30 (m, 1H), 2.84-2.96 (4H), 3.18 (dd, J=11.2, 3.2, 1H), 3.29-3.36 (m, 1H), 3.44 (dd, J=10.0, 6.0), 3.56 (dd, J=10.0, 4.4, 1H); ESI-MS 292 (M+H); HPLC A: 3.44 min.

### PYRROLIDINE 2

3-(R)-(tert-Butyldimethylsilyloxymethyl)-4-(S)-(3-fluoro)phenylpyrrolidine

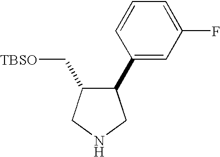

The title compound was prepared using procedures analogous to those described to prepare Pyrrolidine 1, except that trans-(3-fluoro)cinnamic acid was substituted for trans-cinnamic acid in Step A. For the title compound: ¹H NMR (400 Mhz) δ 0.013 (s, 3H), 0.016 (s, 3H), 0.87 (s, 9H), 2.09 (br s, 1H), 2.30-2.37 (m, 1H), 2.88-2.90 (3H), 2.23 (dd, J=8.0, 11.2, 1H), 3.39 (dd, J=6.8, 10.0. 1H), 3.56 (dd, J=6.0, 10.0, 1H), 3.64 (dd, J=5.2, 10.0), 6.86-6.91 (m, 1H), 6.95 (dt, J=12.0, 2.4, 1H), 7.01 (d, J=7.6, 1H), 7.22-7.27 (m, 1H); ESI-MS 310 (M+H); BPLC A: 3.05 min.

### EXAMPLE 1

2-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-2-methylpropionic Acid

Step A: 2-{[(3R,4S)-3-(tert-Butyldimethylsilyloxymethyl)-4-(3-fluorophenyl)pyrrolidin-1-yl]methyl}-2-methylpropionic Acid, Para-methoxybenzyl Ester

A solution of 299 mg (0.96 mmol) of 3-(R)-(t-butyldimethylsilyloxymethyl)-4-(S)-(3-fluoro)phenylpyrrolidine (Prepared as Pyrrolidine 2 above) and 203 mg (0.92 mmol) of 2-formyl-2-methylpropionic acid, para-methoxybenzyl ester (Prepared as Aldehyde 1 above) in 5 mL of CH₂Cl₂ was treated with 274 mg (1.2 mmol) of sodium triacetoxyborohydride. After 45 minutes at rt the reaction was partitioned between 100 mL of CH₂Cl₂ and 100 mL of 1 N NaHCO₃ and the layers were separated. The aqueous layer was extracted with 100 mL of CH₂Cl₂. The combined organic phases were washed with 200 mL of brine, dried over Na₂SO₄ and concentrated. The residue was purified by flash chromatography using 9:1 v/v of hexanes/EtOAc as the eluant to afford the title compound as a colorless oil: R_{F}: 0.59 (4:1 v/v hexanes/EtOAc); ¹H-NMR (500 Mhz) δ 0.02 (s, 6H), 0.87 (s, 9H), 1.23 (s, 6H), 2.25 (m, 1H), 2.45 (m, 1H), 2.61-2.71 (m, 3H), 2.82-2.91 (m, 3H), 3.52-3.55 (m, 2H), 3.79 (s, 3H), 5.05 (ABq, J=12.0, 2H), 6.84-6.87 (m, 3H), 6.99-7.03 (m, 2H), 7.21-7.28 (m, 3H).

Step B: 2-{[(3R,4S)-3-(Hydroxymethyl)-4-(3-fluorophenyl)pyrrolidin-1-yl]methyl}-2-methylpropionic Acid, Para-methoxybenzyl Ester

A solution of 345 mg (0.67 mmol) of 2-{[(3R,4S)-3-(t-butyldimethylsilyloxymethyl)-4-(3-fluorophenyl)pyrrolidin-1-yl]methyl}-2-methylpropionic acid, para-methoxybenzyl ester (from Step A) and 2 mL (2.0 mmol) of 1 M tetrabutylammonium fluoride in THF were stirred at rt for 4.5 hours. The reaction mixture was adsorbed onto silica gel and volatiles were removed under reduced pressure. The residue was purified by flash chromatography using 9:1 v/v of hexanes/EtOAc as the eluant to afford the title compound as a colorless oil: R_{F}: 0.59 (4:1 v/v hexanes/EtOAc); ¹H-NMR (500 Mhz) δ 1.23 (s, 3H), 1.24 (s, 3H), 2.23 (m, 1H), 2.42-2.48 (m, 2H), 2.64-2.70 (m, 3H), 2.83 (m, 1H), 3.04-3.07 (m, 2H), 3.56 (m, 1H), 3.66 (m, 1H), 3.79 (s, 3H), 5.07 (ABq, J 11.9, 2H), 6.84-7.01 (m, 5H), 7.22-7.31 (m, 3H).

Step C: 2-{[(3R,4S)-3-Formyl-4-(3-fluorophenyl)pyrrolidin-1-yl]methyl}-2-methylpropionic Acid, para-methoxybenzyl Ester

The title compound was prepared from 2-{[(3R,4S)-3-(hydroxymethyl)-4-(3-fluorophenyl)pyrrolidin-1-yl]methyl}-2-methylpropionic acid, para-methoxybenzyl ester (from Step B) using a procedure analogous to that described for Aldehyde 1, Step B. R_{F}: 0.66 (3:2 v/v hexanes/EtOAc); ¹H-NMR (500 Mhz) δ 1.23 (s, 6H), 2.63-2.73 (m, 3H), 2.84-3.00 (m, 5H), 3.47 (m, 11H), 3.79 (s, 3H), 5.06 (ABq, J=12.0, 2H), 6.84-7.02 (m, 5H), 7.23-7.30 (m, 3H), 9.60 (d, J=1.9, 1H).

Step D: 2-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-2-methylpropionic Acid, Para-methoxybenzyl Ester

The title compound was prepared from 4-(3-benzyl-1-ethyl-1H-pyrazol-5-yl)piperidine, trifluoro-acetic acid salt (Prepared as Piperidine 1 above) and 2-{[(3R,4S)-3-formyl-4-(3-fluorophenyl)pyrrolidin-1-yl]methyl}-2-methylpropionic acid, paramethoxybenzyl ester (from Step C) using a procedure analogous to that described in Example 1, Step A. R_{F}: 0.24 (3:2 v/v hexanes/EtOAc); 1H-NMR (500 Mhz) δ 1.24 (s, 6H), 1.40-1.96 (m, 9H), 2.27-2.45 (m, 5H), 2.62-2.72 (m, 3H), 2.85-2.96 (m, 5H), 3.77 (s, 3H), 3.95 (s, 2H), 4.03 (q, J=7.2, 2H), 5.07 (ABq, J=12.0, 2H), 5.72 (s, 1H), 6.83-6.88 (m, 3H), 7.01-7.05 (m, 3H), 7.18-7.32 (m, 7H).

Step E: 2-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-2-methylpropionic Acid

A solution of 25 mg (0.038 mmol) of 2-{[(3S,4S)-3-[(4-{3-benzyl-1-ethyl-1H-pyrazol-5-yl}piperidin-1-yl)methyl]-4-(3-fluorophenyl)pyrrolidin-1-yl]methyl}-2-methylpropionic acid, para-methoxybenzyl ester (from Step D) in 1 mL formic acid was warmed to 55° C. for 2.25 h. The reaction was concentrated under reduced pressure. The residue was purified by flash chromatography using a gradient of 100 CH₂Cl₂ and 95:5:1 v/v/v CH₂Cl₂/MeOH/NH₄OH as the eluant to afford the title compound: R_{F}: 0.44 (90:10:1 v/v/v CH₂Cl₂/MeOH/NH₄OH); ¹H-NMR (500 MHz, CD₃OD): δ 1.22 (s, 3H), 1.25 (s, 3H), 1.33 (t, J=7.1, 3H), 1.43 (m, 1H), 1.56 (m, 1H), 1.68-1.78 (m, 2H), 1.98 (m, 1H), 2.10 (m, 1H), 2.40 (m, 1H), 2.51-2.58 (m, 2H), 2.76-2.80 (m, 2H), 2.97 (m, 1H), 3.17-3.42 (m, 5H), 3.68-3.74 (m, 2H), 3.85 (s, 2H), 4.02 (q, J=7.1, 2H), 5.73 (s, 1H), 6.97 (m, 1H), 7.14-7.36 (m, 8H). ESI-MS 547.4 (M+H); HPLC LC 2: 2.13 min.

### Example 2

2-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-2-ethylbutyric Acid

Step A: 2-{[(3R,4S)-3-(tert-Butyldimethylsilyloxymethyl)-4-(3-fluorophenyl)pyrrolidin-1-yl]methyl}-2-ethylbutyric Acid, Benzyl Ester

The title compound was prepared from 2-ethyl-2-formylbutyric acid, benzyl ester (Prepared as Aldehyde 3 above) and 3-(R)-(tert-butyldimethylsilyloxymethyl)-4-(S)-(3-fluoro)phenylpyrrolidine (Prepared as Pyrrolidine 2 above) using a procedure analogous to that described in Example 1, Step A. R_{F}: 0.37 (19:1 v/v hexanes/EtOAc); ¹H-NMR (500 Mhz) δ 0.02 (s, 6H), 0.79-0.87 (m, 15H), 1.66-1.76 (m, 4H), 2.25 (m, 1H), 2.44 (m, 1H), 2.64-2.90 (m, 6H), 3.50-3.56 (m, 2H), 5.12 (ABq, J=12.4, 2H), 6.87 (m, 1H), 6.99-7.03 (m, 2H), 7.19-7.37 (m, 6H).

Step B: 2-{[(3R,4S)-3-(Hydroxymethyl)-4-(3-fluorophenyl)pyrrolidin-1-yl]methyl}-2-ethylbutyric Acid, Benzyl Ester

The title compound was prepared from 2-{[(3R,4S)-3-(tert-butyldimethylsilyloxymethyl)-4-(3-fluorophenyl)pyrrolidin-1-yl]methyl}-2-ethylbutyric acid, benzyl ester (from Step A) using a procedure analogous to that described in Example 1, Step B. R_{F}: 0.48 (3:2 v/v hexanes/EtOAc); 1H-NMR (500 Mhz) δ 0.80 (t, J=7.4, 6H), 1.63-1.77 (m, 4H), 2.23 (m, 1H), 2.39-2.48 (m, 2H), 2.66 (m, 1H), 2.72 (s, 2H), 2.82 (m, 1H), 3.04-3.10 (m, 2H), 3.55-3.68 (m, 2H), 5.13 (ABq, J=12.4, 2H), 6.87-7.01 (m, 3H), 7.21-7.38 (m, 6H).

Step C: 2-{[(3R,4S)-3-Formyl-4-(3-fluorophenyl)pyrrolidin-1-yl]methyl}-2-ethylbutyric Acid, Benzyl Ester

The title compound was prepared from 2-{[(3R,4S)-3-(hydroxymethyl)-4-(3-fluorophenyl)pyrrolidin-1-yl]methyl}-2-ethylbutyric acid, benzyl ester (from Step B) using a procedure analogous to that described for Aldehyde 1, Step B. R_{F}: 0.45 (4:1 v/v hexanes/EtOAc); ¹H-NMR (500 Mhz) δ 0.79-0.84 (m, 6H), 1.62-1.76 (m, 4H), 2.63 (dd, J=9.1, 6.5, 1H), 2.74 (ABq, J=13.5, 2H), 2.82-2.99 (m, 4H), 3.49 (m, 1H), 5.13 (ABq, J=12.3, 2H), 6.90-7.02 (m, 3H), 7.23-7.38 (m, 6H), 9.61 (d, J=2.0, 1H).

Step D: 2-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-2-ethylbutyric Acid, Benzyl Ester

The title compound was prepared from 2-{[(3R,4S)-3-formyl-4-(3-fluorophenyl)pyrrolidin-1-yl]methyl}-2-ethylbutyric acid, benzyl ester (from Step C) and 4-(3-benzyl-1-ethyl-1H-pyrazol-5-yl)piperidine, trifluoro-acetic acid salt (Prepared as Piperidine 1 above) using a procedure analogous to that described in Example 1, Step A. R_{F}: 0.42 (1:1 v/v hexanes/EtOAc); ¹H-NMR (500 Mhz) δ 0.79-0.83 (m, 6H), 1.40-1.95 (m, 13H), 2.25-2.44 (m, 5H), 2.65-2.95 (m, 8H), 3.94 (s, 2H), 4.02 (q, J=7.3, 2H), 5.12 (ABq, J=12.3, 2H), 5.71 (s, 1H), 6.86 (m, 1H), 7.01-7.05 (m, 2H), 7.19-7.37 (m, 11H).

Step E: 2-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-2-ethylbutyric Acid

A mixture of 44 mg (0.066 mmol) of 2-{[(3S,4S)-3-[(4-13-benzyl-1-ethyl-1H-pyrazol-5-yl}piperidin-1-yl)methyl]-4-(3-fluorophenyl)pyrrolidin-1-yl]methyl}-2-ethylbutyric acid, benzyl ester (from Step D) and 16.5 mg of 10% palladium on carbon in 1 mL of MeOH was hydrogenated at rt under a balloon of hydrogen for 2.5 hours. The reaction was filtered and concentrated under reduced pressure. The residue was purified by flash chromatography using a gradient of 97/3 V/v CH₂Cl₂/MeOH, 90: 10 V/v CH₂Cl₂/MeOH and 90:10:1 v/v/v CH₂Cl₂/MeOH/NH₄OH as the eluant to afford the title compound: Rp: 0.42 (90:10:1 v/v/v CH₂Cl₂/MeOH/NH₄OH); ¹H-NMR (500 MHz, CD₃OD): δ 0.87-0.91 (m, 6H), 1.31-1.79 (m, 10H), 1.97-2.12 (m, 2H), 2.42 (m, 1H), 2.51-2.58 (m, 2H), 2.72-2.80 (m, 2H), 2.97 (m, 1H), 3.20-3.36 (m, 6H), 3.61-3.66 (m, 2H), 3.85 (s, 2H), 4.00-4.04 (m, 2H), 5.73 (s, 1H), 6.96 (m, 1H), 7.14-7.35 (m, 8H). ESI-MS 575.5 (M+H); HPLC LC2: 2.27 min.

### Example 3

(2R,2S)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-3-methylbutyric Acid

Step A: (2R/2S)-{[(3R,4S)-3-(tert-Butyldimethylsilyloxymethyl)-4-(3-fluorophenyl)pyrrolidin-1-yl]methyl}-3-methylbutyric Acid, Benzyl Ester

The title compound was prepared from 2-formyl-3-methylbutyric acid, benzyl ester (Prepared as Aldehyde 4 above) and 3-(R)-(t-butyldimethylsilyloxymethyl)-4-(S)-(3-fluoro)phenylpyrrolidine (Prepared as Pyrrolidine 2 above) using a procedure analogous to that described in Example 1, Step A. R_{F}: 0.23 (9:1 v/v hexanes/EtOAc); ¹H-NMR (500 Mhz) δ 0.01-0.03 (m, 6H), 0.84-1.00 (m, 15H), 1.92 (m, 1H), 2.29-2.61 (m, 4H), 2.72-3.00 (m, 5H), 3.52-3.59 (m, 2H), 5.05-5.33 (m, 2H), 6.86 (m, 1H), 7.01-7.03 (m, 2H), 7.18-7.39 (m, 6H).

Step B: (2R/2S)-{[(3R,4S)-3-(Hydroxymethyl)-4-(3-fluorophenyl)pyrrolidin-1-yl]methyl}-3-methylbutyric Acid, Benzyl Ester

The title compound was prepared from (2R/2S)-{[(3R,4S)-3-(t-butyldimethylsilyloxymethyl)-4-(3-fluorophenyl)pyrrolidin-1-yl]methyl}-3-methylbutyric acid, benzyl ester (from Step A) using a procedure analogous to that described in Example 1, Step B. R_{F}: 0.31 (3:2 v/v hexanes/EtOAc); ¹H-NMR (500 Mhz) δ 0.92-0.98 (m, 6H), 1.92 (m, 1H), 2.27-3.26 (m, 10H), 3.54-3.70 (m, 2H), 5.08-5.30 (m, 2H), 6.89 (m, 1H), 6.97-7.02 (m, 2H), 7.20-7.40 (m, 6H).

Step C: (2R/2S)-{[(3R,4S)-3-Formyl-4-(3-fluorophenyl)pyrrolidin-1-yl]methyl}-3-methylbutyric Acid, Benzyl Ester

The title compound was prepared from (2R/2S)-{[(3R,4S)-3-(hydroxymethyl)-4-(3-fluorophenyl)pyrrolidin-1-yl]methyl}-3-methylbutyric acid, benzyl ester (from Step B) using a procedure analogous to that described for Aldehyde 1, Step B. R_{F}: 0.31 (3:2 v/v hexanes/EtOAc); ¹H-NMR (500 Mhz) δ 0.93-0.99 (m, 6H), 1.92 (m, 1H), 2.43-2.55 (m, 2H), 2.72-3.16 (m, 6H), 3.53 (m, 1H), 5.06-5.30 (m, 2H), 6.89-7.02 (m, 3H), 7.21-7.38 (m, 6H), 9.60, 9.63 (2d, J=1.7, 1H).

Step D: (2R/2S)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-3-methylbutyric Acid, Benzyl Ester

The title compound was prepared from (2R/2S)-{[(3R,4S)-3-formyl-4-(3-fluorophenyl)pyrrolidin-1-yl]methyl}-3-methylbutyric acid, benzyl ester (from Step C) and 4-(3-benzyl-1-ethyl-1H-pyrazol-5-yl)piperidine, trifluoro-acetic acid salt (Prepared as Piperidine 1 above) using a procedure analogous to that described in Example 1, Step A. R_{F}: 0.32 (1:1 v/v hexanes/EtOAc); ¹H-NMR (500 Mhz) δ 0.93-0.99 (m, 6H), 1.40-2.06 (m, 10H), 2.22-2.50 (m, 7H), 2.60-3.08 (m, 7H), 3.95 (s, 2H), 4.01-4.06 (m, 2H), 5.08-5.32 (m, 2H), 5.73 (s, 1H), 6.85 (m, 111), 7.02-7.05 (m, 2H), 7.16-7.39 (m, 11H).

Step E: (2R/2S)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-3-methylbutyric Acid

A mixture of 43 mg (0.064 mmol) of (2R/2S)-{[(3S,4S)-3-[(4-{3-benzyl-1-ethyl-1H-pyrazol-5-yl}piperidin-1-yl)methyl]-4-(3-fluorophenyl)pyrrolidin-1-yl]methyl}-3-methylbutyric acid, benzyl ester (from Step D) and 7.5 mg of 10% palladium on carbon in 1 mL of MeOH was hydrogenated at rt under a balloon of hydrogen for 2 hours. The reaction was filtered and concentrated under reduced pressure. The residue was purified by flash chromatography using CH₂Cl₂, then 90:10:1 v/v/v CH₂Cl₂/MeOH/NH₄OH as the eluant to afford the title compound: R_{F}: 0.37 (90:10:1 v/v/v CH₂Cl₂/MeOH/NH₄OH); ¹H-NMR (500 MHz, CD₃OD): δ 0.97-1.05 (m, 6H), 1.27-1.75 (m, 7H), 1.91-2.08 (m, 3H), 2.37-2.56 (m, 4H), 2.74-2.96 (m, 3H), 3.11 (m, 1H), 3.25-3.56 (m, 4H), 3.71-3.84 (m, 4H), 3.99-4.03 (m, 2H), 5.71, 5.72 (2s, 1H), 6.95 (m, 1H), 7.13-7.33 (m, 8H). ESI-MS 561.7 (M+H); HPLC LC2: 2.24 min.

### Example 4

(2R or 2S)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-2,3-dimethylbutyric Acid

Step A: Tigloyl Chloride

A solution 2.09 g (37.2 mmol) of KOH in 15 mL of EtOH was treated with 3.72 g (37.1 mmol) of tiglic acid (Tetrahedron Lett. 1977, 3379-3382). After sonicating the thick suspension for 10 minutes, volatiles were removed under reduced pressure. The white solid was suspended in acetone and filtered. The potassium salt was suspended in Et₂O and cooled to 0° C. Treatment with 16 mL (183 mmol) of oxalyl chloride was followed by addition of 0.05 mL of DMF. The reaction was stirred at 0° C. for 2 h, filtered and concentrated under reduced pressure. The resulting gold oil was concentrated several times from CH₂Cl₂ and used without further purification. ¹H-NMR (500 Mhz) δ 1.90-1.95 (m, 6H), 7.30 (m, 1H).

Step B: Tiglic acid, (R)-α-methylbenzyl Ester

The title compound was prepared from tigloyl chloride (from Step A) and (R)-α-methylbenzyl alcohol using a procedure analogous to that described in Aldehyde 4, Step A. R_{F}: 0.46 (9:1 v/v hexanes/EtOAc); ¹H-NMR (500 Mhz) δ 1.58 (d, J=6.6, 3H), 1.81 (d, J=7.0, 3H), 1.87 (s, 3H), 5.96 (q, J=6.6, 1H), 6.93 (m, 1H), 7.27-7.40 (m, 5H).

Step C: (2R or 2S)-2-isopropyl-2-methyl-3-butenoic Acid, (R)-α-methylbenzyl Ester and (2S or 2R)-2-isopropyl-2-methyl-3-butenoic Acid, (R)-(X-methylbenzyl Ester

The title compound was prepared from tiglic acid, (R)-o:-methylbenzyl ester (from Step B) and isopropyl iodide using a procedure analogous to that described in Aldehyde 4, Step B. R_{F}: 0.61 (9:1 v/v hexanes/EtOAc); Diastereomers were separated by preparative HPLC (Column: Chiralcel OJ; Mobile Phase: 97:3 v/v hexanes/isopropanol; Sample: 10 mg/injection; Flow: 8 ML/min; 220 nm). Diastereomer 1 (Retention Time: 11.15 min.): ¹H-NMR (500 Mhz) δ 0.84 (d, J=6.9, 3H), 0.86 (d, J=6.9, 3H), 1.20 (s, 3H), 1.54 (d, J=6.6, 3H), 2.21 (m, 1H), 5.06-5.16 (m, 2H), 5.90 (q, J=6.6, 1H), 6.03 (m, 1H), 7.27-7.36 (m, 5H). Diastereomer 2 (Retention Time: 14.05 min.): ¹H-NMR (500 Mhz) δ 0.77 (d, J=7.0, 3H), 0.83 (d, J=7.0, 3H), 1.19 (s, 3H), 1.54 (d, J=6.6, 3H), 2.19 (m, 1H), 5.08-5.18 (m, 2H), 5.90 (q, J=6.6, 1H), 6.05 (m, 1H), 7.26-7.39 (m, 5H).

Step D: (2R or 2S)-2, 3-Dimethyl-2-hydroxymethyl-butyric acid, (R)-α-methylbenzyl Ester

The title compound was prepared from (2R or 2S)-2-isopropyl-2-methyl-3-butenoic acid, (R)-α-methylbenzyl ester (Diastereomer 1 from Step C) using a procedure analogous to that described for Aldehyde 4, Step C, except dimethyl sulfide was replaced with NaBH₄ in aqueous ethanol. R_{F}: 0.26 (4:1 v/v hexanes/EtOAc); ¹H-NMR (500 Mhz) δ 0.83 (d, J=6.8, 3H), 0.85 (d, J=6.8, 3H), 1.14 (s, 3H), 1.57 (d, J=6.6, 3H), 2.10 (m, 1H), 2.31 (dd, J=7.1, 6.3, 1H), 3.48 (dd, J=11.2, 6.3, 1H), 3.78 (dd, J=11.2, 7.1, 1H), 5.95 (q, J=6.6, 1H), 7.27-7.38 (m, 5H).

Step E: (2R or 2S)-2, 3-Dimethyl-2-(trifluoromethylsulfonyloxymethyl)-butyric acid, (R)-α-methylbenzyl Ester

A solution of 0.218 mg (0.87 mmol) of (2R or 2S)-2, 3-dimethyl-2-hydroxymethyl-butyric acid, (R)-α-methylbenzyl ester (from Step D) and 0.25 mL (1.3 mmol) of 2, 6-lutidine at −78° C. was treated with 0.22 mL (1.3 mmol) of triflic anhydride. After stirring for 2.5 hours at −78° C., the reaction was quenched with H₂O. The reaction mixture was partitioned between 50 mL of H₂O and 50 mL of CH₂Cl₂. The phases were separated and the aqueous layer was extracted with 50 mL of CH₂Cl₂. The combined organics were dried over Na₂SO₄ and concentrated under reduced pressure. The residue was purified by flash chromatography using 9:1 v/v of hexanes/EtOAc as the eluant to afford the title compound as a colorless film: R_{F}: 0.64 (4:1 v/v hexanes/ EtOAc); ¹H-NMR (500 MHz) δ 0.89 (d, J=7.0, 3H), 0.90 (d, J=7.0, 3H), 1.27 (s, 3H), 1.58 (d, J=6.6, 3H), 2.06 (m, 1H), 4.44 (d, J=9.2, 1H), 4.76 (d, J=9.2, 1H), 5.94 (q, J=6.6, 1H), 7.27-7.39 (m, 5H).

Step F: (2R or 2S)-{[(3R,4S)-3-(tert-Butyldimethylsilyloxymethyl)-4-(3-fluorophenyl)pyrrolidin-1-yl]methyl}-2,3-dimethylbutyric acid, (R)-α-methylbenzyl Ester

A solution of 287 mg (0.75 mmol) of (2R or 2S)-2, 3-dimethyl-2-(trifluoromethylsulfonyloxymethyl)-butyric acid, (R)-α-methylbenzyl ester (from Step E), 0.137 mL (0.78 mmol) of DIEA and 293 mg (90.94 mmol) of 3-(R)-(tert-butyldimethylsilyloxymethyl)-4-(S)-(3-fluoro)phenylpyrrolidine (Prepared as Pyrrolidine 2 above) in 3 mL of 1,2-dichloroethane was heated at 70° C. for 11 h. The reaction was cooled to rt and the volatiles were removed under reduced pressure. The reaction mixture was partitioned between 100 mL of Et₂O and 100 mL of 1 N NaHCO₃. The phases were separated and the aqueous layer was extracted with 100 mL of CH₂Cl₂. The combined organics were dried over Na₂SO₄ and concentrated under reduced pressure. The residue was purified by flash chromatography using 19:1 v/v of hexanes/EtOAc as the eluant to afford the title compound: R_{F}: 0.66 (9:1 v/v hexanes/ EtOAc); ¹H-NMR (500 MHz) δ 0.007 (s, 3H), 0.01 (s, 3H), 0.82-0.92 (m, 15H), 1.17 (s, 3H), 1.57 (d, J=6.6, 3H), 2.13 (m, 1H), 2.41 (m, 1H), 2.50 (d, J=12.8, 1H), 2.60 (m, 1H), 2.69-2.81 (m, 3H), 2.88 (d, J=12.8, 1H), 3.43-3.48 (m, 2H), 5.96 (m, 1H), 6.86 (m, 1H), 6.92-6.97 (m, 2H), 7.17-7.43 (m, 6H).

Step G: (2R or 2S)-{[(3R,4S)-3-(Hydroxymethyl)-4-(3-fluorophenyl)pyrrolidin-1-yl]methyl}-2,3-dimethylbutyric Acid, (R)-α-methylbenzyl Ester

The title compound was prepared from (2R or 2S)-{[(3R,4S)-3-(tert-butyldimethylsilyloxymethyl)-4-(3-fluorophenyl)pyrrolidin-1-yl]methyl}-2,3-dimethylbutyric acid, (R)-α-methylbenzyl ester (from Step F) using a procedure analogous to that described in Example 1, Step B. R_{F}: 0.48 (3:2 v/v hexanes/EtOAc); ¹H-NMR (500 Mhz) δ 0.83 (d, J=6.8, 3H), 0.86 (d, J=6.8, 3H), 1.16 (s, 3H), 1.56 (d, J=6.6, 3H), 2.02 (m, 1H), 2.16 (m, 1H), 2.32 (m, 1H), 2.40 (m, 1H), 2.54 (d, J=12.8, 1H), 2.65-2.73 (m, 2H), 2.83-2.88 (m, 2H), 2.95 (m, 1H), 3.50 (m, 1H), 3.61 (m, 1H), 5.95 (q, J=6.6, 1H), 6.86-6.93 (m, 3H), 7.19-7.39 (m, 6H).

Step H: (2R or 2S)-{[(3R,4S)-3-Formyl-4-(3-fluorophenyl)pyrrolidin-1-yl]methyl}-2,3-dimethylbutyric Acid, (R)-α-methylbenzyl Ester

The title compound was prepared from (2R or 2S)-{[(3R,4S)-3-(hydroxymethyl)-4-(3-fluorophenyl)pyrrolidin-1-yl]methyl}-2,3-dimethylbutyric acid, (R)-α-methylbenzyl ester (from Step G) using a procedure analogous to that described for Aldehyde 1, Step B. R_{F}: 0.50 (4:1 v/v hexanes/EtOAc); ¹H-NMR (500 Mhz) 6 0.85 (d, J=6.8, 3H), 0.86 (d, J=6.8, 3H), 1.16 (s, 3H), 1.56 (d, J=6.6, 3H), 2.02 (m, 1H), 2.51-2.58 (m, 2H), 2.72-2.91 (5H), 3.36 (q, J=6.6, 3H), 5.97 (q, J=6.6, 1H), 6.87-6.94 (m, 3H), 7.21-7.40 (m, 6H), 9.53 (d, J=1.8, 1H).

Step I: (2R or 2S)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-2,3-dimethylbutyric Acid, (R)-α-methylbenzyl Ester

The title compound was prepared from 4-(3-benzyl-1-ethyl-(1H-pyrazol-5-yl))piperidine, trifluoro-acetic acid salt (Prepared as Piperidine 1 above) and (2R or 2S)-{[(3R,4S)-3-formyl-4-(3-fluorophenyl)pyrrolidin-1-yl]methyl}-2,3-dimethylbutyric acid, (R)-α-methylbenzyl ester (from Step H) using a procedure analogous to that described in Example 1, Step A. R_{F}: 0.43 (1:1 v/v hexanes/EtOAc); ¹H-NMR (500 Mhz) δ 0.84-0.88 (m, 6H), 1.17 (s, 3H), 1.40-2.91 (m, 26H), 3.95-4.05 (m, 4H), 5.72 (s, 1H), 5.97 (m, 1H), 6.83-6.98 (m, 3H), 7.16-7.40 (m, 11H).

Step J: (2R or 2S)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-2,3-dimethylbutyric Acid

The title compound was prepared from (2R or 2S)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-2,3-dimethylbutyric acid, (R)-(x-methylbenzyl ester (from Step I) using a procedure analogous to that described in Example 3, Step E. R_{F}: 0.51 (90:10:1 v/v/v CH₂Cl₂/MeOH/NH₄OH); ¹H-NMR (500 MHz, CD₃OD): δ 0.91 (d, J=6.8, 3H), 0.92 (d, J=6.8, 3H), 1.20 (s, 3H:), 1.33 (t, J=7.2, 3H), 1.41 (m, 1H), 1.55 (m, 1H), 1.68 (m, 1H), 1.77 (m, 111), 1.95 (m, 1H), 2.04-2.11 (m, 2H), 2.38 (m, 1H), 2.49-2.57 (m, 2H), 2.74-2.78 (m, 2H), 2.95 (m, 1H), 3.05 (d, J=13.2, 1H), 3.21-3.42 (m, 4H), 3.65-3.74 (m, 2H), 3.85 (s, 2H), 4.02 (q, J=7.2,2H), 5.73 (s, 1H), 6.97 (m, 1H), 7.15-7.36 (m, 8H). ESI-MS 575.5 (M+H); HPLC LC 2: 2.27 min.

### Example 5

(2S or 2R)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-2,3-dimethylbutyric Acid

The title compound was prepared using procedures analogous to those described for Example 4, except Diastereomer 2 from Step C was substituted for Diastereomer 1 in Step D. R_{F}: 0.52 (90:10:1 v/v/v CH₂Cl₂/MeOH/NH₄OH); ¹H-NMR (500 MHz, CD₃OD): δ 0.91 (d, J=7.0, 3H), 0.93 (d, J=7.0, 3H), 1.14 (s, 3H), 1.33 (t, J=7.1, 3H), 1.42 (m, 1H), 1.55 (m, 1H), 1.69 (m, 1H), 1.77 (m, 1H), 1.95-2.10 (m, 3H), 2.40 (m, 1H), 2.49-2.57 (m, 2H), 2.73-2.78 (m, 2H), 2.95 (m, 1H), 3.04 (d, J=13.3, 1H), 3.21-3.42 (m, 4H), 3.65-3.71 (m, 2H), 3.85 (s, 2H), 4.02 (q, J=7.2, 2H), 5.73 (s, 1H), 6.97 (m, 1H), 7.15-7.36 (m, 8H). ESI-MS 575.7 (M+H); HPLC LC 1: 1.55 min.

### Example 6

(2R or 2S)-{[(3S,4S)-3-[(4-{3-[4-Ethoxybenzyl]-1-ethyl-1H-pyrazol-5-yl}piperidin-1-yl)methyl]-4-(3-fluorophenyl)pyrrolidin-1-yl]methyl}-2,3-dimethylbutyric Acid

The title compound was prepared using procedures analogous to those described for Example 4, except Piperidine 13 was substituted for Piperidine 1 in Step I. R_{F}: 0.47 (90:10:1 v/v/v CH₂Cl₂/MeOH/NH₄OH); ¹H-NMR (500 MHz, CD₃OD): δ 0.91 (d, J=6.7, 3H), 0.93 (d, J=6.7, 3H), 1.20 (s, 3H), 1.31-1.45 (m, 7H), 1.55 (m, 1H), 1.68 (m, 1H), 1.76 (m, 1H), 1.95 (m, 1H), 2.03-2.12 (m, 2H), 2.38 (m, 1H), 2.48-2.56 (m, 2H), 2.74-2.77 (m, 2H), 2.94 (m, 1H), 3.05 (d, J=13.0, 1H), 3.20-3.42 (m, 5H), 3.65-3.74 (m, 2H), 3.77 (s, 2H), 3.96-4.03 (m, 4H), 5.70 (s, 1H), 6.78-6.81 (m, 2H), 6.97 (m, 1H), 7.07-7.18 (m, 4H), 7.33 (m, 1H). ESI-MS 620.6 (M+H); HPLC LC 1: 1.64 min.

### Example 7

(2S or 2R)-{[(3S,4S)-3-[(4-{3-[4-Ethoxybenzyl]-1-ethyl-1H-pyrazol-5-yl}piperidin-1-yl)methyl]-4-(3-fluorophenyl)pyrrolidin-1-yl]methyl}-2,3-dimethylbutyric Acid

The title compound was prepared using procedures analogous to those described for Example 5, except Piperidine 13 was used. R_{F}: 0.57 (90: 10:1 v/v/v CH₂Cl₂/MeOH/NH₄OH); ¹H-NMR (500 MHz, CD₃OD): δ 0.91 (d, J=7.0, 3H), 0.93 (d, J=7.0, 3H), 1.14 (s, 3H), 1.28-1.46 (m, 7H), 1.56 (m, 1H), 1.68 (m, 1H), 1.76 (m, 1H), 1.95-210 (m, 3H), 2.40 (m, 1H), 2.50-2.57 (m, 2H), 2.73-2.78 (m, 2H), 2.95 (m, 1H), 3.05 (d, J=13.2, 1H), 3.22-3.42 (m, 4H), 3.65-3.72 (m, 2H), 3.77 (s, 2H), 3.96-4.03 (m, 4H), 5.70 (s, 1H), 6.78-6.81 (m, 2H), 6.97 (m, 1H), 7.08-7.18 (m, 4H), 7.33 (m, 1H). ESI-MS 620.6 (M+H); HPLC LC 1: 1.68 min.

### EXAMPLE 8

(2S or 2R)-{[(3S,4S)-3-[(4-{3-[4-Isopropoxybenzyl]-1-ethyl-1H-pyrazol-5-yl}piperidin-1-yl)methyl]-4-(3-fluorophenyl)pyrrolidin-1-yl]methyl}-2-methylcyclobutylacetic Acid

Step A: (2R/2S)-{[(3R,4S)-3-(t-Butyldimethylsilyloxymethyl)-4-(3-fluorophenyl)pyrrolidin-1-yl]methyl}-2-methylcyclobutylacetic Acid, Benzyl Ester

The title compound was prepared as a mixture of diastereomers from (R/S)-2-formyl-cyclobutyl acetic acid, benzyl ester (prepared as Aldehyde 5 above) and 3-(R)-(tert-butyldimethylsilyloxymethyl)-4-(S)-(3-fluoro)phenylpyrrolidine (Prepared as Pyrrolidine 2 above) using a procedure analogous to that described in Example 1, Step A. R_{F}: 0.51 (9:1 v/v hexanes/EtOAc); ¹H-NMR (500 Mhz) δ 0.01-0.02 (m, 6H), 0.87 (s, 9H), 1.23, 1.24 (2s, 3H), 1.65 (m, 1H), 1.75-1.96 (m, 5H), 2.24 (m, 1H), 2.40-2.50 (m, 2H), 2.63-2.90 (m, 6H), 3.49-3.56 (m, 2H), 5.08-5.16 (m, 2H), 6.87 (m, 1H), 6.98-7.03 (m, 2H), 7.19-7.38 (m, 6H).

Step B: (2S or 2R)-{[(3R,4S)-3-(Hydroxymethyl)-4-(3-fluorophenyl)pyrrolidin-1-yl]methyl}-2-methylcyclobutylacetic acid, benzyl ester and (2R or 2S)-{[(3R,4S)-3-(hydroxymethyl)-4-(3-fluorophenyl)pyrrolidin-1-yl]methyl}-2-methylcyclobutylacetic Acid, Benzyl Ester

The title compound was prepared from (2R/2S)-{[(3R,4S)-3-(tert-butyldimethylsilyloxymethyl)-4-(3-fluorophenyl)pyrrolidin-1-yl]methyl}-2-methylcyclobutylacetic acid, benzyl ester (from Step A) using a procedure analogous to that described in Example 1, Step B. R_{F}: 0.30 (3:2 v/v hexanes/EtOAc); Diastereomers were separated by preparative HPLC (Column: Chiralcel OJ; Mobile Phase: 75:25 v/v hexanes/isopropanol; Flow: 9 mL/min; 220 nm). Diastereomer 1 (Retention Time: 12.0 min.): ¹H-NMR (500 Mhz) δ 1.22 (s, 3H), 1.63-1.95 (m, 6H), 2.24-2.84 (m, 8H), 2.99-3.09 (m, 2H), 3.55-3.68 (m, 2H), 5.12 (ABq, J=12.2, 2H), 6.88-7.00 (m, 3H), 7.21-7.37 (m, 6H). Diastereomer 2 (Retention Time: 19.3 min.): ¹H-NMR (500 Mhz) δ 1.24 (s, 3H), 1.64-1.95 (m, 6H), 2.24 (m, 1H), 2.47-2.81 (m, 7H), 3.08-3.12 (m, 2H), 3.56 (m, 1H), 3.64 (m, 1H), 5.13 (ABq, J=12.4, 2H), 6.87-7.02 (m, 3H), 7.21-7.38 (m, 6H).

Step C: (2S or 2R)-{[(3R,4S)-3-(Formyl)-4-(3-fluorophenyl)pyrrolidin-1-yl]methyl}-2-methylcyclobutylacetic Acid, Benzyl Ester

The title compound was prepared (2S or 2R)-{[(3R,4S)-3-(hydroxymethyl)-4-(3-fluorophenyl)pyrrolidin-1-yl]methyl}-2-methylcyclobutylacetic acid, benzyl ester (from Step B, Diastereomer 1) using a procedure analogous to that described for Aldehyde 1, Step B. R_{F}: 0.44 (4:1 v/v hexanes/EtOAc); ¹H-NMR (500 Mhz) δ 1.22 (s, 3H), 1.63-1.96 (m, 6H), 2.45 (m, 1H), 2.59-2.63 (m, 2H), 2.82-2.97 (m, 5H), 3.47 (m, 1H), 5.14 (ABq, J=12.1, 2H), 6.90-7.01 (m, 3H), 7.23-7.38 (m, 6H), 9.61 (d, J=1.6, 1H).

Step D: (2S or 2R)-{[(3S,4S)-3-[(4-{3-[4-Isopropoxybenzyl]-1-ethyl-1H-pyrazol-5-yl}piperidin-1-yl)methyl]-4-(3-fluorophenyl)pyrrolidin-1-yl]methyl}-2-methyl-cyclobutylacetic Acid, Benzyl Ester

The title compound was prepared from (2S or 2R)-{[(3R,4S)-3-(formyl)-4-(3-fluorophenyl)pyrrolidin-1-yl]methyl}-2-methylcyclobutylacetic acid, benzyl ester (from Step C) and 4-(3-(4-isopropoxybenzyl)-1-(ethyl)-(1H)-pyrazol-5-yl)piperidine (Prepared as Piperidine 14 above) using a procedure analogous to that described in Example 1, Step A. R_{F}: 0.68 (19:1 v/v CH₂Cl₂/MeOH); ¹H-NMR (500 Mhz) δ 1.24 (s, 3H), 1.33 (d, J=6.0, 6H), 1.41 (t, J=7.2, 3H), 1.46-1.96 (m, 12H), 2.25-2.43 (m, 6H), 2.63-2.69 (m, 3H), 2.81-2.89 (m, 5H), 3.88 (s, 2H), 4.02 (q, J=7.2, 2H), 4.51 (sept, J=6.0, 1H), 5.71 (s, 1H), 6.82-6.88 (m, 3H), 7.00-7.04 (m, 2H), 7.16-7.38 (m, 8H).

Step E: (2S or 2R)-{[(3S,4S)-3-[(4-{3-[4-Isopropoxybenzyl]-1-ethyl-1H-pyrazol-5-yl}piperidin-1-yl)methyl]-4-(3-fluorophenyl)pyrrolidin-1-yl]methyl}-2-methyl-cyclobutylacetic Acid

The title compound was prepared from (2S or 2R)-{[(3S,4S)-3-[(4-{3-[4-isopropoxybenzyl]-1-ethyl-1H-pyrazol-5-yl}piperidin-1-yl)methyl]-4-(3-fluorophenyl)pyrrolidin-1-yl]methyl}-2-methyl-cyclobutylacetic acid, benzyl ester (from Step D) using a procedure analogous to that described in Example 3, Step E. R_{F}: 0.53 (90:10:1 V/V/V CH₂Cl₂/MeOH/NH₄OH); ¹H-NMR (500 MHz, CD₃OD): δ 1.20 (s, 3H), 1.26 (d, J=6.0, 6H), 1.32 (t, J=7.2, 3H), 1.43 (m, 1H), 1.55 (m, 1H), 1.67-2.09 (m, 13H), 2.38 (m, 1H), 2.49-2.63 (m, 3H), 2.73-2.78 (m, 2H), 2.93-3.02 (m, 2H), 3.19-3.40 (m, 4H), 3.64-3.71 (m, 3H), 3.77 (s, 2H), 4.01 (q, J=7.2, 2H), 4.52 (sept, J=6.0, 1H), 5.71 (s, 1H), 6.79 (d, J=8.6, 2H), 6.97 (m, 1H), 7.08 (d, J=8.6, 2H), 7.15-7.18 (m, 2H), 7.33 (m, 1H). ESI-MS 645.4 (M+H); HPLC LC 2: 2.53 min.

### Example 9

(2S or 2R)-{[(3S,4S)-3-[(4-{3-[4-Cyclobutoxybenzyl]-1-ethyl-1H-pyrazol-5-yl}piperidin-1-yl)methyl]-4-(3-fluorophenyl)pyrrolidin-1-yl]methyl}-2-methylcyclobutylacetic Acid

The title compound was prepared using procedures analogous to those described for Example 8, except Piperidine 16 was used in Step D. R_{F}: 0.59 (90: 10:1 v/v/v CH₂Cl₂/MeOH/NH₄OH); ¹H-NMR (500 MHz, CD₃OD): δ 1.20 (s, 3H), 1.32 (t, J=7.2, 3H), 1.40 (m, 1H), 1.54 (m, 1H), 1.64-2.12 (m, 12H), 2.37-2.78 (m, 7H), 2.93-3.02 (m, 2H), 3.19-3.41 (m, 6H), 3.64-3.71 (m, 3H), 3.76 (s, 2H), 4.01 (q, J=7.2, 2H), 4.61 (quint, J=7.1, 1H), 5.69 (s, 1H), 6.72 (d, J=8.7, 2H), 6.96 (m, 1H), 7.07 (d, J=8.7, 2H), 7.15-7.18 (m, 2H), 7.33 (m, 1H). ESI-MS 657.5 (M+H); HPLC LC 2: 2.61 min.

### Example 10

(2R or 2S)-{[(3S,4S)-3-[(4-{3-[4-Isopropoxybenzyl]-1-ethyl-1H-pyrazol-5-yl}piperidin-1-yl)methyl]-4-(3-fluorophenyl)pyrrolidin-1-yl]methyl}-2-methylcyclobutylacetic Acid

The title compound was prepared using procedures analogous to those described for Example 8, except Diastereomer 2 (from Step B) was used in Step C. R_{F}: 0.52 (90:10:1 v/v/v CH₂Cl₂/MeOH/NH₄OH); ESI-MS 645.5 (M+H); HPLC LC 2: 2.56 min.

### Example 11

(2R or 2S)-{[(3S,4S)-3-[(4-{3-[4-Cyclobutoxybenzyl]-1-ethyl-1H-pyrazol-5-yl}piperidin-1-yl)methyl]-4-(3-fluorophenyl)pyrrolidin-1-yl]methyl}-2-methylcyclobutylacetic Acid

The title compound was prepared using procedures analogous to those described for Example 10, except Piperidine 16 was used. R_{F}: 0.57 (90:10:1 v/v/v CH₂Cl₂/MeOH(OH); ESI-MS 657.5 (M+H); HPLC LC 2: 2.67 min.

### Examples 12 and 13

Examples 12 and 13 were prepared using a procedure analogous to that described in Example 1 substituting the appropriate piperidine in Step D.

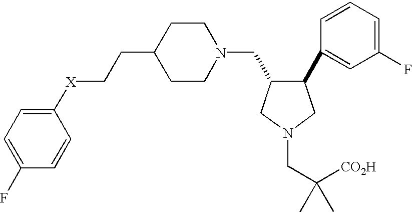

| | | | HPLC | |
|---|---|---|---|---|
| | | HPLC | RT | |
| EXAMPLE # | X | Method | (min) | ESI-MS (M + H) |
| 12 | CF₂ | LC2 | 2.59 | 535.4 |
| | ¹H-NMR(500 MHz, CD₃OD): δ 1.00-1.29(m, | | | |
| | 10H), 1.56-1.63 (m, 2H), 1.83(m, 1H), 1.97(m, 1H), | | | |
| | 2.07-2.17(m, 2H), 2.37(m, 1H), 2.50(m, 1H), 2.70- | | | |
| | 2.74(m, 2H), 2.89(m, 1H), 3.12-3.35(m, 6H), 3.61- | | | |
| | 3.68(m, 2H), 6.99(m, 1H), 7.12-7.17(m, 4H), 7.35 | | | |
| | (m, 1H), 7.47-7.50(m, 2H) | | | |
| 13 | SO₂ | LC2 | 2.00 | 549.5 |

¹H-NMR(500 MHz, CD₃OD): δ 0.98-1.30(m, 9H),
1.51-1.61(m, 4H), 1.79(m, 1H), 1.92(m, 1H), 2.32-
2.49(m, 2H), 2.68-2.73(m, 2H), 2.86(m, 1H), 3.13-
3.36(m, 7H), 3.62-3.70(m, 2H), 6.99(m, 1H), 7.12-
7.16(m, 2H), 7.32-7.39(m, 4H), 7.94-7.97(m, 2H)

### Examples 14-26

Examples 14-26 were prepared using a procedure analogous to that described in Example 2 substituting the appropriate piperidine in Step D.

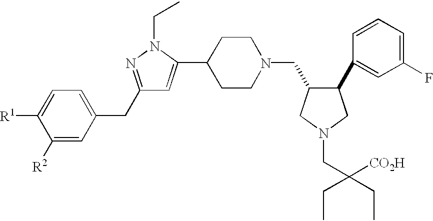

| | | | | HPLC | |
|---|---|---|---|---|---|
| | | | HPLC | RT | ESI-MS |
| EXAMPLE # | R¹ | R² | Method | (min) | (M + H) |
| 14 | OCH(CH₃)₂ | H | LC2 | 2.61 | 633.5 |
| | ¹H-NMR(500 MHz, CD₃OD): δ 0.87-0.91(m, 6H), | | | | |
| | 1.27(d, J=5.9, 6H), 1.32(t, J=7.3, 3H), 1.38-1.78(m, | | | | |
| | 8H), 1.96-2.10(m, 2H), 2.41(m, 1H), 2.50-2.58(m, | | | | |
| | 2H), 2.71-2.79(m, 2H), 2.96(m, 1H), 3.17-3.36(m, | | | | |
| | 5H), 3.61-3.67(m, 2H), 3.77(s, 2H), 4.02(q, J=7.3, | | | | |
| | 2H), 4.52(sept, J=5.9, 1H), 5.72(s, 1H), 6.79(d, J=8.5, | | | | |
| | 2H), 6.97(m, 1H), 7.08(d, J=8.5, 2H), 7.14-7.18(m, | | | | |
| | 2H), 7.33(m, 1H) | | | | |
| 15 | OCH₃ | OCH₃ | LC1 | 1.47 | 635.7 |
| | ¹H-NMR(500 MHz, CD₃OD): δ 0.87-0.91(m, 6H), | | | | |
| | 1.33(t, J=7.1, 3H), 1.41-1.78(m, 8H), 1.96-2.10 | | | | |
| | (m, 2H), 2.40(m, 1H), 2.50-2.58(m, 2H), 2.71-2.79 | | | | |
| | (m, 2H), 2.96(m, 1H), 3.19-3.36(m, 5H), 3.61-3.67 | | | | |
| | (m, 2H), 3.77(s, 3H), 3.78(s, 3H), 3.79(s, 2H), 4.02(q, | | | | |
| | J=7.1, 2H), 5.74(s, 1H), 6.74(m, 1H), 6.81-6.85(m, | | | | |
| | 2H), 6.96(m, 1H), 7.14-7.18(m, 2H), 7.33(m, 1H) | | | | |
| 16 | CH₃ | H | LC2 | 2.16 | 589.4 |
| 17 | H | OCH₂CH₃ | LC2 | 2.19 | 619.4 |
| 18 | OCH₃ | F | LC1 | 1.68 | |
| 19 | OCH₂CH₃ | F | LC2 | 1.77 | 637.7 |
| 20 | O-cyclobutyl | H | LC2 | 2.35 | 646.3 |
| | ¹H-NMR(500 MHz, CD₃OD): δ 0.87-0.91(m, 6H), ), | | | | |
| | 1.32(t, J=7.2, 3H), 1.39-1.84(m, 10H), 1.92-2.12 | | | | |
| | (m, 4H), 2.38-2.44(m, 3H), 2.50-2.57(m, 2H), 2.69- | | | | |
| | 2.79(m, 2H), 2.95(m, 1H), 3.16-3.36(m, | | | | |
| | 5H), 3.61-3.66(m, 2H), 3.76(s, 2H), 4.01(q, J=7.2, | | | | |
| | 2H), 4.61(m, 1H), 5.70(s, 1H), 6.71(d, J=8.7, 2H), | | | | |
| | 6.96(m, 1H), 7.07(d, J=8.7, 2H), 7.14-7.17(m, 2H), | | | | |
| | 7.33(m, 1H) | | | | |
| 21 | tert-Butyl | H | LC2 | 2.58 | 632.3 |
| | ¹H-NMR(500 MHz, CD₃OD): δ 0.87-0.91(m, 6H), | | | | |
| | 1.28(s, 9H), 1.32(t, J=7.2, 3H), 1.35-1.77(m, 8H), | | | | |
| | 1.96-2.10(m, 2H), 2.41(m, 1H), 2.51-2.57(m, 2H), | | | | |
| | 2.70-2.79(m, 2H), 2.96(m, 1H), 3.16-3.36 | | | | |
| | (m, 5H), 3.61-3.67(m, 2H), 3.80(s, 2H), 4.01(q, J= | | | | |
| | 7.2, 2H), 5.72(s, 1H), 6.94(m, 1H), 7.10-7.17(m, 4H), | | | | |
| | 7.28-7.34(m, 3H) | | | | |
| 22 | OCH₃ | H | LC1 | 1.64 | 605 |
| 23 | OCH₂CH₃ | H | LC2 | 2.85 | 619.5 |
| | ¹H-NMR(500 MHz, CD₃OD): δ 0.87-0.91(m, 6H), | | | | |
| | 1.28-1.78(m, 14H), 1.98(m, 1H), 2.08(m, 1H), | | | | |
| | 2.41(m, 1H), 2.50-2.57(m, 2H), 2.71-2.79(m, 2H), | | | | |
| | 2.96(m, 1H), 3.19-3.36(m, 5H), 3.61-3.67(m, | | | | |
| | 2H), 3.77(s, 2H), 3.96-4.03(m, 4H), 5.71(s, 1H), 6.79 | | | | |
| | (d, J=8.7, 2H), 6.97(m, 1H), 7.08(d, J=8.7, 2H), 7.14- | | | | |
| | 7.18(m, 2H), 7.33(m, 1H) | | | | |
| 24 | OCF₃ | H | LC2 | 2.69 | 659.4 |
| | ¹H-NMR(500 MHz, CD₃OD): δ 0.87-0.92(m, 6H), | | | | |
| | 1.32(t, J=7.2, 3H), 1.43-1.79(m, 8H), 1.97(m, 1H), | | | | |
| | 2.08(m, 1H), 2.41(m, 1H), 2.50-2.59(m, 2H), 2.71- | | | | |
| | 2.80(m, 2H), 2.96(m, 1H), 3.20-3.36(m, | | | | |
| | 5H), 3.61-3.67(m, 2H), 3.89(s, 2H), 4.03(q, | | | | |
| | J=7.2, 2H), 5.78(s, 1H), 6.97(m, 1H), 7.14- | | | | |
| | 7.18(m, 4H), 7.28-7.36(m, 3H) | | | | |
| 25 | Iso-Propyl | H | LC2 | 2.72 | 617.4 |

| | | | | |
|---|---|---|---|---|
| 26 | OCH₂O | LC2 | 2.27 | 619.4 |
| | (Methylenedioxy) | | | |

### Examples 27-29

Examples 27-29 were prepared using a procedure analogous to that described in Example 2, except substituting the appropriate piperidine in Step D and reversing Steps D and E to avoid a catalytic hydrogenation in the presence of a reducible heterocycle, such as a thiazole.

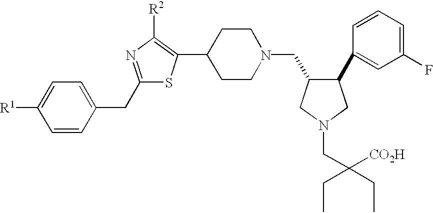

| | | | | HPLC | |
|---|---|---|---|---|---|
| | | | HPLC | RT | ESI-MS |
| EXAMPLE # | R¹ | R² | Method | (min) | (M + H) |
| 27 | OCF₃ | CH₂CH₃ | LC2 | 3.41 | 676.4 |
| | ¹H-NMR(500 MHz, CD₃OD): δ 0.87-0.93(m, 6H), | | | | |
| | 1.17(t, J=7.5, 3H), 1.37-1.81(m, 8H), 2.00-2.15(m, | | | | |
| | 2H), 2.42-2.84(m, 7H), 2.98(m, 1H), 3.17-3.36(m, | | | | |
| | 5H), 3.62-3.67(m, 2H), 4.25(s, 2H), 6.97 | | | | |
| | (m, 1H), 7.14-7.23(m, 4H), 7.32-7.39(m, 3H) | | | | |
| 28 | OCH₂CH₃ | CH₂CH₃ | LC2 | 2.27 | 636.4 |
| | ¹H-NMR(500 MHz, CD₃OD): δ 0.87-0.92(m, 6H), | | | | |
| | 1.17(t, J=7.6, 3H), 1.36(t, J=7.0, 3H), 1.42(m, 1H), | | | | |
| | 1.49-1.79(m, 7H), 2.01-2.17(m, 2H), 2.44(m, 1H), | | | | |
| | 2.55-2.64(m, 3H), 2.72-2.83(m, 3H), 2.98(m, 1H), | | | | |
| | 3.19-3.35(m, 5H), 3.61-3.65(m, 2H), 4.00(q, J=7.0, | | | | |
| | 2H), 4.12(s, 2H), 6.84-6.87(m, 2H), 6.97(m, 1H), | | | | |
| | 7.14-7.18(m, 4H), 7.33(m, 1H) | | | | |
| 29 | OCF₃ | H | LC2 | 3.07 | 648.5 |

¹H-NMR(500 MHz, CD₃OD): δ 0.87-0.91(m, 6H),
1.48-2.14(m, 10H), 2.42(m, 1H), 2.54(m, 1H), 2.72-
2.81(m, 3H), 2.97(m, 1H), 3.20-3.37(m, 5H), 3.62-
3.67(m, 2H), 4.28(s, 2H), 6.98(m, 1H),
7.14-7.22(m, 4H), 7.32-7.39(m, 4H)

### Examples 30-32

Examples 30-32 were prepared using a procedure analogous to that described in Example 2, except substituting the appropriate piperidine in Step D.

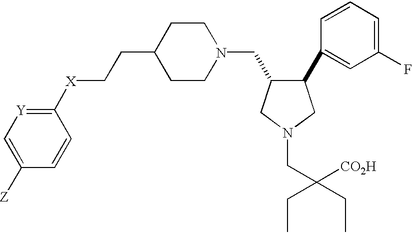

| | | | | | HPLC | |
|---|---|---|---|---|---|---|
| | | | | HPLC | RT | ESI-MS |
| EXAMPLE # | X | Y | Z | Method | (min) | (M + H) |
| 30 | SO₂ | CH | F | LC1 | 1.47 | 577.5 |
| | ¹H-NMR(500 MHz, CD₃OD): δ 0.86-0.90(m, 6H), | | | | | |
| | 1.01-1.31(m, 3H), 1.51-1.97(m, 10H), 2.36(m, | | | | | |
| | 1H), 2.48(m, 1H), 2.66-2.72(m, 2H), 2.87(m, 1H), | | | | | |
| | 3.13-3.21(m, 6H), 3.30(m, 1H), 3.56-3.63(m, | | | | | |
| | 2H), 6.98(m, 1H), 7.11-7.15(m, 2H), 7.31-7.38(m, | | | | | |
| | 3H), 7.93-7.97(m, 2H) | | | | | |
| 31 | CF₂ | N | CF₃ | LC2 | 2.37 | 614.3 |
| | ¹H-NMR(500 MHz, CD₃OD): δ 0.86-0.90(m, 6H), | | | | | |
| | 1.04-1.32(m, 5H), 1.59-1.73(m, 6H), 1.88(m, 1H), | | | | | |
| | 2.01(m, 1H), 2.27-2.43(m, 3H), 2.53(m, 1H), 2.67- | | | | | |
| | 2.93(m, 3H), 3.11-3.30(m, 5H), 3.56-3.59(m, 2H), | | | | | |
| | 6.98(m, 1H), 7.13-7.16(m, 2H), 7.33(m, 1H), 7.86 | | | | | |
| | (d, J=8.3, 1H), 8.25(m, 1H), 8.94(br s, 1H) | | | | | |
| 32 | SO₂ | CH | OCH₂CH₃ | LC1 | 1.58 | 603.5 |

¹H-NMR(500 MHz, CD₃OD): δ 0.88-0.90(m, 6H),
0.98-1.30(m, 3H), 1.41(t, J=7.0, 3H), 1.49-1.96
(m, 10H), 2.36(m, 1H), 2.48(m, 1H), 2.65-2.71(m,
2H), 2.87(m, 1H), 3.11-3.20(m, 6H), 3.29(m, 1H),
3.56-3.62(m, 2H), 4.13(q, J=7.0, 2H), 6.97(m, 1H),
7.08-7.15(m, 4H), 7.33(m, 1H), 7.79(d, J=9.0, 2H)

### Examples 33-35

Examples 33-35 were prepared using a procedure analogous to that described in Example 2, except using Aldehyde 7 and the appropriate piperidines.

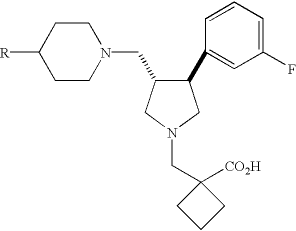

| | | | HPLC | |
|---|---|---|---|---|
| | | | RT | ESI-MS |
| EXAMPLE # | R | HPLC Method | (min) | (M + H) |
| 33 | 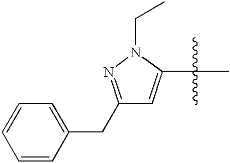 | LC2 | 1.46 | 559.8 |
| 34 | 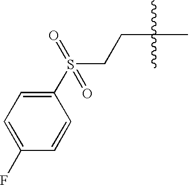 | LC2 | 1.35 | 561.4 |
| 35 | 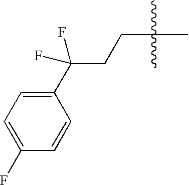 | LC2 | 1.66 | 547.4 |

### Examples 36-55A

Examples 36-55a were prepared using a procedure analogous to that described in Example 2, except using Aldehyde 8 and the appropriate piperidines.

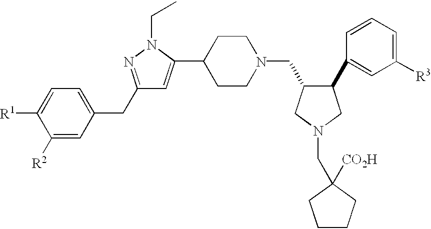

| | | | | | HPLC | |
|---|---|---|---|---|---|---|
| EX- | | | | HPLC | RT | ESI-MS |
| AMPLE # | R1 | R2 | R3 | Method | (min) | (M + H) |
| 36 | H | H | F | LC2 | 1.49 | 573.5 |
| | ¹H NMR(500 MHz, CDCl₃): δ 0.89-3.40(30H), 3.94(s, | | | | | |
| | 2H), 4.02 (q, J=7.1 Hz, 2H), 5.70(s, 2H), 6.92-7.32(9H) | | | | | |
| 37 | OCH₂CH₃ | H | F | LC2 | 1.66 | 617.6 |
| | ¹H NMR(500 MHz, CDCl₃): δ 1.28-3.37(33H), 3.87(s, | | | | | |
| | 2H), 4.00-4.04(4H), 5.68(s, 1H), 6.82-7.32(8H) | | | | | |
| 38 | OiPr | H | F | LC2 | 1.69 | 631.6 |
| | ¹H NMR(500 MHz, CDCl₃): δ 1.31-3.37(36H), 3.87(s, | | | | | |
| | 2H), 4.01(q, J=7.3 Hz, 2H), 4.49-4.54(1H), 5.69(s, 1H), | | | | | |
| | 6.82-7.31(8H) | | | | | |
| 39 | isoPropyl | H | F | LC2 | 1.82 | 615.6 |
| 40 | H | OCH₃ | F | LC2 | 1.53 | 603.6 |
| | ¹H NMR(500 MHz, CDCl₃): δ 1.38-3.37(34H), 3.79(s, | | | | | |
| | 3H), 3.91 (s, 2H), 4.01(q, J=7.3 Hz, 2H), 5.72(s, 1H), | | | | | |
| | 6.75-7.31(8H) | | | | | |
| 41 | H | OCH₂CH₃ | F | LC1 | 2.16 | 618.2 |
| 42 | CH₃ | H | F | LC1 | 2.16 | 588.2 |
| 43 | O- | H | F | LC1 | 2.35 | 644.1 |
| | cyclobutyl | | | | | |
| | ¹H NMR(500 MHz, CDCl₃): δ 1.27-3.36(36H), 3.85(s, | | | | | |
| | 2H), 4.00(q, J=7.2 Hz, 2H), 4.57-4.63(1H), 5.67(s, 1H), | | | | | |
| | 6.72-7.29(8H) | | | | | |
| 44 | tert-Butyl | H | F | LC1 | 2.51 | 630.2 |

¹H NMR(500 MHz, CDCl₃): δ 1.31(s, 9H), 1.34-3.36
(40H), 3.89(s, 2H), 4.00(q, J=7.2 Hz, 2H), 5.71(s, 1H),
6.89-7.32(8H)

| 45 | OCH₃ | F | F | LC1 | 2.05 | 622.1 |
|---|---|---|---|---|---|---|
| 46 | OCH₂CH₃ | F | F | LC1 | 2.19 | 636.1 |
| 47 | OCH₃ | H | F | LC1 | 2.19 | 603.3 |
| 48 | CH₂CH₂O | | F | LC1 | 2.19 | 615.4 |
| | (Benzofuran-6-yl) | | | | | |
| 49 | O- | H | F | LC1 | 2.45 | 629.5 |
| | cyclopropyl | | | | | |
| 50 | OiPr | H | H | LC1 | 1.58 | 613.6 |
| | ¹H-NMR(500 MHz, CD₃OD): δ 1.26(d, J=5.9, 6H), 1.32 | | | | | |
| | (t, J=7.2, 3H), 1.40-2.22(m, 14H), 2.40(m, 1H), 2.49- | | | | | |
| | 2.56(m, 2H), 2.74-2.79(m, 2H), 2.94(m, 1H), 3.18-3.40 | | | | | |
| | (m, 5H), 3.67-3.73(m, 2H), 3.77(s, 2H), 4.01(q, J=7.2, | | | | | |
| | 2H), 4.51(m, 1H), 5.71(s, 1H), 6.79(d, J=8.6, 2H), 7.08 | | | | | |
| | (d, J=8.6, 2H), 7.22-7.36(m, 5H) | | | | | |
| 51 | O- | H | H | LC2 | 2.53 | 625.4 |
| | cyclobutyl | | | | | |
| | ¹H-NMR(500 MHz, CD₃OD): δ 1.32(t, J=7.2, 3H), | | | | | |
| | 1.39-2.22(m, 17H), 2.37-2.56(m, 5H), 2.74-2.78(m, | | | | | |
| | 2H), 2.94(m, 1H), 3.17-3.40(m, 6H), 3.64-3.73(m, 2H), | | | | | |
| | 3.76(s, 2H), 4.01(q, J=7.2, 2H), 4.61(m, 1H), 5.70(s, | | | | | |
| | 1H), 6.72(d, J=8.6, 2H), 7.07(d, J=8.6, 2H), 7.22-7.36 | | | | | |
| | (m, 5H) | | | | | |
| 52 | OCH₂CH₃ | H | H | LC2 | 2.19 | 599.4 |
| | ¹H-NMR(500 MHz, CD₃OD): δ 1.28-2.22(m, 20H), | | | | | |
| | 2.39(m, 1H), 2.49-2.56(m, 2H), 2.74-2.79(m, 2H), | | | | | |
| | 2.94(m, 1H), 3.17-3.40(m, 5H), 3.66-3.73(m, 2H), 3.77 | | | | | |
| | (s, 2H), 3.99-4.03(m, 4H), 5.70(s, 1H), 6.80(d, J=8.7, | | | | | |
| | 2H), 7.08(d, J=8.7, 2H), 7.22-7.35(m, 5H) | | | | | |
| 53 | OCF₃ | H | H | LC2 | 2.56 | 639.4 |
| | ¹H-NMR(500 MHz, CD₃OD): δ 1.33(t, J=7.2, 3H), | | | | | |
| | 1.42-2.23(m, 13H), 2.40(m, 1H), 2.50-2.59(m, 2H), | | | | | |
| | 2.74-2.80(m, 2H), 2.96(m, 1H), 3.18-3.41(m, 6H), 3.68- | | | | | |
| | 3.74(m, 2H), 3.88(s, 2H), 4.02(q, J=7.2, 2H), 5.77(s, | | | | | |
| | 1H), 7.15-7.36(m, 9H) | | | | | |
| 54 | OCH₂O | | F | LC1 | 2.83 | 617.3 |
| | (Methylenedioxy) | | | | | |
| 55 | OCH₃ | OCH₃ | F | LC1 | 2.11 | 633.4 |
| 55a | OCF₃ | H | F | LC1 | 2.64 | 657.5 |

### Examples 56-60

Examples 56-60 were prepared using a procedure analogous to that described in Example 2 using Aldehyde 8 and the appropriate piperidine and reversing Steps D and E to avoid a catalytic hydrogenation in the presence of a reducuble heterocycle, such as a thiazole.

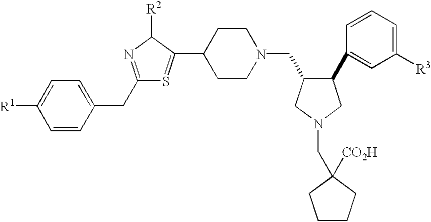

| | | | | HPLC | |
|---|---|---|---|---|---|
| | | | HPLC | RT | ESI-MS |
| EXAMPLE # | R¹ | R² | Method | (min) | (M + H) |
| 56 | OCF₃ | CH₂CH₃ | LC2 | 1.84 | 674.3 |
| | ¹H NMR(500 MHz, CDCl₃): δ 0.84-3.70(32H), | | | | |
| | 4.23(s, 2H), 6.90-7.35(8H) | | | | |
| 57 | OCF₃ | H | LC2 | 2.01 | 646.6 |
| | ¹H NMR(500 MHz, CDCl₃): δ 0.86-3.38(27H), 4.27 | | | | |
| | (s, 2H), 6.92-7.36(9H) | | | | |
| 58 | OCH₂CH₃ | H | LC2 | 1.64 | 606.5 |
| | ¹H NMR(500 MHz, CDCl₃): δ 0.88-3.70(30H), 4.01(q, | | | | |
| | J=7.1 Hz, 2H), 4.18(s, 2H), 6.84-7.32(9H) | | | | |
| 59 | OCH₂CH₃ | CH₂CH₃ | LC2 | 1.68 | 634.6 |
| | ¹H NMR(500 MHz, CDCl₃): δ 0.87-3.35(35H), 4.01(q, | | | | |
| | J=7.0 Hz, 2H), 4.16(s, 2H), 6.85-7.29(8H) | | | | |
| 60 | Cl | H | LC1 | 2.24 | 596.2 |

¹H NMR(500 MHz, CDCl₃): δ 0.88-3.70(27H), 4.22
(s, 2H), 6.91-7.33(9H)

### Examples 61-69B

Examples 61-69b were prepared using a procedure analogous to that described in Example 2 using Aldehyde 8 and the appropriate piperidines.

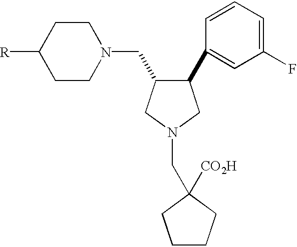

| | | | HPLC | |
|---|---|---|---|---|
| | | HPLC | RT | ESI-MS |
| EXAMPLE # | R | Method | (min) | (M + H) |
| 61 | 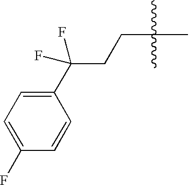 | LC2 | 1.73 | 561.6 |
| | ¹H NMR(500 MHz, CDCl₃): δ 0.88-3.34(31H), | | | |
| | 6.86-7.44(8H) | | | |
| 62 | 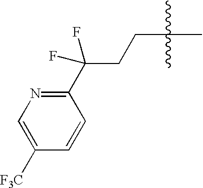 | LC2 | 1.75 | 612.5 |
| | ¹H NMR (500 MHz, CDCl₃): δ 0.88-3.34(31H), | | | |
| | 6.86-6.95(2H), 7.01(d, J=7.7 Hz, 1H), 7.27(q, J=7.7 | | | |
| | Hz, 1H), 7.76(d, J=8.2 Hz, 1H), 8.05(d, J=8.2Hz, 1H), | | | |
| | 8.91(s, 1H) | | | |
| 63 | 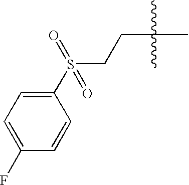 | LC2 | 1.47 | 575.6 |
| | ¹H NMR(500 MHz, CDCl₃): δ 0.88-3.90(31H), | | | |
| | 6.86-7.95(8H) | | | |
| 64 | 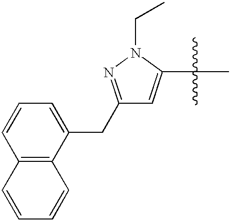 | LC1 | 2.29 | 624.2 |
| 65 | 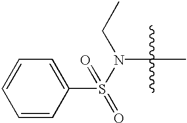 | LC2 | 2.19 | 572.3 |
| 66 | 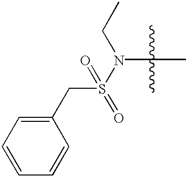 | LC2 | 2.21 | 586.3 |
| 67 | 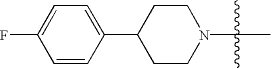 | LC2 | 2.08 | 483.3 |
| 68 | 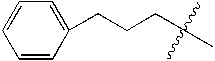 | LC1 | 1.79 | 507.5 |
| 69 | 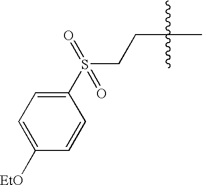 | | | |
| 69a | 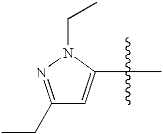 | LC1 | 1.57 | 511.4 |
| 69b | 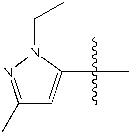 | LC1 | 1.39 | 497.4 |

### Examples 70-87

Examples 70-87 were prepared using a procedure analogous to that described in Example 2 using Aldehyde 6 and the appropriate piperidines.

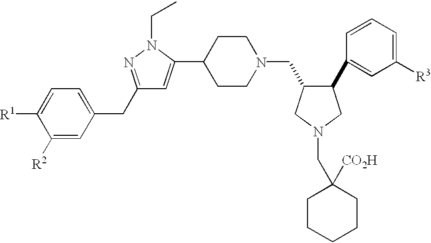

| | | | | | HPLC | |
|---|---|---|---|---|---|---|
| EX- | | | | HPLC | RT | ESI-MS |
| AMPLE # | R¹ | R² | R³ | Method | (min) | (M + H) |
| 70 | OCH₂CH₃ | H | F | LC2 | 1.79 | 631.6 |
| | ¹H NMR(500 MHz, CDCl₃): δ 1.26-3.35(35H), | | | | | |
| | 3.85(s, 2H), 4.00(q, J=6.8 Hz, 4H), 5.66(s, 2H), | | | | | |
| | 6.81-7.28(8H) | | | | | |
| 71 | OCF₃ | H | F | LC2 | 1.99 | 671.6 |
| | ¹H NMR(500 MHz, CDCl₃): δ 1.34-3.43(32H), | | | | | |
| | 3.99(s, 2H), 4.08 (q, J=7.1 Hz, 2H), 5.77(s, 1H), | | | | | |
| | 6.96-7.36(8H) | | | | | |
| 72 | H | H | F | LC2 | 2.08 | 587.4 |
| | ¹H NMR(500 MHz, CDCl₃): δ 1.26-3.35(32H), | | | | | |
| | 3.92(s, 2H), 4.00(q, J=7.3 Hz, 2H), 5.68(s, 1H), | | | | | |
| | 6.89-7.30(9H) | | | | | |
| 73 | OCH₃ | H | F | LC1 | 2.29 | 617.5 |
| 74 | CH₃ | H | F | LC1 | 2.43 | 601.4 |
| 75 | OCH₃ | OCH₃ | F | LC1 | 2.16 | 647.6 |
| | ¹H NMR(500 MHz, CDCl₃): δ 1.26-3.35(32H), | | | | | |
| | 3.83-3.86(8H), 4.00(q, J=7.1 Hz, 2H), 5.69(s, 1H), | | | | | |
| | 6.80-7.28(7H) | | | | | |
| 76 | OCH₃ | F | F | LC1 | 2.32 | 635.5 |
| 77 | OCH₂CH₃ | F | F | LC1 | 2.48 | 649.6 |
| 78 | tert-Butyl | H | F | LC1 | 2.83 | 643.5 |
| | ¹H NMR(500 MHz, CDCl₃): δ 1.27-3.35(41H), | | | | | |
| | 1.31(s, 9H), 3.89(s, 2H), 4.00(q, J=7.3 Hz, 2H), | | | | | |
| | 5.71(s, 1H), 6.88-7.31(8H) | | | | | |
| 79 | CH₂CH₂O | | F | LC1 | 2.29 | 629.5 |
| | (Benzofuran-6-yl) | | | | | |
| 80 | OiPr | H | F | LC1 | 2.59 | 659.5 |
| 81 | O- | H | F | LC1 | 2.51 | 643.5 |
| | cyclopropyl | | | | | |
| 82 | O- | H | H | LC1 | 1.69 | 639.7 |
| | cyclobutyl | | | | | |
| | ¹H-NMR(500 MHz, CD₃OD): δ 1.24-1.83(m, | | | | | |
| | 14H), 1.94-2.14(m, 7H), 2.39-2.45(m, 3H), 2.51- | | | | | |
| | 2.58(m, 2H), 2.74-2.82(m, 2H), 2.97(m, 1H), 3.15- | | | | | |
| | 3.40(m, 7H), 3.63-3.70(m, 2H), 3.76(s, 2H), 4.01 | | | | | |
| | (q, J=7.1, 2H), 4.62(m, 1H), 5.70(s, 1H), 6.72(d, | | | | | |
| | J=8.6, 2H), 7.07(d, J=8.6, 2H), 7.22-7.36(m, 5H) | | | | | |
| 83 | OiPr | H | H | LC1 | 1.64 | 627 |
| | ¹H-NMR(500 MHz, CD₃OD): δ 1.24-1.77(m, | | | | | |
| | 21H), 1.93-2.13(m, 4H), 2.41(m, 1H), 2.51-2.57 | | | | | |
| | (m, 2H), 2.74-2.81(m, 2H), 2.96(m, 1H), 3.16- | | | | | |
| | 3.40(m, 5H), 3.67-3.70(m, 2H), 3.77(s, 2H), 4.01 | | | | | |
| | (q, J=7.1, 2H), 4.51(m, 1H), 5.71(s, 1H), 6.79(d, | | | | | |
| | J=8.6, 2H), 7.08(d, J=8.6, 2H), 7.22-7.36(m, 5H) | | | | | |
| 84 | OCF₃ | H | H | LC2 | 2.67 | 653.4 |
| 85 | OCH₂CH₃ | H | H | LC2 | 2.40 | 613.3 |
| | ¹H-NMR(500 MHz, CD₃OD): δ 1.31-1.77(m, | | | | | |
| | 17H), 1.91-2.11(m, 4H), 2.39(m, 1H), 2.49-2.56 | | | | | |
| | (m, 2H), 2.73-2.80(m, 2H), 2.95(m, 1H), 3.15- | | | | | |
| | 3.39(m, 6H), 3.65-3.70(m, 2H), 3.77(s, 2H), 3.96- | | | | | |
| | 4.03(m, 4H), 5.70(s, 1H), 6.80(d, J=8.6, 2H), 7.08 | | | | | |
| | (d, J=8.6, 2H), 7.22-7.36(m, 5H) | | | | | |
| 86 | O- | H | F | LC1 | 2.64 | 657.5 |
| | cyclobutyl | | | | | |
| 87 | OCH(CH₃)₂ | H | F | LC1 | 2.27 | 646.2 |

### Example 88

Example 88 was prepared using a procedure analogous to that described in Example 1 using Aldehyde 6 and Piperidine 31, except Steps D and E were reversed to avoid a catalytic hydrogenation in the presence of a reducible heterocycle, such as a thiazole.

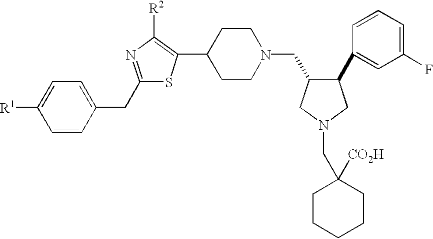

| | | | | HPLC | |
|---|---|---|---|---|---|
| | | | HPLC | RT | ESI-MS |
| EXAMPLE # | R¹ | R² | Method | (min) | (M + H) |
| 88 | OCF₃ | H | LC2 | 1.97 | 660.6 |

¹H NMR(500 MHz, CDCl₃): δ 1.19-3.28(29H), 4.18
(s, 2H), 6.83-7.27(9H)

### Examples 89-93A

Examples 89-93a were prepared using a procedure analogous to that described in Example 2 using Aldehyde 6 and the appropriate piperidines.

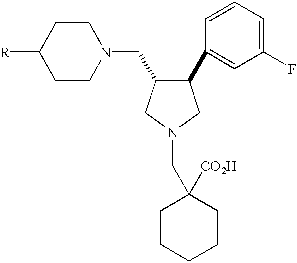

| | | | HPLC | |
|---|---|---|---|---|
| | | HPLC | RT | ESI-MS |
| EXAMPLE # | R | Method | (min) | (M + H) |
| 89 | 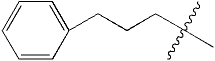 | LC1 | 1.84 | 521.5 |
| 90 | 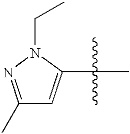 | LC1 | 1.5 | 511.4 |
| 91 | 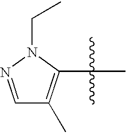 | LC2 | 1.89 | 511.4 |
| 92 | 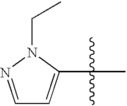 | LC1 | 1.52 | 497.4 |
| 93 | 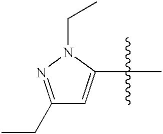 | LC1 | 1.65 | 525.4 |
| 93a | 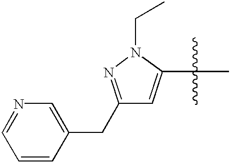 | LC-1 | 1.89 | 588.3 |

### Examples 94-106

Examples 94-106 were prepared using a procedure analogous to that described in Example 2 using Aldehyde 9 and the appropriate piperidines.

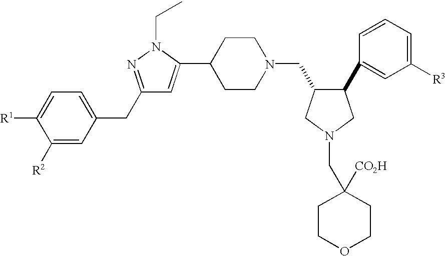

| | | | | | HPLC | |
|---|---|---|---|---|---|---|
| EX- | | | | HPLC | RT | ESI-MS |
| AMPLE # | R¹ | R² | R³ | Method | (min) | (M + H) |
| 94 | H | H | F | LC1 | 2.05 | 589.3 |
| | ¹H NMR (500 MHz, CDCl₃): δ 1.37-3.82 (30H), 3.92 | | | | | |
| | (s, 2H), 4.00 (q, J = 7.1 Hz, 2H), 5.67 (s, 1H), | | | | | |
| | 6.88-7.30 (9H) | | | | | |
| 95 | OiPr | H | F | LC1 | 2.29 | 647.5 |
| 96 | OCH₂CH₃ | H | F | LC1 | 2.19 | 633.4 |
| | ¹H NMR (500 MHz, CDCl₃): δ 1.27-3.85 (35H), | | | | | |
| | 3.97-4.02 (4H), 5.65 (s, 1H), 6.81-7.27 (8H) | | | | | |
| 97 | OCH₃ | H | F | LC1 | 2.05 | 619.3 |
| 98 | O-cyclobutyl | H | F | LC1 | 2.43 | 659.4 |
| 99 | CH₂CH₂O | | F | LC1 | 2.03 | 631.4 |
| | (Benzofuran-6-yl) | | | | | |
| 100 | OCF₃ | H | F | LC1 | 2.48 | 673.3 |
| 101 | OCF₃ | H | H | LC1 | 2.43 | 655.4 |
| 102 | OiPr | H | H | LC1 | 2.27 | 629.4 |
| 103 | OCH₂CH₃ | H | H | LC1 | 2.08 | 615.3 |
| 104 | H | H | H | LC1 | 2.00 | 571.4 |
| 105 | OCH₃ | H | H | LC1 | 1.97 | 601.3 |
| 106 | O-cyclobutyl | H | H | LC1 | 2.37 | 641.5 |

### Example 107

1-{[(3S,4S)-3-[(4-{N′-Benzyl-N″-cyano-N-ethyl-guanidin-N-yl}piperidin-1-yl)methyl]-4-(3-fluorophenyl)pyrrolidin-1-yl]methyl}cyclopentanecarboxylic Acid

The title compound was prepared from N′-benzyl-N″-cyano-N-ethyl-N-(piperidin-4-yl)guanidine (Prepared as Piperidine 32 above), 3-(R)-(tert-butyldimethylsilyloxymethyl)-4-(S)-(3-fluoro)phenylpyrrolidine (Prepared as Pyrrolidine 2 above) and 1-formylcyclopentane carboxylic acid, benzyl ester (Prepared as Aldehyde 8 above) using procedures analogous to those described for Example 2, except Steps D and E were reversed. R_{F}: 0.30 (90:10:1 v/v/v CH₂Cl₂/MeOH/NHOH); ¹H-NMR (500 MHz, CD₃OD): δ 1.09 (t, J=7.1, 3H), 1.46-2.02 (m, 12H), 2.14-2.21 (m, 2H), 2.37 (m, 1H), 2.50 (m, 1H), 2.72-2.76 (m, 2H), 2.93 (m, 1H), 3.22-3.73 (m, 9H), 4.01 (m, 1H), 4.64 (s, 2H), 7.00 (m, 1H), 7.16-7.38 (m, 8H). ESI-MS 589.3 (M+H); HPLC LC 2: 2.08 min.

### Example 108

1-[{[(3S,4S)-3-[(4-{(E/Z)-N^{1′}-Benzyl-2-nitro-N^{1″}-ethene-1,1-diamin-N^{1″}-yl}piperidin-1-yl)methyl]-4-(3-fluorophenyl)pyrrolidin-1-yl]methyl}cyclopentanecarboxylic Acid

The title compound was prepared from (E/Z)-N^{1′}-benzyl-2-nitro-N^{1″}-(piperidin-4-yl)ethene-1,1-diamine (Prepared as Piperidine 33 above), 3-(R)-(tert-butyldimethylsilyloxymethyl)-4-(S)-(3-fluoro)phenylpyrrolidine (Prepared as Pyrrolidine 2 above) and 1-formylcyclopentane carboxylic acid, benzyl ester (Prepared as Aldehyde 8 above) using procedures analogous to those described for Example 2, except Steps D and E were reversed. R_{F}: 0.30 (90:10:1 v/v/v CH₂Cl₂/MeOH/NH₄OH); ¹H-NMR (500 MHz, CD₃OD): δ 1.36-3.68 (m, 27H), 4.44-4.47 (m, 2H), 6.62 (m, 1H), 7.00-7.39 (m, 9H). ESI-MS 580.5 (M+H); HPLC LC 1: 1.31 min; ESI-MS 580.5 (M+H); HPLC LC 1: 1.53 min.

### Example 109

1-{[(3S,4S)-3-[(4-{3-Benzyl-1-ethyl-1H-pyrazol-5-yl}piperidin-1-yl)methyl]-4-phenylpyrrolidin-1-yl]methyl}cyclopentanephosphonic Acid, Diethyl Ester

Step A: 1-(tert-Butoxycarbonyl)cyclopentanephosphonic Acid, Diethyl Ester

To a solution of 2.82 g (7.9 mmol) of benzyl, tributylammonium chloride in 25 mL of 5 N NaOH was added 1.9 mL (8.0 mmol) of tert-butyl diethylphosphonoacetate in 5 mL of 1,4-dibromobutane. After stirring at rt overnight, the reaction was diluted with 100 mL of CH₂Cl₂ and was washed with 6×100 mL of H₂O and 2×100 mL of brine. The organic layer was dried over MgSO₄ and concentrated under reduced pressure. The residue was purified on a 40M Biotage column using 4:1 v/v of hexanes/acetone as the eluant to afford the title compound as a colorless oil: R_{F}: 0.40 (7:3 v/v hexanes/acetone); ¹H-NMR (500 Mhz) δ 1.30 (t, J=7.1, 61), 1.45 (s, 9H), 1.55-1.62 (m, 2H), 1.66-1.71 (m, 2H), 2.00-2.10 (m, 2H), 2.29-2.35 (m, 2H), 4.09-4.15 (m, 4H). Step B: 1-(Carboxylic acid)cyclopentanephosphonic acid, diethyl ester A solution of 1.01 g (3.2 mmol) of 1-(tert-butoxycarbonyl)cyclopentanephosphonic acid, diethyl ester (from Step A) in 6 mL of CH₂Cl₂ at −10° C. (EtOH/ice) was treated with 4 mL of trifluoroacetic acid. After 3.5 hours, volatiles were removed under reduced pressure. The crude product was concentrated from CH₂Cl₂ several times and used without further purification; ¹H-NMR (500 Mhz) δ 1.36 (t, J=7.0, 6H), 1.47-1.77 (m, 4H), 2.10-2.21 (m, 2H), 2.35-2.42 (m, 2H), 4.18-4.25 (m, 4H).

Step C: 1-(Methoxycarbonyl)cyclopentanephosphonic Acid, Diethyl Ester

A solution of 1-(carboxylic acid)cyclopentanephosphonic acid, diethyl ester (3.2 mmol, from Step B) in 10 mL of 7:2 v/v benzene/MeOH was treated with 3.5 mL of 2 M trimethylsilyldiazomethane in hexane. After 30 minutes at rt, volatiles were removed under reduced pressure. The residue was purified on a 40S Biotage column using 7:3 v/v of hexanes/acetone as the eluant to afford the title compound as a colorless oil: R_{F}: 0.31 (7:3 v/v hexanes/acetone); ¹H-NMR (500 Mhz) δ 1.33 (t, J=7.1, 6H), 1.47-1.74 (m, 4H), 2.09-2.18 (m, 2H), 2.34-2.41 (m, 2H), 3.75 (s, 3H), 4.12-4.18 (m, 4H).

Step D: 1-Formylcyclopentanephosphonic Acid, Diethyl Ester

The title compound was prepared from 1-(methoxycarbonyl)cyclopentanephosphonic acid, diethyl ester (from Step C) using a procedure analogous to that described for Aldehyde 3, Step B. R_{F}: 0.62 (7:3 v/v hexanes/acetone); ¹H-NMR (500 Mhz) δ 1.30 (t, J=7.1, 6H), 1.47-1.52 (m, 2H), 1.62-1.73 (m, 2H), 1.98-2.08 (m, 2H), 2.21-2.27 (m, 2H), 4.09-4.16 (m, 4H), 9.55 (s, 1H).

Step E: 1-{[(3R,4S)-3-(tert-Butyldimethylsilyloxymethyl)-4phenylpyrrolidin-1-yl]methyl}cyclopentanephosphonic Acid, Diethyl Ester

The title compound was prepared from 1-formylcyclopentanephosphonic acid, diethyl ester (from Step D) and 3-(R)-(tert-butyldimethylsilyloxymethyl)-4-(S)-phenylpyrrolidine (Prepared as Pyrrolidine 1 above) using a procedure analogous to that described in Example 1, Step A. R_{F}: 0.43 (7:3 v/v hexanes/acetone); ¹H-NMR (500 Mhz) δ 0.012 (s, 3H), 0.013 (s, 3H), 0.87 (s, 9H), 1.32 (t, J=7.1, 6H), 1.68-1.78 (m, 6H), 2.03-2.10 (m, 2H), 2.35 (m, 1H), 2.69-2.82 (m, 4H), 2.94 (m, 1H), 3.01 (m, 1H), 3.10 (m, 1H), 3.53-3.63 (m, 2H), 4.10-4.16 (m, 4H), 7.18 (m, 1H), 7.26-7.32 (m, 4H).

Step F: 1-{[(3R,4S)-3-(Hydroxymethyl)-4-phenylpyrrolidin-1-yl]methyl}cyclopentanephosphonic Acid, Diethyl Ester

The title compound was prepared from 1-{[(3R,4S)-3-(tert-butyldimethylsilyloxymethyl)-4phenylpyrrolidin-1-yl]methyl}cyclopentanephosphonic acid, diethyl ester (from Step E) using a procedure analogous to that described in Example 1, Step B. R_{F}: 0.50 (19:1 v/v CH₂Cl₂/MeOH); ¹H-NMR (500 Mhz) δ 1.29-1.49 (m, 7H), 1.59-1.75 (m, 5H), 2.11-2.19 (m, 2H), 2.30 (m, 1H), 2.41-2.48 (m, 2H), 2.66 (m, 1H), 2.85 (m, 1H), 3.02 (m, 1H), 3.25-3.39 (m, 3H), 3.56 (m, 1H), 3.78 (m, 1H), 4.05-4.25 (m, 4H), 7.18 (m, 1H), 7.25-7.30 (m, 4H).

Step G: 1-{[(3R,4S)-3-Formyl-4-phenylpyrrolidin-1-yl]methyl}cyclopentanephosphonic Acid, Diethyl Ester

The title compound was prepared from 1-{[(3R,4S)-3-(hydroxymethyl)-4-phenylpyrrolidin-1-yl]methyl}cyclopentanephosphonic acid, diethyl ester (from Step F) using a procedure analogous to that described for Aldehyde 1, Step B. R_{F}: 0.63 (19:1 v/v CH₂Cl₂/MeOH); ¹H-NMR (500 Mhz) δ 1.26-3.66 (m, 22H), 4.07-4.26 (m, 4H), 7.21-7.33 (m, 5H), 9.72 (d, J=1.8, 1H).

Step H: 1-{[(3S,4S)-3-[(4-{3-Benzyl-1-ethyl-1H-pyrazol-5-yl}piperidin-1-yl)methyl]-4-phenylpyrrolidin-1-yl]methyl}cyclopentanephosphonic Acid, Diethyl Ester

The title compound was prepared from 1-{[(3R,4S)-3-formyl-4-phenylpyrrolidin-1-yl]methyl}cyclopentanephosphonic acid, diethyl ester (from Step G) and 4-(3-benzyl-1-ethyl-1H-pyrazol-5-yl)piperidine, trifluoro-acetic acid salt (Prepared as Piperidine 1 above) using a procedure analogous to that described in Example 1, Step A. R_{F}: 0.36 (19:1 v/v CH₂Cl₂/MeOH); 1H-NMR (500 Mhz) δ 1.27-2.10 (m, 23H), 2.38-2.43 (m, 4H), 2.64-3.13 (m, 9H), 3.94 (s, 2H), 4.02 (q, J=7.2, 2H), 4.11-4.16 (m, 4H), 7.16-7.32 (m, 10H); ESI-MS 647.4 (M+H); HPLC LC 2: 2.29 min.

### Example 110

1-{[(3S,4S)-3-[(4-{3-Benzyl-1-ethyl-1H-pyrazol-5-yl}piperidin-1-yl)methyl]-4-phenylpyrrolidin-1-yl]methyl}cyclopentanephosphonic Acid, Monoethyl Ester and

1-{[(3S,4S)-3-[(4-{3-benzyl-1-ethyl-1H-pyrazol-5-yl}piperidin-1-yl)methyl]-4-phenylpyrrolidin-1-yl]methyl}cyclopentanephosphonic Acid

A solution of 1-{[(3S,4S)-3-[(4-{3-benzyl-1-ethyl-1H-pyrazol-5-yl}piperidin-1-yl)methyl]-4-phenylpyrrolidin-1-yl]methyl}cyclopentanephosphonic acid, diethyl ester (Prepared as Example 109 above) in 4 mL of 2 N HCl was refluxed for 48 hours to give a mixture of the monoacid and diacid. Volatiles were removed under reduced pressure. The residue was purified by flash chromatography using 90:10:1 v/v/v CH₂Cl₂/MeOH/NH₄OH and 80:20:2 v/v/v CH₂Cl₂/MeOH/NHOH as the eluant to afford the title compounds. 1-{[(3S,4S)-3-[(4-{3-benzyl-1-ethyl-1H-pyrazol-5-yl}piperidin-1-yl)methyl]-4-phenylpyrrolidin-1-yl]methyl}cyclopentanephosphonic acid, monoethyl ester: ¹H-NMR (500 MHz, CD₃OD): δ 1.26-2.19 (m, 17H), 2.70-3.60 (m, 16H), 3.85 (s, 2H), 4.01-4.09 (m, 4H), 5.83 (s, 1H), 7.14-7.45 (m, 10H); ESI-MS 619.3 (M+H); HPLC LC 2: 2.19 min. 1-{[(3S,4S)-3-[(4-{3-benzyl-1-ethyl-1H-pyrazol-5-yl}piperidin-1-yl)methyl]-4-phenylpyrrolidin-1-yl]methyl}cyclopentanephosphonic acid: ¹H-NMR (500 MHz, CD₃OD): δ 1.33-2.17 (m, 14H), 2.60-3.66 (m, 16H), 3.85 (s, 2H), 4.05-4.09 (m, 2H), 5.81 (s, 1H), 7.14-7.45 (m, 10H); ESI-MS 591.3 (M+H); HPLC LC 2: 2.00 min.

### Example 111

(3S or 3R)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-tetrahydro-3-furoic Acid

Step A: 3,3-Bis-Benzyloxycarbonyl-tetrahydrofuran

A solution of 2.05 mL (8.2 mmol) of dibenzyl malonate in 160 mL of toluene was treated with 1.06 g (8.2 mmol) of (2-chloroethyl)-chloromethyl ether, 2.27 g (16.4 mmol) of potassium carbonate, 4.33 g (16.4 mmol) of 18-crown-6 and 908 mg (2.5 mmol) of tetrabutylammonium iodide. The reaction was warmed to 100 ° C. for 4 hours. After cooling to rt, the reaction was poured into 200 mL of EtOAc and washed with 200 mL of 1 N NaHCO₃. After separating phases the aqueous layer was extracted with 200 mL of EtOAc. The combined organic layers were dried over Na₂SO₄ and concentrated under reduced pressure. The residue was purified on a 40S Biotage column using 9:1 v/v of hexanes/acetone as the eluant to afford the title compound as a colorless oil: R_{F}: 0.45 (4:1 v/v hexanes/acetone); ¹H-NMR (500 Mhz) δ 2.53 (t, J 7.0, 2H), 3.91 (t, J=7.0, 2H), 4.24 (s, 211), 5.16 (s, 4H), 7.24-7.37 (m, 10H).

Step B: 3-Benzyloxycarbonyl-3-formyltetrahydrofuran

The title compound was prepared from 3,3-bis-benzyloxycarbonyl-tetrahydrofuran (from Step A) using a procedure analogous to that described for Aldehyde 3, Step B. R_{F}: 0.45 (4:1 v/v hexanes/acetone); ¹H-NMR (500 Mhz) δ 2.45 (t, J=7.0, 2H), 3.81-3.96 (m, 2H), 4.06 (d, J=9.5, 1H), 4.26 (d, J=9.5, 1H), 5.24 (s, 2H), 7.27-7.42 (m, 5H), 9.72 (s, 1H).

Step C: (3S or 3R)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-tetrahydro-3-furoic Acid, Benzyl Ester and (3R or 3S)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-tetrahydro-3-furoic Acid, Benzyl Ester

The title compounds were prepared from 3-benzyloxycarbonyl-3-formyltetrahydrofuran (from Step B) and 3-(R)-(tert-butyldimethylsilyloxymethyl)-4-(S)-(3-fluoro)phenylpyrrolidine (Prepared as Pyrrolidine 2 above) using procedures analogous to that described in Example 1, Steps A to D. Diastereomers were separated by preparative HPLC (Column: Chiralcel OD; Mobile Phase: 62:38 v/v hexanes/isopropanol; Flow: 9 mL/min; 220 nm). Diastereomer 1 (Retention Time: 14.2 min.): 1H-NMR (500 Mhz) δ 1.30-2.94 (m, 24H), 3.67 (d, J=8.9, 1H), 3.82-3.91 (m, 2H), 3.94 (s, 2H), 4.02 (q, J=7.3, 2H), 4.23 (d, J=8.9, 1H), 5.19 (ABq, J=12.3, 2H), 5.71 (s, 1H), 6.86 (m, 1H), 6.97-7.01 (m, 2H), 7.18-7.37 (m, 11H). Diastereomer 2 (Retention Time: 19.9 min.): ¹H-NMR (500 Mhz) δ 1.30-2.04 (m, 10H), 2.27-2.95 (m, 14H), 3.66 (d, J=8.9, 1H), 3.82-3.91 (m, 2H), 3.94 (s, 2H), 4.02 (q, J=7.3, 2H), 4.22 (d, J=9.0, 1H), 5.19 (ABq, J=12.4, 2H), 5.71 (s, 1H), 6.86 (m, 1H), 6.97-7.02 (m, 2H), 7.17-7.38 (m, 11H).

Step D: (3S or 3R)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-tetrahydro-3-furoic Acid

The title compound was prepared from (3S or 3R)-{[(3S,4S)-3-[(4-{3-benzyl-1-ethyl-1H-pyrazol-5-yl}piperidin-1-yl)methyl]-4-(3-fluorophenyl)pyrrolidin-1-yl]methyl}-tetrahydro-3-furoic acid, benzyl ester (Step C, Diastereomer 1) using a procedure analogous to that described in Example 2, Step E. 1H-NMR (500 MHz, CD₃OD): δ 1.21-4.12 (m, 32H), 5.74 (s, 1H), 6.95 (m, 1H), 7.14-7.34 (m, 8H); ESI-MS 575.5 (M+H); HPLC LC 2: 2.16 min.

### Example 112

(3R or 3S)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-tetrahydro-3-furoic Acid

The title compound was prepared from (3R or 3S)-{[(3S,4S)-3-[(4-{3-benzyl-1-ethyl-1H-pyrazol-5-yl}piperidin-1-yl)methyl]-4-(3-fluorophenyl)pyrrolidin-1-yl]methyl}-tetrahydro-3-furoic acid, benzyl ester (Example 111, Step C, Diastereomer 2) using a procedure analogous to that described in Example 2, Step E. ¹H-NMR (500 MHz, CD₃OD): δ 1.23-4.09 (m, 32H), 5.74 (s, 1H), 6.95 (m, 1H), 7.14-7.34 (m, 8H); ESI-MS 575.5 (M+H); HPLC LC 2: 2.24 min.

### Example 113

(2S or 2R)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-tetrahydro-2-furoic Acid

Step A: Diazomalonic Acid, Dibenzyl Ester

A solution of 4.4 mL (17.6 mmol) of dibenzyl malonate and 6.19 g (17.6 mmol) of para-dodecylbenzene sulfonyl azide (Syn. Comm. 1981, 11, 947-956) in CH₃CN at 0° C. was treated with 2.45 mL (17.6 mmol) of triethylamine. The reaction was warmed to rt and stirred for 17.5 hours. The reaction was poured into 200 mL of Et₂O and washed with 200 mL of H₂O, 200 mL of 2 N HCl and 200 mL of brine. The organic layer was dried over MgSO₄ and concentrated under reduced pressure. The residue was purified on a 40M Biotage column using 3:1 v/v of hexanes/Et₂O. The product was recrytallized from 20 mL of 3:1 v/v hexanes/Et₂O to afford the title compound as white crystals: R_{F}: 0.35 (7:3 v/v hexanes/Et₂O); ¹H-NMR (500 Mhz) δ 5.30 (s, 4H), 7.27-7.41 (m, 10H).

Step B: (3-Chloro-1-propoxy)malonic Acid, Dibenzyl Ester

A solution of 1.57 g (5.0 mmol) of diazomalonic acid, dibenzyl ester (from Step A) and 2.1 mL (25 mmol) of 3-chloro-1-propanol in 4 mL of benzene was added to a suspension 67.5 mg (0.15 mmol) of rhodium (II) acetate dimer in 3 mL of benzene. The reaction was warmed to 55° C. for 2.5 hours. After cooling to rt, volatiles were removed under reduced pressure. The residue was partitioned between 200 mL of EtOAc and 200 mL of H₂O. After separarting layers, the organic layer was washed with 100 mL of brine, dried over Na₂SO₄ and concentrated under reduced pressure. The residue was purified on a 40M Biotage column using 4:1 v/v of hexanes/EtOAc to afford the title compound as a colorless oil: R_{F}: 0.29 (7:3 v/v hexanes/Et₂O); ¹H-NMR (500 Mhz) δ 2.07-2.12 (m, 2H), 3.67 (d, J=6.3, 2H), 3.76 (d, J=5.9, 2H), 4.60 (s, 1H), 5.22 (s, 4H), 7.27-7.39 (m, 10H).

Step C: 2,2-Bis-Benzyloxycarbonyl-tetrahydrofuran

A solution of 1.29 g (3.4 mmol) of (3-chloro-1-propoxy)malonic acid, dibenzyl ester, 1.12 g (3.4 mmol) of cesium carbonate and 256 mg (0.68 mmol) of tetrabutylammonium iodide in 8 mL of DMF was stirred at rt overnight. The reaction was poured into 200 mL of Et₂O, washed with 2×200 mL of brine, dried over MgSO₄ and concentrated under reduced pressure. The residue was purified on a 40M Biotage column using 7:3 v/v of hexanes/ Et₂O to afford the title compound as a colorless oil: R_{F}: 0.23 (7:3 v/v hexanes/Et₂O); ¹H-NMR (500 Mhz) δ 2.01 (m, 2H), 2.47 (t, J=7.3, 2H), 4.08 (t, J=6.7, 2H), 5.18 (ABq, J=12.2, 4H), 7.26-7.33 (m, 10H).

Step D: 2-Benzyloxycarbonyl-tetrahydro-2-furoic Acid

A mixture of 1.14 g (3.3 mmol) of 2,2-bis-benzyloxycarbonyl-tetrahydrofuran (from Step C) and 65 mg of 10% palladium on carbon in 10 mL of MeOH was hydrogenated at rt under a balloon of hydrogen for 1.0 hour. The reaction was filtered and concentrated under reduced pressure. The residue was purified by flash chromatography using a gradient of 19:1 v/v CH₂Cl₂/MeOH, 19:1:0.5 v/v/v CH₂Cl₂/MeOH/HOAc and 9:1:0.1 v/v/v CH₂Cl₂/MeOH/HOAc to afford the title compound as colorless oil. R_{F}: 0.54 (9:1:0.1 v/v/v CH₂Cl₂/MeOH/HOAc); ¹H-NMR (500 Mhz) δ 1.97-2.06 (m, 2H), 2.44 (m, 1H), 2.58 (m, 1H), 4.04-4.12 (m, 2H), 5.24 (ABq, J=12.3, 2H), 7.27-7.37 (m, 5H), 9.08 (br s, 1H).

Step E: 2-(Hydroxymethyl)-tetrahydro-2-furoic Acid

A solution of 288 mg (1.1 mmol) of 2-benzyloxycarbonyl-tetrahydro-2-furoic acid (from Step D) in 2.5 mL of EtOH at 0° C. was treated with 64.5 mg (1.1 mmol) of KOH. After 1 hour volatiles were removed under reduced pressure. The residue was suspended in 3.8 mL of benzene and cooled to 0° C. The solution was treated with 0.2 mL (2.3 mmol) of oxalyl chloride and 5 drops of DMF. The reaction was warmed to rt and stirred for 2 hours. The reaction was placed in the freezer for 3 days. Solids were removed by filtration through a plug of glass wool. The filtrate was concentrated under reduced pressure and used without further purification.

A solution of 248 mg (0.9 mmol) of the acid chloride (prepared in the previous paragraph) in 1.8 mL of THF at −78° C. was treated with 2.25 mL (0.9 mmol) of 0.4 M lithium tri-tert-butoxyaluminohydride in THF. After stirring for 45 min, the reaction was quenched with 10% citric acid. The reaction was warmed to rt and partitioned between EtOAc and 10% citric acid. After separating phases, the aqueous layer was extracted twice with EtOAc. The combined organic layers were washed with 1 N NaHCO₃, brine and dried over over Na₂SO₄. The residue was purified by flash chromatography using 1:1 v/v hexanes/EtOAc as the eluant to afford the title compound as a colorless oil. R_{F}: 0.26 (1:1 v/v hexanes/EtOAc); ¹H-NMR (500 Mhz) δ 1.91-1.99 (m, 2H), 2.04 (m, 1H), 2.14 (m, 1H), 2.25 (m, 1H), 3.70 (d, J=11.5, 1H), 3.87 (d, J=11.5, 1H), 4.00-4.14 (m, 2H), 5.22 (ABq, J=12.5, 2H), 7.27-7.39 (m, 5H).

Step F: 2-Formyl-tetrahydro-2-furoic Acid

The title compound was prepared from 2-(hydroxymethyl)-tetrahydro-2-furoic acid (from Step E) using a procedure analogous to that described in Aldehyde 1, Step B. R_{F}: 0.31 (3:2 v/v hexanes/EtOAc); 1H-NMR (500 Mhz) δ 1.86-2.06 (m, 2H), 2.29 (m, 1H), 2.41 (m, 1H), 4.05-4.13 (m, 2H), 5.24 (s, 2H), 7.27-7.40 (m, 5H), 9.62 (s, 1H).

Step G: (3S,4S)-3-[(4-{3-Benzyl-1-ethyl-1H-pyrazol-5-yl}piperidin-1-yl)methyl]-4-(3-fluorophenyl)pyrrolidine

The title compound was prepared from (3R, 4S)-tert-butoxycarbonyl-3-(formyl)-4-(3-fluorophenyl)pyrrolidine (from Example 127, Step C) and 4-(3-benzyl-1-ethyl-1H-pyrazol-5-yl)piperidine, trifluoro-acetic acid salt (Prepared as Piperidine 1 above) using procedures analogous to Example 1, Step A and Piperidine 34, Step C. R_{F}: 0.29 (90:10:1 v/v/v CH₂Cl₂/MeOH/NH₄OH).

Step H: (2S or 2R)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-tetrahydro-2-furoic Acid, Benzyl Ester and (2R or 2S)-{[(3S,4S)-3-[(4-{3-benzyl-1-ethyl-1H-pyrazol-5-yl}piperidin-1-yl)methyl]-4-(3-fluorophenyl)pyrrolidin-1-yl]methyl}-tetrahydro-2-furoic Acid, Benzyl Ester

The title compounds were prepared from 2-formyl-tetrahydro-2-furoic acid (from Step F) and (3S,4S)-3-[(4-{3-benzyl-1-ethyl-1H-pyrazol-5-yl}piperidin-1-yl)methyl]-4-(3-fluorophenyl)pyrrolidine (from Step G) using a procedure analogous to that described in Example 1, Step A. Diastereomers were separated by preparative HPLC (Column: Chiralcel OD; Mobile Phase: 85:15 v/v hexanes/isopropanol; Flow: 9 mL/min; 220 nm). Diastereomer 1 (Retention Time: 19.2 min.): 1H-NMR (500 Mhz) δ 1.40-3.19 (m, 26H), 3.95-4.05 (m, 6H), 5.24 (ABq, J=12.3, 2H), 5.72 (s, 1H), 6.83 (m, 1H), 7.00-7.02 (m, 2H), 7.15-7.40 (m, 11H). Diastereomer 2 (Retention Time: 22.9 min.): ¹H-NMR (500 Mhz) δ 1.40-3.16 (m, 26H), 3.95-4.05 (m, 6H), 5.22 (ABq, J=12.4, 2H), 5.72 (s, 1H), 6.84 (m, 1H), 7.03-7.05 (m, 2H), 7.16-7.37 (m, 11H).

Step I: (2S or 2R)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-tetrahydro-2-furoic Acid

The title compound was prepared from (2S or 2R)-{[(3S,4S)-3-[(4-{3-benzyl-1-ethyl-1H-pyrazol-5-yl}piperidin-1-yl)methyl]-4-(3-fluorophenyl)pyrrolidin-1-yl]methyl}-tetrahydro-2-furoic acid, benzyl ester (Step H, Diastereomer 1) using a procedure analogous to that described in Example 2, Step E. R_{F}: 0.15 (90: 10:1 V/V/V CH₂Cl₂/MeOH/NH₄OH). ¹H-NMR (500 MHz, CD₃OD): δ 1.31-4.08 (m, 33H), 5.72 (s, 1H), 6.94 (m, 1H), 7.09-7.33 (m, 8H); ESI-MS 575.5 (M+H); HPLC LC 1: 1.40 min.

### EXAMPLE 114

(2R or 2S)-{[(3S,4S)-3-[(4-{3-Benzyl-1-ethyl-1H-pyrazol-5-yl}piperidin-1-yl)methyl]-4-(3-fluorophenyl)pyrrolidin-1-yl]methyl}-tetrahydro-2-furoic Acid

The title compound was prepared from (2R or 2S)-{[(3S,4S)-3-[(4-{3-benzyl-1-ethyl-1H-pyrazol-5-yl}piperidin-1-yl)methyl]-4-(3-fluorophenyl)pyrrolidin-1-yl]methyl}-tetrahydro-2-furoic acid, benzyl ester (Example 113, Step H, Diastereomer 2) using a procedure analogous to that described in Example 2, Step E. R_{F}: 0.18 (90:10:1 v/v/v CH₂Cl₂/MeOH/NH₄OH). 1H-NMR (500 MHz, CD₃OD): δ 1.32 (t, J=7.2, 3H), 1.37-3.66 (m, 24H), 3.84 (s, 2H), 3.93 (m, 1H), 4.02 (q, J=7.2, 2H), 4.09 (m, 1H), 5.71 (s, 11H), 6.93 (m, 1H), 7.13-7.31 (m, 8H); ESI-MS 575.3 (M+H); HPLC LC 2: 2.16 min.

### EXAMPLE 115

1-{[(trans, 3R/S,4R/S)-3-[(4-{3-Benzyl-1-ethyl-1H-pyrazol-5-yl}piperidin-1-yl)methyl]-4-(5-methylfuran-3-yl)pyrrolidin-1-yl]methyl}cyclopentanecarboxylic Acid

Step A: (E)-N-Methoxy-N-methyl-3-(5-methylfuran-3-yl)-propenamide

A solution of 3.97 g (36 mmol) of 5-methyl-3-furaldehyde (J. Org. Chem. 1992, 57, 3126) and 15.7 g (43 mmol) of N-methoxy-N-methyl-2-(triphenylphosphoranylidene)acetamide in 70 mL of toluene was warmed at 60° C. for 1.75 hours. The reaction was cooled to rt and concentrated under reduced pressure. The residue was purified on a 40M Biotage column using 17:3 v/v of hexanes/acetone to afford a product, which was recrytallized twice from 9:1 v/v hexanes/EtOAc to yield the title compound as needles: R_{F}: 0.28 (4:1 v/v hexanes/acetone); ¹H-NMR (500 Mhz) δ 2.30 (s, 3H), 3.28 (s, 3H), 3.74 (s, 3H), 6.24 (s, 1H), 6.67 (d, J=15.5, 1H), 7.50 (s, 1H), 7.56 (d, J=15.5, 1H).

Step B: (trans, 3R/S,4R/S)-1-Benzyl-3-(N-methoxy-N-methyl-carboxamido)-4(5-methylfuran-3-yl)pyrrolidine

The title compound was prepared from (E)-N-methoxy-N-methyl-3-(5-methylfuran-3-yl)-propenamide (from Step A) and N-methoxymethyl-N-trimethylsilylmethyl benzyl amine using a procedure analogous to that described for Pyrrolidine 1, Step B. R_{F}: 0.17 (17:3 v/v CH₂Cl₂/acetone). 1H-NMR (500 Mhz) δ 2.24 (s, 3H), 2.68-2.75 (m, 2H), 2.92-3.17 (m, 3H), 3.18 (s, 3H), 3.54 (s, 3H), 3.63-3.84 (m, 311), 5.97 (s, 1H), 7.12 (s, 1H), 7.23-7.38 (m, 5H).

Step C: (trans, 3R/S,4R/S)-1-Benzyl-3-formyl-4-(5-methylfuran-3-yl)pyrrolidine

A solution of 440 mg (1.3 mmol) of (trans, 3R/S,4R/S)-1-benzyl-3-(N-methoxy-N-methyl-carboxamido)-4-(5-methylfuran-3-yl)pyrrolidine in 27 mL of toluene at −78° C. was treated with 2.0 mL (2.0 mmol) of 1 M Dibal in toluene. After 50 minutes the reaction was quenched with saturated Rochelle salts and warmed to rt.

The mixture was partitioned between 50 mL of CH₂Cl₂ and 50 mL of saturated Rochelle salts. After separating phases, the aqueous layer was extracted with 2×50 mL of CH₂Cl₂. The combined organic phases were washed with brine, dried over Na₂SO₄ and concentrated under reduced pressure. The residue was purified by flash chromatography using 17:3 v/v hexane/acetone as the eluant to afford the title compound as a colorless oil. R_{F}: 0.60 (3:2 v/v hexanes/acetone). ¹H-NMR (500 Mhz) δ 2.26 (s, 3H), 2.43 (t, J=8.5, 1H), 2.72 (t, J=9.2, 1H), 2.86 (m, 1H), 3.11-3.16 (m, 2H), 3.50 (m, 1H), 3.67 (ABq, J=13.0, 2H), 5.93 (s, 1H), 7.14 (s, 1H), 7.25-7.39 (m, 5H), 9.70 (d, J=2.1, 1H).

Step D: (trans, 3R/S,4R/S)-1-Benzyl-3-[(4-{3-benzyl-1-ethyl-1H-pyrazol-5-yl}piperidin-1-yl)methyl]-4-(5-methylfuran-3-yl)pyrrolidine

The title compound was prepared from (trans, 3R/S,4R/S)-1-benzyl-3-formyl-4-(5-methylfuran-3-yl)pyrrolidine (from Step C) and 4-(3-benzyl-1-ethyl-1H-pyrazol-5-yl)piperidine, trifluoro-acetic acid salt (Prepared as Piperidine 1 above) using a procedure analogous to that described in Example 1, Step A. R_{F}: 0.38 (3:2 v/v hexanes/acetone); ¹H-NMR (500 Mhz) δ 1.43 (t, J=7.2, 3H), 1.59-1.65 (m, 2H), 1.74-1.79 (m, 2H), 1.91-2.03 (m, 2H), 2.25-2.29 (m, 4H), 2.37-2.48 (m, 4H), 2.56 (m, 1H), 2.76-2.98 (m, 5H), 3.65 (ABq, J 13.0, 2H), 3.94 (s, 2H), 4.04 (q, J=7.2, 2H), 5.72 (s, 1H), 5.95 (s, 1H), 7.10 (s, 1H), 7.21-7.36 (m, 10H).

Step E: (trans, 3R/S,4R/S)-3-[(4-{3-Benzyl-1-ethyl-1H-pyrazol-5-yl}piperidin-1-yl)methyl]-4-(5-methylfuran-3-yl)pyrrolidine The title compound was prepared from (trans, 3R/S,4R/S)-1-benzyl-3-[(4-{3-benzyl-1-ethyl-1H-pyrazol-5-yl}piperidin-1-yl)methyl]-4-(5-methylfuran-3-yl)pyrrolidine (from Step D) using a procedure analogous to that described for Pyrrolidine 1, Step E. R_{F}: 0.22 (90:10:1 v/v/v CH₂Cl₂/MeOH/NH₄OH); ¹H-NMR (500 Mhz) δ 1.42 (t, J=7.2, 3H), 1.57-1.66 (m, 2H), 1.76-1.80 (m, 2H), 1.91-2.08 (m, 2H), 2.24-2.48 (m, 7H), 2.76-2.96 (m, 5H), 3.33-3.38 (m, 2H), 3.93 (s, 2H), 4.04 (q, J=7.2, 2H), 4.53 (br m, 1H), 5.72 (s, 1H), 5.91 (s, 1H), 7.13 (s, 1H), 7.18-7.30 (m, 5H).

Step F: 1-{[(trans, 3R/S,4R/S)-3-[(4-{3-Benzyl-1-ethyl-1H-pyrazol-5-yl}piperidin-1-yl)methyl]-4-(5-methylfuran-3-yl)pyrrolidin-1-yl]methyl}cyclopentanecarboxylic Acid

The title compound was prepared from (trans, 3R/S,4R/S)-3-[(4-{3-benzyl-1-ethyl-1H-pyrazol-5-yl}piperidin-1-yl)methyl]-4-(5-methylfuran-3-yl)pyrrolidine (from Step E) and 1-formylcyclopentane carboxylic acid, benzyl ester (Prepared as Aldehyde 8 above) using procedures analogous to that described in Example 1, Step A and Example 3, Step E. ¹H-NMR (500 MHz, CD₃OD): δ 1.35 (t, J=7.2, 3H), 1.44-2.64 (m, 20H), 2.87-3.64 (m, 10H), 3.85 (s, 2H), 4.05 (q, J=7.2, 2H), 5.78 (s, 1H), 6.07 (s, 1H), 7.14-7.25 (m, 5H), 7.30 (s, 1H); ESI-MS 559.6 (M+H); HPLC LC 1: 1.55 min.

### Example 116

1-{[(trans, 3R/S,4R/S)-3-[(4-{3-Benzyl-1-ethyl-1H-pyrazol-5-yl}piperidin-1-yl)methyl]-4-(1,2,5-thiadiazol-3-yl)pyrrolidin-1-yl]methyl}cyclopentanecarboxylic Acid

Step A: (trans, 3R/S,4R/S)-3-(N-Methoxy-N-methyl-carboxamido)-4-(1,2,5-thiadiazol-3-yl)pyrrolidine, hydrochloride Salt

A solution of 150 mg (0.45 mmol) of (trans, 3R/S,4R/S)-1-benzyl-3-(N-methoxy-N-methyl-carboxamido)-4-(1,2,5-thiadiazol-3-yl)pyrrolidine (Prepared by analogous procedures described for Example 115, Steps A and B) in 3 mL of 1,2-dichloroethane at rt was treated with 0.075 mL (0.69 mmol) of 1-chloroethyl chloroformate. After 3 hours volatiles were removed under reduced pressure. The residue was dissolved in 5 mL of MeOH and warmed to 65° C. for 45 minutes. The reaction was cooled to rt and volatiles removed under reduced pressure. The crude product was used without further purification.

Step B: 1-{[(trans, 3R/S,4R/S)-3-(N-Methoxy-N-methyl-carboxamido)-4-(1,2,5-thiadiazol-3-yl)pyrrolidin-1-yl]methyl}cyclopentanecarboxylic Acid, Para-methoxybenzyl Ester

The title compound was prepared from (trans, 3R/S,4R/S)-3-(N-methoxy-N-methyl-carboxamido)-4-(1,2,5-thiadiazol-3-yl)pyrrolidine, hydrochloride salt (from Step A) and 1-formylcyclopentane carboxylic acid, para-methoxybenzyl ester (Prepared as Aldehyde 10 above) using a procedure analogous to that described in Example 1, Step A. R_{F}: 0.30 (3:2 v/v hexanes/EtOAc); ¹H-NMR (500 Mhz) 8 1.52-1.61 (m, 7H), 2.14-2.18 (m, 2H), 2.69-2.94 (m, 5H), 3.16-3.20 (m, 4H), 3.54 (s, 3H), 3.77 (s, 3H), 4.05 (m, 1H), 5.04 (ABq, J=12.0, 2H), 6.82 (d, J=8.7, 2H), 7.26 (d, J=8.7, 2H), 8.43 (s, 1H).

Step C: 1-{[(trans, 3R/S,4R/S)-3-Formyl-4-(1,2,5-thiadiazol-3-yl)pyrrolidin-1-yl]methyl}cyclopentanecarboxylic Acid, Para-methoxybenzyl Ester

A solution of 83 mg (0.18 mmol) of 1-{[(trans, 3R/S,4R/S)-3-(N-methoxy-N-methyl-carboxamido)-4-(1,2,5-thiadiazol-3-yl)pyrrolidin-1-yl]methyl}cyclopentanecarboxylic acid, para-methoxybenzyl ester (from Step B) in 2 mL of THF at −78° C. was treated with 0.2 mL (0.2 mmol) of 1 M Dibal in CH₂Cl₂. After 45 minutes the reaction was quenched with saturated Rochelle salts. The reaction was warmed to rt and partitioned between 25 mL of Et₂O and 25 mL of H₂O. After separating phases, the aqueous layer was extracted with 25 mL of Et₂O. The combined organic phases were washed with 50 mL of brine, dried over MgSO₄ and concentrated under reduced pressure. The residue was purified by flash chromatography using 3:1 v/v hexane/EtOAc as the eluant to afford the title compound. R_{F}: 0.50 (3:2 v/v hexanes/EtOAc). ¹H-NMR (500 Mhz) δ 1.51-1.64 (m, 6H), 2.14-2.20 (m, 2H), 2.65-2.87 (m, 4H), 3.03-3.14 (m, 3H), 3.79 (s, 3H), 3.91 (m, 1H), 5.05 (ABq, J=12.0, 2H), 6.85 (d, J=8.7, 2H), 7.28 (d, J=8.7, 2H), 8.40 (s, 11H), 9.62 (d, J=1.1, 1H).

Step D: 1-{[(trans, 3R/S,4R/S)-3-[(4-{3-Benzyl-1-ethyl-1H-pyrazol-5-yl}piperidin-1-yl)methyl]-4-(1,2,5-thiadiazol-3-yl)pyrrolidin-1-yl]methyl}cyclopentanecarboxylic Acid

The title compound was prepared from 1-{[(trans, 3R/S,4R(S)-3-formyl-4-(1,2,5-thiadiazol-3-yl)pyrrolidin-1-yl]methyl}cyclopentanecarboxylic acid, para-methoxybenzyl ester (from Step C) and 4-(3-benzyl-1-ethyl-1H-pyrazol-5-yl)piperidine, trifluoro-acetic acid salt (Prepared as Piperidine 1 above) using procedures analogous to that described in Example 1, Steps D and E. R_{F}: 0.49 (90:10:1 v/v/v CH₂Cl_{2/}MeOH/NH₄OH). ¹H-NMR (500 MHz, CD₃OD): δ 1.33-1.83 (m, 13H), 2.13-2.29 (m, 4H), 2.60-2.68 (m, 3H), 2.89-3.23 (m, 6H), 3.48-3.81 (m, 4H), 3.85 (s, 2H), 4.04 (q, J=7.1, 2H), 5.76 (s, 1H), 7.14-7.26 (m, 5H), 8.66 (s, 1H); ESI-MS 563.4 (M+H); HPLC LC 2: 1.97 min.

### Examples 117-126

Examples 117-126 were prepared using procedures analogous to that described in Examples 115 and 116. Procedures in Example 116 were used to avoid a catalytic hydrogenation in the presence of reducible heterocycles, such as a thiazole and thiophene.

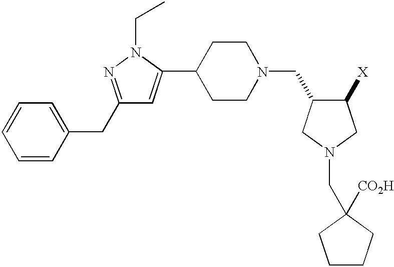

| EX- | | | HPLC RT | ESI-MS |
|---|---|---|---|---|
| AMPLE # | X | HPLC Method | (min) | (M + H) |
| 117 | 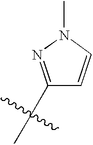 | LC1 | 1.97 | 559.4 |
| 118 | 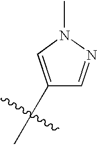 | LC1 | 2.03 | 559.4 |
| 119 | 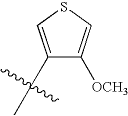 | LC1 | 2.03 | 591.2 |
| 120 | 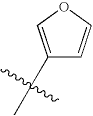 | LC2 | 2.05 | 545.4 |
| 121 | 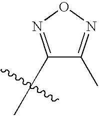 | LC2 | 2.08 | 561.4 |
| 122 | 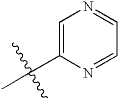 | LC2 | 1.97 | 557.4 |
| 123 | 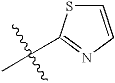 | LC2 | 2.19 | 562.4 |
| 124 | 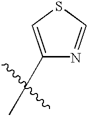 | LC2 | 1.87 | 562.3 |
| 125 | 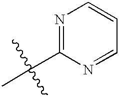 | LC2 | 1.73 | 557.4 |
| 126 | 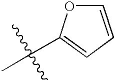 | LC1 | 2.00 | 545.3 |

### Example 127

1-{[(3S,4S)-3-[1-(R)-(4-{3-Benzyl-1-ethyl-1H-pyrazol-5-yl}piperidin-1-yl)ethyl]-4-(3-fluorophenyl)pyrrolidin-1-yl]methylcyclohexane-1-carboxylic Acid

Step A: 1-t-Butoxycarbonyl-3-(R)-(tert-butyldimethylsilyloxymethyl)-4-(S)-(3-fluorophenyl)pyrrolidine

A solution of 7.0 g (22.7 mmol) 3-(R)-(tert-butyldimethylsilyloxymethyl)-4-(S)-(3-fluoro)phenylpyrrolidine (Prepared as Pyrrolidine 2 above) in 75 mL of CH₂Cl₂ at 0° C. was treated with 7.5 mL (43.1 mmol) of N,N-diisopropylethylamine and 8.9 g (40.8 mmol) of di-tert-butyl dicarbonate. The resulting mixture was warmed to rt and stirred for 20 h. The reaction was partitioned between 200 mL Et₂O and 100 mL of H₂O and the layers were separated. The organic layer was dried over MgSO₄. The mixture was filtered and the filtrate was concentrated. Chromatography on 175 g of silica using 9:1 hexanes/diethyl ether (3 L) as the eluant to afford the title compound: ¹H NMR (500 MHz, CDCl₃): δ 0.86-0.87 (6H), 1.53 (s, 9H), 2.40 (1H), 3.16-3.86 (7H), 6.91-7.32 (4H).

Step B: 1-tert-Butoxycarbonyl-3-(R)-(hydroxymethyl)-4-(S)-(3-fluorophenyl)pyrrolidine

A solution of 9.3 g (22.7 mmol) 1-t-butoxycarbonyl-3-(R)-(tert-butyldimethylsilyloxymethyl)-4-(S)-(3-fluoro)phenylpyrrolidine (from Step A) in 50 mL of THF at 0° C. was treated with 29 mL of 1.0 M tetrabutylammonium fluoride solution in THF. The resulting mixture was warmed to rt and stirred for 20 h. The reaction mixture was partitioned between 200 mL of ether and 100 mL of 50% sat'd NaHCO₃ and the layers were separated. The organic layer was dried over MgSO₄ and concentrated. Purification by Biotage Flash 75 using a 75 L cartridge and 6.0 L of 9:1 v/v heptane/ethyl acetate as the eluant afforded the title compound: ¹H NMR (500 MHz, CDCl₃): δ 1.47 (9H), 2.48 (1H), 3.10-3.89 (7H), 6.92-7.33 (4H).

Step C: 1-tert-Butoxycarbonyl-3-(R)-(formyl)-4-(S)-(3-fluorophenyl)pyrrolidine

A solution of 4.3 mL (49.8 mmol) of oxalyl chloride in 90 1mL of CH₂Cl₂ at −78° C. was treated with 5.4 mL (75.7 mmol) of DMSO maintaining the temperature at less than -60° C. The resulting mixture was stirred cold for 5 min. A solution of 6.4 g (21.6 mmol) of 1-tert-butoxycarbonyl-3-(R)-(hydroxymethyl)-4-(S)-(3-fluoro)phenylpyrrolidine (from Step B) in 10 mL of CH₂Cl₂ was added maintaining the temperature at less than −60° C. The resulting mixture was stirred cold for 60 min. The mixture was treated with 30 mL (173.1 mmol) of N,N-diisopropylethylamine maintaining the temperature at less than −60° C. The reaction was warmed to 0° C., stirred for 20 min and quenched with 20 mL 0.5 N KHSO₄. The mixture was partitioned between 250 mL of CH₂Cl₂ and 100 mL of H₂O and the layers were separated. The aqueous layer was extracted with 250 mL of CH₂Cl₂. The combined organic phases were dried over MgSO₄ and concentrated. Purification by Biotage Flash 75 using 75 L cartridge and 6.0 L of 9:1 v/v heptane/ethyl acetate as the eluant afforded the title compound: ¹H NMR (500 MHz, CDCl₃): δ 1.48 (s, 9H), 2.48 (1H), 3.17-3.89 (6H), 6.96-7.35 (4H), 9.67 (s, 1H).

Step D: 1-tert-Butoxycarbonyl-3-(S)-[1-(R/S)-(4-{3-Benzyl-1-ethyl-1H-pyrazol-5-yl}piperidin-1-yl)-1-cyanomethyl]-4-(S)-(3-fluorophenyl)pyrrolidine

A mixture of 587 mg (2.0 mmol) of 1-tert-butoxycarbonyl-3-(R)-formyl-4-(S)-(3-fluorophenyl)pyrrolidine (from Step C), 540 mg (2.0 mmol) of Piperidine 1, 0.35 mL (2.6 mmol) of trimethylsilyl cyanide and 213 mg (2.0 mmol) of lithium perchlorate was heated at reflux for 2 h. The reaction mixture was cooled and partitioned between 75 mL of ether and 25 mL of 1 N NaOH. The organic layer was separated, dried over MgSO₄ and concentrated. Flash chromatography on 25 g of silica gel using 4:1 v/v hexanes/EtOAc, then 2:1 v/v hexanes/EtOAc as the eluant afforded the title compound as a mixture of diastereomers.

Step E: 1-tert-Butoxycarbonyl-3-(S)-[1-(S)-(4-{3-Benzyl-1-ethyl-1H-pyrazol-5-yl}piperidin-1-yl)ethyl]-4-(S)-(3-fluorophenyl)pyrrolidine

A solution of 143 mg (0.25 mmol) of 1-tert-butoxycarbonyl-3-(S)-[1-(R/S)-(4-{3-benzyl-1-ethyl-1H-pyrazol-5-yl}piperidin-1-yl)-1-cyanomethyl]-4-(S)-(3-fluorophenyl)pyrrolidine (from Step D) at 0° C. was treated with 2.0 mL of 1.4 M methylmagnesium bromide solution in THF/toluene. The cooling bath was removed and the mixture was stirred at rt for 20 h. The reaction was quenched with 10 mL of sat'd NH₄Cl and extracted with 40 mL of ether. The extract was washed with 10 mL of 1.0 N NaOH, 10 mL of brine, dried over MgSO₄ and concentrated. Flash chromatography on 12 g of silica gel using 3:1 v/v hexanes/EtOAc afforded pure product and also product contaminated (x-methyl epimer. For the title compound: ¹H NMR (500 MHz, CDCl₃). 8 0.28-0.32 (m, 1H), 0.89 (d, 3H), 1.24-1.34 (m, 2H), 1.36 (t, J=7.5, 3H), 1.45 (s, 9H), 1.93 (app t, J=12.0, 111), 2.23-2.58 (6H), 3.01-3.10 (m, 1H), 3.16 (app q, J=8.5, 1H), 3.22-3.34 (m, 1H), 3.56-3.82 (2H), 3.93 (s, 2H), 3.96 (q, J=7.5, 2H), 5.48 (s, 1H), 6.50-7.40 (7H).

Step F: Benzyl 1-{[(3S,4S)-3-[1-(R)-(4-{3-Benzyl-1-ethyl-1H-pyrazol-5-yl}piperidin-1-yl)ethyl]-4-(3-fluorophenyl)pyrrolidin-1-yl]methylcyclohexane-1-carboxylate

A solution of 67 mg (0.12 mmol) of 1-t-butoxycarbonyl-3-(S)-[1-(S)-(4-1 3-benzyl-1-ethyl-1H-pyrazol-5-yl}piperidin-1-yl)ethyl]-4-(S)-(3-fluorophenyl)pyrrolidine (from Step E) in 1.0 N HCl in MeOH was stirred at rt for 20 h. The solution was concentrated. The resulting solid, 55 mg (0.22 mmol) of Aldehyde 6, 0.1 mL of TEA and 125 mg (0.6 mmol) of sodium triacetoxyborohydride were dissolved in 4 mL of acetonitrile and the resulting solution was stirred at rt for 20 h. A second portion (55 mg) of Aldehyde 6 was added and the resutling solution was stirred for 24 h. The reaction mixture was concentrated. The residue was partitioned between 25 mL of ether and 15 mL of 1.0 N NaOH and the layers were separated. The organic layer was dried over MgSO₄ and concentrated. Flash chromatography on 6 g of silica gel using 4:1 v/v hexanes/EtOAc afforded the title compound:

Step G: 1-{[(3S,4S)-3-[1-(R)-(4-{3-Benzyl-1-ethyl-1H-pyrazol-5-yl}piperidin-1-yl)ethyl]-4-(3-fluorophenyl)pyrrolidin-1-yl]methylcyclohexane-1-carboxylic Acid

The title compound was prepared from 1-{[(3S,4S)-3-[1-(R)-(4-{3-benzyl-1-ethyl-1H-pyrazol-5-yl}piperidin-1-yl)ethyl]-4-(3-fluorophenyl)pyrrolidin-1-yl]methylcyclohexane-1-carboxylate (from Step F) using a procedure analogous to that described in Example 2, Step E. For the title compound: ESI-MS 601 (M+H); LC-1: 2.37 min.

### Examples 128-137

The compounds in Examples 128-132 were prepared using procedures analogous to those described in Example 127 substituting the appropriate Piperidine in Step D, organometallic reagent in Step E and Aldehyde in Step F. Examples 133-137 were prepared analogously substituting Pyrrolidine 1 in Example 127, Step A. In cases where the diastereomers obtained in Step E were not separable using flash chromatography on silica gel, they were separated by preparative HPLC (Chiralcelo OD 2×25 cm column, 90:10 v/v hexanes/iPrOH eluant).

| | | | | HPLC RT | ESI-MS |
|---|---|---|---|---|---|
| EXAMPLE | Piperidine | R-Met | Aldehyde | (min) | (M + H) |
| 128 | 40 | MeMgBr | 8 | 2.43 | 581 |
| | | | | LC1 | |
| 129 | 40 | MeMgBr | 6 | 2.40 | 595 |
| | | | | LC1 | |
| 130 | 13 | MeMgBr | 6 | 2.43 | 645 |
| | | | | LC1 | |
| | ¹H NMR (500 MHz, CD₃OD). δ 0.40 (dq, J = 3.5, 12.5, 1H), 0.95 (d, J = | | | | |
| | 6.0, 3H), 1.24 (app d, J = 12.5, 1H), 1.30 (t, J = 7.0, 3H), 1.34-1.67 | | | | |
| | (10H), 1.36 (t, J = 7.0, 3H), 1.96-2.11 (3H), 2.24 (app d, 1H), 2.33- | | | | |
| | 2.38 (m, 1H), 2.51 (app t, J = 10, 1H), 2.63-2.71 (3H), 3.10-3.29 (4H), | | | | |
| | 3.38 (app q, J = 8.5, 1H), 3.51-3.55 (m, 1H), 3.60 (app t, J = 9.0, 1H), | | | | |
| | 3.78 (s, 2H), 3.96 (q, J = 7.0, 2H), 4.01 (q, J = 7.0, 2H), 5.52 (s, 1H), | | | | |
| | 6.75-6.79 (m, 1H), 6.86 (app d, J = 9.0, 2H), 7.11-7.20 (5H). | | | | |
| 131 | 1 | EtMgBr | 6 | 2.40 | 615 |
| | | | | LC1 | |
| 132 | 1 | AllylZnBr | 6 | 2.51 | 629 |
| | | (α-propyl) | | | |
| 133 | 1 | MeMgBr | 8 | 2.16 | 569.4 |
| | | | | LC1 | |
| | ¹H NMR (500 MHz, CDCl₃₎: δ 0.90 (d, J = 6.4 Hz, 3H), 0.53-3.34 | | | | |
| | (32H), 3.94-3.98 (4H), 5.53 (s, 1H), 7.06-7.35 (10H) | | | | |
| 134 | 4-(3- | MeMgBr | 8 | 2.64 | 503.3 |
| | phenylprop | | | LC1 | |
| | yl)piperidm | | | | |
| | e | | | | |
| | ¹H NMR (500 MHz, CDCl₃): δ 0.39-3.40 (35H), 7.14-7.31 (10H) | | | | |
| 135 | 4-(3- | MeMgBr | 6 | 2.29 | 517.4 |
| | phenylprop | | | LC1 | |
| | yl)piperidm | | | | |
| | e | | | | |
| | ¹H NMR (500 MHz, CDCl₃): δ 0.41-3.39 (37H), 7.14-7.29 (10H) | | | | |
| 136 | 4-(4- | MeMgBr | 6 | 1.93 | 493.5 |
| | fluoropheny | | | LC1 | |
| | 1)-piperidine | | | | |
| | ¹H NMR (500 MHz, CD₃OD). δ 0.67 (m, 1H), 1.00 (d, J = 6.2, 3H), | | | | |
| | 1.20-1.66 (m, 11H), 2.00-2.05 (m, 3H), 2.23-2.30 (m, 2H), 2.50 (m, | | | | |
| | 1H), 2.69-2.79 (m, 3H), 3.20-3.34 (m, 4H), 3.43 (m, 1H), 3.56-3.68 | | | | |
| | (m, 2H), 6.92-6.97 (m, 2H), 7.03-7.06 (m, 2H), 7.23 (m, 1H), 7.33- | | | | |
| | 7.42 (m, 4H) | | | | |
| 137 | 4-(4- | MeMgBr | 8 | 2.61 | 479.3 |
| | fluoropheny | | | LC2 | |
| | 1)-piperidine | | | | |

¹H NMR (500 MHz, CD₃OD). δ 0.66 (m, 1H), 1.00 (d, J = 6.4, 3H),
1.22 (m, 1H), 1.46-1.87 (m, 8H), 2.00 (m, 1H), 2.12-2.28 (m, 4H), 2.51
(m, 1H), 2.67-2.79 (m, 3H), 3.23-3.34 (m, 4H), 3.47 (m, 1H), 3.58-
3.71 (m, 2H), 6.92-6.97 (m, 2H), 7.03-7.07 (m, 2H), 7.24 (m, 1H),
7.34-7.42 (m, 4H)

### Example 138

1-{[(3S,4S)-3-[(4-{3-[3,4-Dimethoxypheny]propyl}piperidin-1-yl)methyl]-4-(3-fluorophenyl)pyrrolidin-1-yl]methyl}cyclohexanecarboxylic Acid

The title compound was prepared using a procedure analogous to that described in Example 2 using Aldehyde 6 and Piperidine 40. 1H NMR (500 MHz, CD₃OD) δ 1.18-1.78 (22H) 2.02-2.11 (m, 2H), 2.18-2.22 (m, 1H), 2.5-2.56 (m, 2H), 2.87-2.92 (m, 11H), 3.01-3.2 (511), 3.48-3.57 (m, 2H), 3.78 (s, 3H), 3.8 (s, 3H), 6.69 (d, 1H), 6.77 s, 1H), 6.84 (d, 1H), 6.98-7.03 (m, 1H), 7.13-7.2 (m, 2H), 7.33-7.4 (m, 1H) ESI-LC/MS (M+H) calc. 581.37; obs. 581.35.

### Example 139

1-{[(3S,4S)-3-{[4-(3-Ethyl-1-{4-[methylsulfonyl]benzyl}-1H-pyrazol-4-yl)piperidin-1-yl]methyl}-4-(3-fluorophenyl)pyrrolidin-1-yl]methyl}cyclohexanecarboxylic Acid

The title compound was prepared using a procedure analogous to that described in Example 88 using Aldehyde 6 and Piperidine 5. 1H NMR (500 MHz, CD₃OD) δ 1.1-1.12 (t, 3H), 1.27-1.38 (5H), 1.43-1.59 (7H), 1.7-1.77 (m, 2H), 1.95-2.09 (3H), 2.17 (t, 1H), 2.38 (tt, 1H), 2.44-2.6 (6H), 2.62-2.69 (m, 1H), 2.82-2.84 (d, 1H), 2.98-3.0 (d, 1H), 3.01 (s, 3H), 3.06-3.2 (4H), 3.50-3.54 (m, 2H), 5.27 (s, 2H), 6.9-6.92 (m, 1H), 7.08-7.12 (m, 2H), 7.7-7.31 (m, 3H), 7.37 (s, 1H), 7.82-7.84 (d, 2H). ESI-LC/MS (M+H) calc. 665.35; obs. 665.4.

### Example 140

1-{[(3S,4S)-3-{[4-(1-{Cyclopropylmethyl}-3-ethyl-1H-pyrazol-4-yl)piperidin-1-yl]methyl}-4-(3-fluorophenyl)pyrrolidin-1-yl]methyl}cyclohexane Carboxylic Acid

Step A: 1-Cyclopropylmethyl-3-ethyl-4-iodo Pyrazole

The title compound was prepared from 1 g (5.5 mmoL) of 3-ethyl-4iodopyrazole (from Piperidine 5, Step B) and 598 mg (5.5 mmol) of bromomethylcyclopropane using the procedure described in Piperidine 5, Step C. Flash chromatography (6/1 hexanes/EtOAc) gave the desired product as a mixture of isomers that was used directly in the next step.

Step B: 1-(Cyclopropymethyl)-3-ethyl-4-(piperid-4-yl)pyrazole trifluoroacetate and 2-(cyclopropylmethyl)-3-ethyl-4-piperid-4-yl)pyrazole

A solution of 2.86 mL (5.72 mmol) of 2 M isopropyl magnesium chloride in TBF) was cooled to −10° C. and a solution of 1.22 g (4.4 mmol) 1-cyclopropylmethyl-3-ethyl-4-iodopyrazole in 5 ml THF was added. After 1 h a solution of 976 mg (4.2 mmol) N-tert-butoxylcarbonylpiperid-4- in 5 mL THF was added and the mixture was stirred at −5° C. for 20 min then rt for 5 h. The mixture was quenched with sat'd ammonium chloride and 1 M HCl then extracted with methylene chloride (4×). The organic portions were combined and stirred over magnesium sulfate for 48 h. The solvent was removed and flash chromatography (4/1 hexanes/EtOAc) gave the two isomeric products. The individual isomers were hydrogenated (10% palladium on carbon, 1 atm hydrogen, 2 h) in methanol then de-protected by stirring in 1/1 TFA/CH₂Cl₂ for 1 h. Removal of solvent and drying under vacuum gave the desired (1 substituted) isomer and also the undesired (2 substituted) isomer. The substitution pattern was established by NOE experiments. ¹H NMR (500 MHz, CD₃OD, for the desired isomer). δ 0.41-0.42 (m, 2H), 0.62-0.64 (m, 2H), 1.25-1.33 (m, 4H), 1.83-1.87 (m, 2H), 2.06-2.1 (d, 2H), 2.71-2.76 (q, 2H), 2.86-2.94 (m, 1H), 3.1-3.17 (m, 2H), 3.47-3.49 (m, 2H), 4.02-4.03 (d, 2H), 7,76 (s, 1H).

Step C: 1-{[(3S,4S)-3-{[4-(1-{Cyclopropylmethyl}-3-ethyl-1H-pyrazol-4-yl)piperidin-1-yl}methyl}-4-(3-fluorophenyl)pyrrolidin-1-yl]methyl}cyclohexane Carboxylic Acid

The title compound was prepared from 1-(cyclopropymethyl)-3-ethyl-4-(piperid-4-yl)pyrazole trifluoroacetate (from Step B) and Aldehyde 6 using a similar procedure described in Example 88. 1H NMR (500 MHz, CD₃OD). δ 0.34-0.36 (m, 2H), 0.57-0.60 (m, 2H), 1.18-1.24 (m, 4H), 1.42-1.64 (m, 8H), 1.82-1.92 (m, 2H), 2.03-2.06 (m, 2H), 2.13-2.16 (m, 2H), 2.57-2.62 (q, 2H), 2.72-2.78 (m, 1H), 2.87-2.93 (m, 1H), 3.10-3.19 (m, 3H), 3.35-3.44 (m, 2H), 3.47-3.51 (m, 1H), 3.56-3.61 (m, 2H), 3.64 (s, 2H), 3.64-3.74 (m, 1H), 3.88-3.89 (d, 2H), 3.91-3.95 (m, 1H), 4.05-4.13 (m, 1H), 7.13-7.16 (m, 1H), 7.25-7.28 (m, 2H), 7.43 (s, 1H), 7.45-7.49 (m, 1H). ESI-LC/MS (M+H) calc. 551.37; obs. 551.4.

### EXAMPLE 141

1-{[(3S,4S)-3-{[4-(5-Benzylpyridin-3-yl)piperidin-1-yl]methyl}-4-(3-fluorophenyl)pyrrolidin-1-yl]methyl}cyclohexanecarboxylic acid

The title compound was prepared using a procedure analogous to that described in Example 88 using Aldehyde 6 and Piperidine 4. The product was purified by semi-prep RP HPLC (5%→65% acetonitrile/water with 0.1% TFA, C-18 stationary phase) to give the product. ¹H NMR (500 MHz, CD₃OD) δ 1.4-1.65 (8H), 2.02-2.19 (m, 6H), 2.89-2.98 (m, 1H), 3.01-3.21 (5H), 3.34-3.46 (m, 2H), 3.54-3.65 (m, 3H), 3.63 (s, 2H), 3.73 (bs, 1H), 3.91-3.94 (m, 1H), 4.08-4.12 (m, 1H), 4.16 (s, 2H), 7.1-7.14 (m, 1H), 7.23-7.28 (m, 5H), 7.31-7.34 (m, 2H), 7.43-7.48 (m, 1H), 8.15 (s, 1H), 8.53 (s, 1H), 8.54 (s, 1H). ESI-LC/MS (M+H) calc. 570.34; obs. 570.5.

### Example 142

1-{[(3S,4S)-3-{[4-(Ethyl {pyrimidin-2-yl}amino)piperidin-1-yl]methyl}-4-(3-fluorophenyl)pyrrolidin-1-yl]methyl}cyclohexanecarboxylic Acid Step A: 4-Amino-1-tert-butoxycarbonylpiperidine

A solution of 20 g (100 mmol) of 1-tert-Butoxycarbonylpiperid-4-one, 11 mL (100 mmol) of benzylamine and 32 g (150 mmol) of sodium triacetoxyborohydride in 400 mL 1,2-dicloroethane were stirred together for 3 h. The resulting mixture was diluted with 1 L of EtOAc and washed with 1M aqueous NaOH (500 mL) followed by sat'd aqueous NaCl (500 mL). The organic phase was dried over Na₂SO₄ and concentrated to afford 30.1 grams of 4-N-benzylamino-1-tert-butoxycarbonyl piperidine as a viscous oil. The oil was dissolved in 400 mL MeOH and ammonium formate (39 grams, 600 mmol) was added. The vessel was purged with nitrogen and 6.5 grams 10% palladium on carbon (6 mmol) was added. The mixture was refluxed for 1 h then filtered through celite and concentrated. Drying under vaccum afforded the title compound. ¹H NMR (300 MHz, CDCl₃). δ 1.15-1.3 (m, 2H), 1.43 (s, 911), 1.7-1.9 (m, 4H), 2.65-2.72 (m, 3H), 3.95-4.1 (m, 2H).

Step B: 4-N-(Pyrimid-2-yl)amino-1-tert-butoxycarbonylpiperidine

A mixture of 1.9 g (9.5 mmol) of 4-amino-1-tert-butoxycarbonylpiperidine (from Step A), 1.1 g (9.5 mmol) of 2-chloropyrimidine and 3.3 mL (19 mmol) N,N-diisopropyl-ethylamine in 10 mL of isopropanol was refluxed for 24 h. The mixture was cooled, diluted with 100 mL CH₂Cl₂ and washed with water and sat'd aqueous NaCl. The organic phase was dried over MgSO₄ and concentrated. Flash chromatography (60 grams silica, 1/1 hexane/EtOAc eluent) m afforded the title compound. ¹H NMR (300 MHz, CDCl₃). δ 1.31-1.45 (m, 2H), 1.44 (s, 9H), 2.0-2.1 (m, 2H), 2.9-3.0 (m, 2H), 3.9-4.1 (m, 3H), 5.0-5.05 (m, 1H), 6.5-6.58, (t 1H), 8.15-8.2 (d, 2H).

Step C: 4-(N-(Pyrimid-2-yl)-N-ethyl)aminopipenidine, Hydrochloride Salt

A solution of 350 mg (1.35 mmol) of 4-N-(pyrimid-2-yl)amino-1-tert-butoxycarbonylpiperidine (350 mg, 1.35 mmol, from Step B) and 1 mL of ethyliodide in 5 mL DMF was treated with 503 mg (12.5 mmol) of 60% sodium hydride. The mixture was stirred for 12 h then diluted with ethyl acetate and washed with water (2×) and sat'd NaCl. The organic portion was dried over sodium sulfate and concentrated. Flash chromatography (2/1 hexane/EtOAc) gave the BOC protected product which was converted to the hydrochloride salt by heating in methanolic HCl for 2h. Removal of the solvent and drying under vacuum gave the desired product. ¹H NMR (300 MHz, CD₃OD) δ 1.33 (t, 3H), 2.1-2.13 (m, 2H), 2.25-2.35 (m, 2H), 3.2-3.3 (m, 2H), 3.55-3.59 (m, 2H), 3.7-3.75 (m, 2H), 4.87-4.93 (m, 1H, obscured by solvent), 7.1 (bs, 1H), 8.7 (bs, 1H).

Step D: 1-{[(3S,4S)-3-{[4-(Ethyl {pyrimidin-2-yl}amino)piperidin-1-yl]methyl}-4-(3-fluorophenyl)pyrrolidin-1-yl]methyl}cyclohexanecarboxylic Acid

The title compound was prepared using a procedure analogous to that described in Example 88 using Aldehyde 6 and 4-(N-(pyrimid-2-yl)-N-ethyl)aminopiperidine, hydrochloride salt (from Step C). ¹H NMR (500 MHz, CD₃OD) δ 1.15-1.18 (t, 3H), 1.42-1.66 (8H), 1.92-1.95 (m, 2H), 2.12-2.16 (m, 2H), 2.22-2.3 (m, 2H), 2.9-2.98 (m, 1H), 3.12-3.2 (m, 3H), 3.35-3.43 (m, 2H), 3.51-3.78 (m, 6H), 3.65 (s, 2H), 3.92-3.92 (m, 1H), 4.04-4.412 (bs, 1H), 4.65-4.72 (m, 1H), 6.61-6.63 (t, 1H), 7.12-7.15 (m, 1H), 7.24-7.27 (m, 2H), 7.44-7.49 (m, 1H), 8.31-8.32 (m, 2H). ESI-LC/MS (M+H) calc. 524.33; obs. 524.3.

### Examples 143-164

Examples 143-164 were prepared by analogous procedures described above using the appropriate aldehydes and piperidines. The piperidines were prepared by analogous procedures described in Examples 139-142.

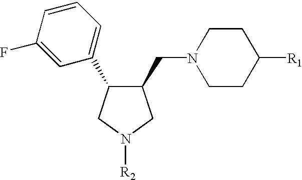

| | | | ESI-MS |
|---|---|---|---|
| EXAMPLE # | R₁ | R₂ | M/z (M + H) |
| 143 | 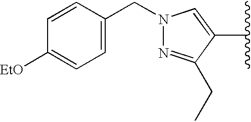 | 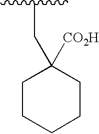 | 631.4 |
| 144 | 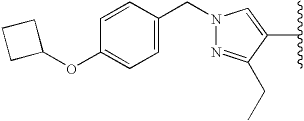 | 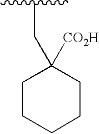 | 658.5 |
| 145 | 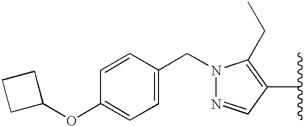 | 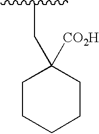 | 658.5 |
| 146 | 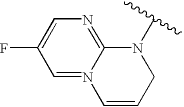 | 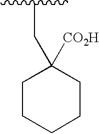 | 554.4 |
| 147 | 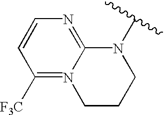 | 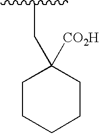 | 606.4 |
| 148 | 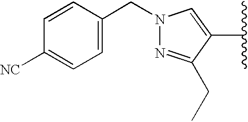 | 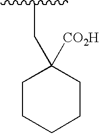 | 612.5 |
| 149 | 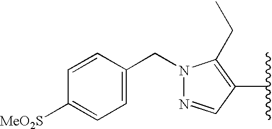 | 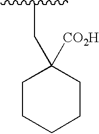 | 665.4 |
| 150 | 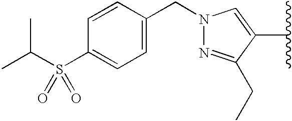 | 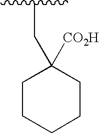 | 693.5 |
| 151 | 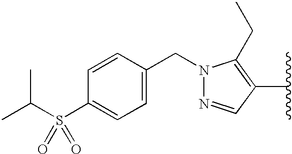 | 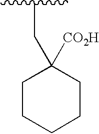 | 693.5 |
| 152 | 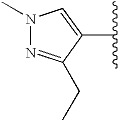 | 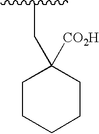 | 511.4 |
| 153 | 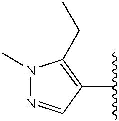 | 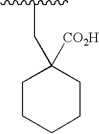 | 511.4 |
| 154 | 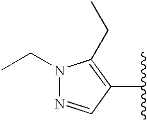 | 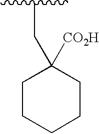 | 525.3 |
| 155 | 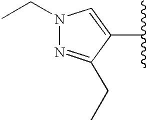 | 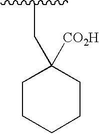 | 525.3 |
| 156 | 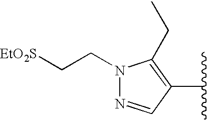 | 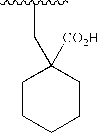 | 617.4 |
| 157 | 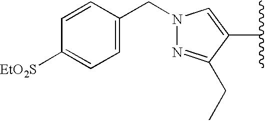 | 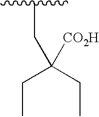 | 667.5 |
| 158 | 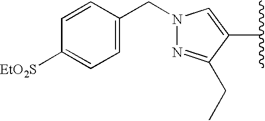 | 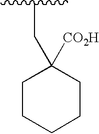 | 679.5 |
| 159 | 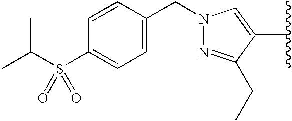 | 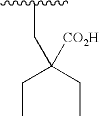 | 681.5 |
| 160 | 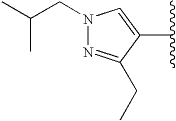 | 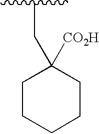 | 553.4 |
| 161 | 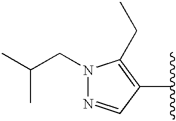 | 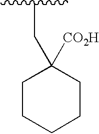 | 553.4 |
| 162 | 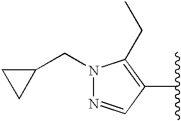 | 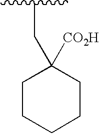 | 551.4 |
| 163 | 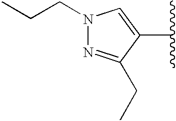 | 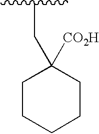 | 539.4 |
| 164 | 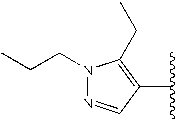 | 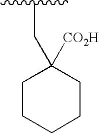 | 539.4 |

### Example 165

1-(((3S,4S)-3-((4-(3,3-Difluoro-3-(3-pyridyl)propyl)piperidin-1-yl)methyl)-4-(3-fluorophenyl)pyrrolidin-1-yl)methyl)cyclohexanecarboxylic Acid

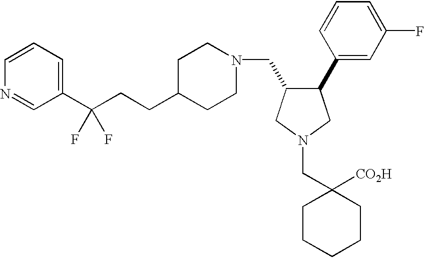

The title compound was prepared using procedures analogous to that described in Example 2 using Aldehyde 6 and Piperidine 7. ¹H NM (500 MHz, CD₃OD) δ 8.69-8.65 (bs, 1H), 8.65-8.61 (bs, 1H), 7.96 (d, J=8, 1H), 7.53 (dd, J=8, 5, 1H), 7.34 (td, J=8, 6, 1H), 7.17-7.10 (m, 2H), 6.98 (td, J=8, 2, 1H), 3.62-3.52 (m, 2H), 3.26 (t, J=10, 1H), 3.21-3.10 (m, 4H), 2.92 (d, J=11, 1H), 2.77 (d, J=11, 1H), 2.72-2.63 (m, 1H), 2.53 (dd, J=13, 10, 1H), 2.41 (dd, J=13, 5, 1H), 2.26-2.14 (m, 2H), 2.06-1.98 (m, 3H), 1.89 (td, J=12, 2, 1H), 1.69-1.49 (m, 7H), 1.46-1.20 (m, 6H), 1.14 (qd, J=12, 4, 1H), 1.08 (qd, J=12, 4, 1H); ESI-MS 558.4 (M+H); BPLC A: 1.88 min.

### Example 166

1-(((3S,4S)-3-((4-(3,3-Difluoro-3-(3-pyridyl)propyl)piperidin-1-yl)methyl)-4-(3-fluorophenyl)pyrrolidin-1-yl)methyl)cyclopentanecarboxylic Acid

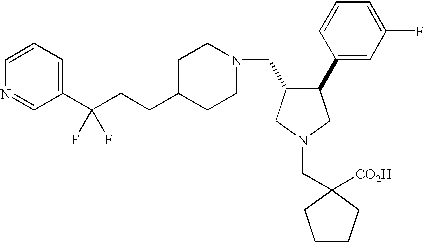

The title compound was prepared using procedures analogous to that described in Example 2 using Aldehyde 8 and Piperidine 7. ¹H NMR (500 MHz, CD₃OD) δ 8.67 (s, 1H), 8.63 (d, J=4, 1H), 7.95 (d, J=8, 1H), 7.53 (dd, J=8, 5, 1H), 7.35 (q, J=7, 1H), 7.18-7.11 (m, 2H), 7.00 (td, J=8, 2, 1H), 3.66 (dd, J=11, 8, 1H), 3.60 (dd, J=11, 8, 1H), 3.34-3.16 (m, 5H), 2.89 (d, J=11, 1H), 2.76-2.66 (m, 2H), 2.50 (dd, J=13, 10, 1H), 2.38 (dd, J=13, 5, 1H), 2.25-2.10 (m, 4H), 1.97 (td, J=12, 2, 1H), 1.88-1.77 (m, 3H), 1.72-1.44 (m, 6H), 1.34-1.27 (m, 2H), 1.27-1.17 (m, 1H), 1.13 (qd, J=12, 4, 1H), 1.05 (qd, J=12, 4, 1H); ESI-MS 544.5 (M+H); HPLC A: 1.18 min.

### Example 167

1-(((3S,4S)-3-(((1α,5α,6α)-6-(3-Benzyl-1-ethyl-(1H)-pyrazol-5-yl)-3-azabicyclo[3.1.0]hexan-3-yl)methyl)-4-(3-fluorophenyl)pyrrolidin-1-yl)m ethyl)cyclohexanecarboxylic Acid

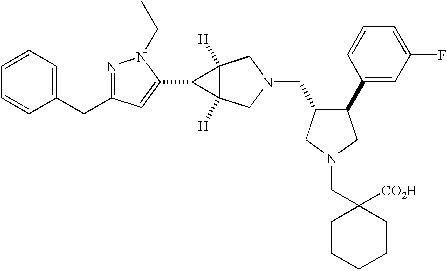

Step A: (1α,5α,6α)-6-Acetyl-3-(benzyloxycarbonyl)-3-azabicyclo[3.1.0]hexane

O,N-Dimethylhydroxylamine hydrochloride (203 mg, 2.08 mmol) was added to a solution of (1α,5α,6α)-3-(benzyloxycarbonyl)-3-azabicyclo[3.1.0]hexane-6-carboxylic acid (454 mg, 1.74 mmol, for preparation see K. E. Brighty and M. J. Castaldi, Synlett, 1996, 1097-1099) in dioxane (9.0 mL) containing 3A molecular sieves. N,N-Diisopropylethylamine (0.37 mL, 270 mg, 2.1 mmol), 4-(dimethylamino)pyridine (51 mg, 0.42 mmol), and 1-(3-(dimethylamino)propyl)-3-ethylcarbodiimide hydrochloride (433 mg, 2.26 mmol) were added and the mixture was stirred at RT overnight. The reaction mixture was partitioned between EtOAc (50 mL ) and 1.0 N aq. HCl (50 mL), and the aqueous layer was extracted with EtOAc (50 mL). The organic layers were washed in succession with saturated aq. NaCl (50 mL), dried (Na₂SO₄), decanted and evaporated to give (1α,5α,6α)-3-(benzyloxycarbonyl)-3-azabicyclo[3.1.0]hexane-6-N-methyl-N-methoxycarboxamide as an amber syrup.

Methylmagnesium bromide (1.4 M solution in THF, 1.0 mL, 1.4 mmol) was added to a solution of (1α,5α,6α)-3-(benzyloxycarbonyl)-3-azabicyclo[3.1.0]hexane-6-N-methyl-N-methoxycarboxamide (359 mg, 1.18 mmol) in THF (7.0 mL) cooled in a −70° C. bath. The bath was allowed to warm to 10° C. over 2 h. The mixture was partitioned between Et₂O (50 mL) and 1.0 N aq. HCl (50 mL). The aqueous layer was extracted with Et₂O (50 mL) and the organic layers were washed in succession with saturated aq. NaCl (50 mL), dried (Na₂SO₄), decanted, and evaporated to give the crude product. Purification by flash column chromatography on silica gel, eluting with 80:20 v/v to 70:30 v/v hexanes/EtOAc gave the title compound as a colorless syrup. For the title compound: ¹H NMR (500 MHz, CDCl₃) δ 7.40-7.31 (m, 5H), 5.13 (s, 2H), 3.77 (d, J 12, 1H), 3.72 (d, J 12, 1H)3.56-3.48 (m, 2H), 2.27 (m, 3H), 2.12 (t, J=2, 2H), 1.83 (t, J=2, 11); ESI-MS 260.0 (M+H); ]]HPLC A: 1.92 min.

Step B: (1α,5α,6α)-3-(Benzyloxycarbonyl)-6-(1,3-dioxo-4-phenylbutyl)-3-azabicyclo[3.1.0]hexane

A solution of (1α,5α,6α)-6-acetyl-3-(benzyloxycarbonyl)-3-azabicyclo[3.1.0]hexane (203 mg, 0.78 mmol, from Step A) and methyl phenylacetate (0.22 mL, 230 mg, 1.6 mmol) in TUF (0.30 mL) was dried over 3 A molecular sieves. The solution was then added over 20 min to a stirred suspension of NaH (62 mg of 60% oil dispersion, 1.6 mmol) in THF (0.70 mL) at 0° C. Additional TUF rinses (2×0.10 mL) were used to complete the transfer. The bath was allowed to slowly warm to rt. After 4 h, the reaction mixture was diluted into Et₂O (30 mL) and washed with 1.0 N aq. HCl (30 mL) followed by saturated aq. NaCl (30 ML). The aqueous layers were extracted in succession with Et₂O (2×30 μL) and the combined organic layers were dried (Na₂SO₄), decanted, and evaporated to crude product. Purification by flash column chromatography on silica gel, eluting with 90:10 v/v to 60:40 v/v CH₂Cl₂/EtOAc gave the title compound. For the title compound: ¹H NMR (500 MHz, CDCl₃) δ 7.41-7.25 (m, 10H), 5.50 (s, 1H), 5.12 (s, 2H), 3.75 (d, J=12, 1H), 3.70 (d, J=12, 1H), 3.58 (s, 2H), 3.55-3.48 (m, 2H), 2.20-2.18 (m, 2H), 1.45 (t, J=3, 1H); ESI-MS 378.2 (M+H).

Step C: (1α,5α,6α)-6-(3-Benzyl-1-ethyl-(1H)-pyrazol-5-yl)-3-(benzyloxycarbonyl)-3-azabicyclo[3.1.0]hexane

A solution of (1α,5α,6α)-3-(benzyloxycarbonyl)-6-(1,3-dioxo-4-phenylbutyl)-3-azabicyclo[3.1.0]hexane (187 mg, 0.50 mmol, from Step B) in CH₃OH (1.0 mL) was added over 20 min to a stirred suspension of ethylhydrazine oxalate (82 mg, 0.55 mmol) in CH₃OH (4.0 mL) at 50° C. Additional CH₃OH rinses (2×0.50 mL) were used to complete the transfer, and the resulting mixture was heated to 60° C. for 20 h. The reaction mixture was concentrated and the residue was partitioned between EtOAc (30 mL) and saturated aq. NaHCO₃ (30 mL). The aqueous layer was extracted with EtOAc (2×30 mL). The combined organic layers were washed with saturated aq. NaCl (30 mL), dried (Na₂SO₄), decanted, and evaporated to give a light brown syrup. Purification by flash column chromatography on silica gel, eluting with 90:10 v/v CH₂Cl₂/EtOAc gave the title compound as a colorless syrup. For the title compound: ¹H NMR (500 MHz, CDCl₃) δ 7.40-7.18 (m, 10H), 5.54 (s, 1H), 5.17 (d, J=12, 1H), 5.13 (d, J=12, 1H), 4.15 (q, J=7, 2H), 3.92 (s, 2H), 3.83 (d, J=12, 1H), 3.77 (d, J=12, 1H), 3.57-3.50 (m, 2H), 1.83-1.76 (m, 2H), 1.55 (t, J=3, 1H), 1.44 (t, J=7, 3H); ESI-MS 402.2 (M+H); HPLC A: 2.47 min. Later column fractions yielded 54 mg of the isomeric product (1α,5α,6α)-6-(5-benzyl-1-ethyl-(1H)-pyrazol-3-yl)-3-(benzyloxycarbonyl)-3-azabicyclo[3.1.0]hexane as a colorless syrup.

Step D: (1α,5α,6α)-6-(3-Benzyl-1-ethyl-(1H)-pyrazol-5-yl)-3-azabicyclo[3.1.0]hexane

A mixture of (1α,5α,6α)-6-(3-benzyl-1-ethyl-(1H)-pyrazol-5-yl)-3-(benzyloxycarbonyl)-3-azabicyclo[3.1.0]hexane (20 mg, 0.050 mmol, from Step C) and 10% palladium on carbon (6 mg) in ethanol (1.0 mL) was stirred under hydrogen at atmospheric pressure for 1 h. The mixture was filtered and the catalyst was rinsed with CH₃0H (5 mL). Evaporation of the filtrate gave the title compound as a colorless syrup. For the title compound: ¹H NMR (500 MHz, CD₃OD) δ 7.24 (t, J=7, 2H), 7.18 (d, J=7, 2H), 7.15 (t, J=7, 1H), 5.64 (s, 1H), 4.14 (q, J=7, 2H), 3.83 (s, 21H), 3.10 (d, J=12, 2H), 2.86 (d, J=12, 2H), 1.71 (s, 2H), 1.37 (t, J=7, 3H); ESI-MS 268.1 (M+H); HPLC A: 1.24 min.

Step E: 1-(((3S,4S)-3-(((1α,5α,6α)-6-(3-Benzyl-1-ethyl-(1H)-pyrazol-5-yl)-3-azabicyclo[3.1.0]hexan-3-yl)methyl)-4-(3-fluorophenyl)pyrrolidin-1-yl)methyl)cyclohexanecarboxylic Acid

The title compound was prepared using procedures analogous to that described in Example 2 using Aldehyde 6 and (1α,5α,6α)-6-(3-benzyl-1-ethyl-(1H)-pyrazol-5-yl)-3-azabicyclo[3.1.0]hexane (from Step D). ¹H NMR (500 MHz, CD₃OD) δ 7.36 (td, J=9, 7, 1H), 7.23 (t, J=8, 2H), 7.20-7.12 (m, 5H), 6.99 (td, J=9, 2, 1H), 5.56 (s, 1H), 4.07 (q, J=7, 2H), 3.81 (s, 2H), 3.66 (dd, J=11, 8, 1H), 3.61 (dd, J=11, 8, 1H), 3.35-3.28 (m, 1H), 3.24-3.16 (m, 4H), 3.13 (d, J=9, 1H), 2.89 (d, J=9, 1H), 2.66 (sextet, J=8, 1H), 2.54 (d, J=8, 2H), 2.32 (dd, J=9, 4, 1H), 2.28 (dd, J=9, 4, 1H), 2.06-1.98 (m, 2H), 1.86 (t, J=3, 1H), 1.69-1.48 (m, 7H), 1.46-1.13 (m, 2H), 1.15 (t, J=7, 3H); ESI-MS 585.6 (M+H); HPLC A: 1.70 min.

### Example 168

1-(((3S,4S)-3-(((1α,5α,6α)-6-(3-Phenylpropyl)-3-azabicyclo[3.1.0]hexan-3-yl)methyl)-4-(3-fluorophenyl)pyrrolidin-1-yl)methyl)cyclohexanecarboxylic Acid

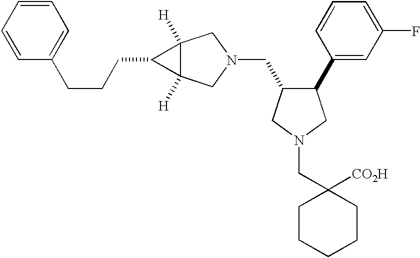

Step A: (1α,5α,6α)-3-(tert-Butoxycarbonyl)-6-(hydroxymethyl)-3-azabicyclo[3.1.0]hexane

A solution of di-tert-butyl dicarbonate (967 mg, 4.42 mmol) in CH₂Cl₂ (3.0 mL) was added to a stirred solution of (1α,5α,6α)-6-(hydroxymethyl)-3-azabicyclo[3.1.0]hexane (456 mg, 4.02 mmol, for preparation see K. E. Brighty and M. J. Castaldi, Synlett, 1996, 1097-1099) in CH₂Cl₂ (7.0 mL) at RT. After 15 h, the solution was partitioned between 2 N aq. HCl (50 mL) and EtOAc (50 mL ). The organic layer was washed with saturated aq. NaHCO₃ (50 mL) and saturated aq. NaCl (50 mL). The aqueous layers were extracted in succession with EtOAc (2×50 mL). The organic layers were dried (Na₂SO₄), decanted, and evaporated. The crude product was purified by flash column chromatography on silica gel, eluting with 80:20 v/v to 0:100 hexane/EtOAc to give the title compound as a colorless syrup. For the title compound: ¹H NMR (500 MHz, CDCl₃) δ 3.60 (d, J=11, 2H), 3.53 (d, J=6, 2H), 3.36 (d, J=11, 2H), 1.44 (s, 11H), 0.96 (tt, J=6, 3, 1H); ESI-MS 158.1 (M+H-56); HPLC A: 2.09 min.

Step B: (1α,5α,6α)-3-(tert-Butoxycarbonyl)-3-azabicyclo [3.1 .0]hexane-6-carboxaldehyde

A solution of DMSO (1.0 mL, 1.1 g, 7.0 mmol) in CH₂Cl₂ (0.6 mL) was added dropwise to a solution of oxalyl chloride (0.61 mL, 0.89 g, 7.0 mmol) in CH₂Cl₂ (6.4 mL) cooled in a dry ice/i-PrOH bath. After 5 min, (1α,5α,6α)-3-(t-butoxycarbonyl)-6-(hydroxymethyl)-3-azabicyclo[3.1.0]hexane (600 mg, 2.81 mmol, from Step A) was added slowly in CH₂Cl₂ (6.4 mL). After another 15 min, N,N-diisopropylethylamine (4.9 mL , 3.6 g, 28 mmol) was added and the reaction was allowed to warm to −40° C. over 1.5 h. The reaction mixture was poured into water (50 mL) and extracted with CH₂Cl₂ (3×50 mL). The combined organic layers were washed with saturated aq. NaCl (50 mL), dried (Na₂SO₄), decanted, and evaporated. Purification by flash column chromatography on silica gel, eluting with hexanes followed by 80:20 to 50:50 v/v hexanes/EtOAc gave the title compound as a colorless syrup. For the title compound: ¹H NMR (500 MHz, CDCl₃) δ 9.44 (d, J=4, 1H), 3.72 (d, J=11, 1H), 3.64 (d, J=11, 1H), 3.51-3.44 (m, 2H), 2.24-2.19 (m, 2H), 1.82 (q, J=4, 1H), 1.45 (s, 9H).

Step C: (1α,5α,6α)-3-(tert-Butoxycarbonyl)-6-(E-3-oxo-3-phenylprop-1-enyl)-3-azabicyclo[3.1.0]hexane

Diethyl (2-oxo-2-phenylethyl)phosphonate (0.126 mL, 149 mg, 0.58 mmol) was added dropwise to a stirred suspension of sodium hydride (22 mg of 60% oil dispersion, 0.55 mmol) in THF (3.0 mL) at RT. After 15 min, (1α,5α,6α)-3-(tert-butoxycarbonyl)-3-azabicyclo[3.1.0]hexane-6-carboxaldehyde (97 mg, 0.46 mmol, from Step B) dissolved in THF (1.5 mL) was added to the clear solution. After stirring 1.5 at RT, the mixture was poured into 2.5 N NaOH (20 mL) and extracted with Et₂O (3×20 mL ). The organic layers were washed with saturated aq. NaCl (20 mL), dried (Na₂SO₄), decanted, and evaporated. Purification by flash column chromatography on silica gel, eluting with 90:10 v/v to 85:15 v/v hexanes/EtOAc, gave the title compound as a white solid. For the title compound: ¹H NMR (500 MHz, CDCl₃) δ 7.95 (d, J=8, 2H), 7.57 (t, J=8, 1H), 7.48 (t, J=8, 2H), 7.00 (d, J=15, 1H), 6.65 (dd, J=15, 10, 1H), 3.80-3.61 (m, 2H), 3.47 (d, J=11, 2H), 1.90-1.84 (bs, 2H), 1.58 (dt, J=10, 3, 1H), 1.48 (s, 9H); ESI-MS 314.2 (M+H); HPLC A: 2.60 min.

Step D: (1α,5α,6α)-3-(tert-Butoxycarbonyl)-6-(3-phenylpropyl)-3-azabicyclo[3.1.0]hexane

A mixture of (1α,5α,6α)-3-(tert-butoxycarbonyl)-6-(E-3-oxo-3-phenylprop-1-enyl)-3-azabicyclo[3.1.0]hexane (71 mg, 0.23 mmol, from Step C) and 5% palladium on carbon (7 mg) in 95% ethanol (5.0 mL) was stirred under hydrogen at atmospheric pressure for 5 h. The mixture was filtered and the catalyst was rinsed with 95% EtOH. Evaporation of the filtrate gave a mixture of the title compound and (1α,5α,6α)-3-(t-butoxycarbonyl)-6-(3-hydroxy-3-phenylpropyl)-3-azabicyclo[3.1.0]hexane. This material was combined with additional product (14 mg) obtained similarly and resubjected to hydrogenation using 10% palladium on carbon (8 mg) in 95% ethanol (5.0 mL). Purification of the crude product by flash column chromatography on silica gel, eluting with 90:10 v/v to 95:5 v/v hexanes/EtOAc, gave the title compound as a colorless syrup. For the title compound: ¹H NMR (500 MHz, CDCl₃) δ 7.29 (t, J=7, 2H), 7.22-7.16 (m, 3H), 3.61-3.42 (m, 2H), 3.32 (d, J=11, 211), 2.64 (t, J=7, 2H), 1.73 (quintet, J=7, 2H), 1.45 (s, 9H), 1.29 (q, J=7, 2H), 1.25-1.21 (bs, 2H), 0.55 (tt, J 3, 7, 1H); ESI-MS 246.0 (M+H−56); HPLC A: 3.23 min.

Step E: (1α,5α,6α)-6-(3-Phenylpropyl)-3-azabicyclo[3.1.0]hexane

Iodotrimethylsilane (0.065 mL, 90 mg, 0.45 mmol) was added to a solution of (1α,5α,6α)-3-(tert-butoxycarbonyl)-6-(3-phenylpropyl)-3-azabicyclo[3.1.0]hexane (68 mg, 0.23 mmol, from Step D) in CHCl₃. After stirring for 30 min at RT, the solution was poured into a mixture saturated aq. NaHCO₃ (20 mL) and saturated aq. NaCl (10 mL), and extracted with Et₂O (3×30 mL. The organic layers were washed in succession with saturated aq. NaCl (30 mL), dried (Na₂SO₄), decanted, and evaporated to give 41 mg of colorless product. For the title compound: ¹H NMR (500 MHz, CD₃OD) δ 7.23 (t, J=7, 2H), 7.15 (d, J=7, 2H), 7.12 (t, J=7, 1H), 2.91 (d, J=11, 2H), 2.74 (bd, J=11, 2H), 2.61 (t, J=7, 2H), 1.69 (quintet, J=7, 2H, 1.27 (q, J=7, 2H), 1.20-.15 (m, 2H), 0.60, tt, J=7, 3, 1H); ESI-MS 202.4 (M+H); HPLC A: 2.24 min.

Step F: 1-(((3S,4S)-3-(((1α,5α,6α)-6-(3-Phenylpropyl)-3-azabicyclo[3.1.0]hexan-3-yl)methyl)-4-(3-fluorophenyl)pyrrolidin-1-yl)methyl)cyclohexanecarboxylic Acid

The title compound was prepared using procedures analogous to that described in Example 2 using Aldehyde 6 and (1α,5α,6α)-6-(3-phenylpropyl)-3-azabicyclo[3.1.0]hexane (from Step E). ¹H NMR (500 MHz, CD₃OD) δ 7.34 (td, J=9, 7, 1H), 7.23 (t, J=8, 2H), 7.16-7.09 (m, 5H), 6.96 (td, J=8, 2, 1H), 3.62 (dd, J=11, 8, 1H), 3.55 (dd, J=11, 8, 1H), 3.32 (m, 1H), 3.22-3.13 (m, 4H), 2.99 (d, J=9, 1H), 2.80 (d, J=9, 1H), 2.62-2.46 (m, 5H), 2.32-2.26 (m, 2H), 2.06-1.97 (m, 2H), 1.69-1.48 (m, 7H), 1.46-1.27 (m, 3H)1.23-1.03 (m, 4H), 0.91-0.85 (m, 1H); ESI-MS 519.3 (M+H); HPLC A: 2.51 min.

### Example 169

1-(((3S,4S)-3-((7-(3-Benzyl-1-ethyl-(1H)-pyrazol-5-yl)-3-azabicyclo[3.3.0]octan-3-yl)methyl]-4-(3-fluorophenyl)pyrrolidin-1-yl)methyl) cyclohexanecarboxylic Acid

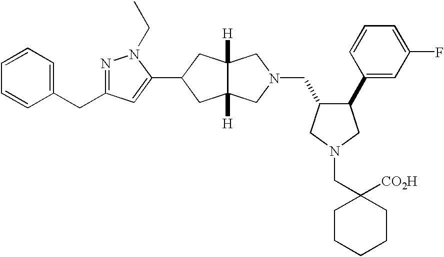

Step A: 3-(tert-Butoxycarbonyl)-7-(carbomethoxy)-3-azabicyclo[3.3.0]octane

Water (0.24 mL, 0.24 g, 13 mmol) and lithium chloride (440 mg, 10.4 mmol) were added to a solution of 3-(tert-butoxycarbonyl)-7,7-di(carbomethoxy)-3-azabicyclo[3.3.0]octane (2.96 g, 9.04 mmol, for preparation see D. L. Flynn and D. L. Zabrowski, J. Org. Chem., 1990, 55, 3673-3674) in dry DMSO (14.7 mL). The mixture was heated (oil bath temperature 180° C.) for 2.25 h. The mixture was allowed to cool to RT and partitioned using a mixture of water (60 mL ), saturated aq. NaCl (60 mL), and Et₂O (60 mL). The aqueous layer was extracted with two portions of Et₂O (60 mL, then 30 mL). The organic layers were washed in succession with saturated aq. NaCl (30 mL), dried (Na₂SO₄), decanted, and evaporated to give an amber oil. ¹H NMR of the crude product showed approximately equal amounts of two stereoisomers of the title compound. Samples of the separated isomers were obtained by flash column chromatography on silica gel, eluting with 85:15 v/v . hexanes/EtOAc. For the high R_{f} isomer of the title compound: ¹H NMR (500 MHz, CDCl₃) δ 3.69 (s, 3H), 3.59-3.51 (m, 2H), 3.19-3.08 (m, 2H), 3.00 (quintet, J=8, 1H), 2.84-2.75 (m, 2M), 2.10 (dt, J=10, 8, 2H), 1.47 (s, 9H); ESI-MS 270.2 (M+H); HPLC A: 3.09 min. For the low R_{f} isomer of the title compound: ¹H NMR (500 MHz, CDCl₃) δ 3.69 (s, 3H), 3.48 (dd, J=12, 8, 2H), 3.27 (dd, J=12, 2, 2H), 2.86 (tt, J=10, 8, 1H), 2.70-2.60 (m, 2H), 2.20 (dt, J=13, 8, 2H), 1.73 (ddd, J=13, 10, 8, 2H), 1.47 (s, 9H); ESI-MS 270.2 (M+H); HPLC A: 3.06 min.

Step B: 3-(tert-Butoxycarbonyl)-3-azabicyclo[3.3.0]octane-7-carboxylic Acid

Aqueous 3.9 N potassium hydroxide solution (1.15 mL, 4.5 mmol) was added to a solution of 3-(tert-butoxycarbonyl)-7-(carbomethoxy)-3-azabicyclo[3.3.0]octane (999 mg, 3.74 mmol, a 1:2 mixture of high R_{f} and low R_{f} isomers from Step A) in 95% EtOH (13 mL). After stirring overnight at RT, the mixture was concentrated to approximately 5 mL, poured into 2 N aq. HCl (50 mL) and extracted with EtOAc (3×50 mL). The organic layers were washed in succession with saturated aq. NaCl (25 mL), dried (Na₂SO₄), decanted, and evaporated to give the title compound as a colorless solid. For the title compound (isolated as a 65:35 mixture of stereoisomers): ¹H NMR (500 MHz, CD₃OD) major isomer peaks at 6 3.48-3.40 (m, 2H), 3.25 (dd, J=11, 4, 2H), 2.87 (tt, J=9, 8, 1H), 2.73-2.64 (m, 2H), 2.25-2.16 (m, 2H), 1.66 (ddd, J=16, 10, 7, 2H), 1.45 (s, 9H); minor isomer peaks at 6 3.51 (dd, J=11, 8, 2H), 3.13 (dd, J=11, 4, 2H), 2.96 (quintet, J=8, 1H), 2.83-2.76 (m, 2H), 2.09 (dt, J=13, 8, 2H), 1.78 (ddd, J=13, 8, 2, 2H), 1.45 (s, 9H); ESI-MS 200.2 (M+H−56); HPLC A: 2.39 min.

Steps C-D: 3-(tert-Butoxycarbonyl)-7-(1,3-dioxo-4-phenylbutyl)-3-azabicyclo[3.3.0]octane (high R_{f} isomer)

The title compound was prepared using procedures analogous to those described in Example 167, Steps A-B, substituting 3-(tert-butoxycarbonyl)-3-azabicyclo[3.3.0]octane-7-carboxylic acid (from Step B above) for (1α,5α,6α)-3-(benzyloxycarbonyl)-3-azabicyclo[3.1.0]hexane-6-carboxylic acid in Step A. Purification by flash column chromatography on silica gel, eluting with 95:5 to 85:15 v/v hexanes/EtOAc gave the high R_{f} isomer of the title compound: ¹H NMR (500 MHz, CDCl₃) δ 7.36 (t, J=7, 2H), 7.32-7.24 (m, 3H), 3.61 (s, 1H), 3.54 (dd, J=11, 8, 2H), 3.12 (dd, J=8, 4, 2H), 2.88 (quintet, J=8, 1H), 2.83-2.75 (m, 2H), 2.02 (dt, J=13, 8, 2H), 1.74 (ddd, J=13, 8, 2, 2H), 1.47 (s, 9H); ESI-MS 316.2 (M+H-56); HPLC A: 4.23 min. Later column fractions contained a mixture of high R_{f} and low R_{f} stereoisomers.

Step E: 7-(3-Benzyl-1-ethyl-(1H)-pyrazol-5-yl)-3-(tert-butoxycarbonyl)-3-azabicyclo[3.3.0]octane

The title compound was prepared using procedures analogous to those described in Example 167, Step C, substituting 3-(tert-butoxycarbonyl)-7-(1,3-dioxo-4-phenylbutyl)-3-azabicyclo[3.3.0]octane (high R_{f} isomer, from Step D) for (1α,5α,6α)-3-(benzyloxycarbonyl)-6-(1,3-dioxo-4-phenylbutyl)-3-azabicyclo[3.1.0]hexane. Purification by flash column chromatography on silica gel, eluting with 95:5 to 85:15 v/v CH₂Cl₂/Et₂O gave the title compound as a colorless syrup. For th title compound: ¹H NMR (500 MHz, CDCl₃) δ 7.33-7.25 (m, 4H), 7.21 (t, J=7, 1H), 5.71 (s, 1H), 4.08 (q, J=7, 2H), 3.94 (s, 2H), 3.67-3.57 (bs, 2H), 3.25-3.10 (m, 3H), 2.88-2.80 (m, 2H), 1.98-1.81 (m, 4H), 1.48 (s, 9H), 1.44 (t, J=7, 3H); ESI-MS 396.4 (M+H); HPLC A: 3.62 min. Later column fractions contained a mixture of the title compound and the isomeric product 7-(5-benzyl-1-ethyl-(1H)-pyrazol-3-yl)-3-(tert-butoxycarbonyl)-3-azabicyclo[3.3.0]octane.

Step F: 7-(3-Benzyl-1-ethyl-(1H)-pyrazol-5-yl)-3-azabicyclo[3.3.0]octane

Trifluoroacetic acid (2.0 mL) was added to a solution of 7-(3-benzyl-1-ethyl-(1H)-pyrazol-5-yl)-3-(tert-butoxycarbonyl)-3-azabicyclo[3.3.0]octane (81 mg, 0.21 mmol, from Step E) in CH₂Cl₂ (2.0 mL). After 6 h, the reaction was concentrated and the residue was dissolved in EtOAc (30 mL) and washed with saturated aq. NaHCO₃ (20 mL) followed by saturated aq. NaCl (20 mL). The aqueous layers were extracted with EtOAc (2×30 mL ). The organic layers were dried (Na₂SO₄), decanted, and evaporated to give the title compound as a pale yellow syrup. For the title compound: ¹H NMR (500 MHz, CD₃OD) δ 7.24 (t, J=7, 2H), 7.19 (d, J=7, 2H), 7.15 (t, J=7, 1H), 5.82 (s, 1H), 4.10 (q, J=7, 2H), 3.86 (s, 2H), 3.25-3.17 (m, 2H), 2.84-2.75 (m, 2H), 2.61 (dd, J=12, 6, 2H), 1.86 (dd, J=12, 7, 2H), 1.83-1.75 (m, 2H), 1.37 (t, J=7, 3H); ESI-MS 296.4 (M+H); HPLC A: 1.74 min.

Step G: 1-(((3S,4S)-3-((7-(3-Benzyl-1-ethyl-(1H)-pyrazol-5-yl)-3-azabicyclo[3.3.0]octan-3-yl)methyl]-4-(3-fluorophenyl)pyrrolidin-1-yl)methyl)cyclohexanecarboxylic Acid

The title compound was prepared using procedures analogous to that described in Example 2 using Aldehyde 6 and 7-(3-benzyl-1-ethyl-(1H)-pyrazol-5-yl)-3-azabicyclo[3.3.0]octane (from Step F). ¹H NMR (500 MHz, CD₃OD) δ 7.32 (dd, J=8, 7, 1H), 7.32 (t, J=8, 2H), 7.20-7.10 (m, 5H), 6.96 (td, J=8, 2, 1H), 5.76 (s, 1H), 4.06 (q, J=7, 2H), 3.84 (s, 2H), 3.64-3.55 (m, 4H), 3.29-3.10 (m, 6H), 2.84 (t, J=8, 1H), 2.78-2.60 (m, 5H), 2.52-2.45 (m, 1H), 2.36-2.28 (m, 1H), 2.17 (dd, J=10, 4, 1H), 2.08-1.98 (m, 2H), 1.81 (dd, J=12, 6, 1H), 1.75-1.39 (m, 8H), 1.45-1.27 (m, 4H), 1.36 (t, J=7, 3H); ESI-MS 307.6 (M+H); HPLC A: 2.1 min.

### Examples 170-178

Examples 170-178 were prepared using procedures analogous to that described in Example 2 using Aldehydes 6 or 8, Pyrrolidine 2 and the appropriate piperidine. (Note: The 4-(5,6,7,8-tetrahydroimidazo[1,2-a]pyridin-3-yl)piperidine piece in Example 178 was prepared by hydrogenation of 1-(tert-Butoxycarbonyl)-4-(imidazo[1,2-a]pyridin-3-yl)piperidine using Platinum (IV) oxide under 40 psi of H2 gas in a Parr shaker.)

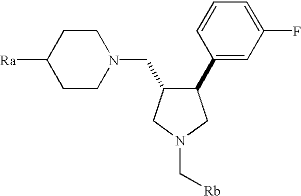

| EX- | | | MS |
|---|---|---|---|
| AMPLE | | | m/Z (M + 1) |
| NO. | Ra | Rb | (HPLC A) |
| 170 | 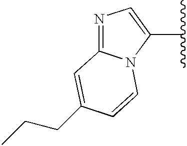 | 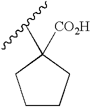 | 547 (1.73 min) |
| 171 | 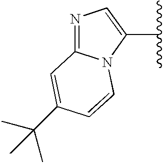 | 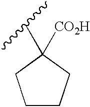 | 561 (1.73 min) |
| 172 | 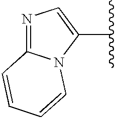 | 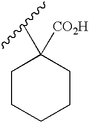 | 519 (1.55 min) |
| 173 | 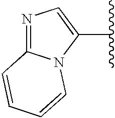 | 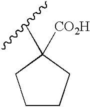 | 505 (1.47 min) |
| 174 | 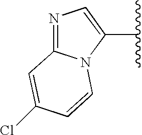 | 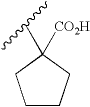 | 539 (1.52 min) |
| 175 | 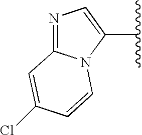 | 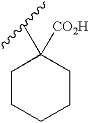 | 553 (1.65 min) |
| 176 | 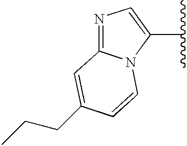 | 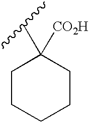 | 561 (1.81 min) |
| 177 | 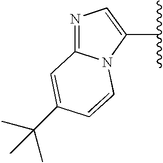 | 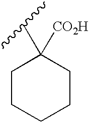 | 575 (1.87 min) |
| 178 | 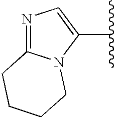 | 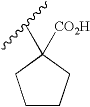 | 509 (1.00 min) |

### Examples 179-180

Examples 179-180 in the Table below were prepared using a procedure analogous to that described in Example 2 using Aldehyde 6 and the appropriate piperidines. The piperidines were prepared using procedures described in WO 00/59502.

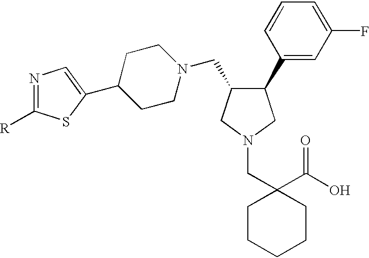

| EXAMPLE NO. | R | ESI-MS (M + H) | HPLC |
|---|---|---|---|
| 179 | Ethyl | 514.2 m/Z | 2.97 min. |
| 180 | i-Butyl | 542.2 m/Z | 3.39 min. |

### Examples 181-182

Examples 181-182 in the Table below were prepared from the corresponding piperidine and aldehyde using the usual reductive amination and hydrogenolysis procedure described for other examples. The piperidines were derived using procedures described in WO 00/59502.

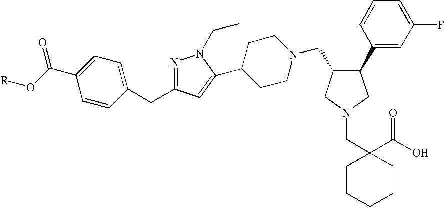

| EXAMPLE NO. | R | ESI-MS (M + H) | HPLC |
|---|---|---|---|
| 181 | Me | 645.2 m/Z | 3.73 min. |
| 182 | H | 631.2 m/Z | 3.37 min. |

### Examples 183-193

Examples 183-193 in the Table below were prepared from the corresponding piperidine and aldehyde using the usual reductive amination and hydrogenolysis procedure described for other examples.

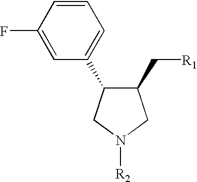

| EXAMPLE | | | ESI-MS |
|---|---|---|---|
| NO. | R₁ | R₂ | M/z (M + H) |
| 183 | 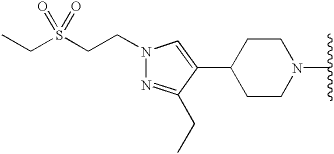 | 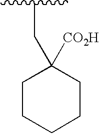 | 617.4 |
| 184 | 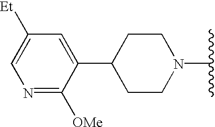 | 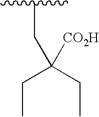 | 526.2 |
| 185 | 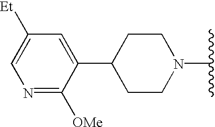 | 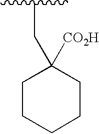 | 538.4 |
| 186 | 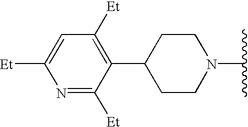 | 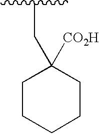 | 564.9 |
| 187 | 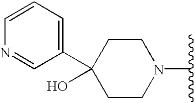 | 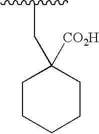 | 496.1 |
| 188 | 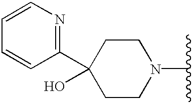 | 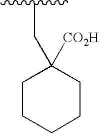 | 496.1 |
| 189 | 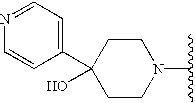 | 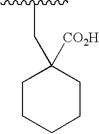 | 496.2 |
| 190 | 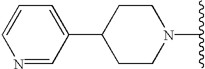 | 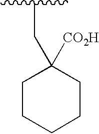 | 480.2 |
| 191 | 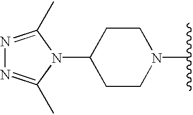 | 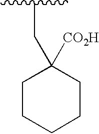 | 498.4 |
| 192 | 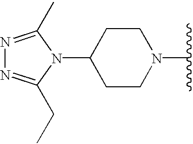 | 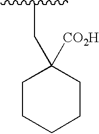 | 512.4 |
| 193 | 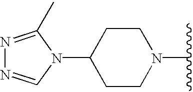 | 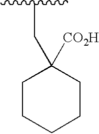 | 484.3 |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A compound of Formula I: 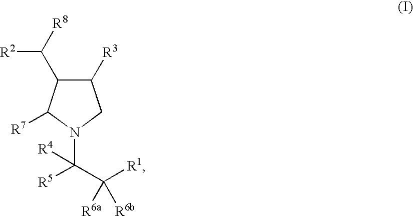
wherein:
R¹ is:
(1) —CO₂H,
(2) —NO₂,
(3) —tetrazolyl,
(4) —hydroxyisoxazole,
(5) —SO₂NHCO—(CO₃ alkyl)—R^{a}, or
(6) —P(O)(OH)(OR^{a});
wherein R^{a} is independently selected from hydrogen, C₁₋₆ alkyl, C₅₋₆ cycloalkyl, benzyl and phenyl, where any one of which except hydrogen is optionally substituted with 1-3 substituents where the substituents are independently selected from halo, C₁₋₃ alkyl, —O—C₁₋₃ alkyl, and —CF₃,
R² is: 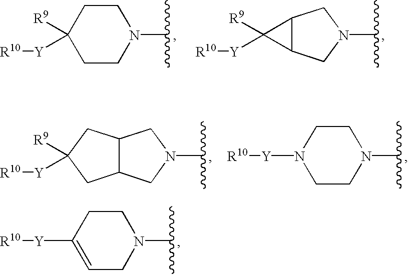
wherein “ [symbol] ” denotes the point of attachment and R⁹ is selected from:
(1) hydrogen,
(2) C₁₋₆ alkyl, which is unsubstituted or substituted with 1-4 substituents where the substituents are independently selected from hydroxy, cyano, and halo,
(3) cyano,
(4) hydroxy, and
(5) halo; and
Y is:
(1) a direct single bond;
(2) —C₁₋₁₀ alkyl- or —(C₀₋₆ alkyl)C₃₋₆cycloalkyl(CO₆ alkyl)—, either of which is optionally substituted with 1-7 substituents independently selected from:
(a) halo,
(b) hydroxy,
(c) —O-C₁₋₃ alkyl,
(d) —CF₃,
(e) —(C₁₋₃ alkyl)hydroxy, and
(f) ethylenedioxy;
(3) —(CO-6 alkyl)—Z¹—(C₀₋₆ alkyl)—, wherein each alkyl is optionally substituted with 1-7 substituents independently selected from:
(a) halo,
(b) hydroxy,
(c) —O-C₁₋₃ alkyl, and
(d) —CF₃;
and where Z¹ is selected from —SO₂—, —N(R^{u})—,
N(R^{u})C(═CHR^{s})N(R^{u})—, —N(R^{u})C(═NR^{s})N(R^{u})—, —S—, —O—, —SO—, —SO₂N(R^{u})—, —N(R^{u})SO₂—, and —PO₂—;
R^{u} is hydrogen, C₁₋₆ alkyl, C₂₋₆ alkenyl, benzyl, phenyl, (CO)C₁₋₆ alkyl, —SO₂-C₁₋₆ alkyl, —SO₂-phenyl, —SO₂-heterocycle, or C₁₋₆ alkyl-C₃₋₆ cycloalkyl; wherein any of which except hydrogen is optionally substituted with 1-3 substituents independently selected from halo, C₁₋₃ alkyl, —O-C₁₋₃ alkyl, and —CF₃;
R^{s} is hydrogen, C₁₋₄ alkyl, —NO₂ or —CN;
(4) —(C₀₋₆ alkyl)—Z²—(CO₆ alkyl)—, wherein each alkyl is optionally substituted with 1-7 substituents independently selected from:
(a) halo,
(b) hydroxy,
(c) —O-C₁₋₃ alkyl, and
(d) —CF₃;
and where:
Z² is selected from —C(═O)—, —C(═O)O—, —OC(═O)—, C(═O)NR^{v}—, —NR^{v}C(═O), —OC(═O)NR^{v}—, —NR^{v}C(═O)O—, and —NR^{w}C(═O)NR^{v}—;
R^{v} is hydrogen, C₁₋₆ alkyl, C₂₋₆ alkenyl, C₂₋₆ alkynyl, benzyl, phenyl, or C₁₋₆ alkyl-C₃₋₆ cycloalkyl; wherein any of which except hydrogen is optionally substituted with 1-3 substituents independently selected from halo, C₁₋₃ alkyl, —O-C₁₋₃ alkyl, and —CF₃; and
R^{w} is hydrogen or C₁₋₆ alkyl;
R¹⁰ is:
phenyl, naphthyl, biphenyl, or heterocycle, any one of which is unsubstituted or substituted with 1-7 of R^{d} where R^{d} is independently selected from:
(a) halo,
(b) cyano,
(c) hydroxy,
(d) C₁₋₆ alkyl, which is unsubstituted or substituted with 1-5 of R^{e} where R^{e} is independently selected from halo, cyano, hydroxy, —O-C₁₋₆ alkyl, —C₃₋₆ cycloalkyl, —CO₂H, —CO₂-(C₁₋₆ alkyl), —CF₃, —SO₂R^{a}, —NR^{a}R^{b} (where R^{a} is independently as defined above and R^{b} is independently selected from the definitions of R^{a}), phenyl, naphthyl, biphenyl, and heterocycle;
wherein phenyl, naphthyl, biphenyl, or heterocycle is unsubstituted or substituted with 1-7 of R^{f} where R^{f} is independently selected from halo, cyano, hydroxy, C1-6 alkyl, C₁₋₆ haloalkyl, —O-C₁₋₆ alkyl, —O-C₁₋₆ haloalkyl, —CO₂H, —CO₂(C₁₋₆ alkyl), —NR^{a}R^{b}, —(C₁₋₆ alkyl)—NR^{a}R^{b}, —SO₂R^{a}, —N(R^{a})SO₂R^{b}, —N(R^{a})COR^{b}, —(C₁₋₆ alkyl)hydroxy, —O-C₃₋₆ cycloalkyl, benzyloxy, phenoxy, and —NO₂,
(e) —O-C₁-6 alkyl, which is unsubstituted or substituted with 1-5 of Re,
(f) —O-phenyl, which is unsubstituted or substituted with 1-5 of R^{f},
(g) —O-heterocycle, which is unsubstituted or substituted with 1-5 of R^{f},
(h) —NO₂,
(i) phenyl,
(j) —CO₂R^{a},
(k) tetrazolyl,
(l) —NR^{a}R^{b},
(m) —NR^{a}—COR^{b},
(n) —NR^{a}—CO₂R^{b},
(o) —CO—NR^{a}R^{b},
(p) —OCO—NR^{a}R^{b},
(q) —NR^{a}CO—NR^{a}R^{b},
(r) —S(O)ₘ—R^{a}, wherein m is an integer selected from 0, 1 and 2,
(s) —S(O)₂—NR^{a}R^{b},
(t) —NR^{a}S(O)₂—R^{b},
(u) —NR^{a}S(O)₂—NR^{a}R^{b},
(v) C₂₋₆ alkenyl,
(w) furanyl, which is unsubstituted or substituted with benzyl which is unsubstituted or substituted with 1-7 of R^{f} wherein R^{f} is independently as defined above,
(x) —C₃₋₆ cycloalkyl, and
(y) —O—C₃₋₆ cycloalkyl;
R³ is phenyl, naphthyl, or heterocycle, any one of which is unsubstituted or substituted with 1-7 substituents where the substituents are independently selected from:
(a) halo,
(b) C₁₋₄ alkyl,
(c) C₁₋₄ haloalkyl,
(d) hydroxy,
(e) —O-C₁₋₄ alkyl,
(f) —O-C₁₋₄ haloalkyl,
(g) —CO₂R^{a},
(h) —NR^{a}R^{b}, and
(i) —CONR^{a}R^{b};
R⁴ is hydrogen, C₁₋₁₀ alkyl, C₃₋₈ cycloalkyl, —(C₁₋₃ alkyl)-C₃₋₈ cycloalkyl, —(C₀₋₂ alkyl)-(C₃₋₈ cycloalkylidenyl)-(C₁₋₂ alkyl), C₂₋₁₀ alkenyl, C₂₋₁₀ alkynyl, cyclohexenyl, phenyl, —(C₁₋₆ alkyl)-phenyl, naphthyl, dihydronaphthyl, tetrahydronaphthyl, octahydronaphthyl, biphenyl, or heterocycle; wherein any one of which except for hydrogen is unsubstituted or substituted with 1-7 of R^{d} where R^{d} is independently as defined above;
R⁵ is hydrogen or C₁₋₆ alkyl, wherein the alkyl is unsubstituted or substituted with 1-7 substituents where the substituents are independently selected from:
(a) halo,
(b) —CF₃,
(c) hydroxy,
(d) C₁₋₃ alkyl,
(e) —O-C₁₋₃ alkyl,
(f) —CO₂R^{a},
(g) —NR^{a}R^{b}, and
(h) —CONR^{a}R^{b};
or alternatively R⁴ and R⁵ together with the carbon atom to which they are attached form a C₃₋₈ cycloalkyl ring which may be unsubstituted or substituted with 1-7 of R^{d};
R^{6a} and R^{6b} are each independently C₁₋₆ alkyl, C₂₋₆ alkenyl, C₂₋₆ alkynyl, C₃₋₈ cycloalkyl, phenyl, naphthyl, or heterocycle; wherein any one of which is unsubstituted or substituted with 1-7 substituents where the substituents are independently selected from:
(a) halo,
(b) C₁₋₄ haloalkyl,
(c) hydroxy,
(d) C₁₋₄ alkyl,
(e) —O-C₁₋₄ alkyl,
(f) —O-C₁₋₄ haloalkyl,
(g) C₃₋₈ cycloalkyl,
(h) —CO₂R^{a},
(i) —NR^{a}R^{b}, and
(j) —CONR^{a}R^{b};
or alternatively R^{6a} and R^{6b} together with the carbon atom to which they are attached form:
(a) a 3- to 8-membered saturated carbocyclic ring, in which one of the ring carbons is optionally a member of a 3- to 8-membered spiro ring containing carbon atoms and optionally 1 or 2 heteroatoms independently selected from nitrogen, oxygen and sulfur;
(b) a 4- to 8-membered monocyclic heterocycle containing from 1 to 3 heteroatoms independently selected from nitrogen, oxygen and sulfur, in which one of the ring carbons is optionally a member of a 3- to 8-membered spiro ring containing carbon atoms and optionally 1 or 2 heteroatoms independently selected from nitrogen, oxygen and sulfur;
(c) a 5- to 8-membered saturated carbocyclic ring to which is fused a C₃₋₈ cycloalkyl, or
(d) a 5- to 8-membered heterocyclic ring containing from 1 to 3 heteroatoms independently selected from nitrogen, oxygen and sulfur, to which is fused a C₃₋₈ cycloalkyl,
wherein the ring system of (a), (b), (c) or (d) is optionally substituted with from 1 to 3 substituents independently selected from halo, C₁₋₄ alkyl, C₁₋₄ haloalkyl, —O-C₁₋₄ alkyl, —O-C₁₋₄ haloalkyl, and hydroxy;
R⁷ is hydrogen or C₁₋₆ alkyl; and
R⁸ is hydrogen or C₁₋₆ alkyl;
and with the proviso that
(A) when R¹⁰ is a heterocycle selected from pyrazolyl and imidazolyl, then the heterocycle is unsubstituted or substituted with 1 or 2 of R^{d}; and
(B) when R¹⁰ is a heterocycle selected from: 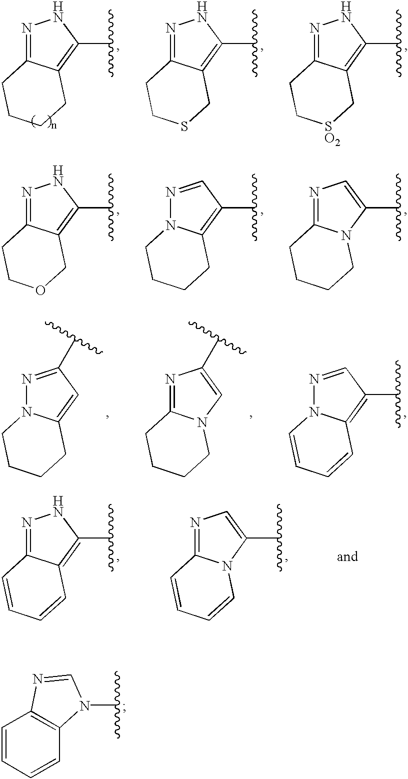
wherein n is an integer equal to zero or 1, then the heterocycle is unsubstituted in the pyrazolyl or imidazolyl ring;
or a pharmaceutically acceptable salt thereof.

2. The compound according to claim 1 wherein R¹ is:
(1) —CO₂H,
(2) —P(O)(OH)₂, or
(3) -tetrazolyl;
or a pharmaceutically acceptable salt thereof.

3. The compound according to claim 1 wherein R¹ is:
(1) —CO₂H, or
(2) -tetrazolyl;
or a pharmaceutically acceptable salt thereof.

4. The compound according to claim 1 wherein R¹ is —CO₂H;
or a pharmaceutically acceptable salt thereof.

5. The compound according to claim 1, wherein R² is: 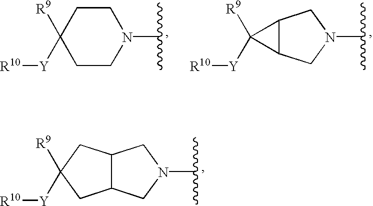
or a pharmaceutically acceptable salt thereof.

6. The compound according to claim 1, wherein R² is: 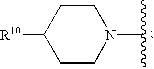
or a pharmaceutically acceptable salt thereof.

7. The compound according to claim 1 wherein R³ is phenyl, thienyl, pyrazolyl, thiazolyl, thiadiazolyl, furanyl, oxadiazolyl, pyrazinyl, pyrimidinyl, or pyridyl, any one of which is unsubstituted or substituted with 1-5 substituents where the substituents are independently selected from:
(a) halo,
(b) —CF₃,
(c) hydroxy,
(d) C₁₋₃ alkyl, and
(e) —O-C₁₋₃ alkyl;
or a pharmaceutically acceptable salt thereof.

8. The compound according to claim 1 wherein R³ is phenyl or thienyl, either of which is unsubstituted or substituted with 1-5 substituents where the substituents are independently selected from:
(a) halo,
(b) —CF₃,
(c) hydroxy, and
(d) C₁₋₃ alkyl;
or a pharmaceutically acceptable salt thereof.

9. The compound according to claim 1 wherein R³ is phenyl or thienyl, wherein the phenyl is optionally substituted with 1-5 substituents independently selected from fluoro and chloro;
or a pharmaceutically acceptable salt thereof.

10. The compound according to claim 1 wherein R³ is unsubstituted phenyl, 3-fluorophenyl, or 3-thienyl;
or a pharmaceutically acceptable salt thereof.

11. The compound according to claim 1 wherein R⁴ and R⁵ are both hydrogen;
or a pharmaceutically acceptable salt thereof.

12. The compound according to claim 1, wherein R^{6a} and R^{6b} are each independently C₁₋₆ alkyl or C₃₋₆ cycloalkyl, either of which is unsubstituted or substituted with 1-7 substituents independently selected from:
(a) halo,
(b) —CF₃,
(c) hydroxy, and
(d) —O-C₁₋₃ alkyl;
or R^{6a} and R^{6b} together with the carbon atom to which they are attached form:
(a) a 3- to 6-membered saturated carbocyclic ring,
(b) a 4- to 6-membered saturated heterocyclic ring containing one oxygen atom, or
(c) a 5- or 6-membered saturated carbocyclic ring to which is fused a C3-6 cycloalkyl;
wherein the ring system of (a), (b), or (c) is optionally substituted with from 1 to 3 substituents selected from halo, C₁₋₄ alkyl, C₁₋₄ haloalkyl, —O-C₁₋₄ alkyl, —O-C₁₋₄ haloalkyl, or hydroxy; or a pharmaceutically acceptable salt thereof.

13. The compound according to claim 1, wherein R^{6a} and R^{6b} are each C₁₋₃ alkyl;
or one of R^{6a} and R^{6b} is C₁₋₃ alkyl, and the other of R^{6a} and R^{6b} is C₃₋₆ cycloalkyl;
or R^{6a} and R^{6b} together with the carbon atom to which they are attached form cyclobutylidenyl, cyclopentylidenyl, cyclohexylidenyl, bicyclo[3.1.0]cyclohexylidenyl, tetrahydropyranylidenyl, or tetrahydrofuranylidenyl;
or a pharmaceutically acceptable salt thereof.

14. The compound according to claim 1, wherein R⁷ is hydrogen;
or a pharmaceutically acceptable salt thereof.

15. The compound according to claim 1, wherein R⁸ is hydrogen;
or a pharmaceutically acceptable salt thereof.

16. The compound according to claim 1, wherein R⁸ is methyl;
or a pharmaceutically acceptable salt thereof.

17. The compound according to claim 1 wherein R⁹ is hydrogen, fluoro, hydroxy or C₁₋₆ alkyl;
or a pharmaceutically acceptable salt thereof.

18. The compound according to claim 1 wherein R⁹ is hydrogen or fluoro;
or a pharmaceutically acceptable salt thereof.

19. The compound according to claim 1 wherein R⁹ is hydrogen;
or a pharmaceutically acceptable salt thereof.

20. The compound according to claim 1, wherein Y is
(1) a direct single bond;
(2) —C₁₋₆ alkyl-, which is optionally substituted with 1-7 substituents independently selected from:
(a) halo,
(b) hydroxy,
(c) —O-C₁₋₃ alkyl, and
(d) —CF₃;
(3) —(C₀₋₂ alkyl)—Z¹—(C₀₋₂ alkyl)-, wherein the alkyl is unsubstituted;
Z¹ is selected from —SO₂—, —N(R^{u})—, —SO—, —SO₂N(R^{u})—, —S—, and —O—; and R^{u} is C₁₋₄ alkyl, C₂₋₅ alkenyl, or C₁₋₃ alkyl-C₃₋₆ cycloalkyl; or
(4) —(C₀₋₂ alkyl)—Z²—(CO₂ alkyl)—, wherein the alkyl is optionally substituted with 1-4 substituents independently selected from:
(a) halo,
(b) hydroxy,
(c) —O-C₁₋₃ alkyl, and
(d) —CF₃;
and wherein
Z² is selected from —C(═O)NR^{v}—, —NR^{v}C(═O)—, —OC(═O)NR^{v}—, —NR^{v}C(═O)O—, and —NR^{w}C(═O)NR^{v}—;
R^{v} is hydrogen, C₁₋₆ alkyl, C₂₋₆ alkenyl, C2-6 alkynyl, benzyl, phenyl, or C₁₋₆ alkyl-C₃₋₆ cycloalkyl; wherein any of which except hydrogen is optionally substituted with from 1 to 3 substituents independently selected from halo, C₁₋₃ alkyl, —O-C₁₋₆ alkyl and —CF₃; and
R^{w} is —H or C₁₋₆ alkyl;
or a pharmaceutically acceptable salt thereof.

21. The compound according to claim 1, wherein Y is
(1) a direct single bond;
(2) —C₂₋₄ alkyl-, which is optionally substituted with 1-6 substituents independently selected from:
(a) halo,
(b) —O-C₁₋₃ alkyl, and
(c) —CF₃;
(3) selected from
—(C₀₋₂ alkyl)—SO₂—(C₀₋₂ alkyl)—,
—(C₀₋₂ alkyl)—SO₂N(R^{u})—(C₀₋₂ alkyl),
—(C₀₋₂ alkyl)—SO—(CO₂ alkyl)—,
—(C₀₋₂ alkyl)—S—(CO₂ alkyl)—,
—(C₀₋₂ alkyl)—O—(CO₂ alkyl)—, and
—(C₀₋₂ alkyl)—N(R^{u})—(CO₂ alkyl)—; and
where R^{u} is C₂₋₄ alkyl, C₂₋₃ alkenyl or C₁₋₂ alkyl-C₁₋₃ cycloalkyl;
(4) —(C₀₋₂ alkyl)—Z²—(CO₂ alkyl)—, wherein the alkyl is not substituted;
and where
Z² is selected from —C(═O)NR^{v}—, —NR^{v}C(═O)—, —OC(═O)NR^{v}—, —NR^{v}C(═O)O—, and —NR^{w}C(═O)NR^{v}—;
R^{v} is hydrogen, C₁₋₃ alkyl, C₂₋₃ alkenyl, or C₂₋₃ alkynyl; and
R^{w} is —H or C₁₋₄ alkyl;
or a pharmaceutically acceptable salt thereof.

22. The compound according to claim 1, wherein Y is
(1) a direct single bond;
(2) C₂₋₄ alkyl, which is optionally substituted with from 1 to 6 fluoros;
(3) selected from:
(a) —SO₂CH₂CH₂—,
(b) —SO₂—N(CH₂CH₃)—,
(c) —CH₂SO₂—N(CH₂CH₃)—,
(d) —SO—CH₂CH₂—,
(e) —SCH₂CH₂—,
(f) —CH₂—O—CH₂—,
(g) —N(CH₂CH₃)—,
(h) —N(CH₂CH₂CH₃)—,
(i) —N(allyl)—, and
(j) —N(CH₂-cyclopropyl)—; or
(4) selected from:
(a) —CH₂OC(═O)—N(C₁₋₄ alkyl)—,
(b) —CH₂—OC((═O)N(allyl)—,
(c) —CH₂NHC(═O)N(C₁₋₄ alkyl)—,
(d) —CH₂NHC(═O)N(allyl), and
(e) —CH₂CH₂NHC(═O)N(CH₂CH₃)—.
or a pharmaceutically acceptable salt thereof.

23. The compound according to claim 1, wherein Y is a direct single bond;
or a pharmaceutically acceptable salt thereof.

24. The compound according to claim 1 wherein R¹⁰ is phenyl, benzoimidazolyl, imidazolyl, pyridoimidazolyl, isoxazolyl, oxazolyl, pyrazolyl, pyridyl, thiazolyl, imidazothiophenyl, indazolyl, tetrahydropyridoimidazolyl, tetrahydroindazolyl, dihydrothiopyranopyrazolyl, dihydrodioxothiopyranopyrazolyl, dihydropyranopyrazolyl, tetrahydropyridopyrazolyl, benzopyrazolyl, pyridopyrazolyl, or triazolyl; any one of which is unsubstituted or substituted with 1-7 substituents where the substituents are independently selected from:
(a) halo,
(b) cyano,
(c) hydroxy,
(d) C₁₋₆ alkyl, which is unsubstituted or substituted with 1-5 of R^{e} where R^{e} is independently selected from halo, cyano, hydroxy, —O-C₁₋₆ alkyl, —C₃₋₅ cycloalkyl, —CO₂H, —CO₂(C16 alkyl), —CF₃, —SO₂R^{a}, —NR^{a}R^{b},
where R^{a} and R^{b} are independently selected from hydrogen, C₁₋₆ alkyl, C₅₋₆ cycloalkyl, benzyl or phenyl, which is unsubstituted or substituted with 1-3 substituents where the substituents are independently selected from halo, C₁₋₃ alkyl, —O-C₁₋₃ alkyl, C₁₋₃ fluoroalkyl, and —O-C₁₋₃ fluoroalkyl,
phenyl, naphthyl, biphenyl, and heterocycle,
wherein the phenyl, naphthyl, biphenyl or heterocycle is unsubstituted or substituted with 1-7 of R^{f} where R^{f} is independently selected from halo, cyano, hydroxy, C₁₋₄ alkyl, —O-C₁₋₄ alkyl, —O-C₃₋₅ cycloalkyl, —CO₂H, —C₀₋₂(C₁₋₆ alkyl), —CF₃, —OCF₃, —SO₂R^{a}, —N(R^{a})SO₂R^{b} and —NR^{a}R^{b},
(e) —O-C₁₋₆ alkyl, which is unsubstituted or substituted with 1-5 of R^{e},
(f) —NO₂,
(g) phenyl,
(h) —CO₂R^{a},
(i) tetrazolyl,
(j) —NR^{a}R^{b},
(k) —NR^{a}—COR^{b},
(l) —NR^{a}—CO₂R^{b},
(m) —CO—NR^{a}R^{b},
(n) —OCO—NR^{a}R^{b},
(o) —NR^{a}CO—NR^{a}R^{b},
(p) —S(O)ₘ—R^{a}, wherein m is an integer selected from 0, 1 and 2,
(q) —S(O)₂NR^{a}R^{b},
(r) —NR^{a}S(O)₂—R^{b},
(s) —NR^{a}S(O)₂—NR^{a}R^{b};
(t) —C₃₋₆ cycloalkyl, and
(u) —O-C₃₋₆ cycloalkyl;
and with the proviso that
(A) when R¹⁰ is a heterocycle selected from pyrazolyl and imidazolyl, then the heterocycle is unsubstituted or substituted with 1 or 2 substituents independently selected from any of substituents (a) to (u) as defined above; and
(B) when R¹⁰ is a heterocycle selected from: 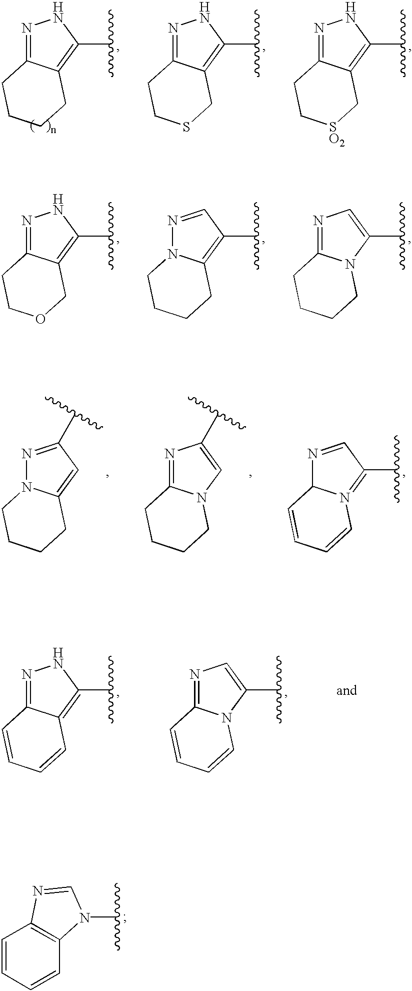
then the heterocycle is unsubstituted in the pyrazolyl or imidazolyl ring, and is either unsubstituted in the other ring or is substituted with 1 or 2 substituents independently selected from any of substituents (a) to (u) as defined above;
or a pharmaceutically acceptable salt thereof.

25. The compound according to claim 1, wherein R¹⁰ is phenyl, benzimidazolyl, imidazolyl, pyridoimidazolyl, isoxazolyl, oxazolyl, pyrazolyl, pyridyl, thiazolyl, imidazothiophenyl, indazolyl, tetrahydropyridoimidazolyl, tetrahydroindazolyl, dihydrothiopyranopyrazolyl, dihydrodioxothiopyranopyrazolyl, dihydropyranopyrazolyl, tetrahydropyridopyrazolyl, or triazolyl; any one of which is unsubstituted or substituted with 1-5 substituents where the substituents are independently selected from:
(a) halo,
(b) cyano,
(c) —NO₂,
(d) —CF₃,
(e) —CHF₂,
(f) —CH₂F,
(g) —CH₂OH,
(h) —CH₂OCH₃,
(i) —(CH₂)₁₋₂SO₂-(C₁₋₂ alkyl)
(j) phenyl,
(k) C₁₋₆ alkyl, which is unsubstituted or substituted with phenyl, which is unsubstituted or substituted with 1-4 of R^{f} where R^{f} is independently selected from halo, cyano, hydroxy, —O-C₁₋₆ alkyl, —O-C₃₋₅ cycloalkyl, —CO₂H, —CO₂(C₁₋₆ alkyl), —CF₃, —OCF₃, —SO₂-(C₁₋₃ alkyl), and —N(R^{a})SO₂—(C₁₋₃ alkyl),
(l) —O-C₁₋₆ alkyl,
(m) —C₃₋₅ cycloalkyl,
(n) —CH₂—(C₃₋₅ cycloalkyl), and
(o) —O-C3-5 cycloalkyl;
and with the proviso that
(A) when R¹⁰ is a heterocycle selected from pyrazolyl and imidazolyl, then the heterocycle is unsubstituted or substituted with 1 or 2 substituents independently selected from any of substituents (a) to (o) as defined above; and
(B) when R¹⁰ is a heterocycle selected from: 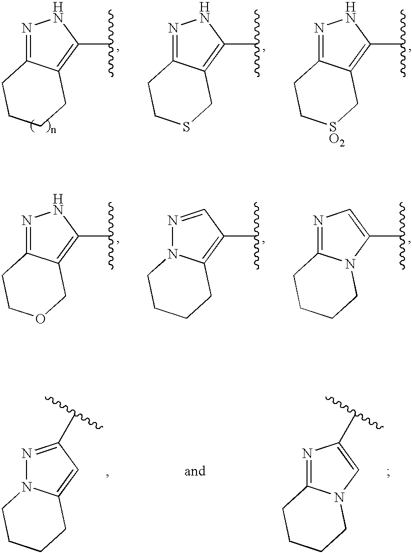
then the heterocycle is unsubstituted in the pyrazolyl or imidazolyl ring, and is either unsubstituted in the other ring or is substituted with 1 or 2 substituents independently selected from any of substituents (a) to (o) as defined above;
or a pharmaceutically acceptable salt thereof.

26. The compound according to claim 1, wherein R¹⁰ is:
(i) pyrazolyl or imidazolyl, either of which is unsubstituted or substituted with 1 or 2 substituents independently selected from:
(a) fluoro,
(b) chloro,
(c) C₁₋₆ alkyl,
(d) —CH₂-phenyl, wherein the phenyl is unsubstituted or substituted with 1 or 2 substituents independently selected from chloro, fluoro, —CN, —C₁₋₃ alkyl, —O-C₁₋₃ alkyl, —O-cyclopropyl, —O-cyclobutyl, —CF₃, —OCF₃, —SO₂—(C₁₋₃ alkyl), and —N(H)SO₂—(C₁₋₃ alkyl),
(e) —CH₂CH₂-phenyl, and
(f) phenyl; or 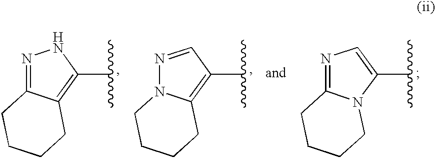
each of which is unsubstituted in the pyrazolyl or imidazolyl ring, and is either unsubstituted in the other ring or is substituted with 1 or 2 substituents independently selected from:
(a) halo,
(b) C₁₋₄ alkyl,
(c) C₁₋₄ haloalkyl,
(d) —OH,
(e) —O-C₁₋₄ alkyl,
(f) —O-C₁₋₄ haloalkyl, and
(g) —CN;
or a pharmaceutically acceptable salt thereof.

27. The compound according to claim 1 which is of the stereochemical configuration: 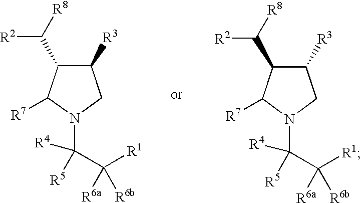
or a pharmaceutically acceptable salt thereof.

28. The compound of claim 1, which is a compound of formula (II): 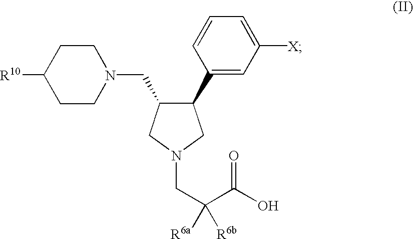
wherein
R^{6a} and R^{6b} are each C₁₋₄ alkyl;
or one of R^{6a} and R^{6b} is C₁₋₄ alkyl, and the other of R^{6a} and R^{6b} is C₃₋₆ cycloalkyl;
or R^{6a} and R^{6b} together with the carbon atom to which they are attached form: 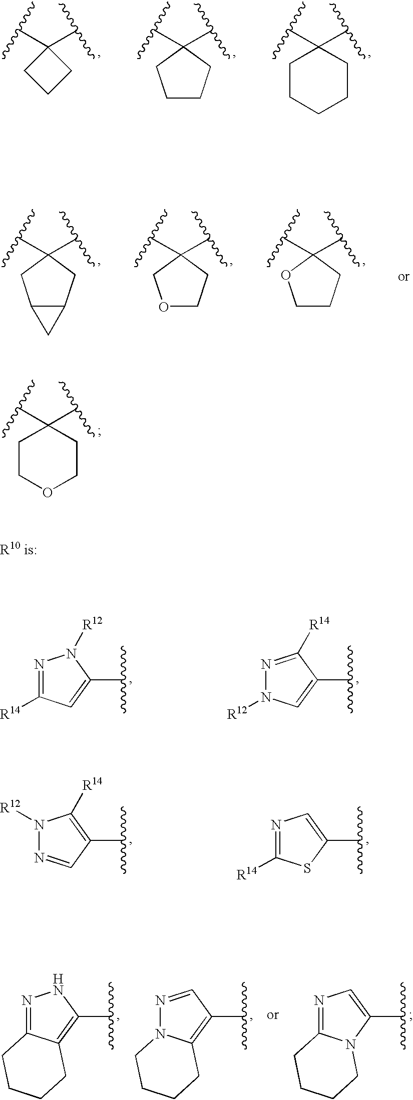
R¹² is hydrogen, C₁₋₄ alkyl, C₁₋₄ fluoroalkyl, —(C₁₋₄ alkyl)—SO₂—(C₁₋₄ alkyl), or —CH₂-phenyl wherein the phenyl is optionally substituted with 1 or 2 substituents independently selected from chloro, fluoro, —CN, —C₁₋₄ alkyl, —O-C₁₋₄ alkyl, —O—cyclopropyl, —O-cyclobutyl, —CF₃, —OCF₃, —SO₂—(C₁₋₄ alkyl), and —NHSO₂—(C₁₋₄ alkyl);
R¹⁴ is hydrogen, —C₁₋₄ alkyl, C₁₋₄ fluoroalkyl, —O-C₁₋₄ alkyl, —O-C₁₋₄ fluoroalkyl, cyclopropyl, cyclobutyl, or —CH₂-phenyl wherein the phenyl is optionally substituted with 1 or 2 substituents independently selected from chloro, fluoro, —CN, —C₁₋₄ alkyl, —O-C₁₋₄ alkyl, —O-cyclopropyl, —O-cyclobutyl, —CF₃, —OCF₃, and —SO₂—(C₁₋₄ alkyl); and
X is hydrogen or fluoro;
or a pharmaceutically acceptable salt thereof.

29. The compound of claim 1, which is a compound of formula (II): 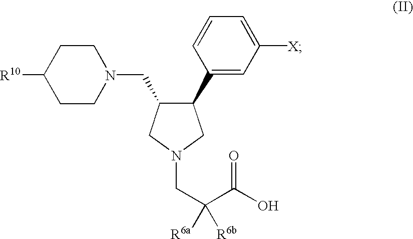
wherein
R^{6a} and R^{6b} are each C₁₋₃ alkyl;
or one of R^{6a} and R^{6b} is C₁₋₃ alkyl, and the other of R^{6a} and R^{6b} is C₃₋₆ cycloalkyl;
or R^{6a} and R^{6b} together with the carbon atom to which they are attached form: 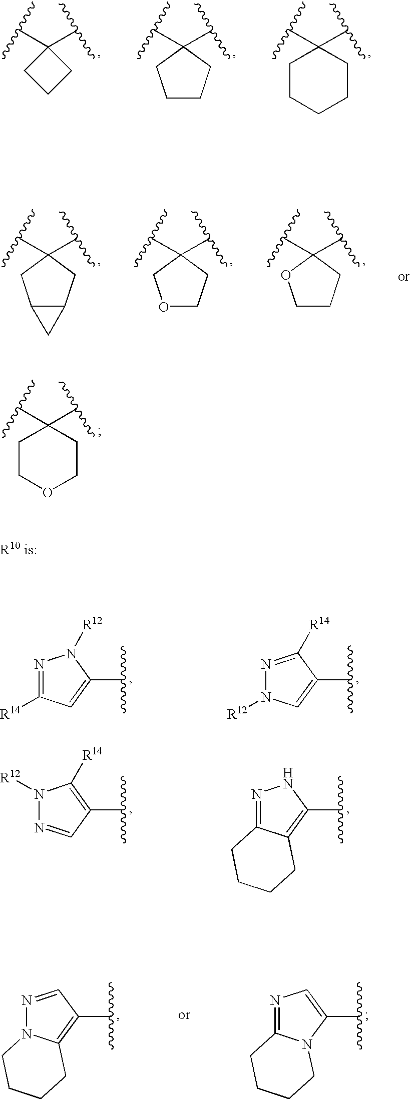
R¹² is hydrogen, C₁₋₃ alkyl, C₁₋₃ fluoroalkyl, or —CH₂-phenyl wherein the phenyl is optionally substituted with 1 or 2 substituents independently selected from chloro, fluoro, —CN, —C₁₋₃ alkyl, —O-C₁₋₃ alkyl, —O-cyclopropyl, —O-cyclobutyl, —CF₃, —OCF₃, —SO₂—(C₁₋₃ alkyl), and —NHSO₂—(C₁₋₃ alkyl); R¹⁴ is hydrogen, —C₁₋₃ alkyl, C₁₋₃ fluoroalkyl, —O-C₁₋₃ alkyl, —O-C₁₋₃ fluoroalkyl, cyclopropyl, cyclobutyl, or —CH₂-phenyl wherein the phenyl is optionally substituted with 1 or 2 substituents independently selected from chloro, fluoro, —CN, —C₁₋₃ alkyl, —O-C₁₋₃ alkyl, —O-cyclopropyl, —O-cyclobutyl, —CF₃, —OCF₃, and —SO₂—(C₁₋₃ alkyl); and
X is hydrogen or fluoro;
or a pharmaceutically acceptable salt thereof.

30. The compound according to claim 29, wherein R¹⁰ is: 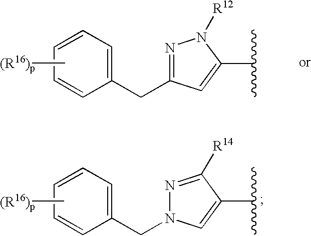
R¹² is C₁₋₃ alkyl;
R¹⁴ is —C₁₋₃ alkyl;
each R¹⁶ is independently chloro, fluoro, —CN, —C₁₋₃ alkyl, —O-C₁₋₃ alkyl, —O-cyclopropyl, —O-cyclobutyl, —CF₃, —OCF₃, or —SO₂—(C₁₋₃ alkyl); and
p is an integer from zero to 3;
or a pharmaceutically acceptable salt thereof.

31. The compound according to claim 30, wherein R¹² and R¹⁴ are both ethyl;
or a pharmaceutically acceptable salt thereof.

32. The compound according to claim 1, which is 1-{[(3S,4S)-3-[(4-{3-ethyl-1-[4-(methylsulfonyl)benzyl]-1H-pyrazol-4-yl}piperidin-1-yl)methyl]-4-(3-fluorophenyl)pyrrolidin-1-yl]methyl}cyclohexanecarboxylic acid; or a pharmaceutically acceptable salt thereof.

33. A pharmaceutical composition which comprises an inert carrier and an effective amount of a compound according to claim 1 or a pharmaceutically acceptable salt thereof.

34. A method for modulating CCR5 chemokine receptor activity in a subject which comprises administering to the subject an effective amount of the compound according to claim 1 or a pharmaceutically acceptable salt thereof.

35. A method for preventing infection by HIV, treating infection by HIV, delaying of the onset of AIDS, or treating AIDS in a patient, which comprises administering to the patient of an effective amount of the compound according to claim 1 or a pharmaceutically acceptable salt thereof.
